(19) United States
(12) Patent Application Publication
(10) Pub. No.: US 2024/0307555 A1
(43) Pub. Date: Sep. 19, 2024

(54) RETARGETED RETROVIRAL VECTORS RESISTANT TO VACCINE-INDUCED NEUTRALIZATION AND COMPOSITIONS OR METHODS OF USE THEREOF

(71) Applicants: The Broad Institute, Inc., Cambridge, MA (US); The General Hospital Corporation, Boston, MA (US)

(72) Inventors: Kepler MEARS, Cambridge, MA (US); Robert MANGUSO, Boston, MA (US); Kathleen YATES, Cambridge, MA (US); Kyrellos IBRAHIM, Cambridge, MA (US); Peter ALLEN, Cambridge, MA (US)

(73) Assignees: The Broad Institute, Inc., Cambridge, MA (US); The General Hospital Corporation, Boston, MA (US)

(21) Appl. No.: 18/667,761

(22) Filed: May 17, 2024

Related U.S. Application Data
(63) Continuation of application No. PCT/US2022/080156, filed on Nov. 18, 2022.
(60) Provisional application No. 63/280,919, filed on Nov. 18, 2021.
(60) Provisional application No. 63/280,926, filed on Nov. 18, 2021.
(60) Provisional application No. 63/359,027, filed on Jul. 7, 2022.

(51) Int. Cl. A61K 48/00; A61K 47/68; A61K 47/69; A61P 35/00; C07K 14/005; C12N 15/86

(52) U.S. Cl. CPC A61K 48/0033; A61K 47/6851; A61K 47/6901; A61P 35/00; C07K 14/005; C12N 15/86; C07K 2319/33; C12N 2740/15043; C12N 2740/15051; C12N 2760/18422

(57) ABSTRACT

The invention features pseudotyped viral particles (e.g., lentiviral or gammaretroviral particles) and compositions and methods of use thereof, where the viral particles comprise a VHH domain.

### CROSS-REFERENCE TO RELATED APPLICATIONS

This application is a continuation under 35 U.S.C. § 111(a) of PCT International Patent Application No. PCT/US2022/080156, filed Nov. 18, 2022, designating the United States and published in English, which claims priority to and the benefit of U.S. Provisional Applications No. 63/359,027, filed Jul. 7, 2022, 63/280,919, filed Nov. 18, 2021, and 63/280,926, filed Nov. 18, 2021, the entire contents of which are incorporated herein by reference.

### SEQUENCE LISTING

The present application contains a Sequence Listing which has been submitted electronically in XML format following conversion from the originally filed TXT format.

The content of the electronic XML Sequence Listing, (Date of creation: Jan. 11, 2023; Size: 196,608 bytes; Name: 167741-038002_PCT-SL.xml), is herein incorporated by reference in its entirety.

### BACKGROUND OF THE INVENTION

Efforts for altering somatic cells in a subject have been hindered by inefficient vector delivery and an inability to target the desired cells specifically. One approach that overcomes this obstacle is the genetic manipulation of cells ex vivo, as is done for T cells in adoptive cell therapy or chimeric antigen receptor (CAR) therapy. T cells are activated using anti-CD3/anti-CD28 and/or cytokine stimulation, followed by lentiviral transduction and transfer into a new animal. However, this process of in vitro expansion changes the T cell state and affects differentiation; moreover, this approach is not easily extendable to other cell types which cannot be expanded ex vivo. This approach also suffers from inefficient engraftment of gene modified cells after transplantation in vivo.

In vivo approaches for altering cells would benefit from effective methods for retargeting vectors to be specific for particular cell types in a subject. However, such methods remain inefficient and/or poorly developed.

Thus, there is a need for improved methods for in vivo delivery of vectors to a target cell.

### SUMMARY OF THE INVENTION

As described below, the present invention features pseudotyped viral particles (e.g., lentiviral or gammaretroviral particles) and compositions and methods of use thereof, where the viral particles comprise a VHH domain.

In one aspect, the invention features a pseudotyped viral particle. The viral particle contains (a) an envelope containing a viral envelope glycoprotein domain or fragment thereof fused to a VHH domain or fragment thereof. The VHH domain or fragment thereof specifically binds an antigen present on a target cell. The viral envelope glycoprotein contains an alteration referenced to a measles virus glycoprotein at an amino acid targeted by a measles virus neutralizing antibody. The viral particle also contains (b) a heterologous polynucleotide.

In another aspect, the invention features a method for delivering a heterologous polynucleotide to a target cell. The method involves contacting a target cell with a pseudotyped viral particle, thereby delivering a heterologous polynucleotide to the target cell. The pseudogyped viral particle contains (a) an envelope containing a viral envelope glycoprotein domain or fragment thereof fused to a VHH domain or fragment thereof. The VHH domain or fragment thereof specifically binds an antigen present on the target cell. The viral envelope glycoprotein contains an alteration referenced to a measles virus glycoprotein at an amino acid targeted by a measles virus neutralizing antibody. The pseudotyped viral particle also contains (b) the heterologous polynucleotide.

In another aspect, the invention features a method for delivering a heterologous polynucleotide to a target cell of a subject. The method involves administering to the subject a pseudotyped viral particle, thereby delivering the heterologous polynucleotide to the subject. The pseudotyped viral particle contains (a) an envelope containing a viral envelope glycoprotein domain or fragment thereof fused to a VHH domain or fragment thereof. The VHH domain or fragment thereof specifically binds an antigen present on the target cell. The viral envelope glycoprotein contains an alteration referenced to a measles virus glycoprotein at an amino acid targeted by a measles virus neutralizing antibody. The pseudotyped viral particle also contains (b) a heterologous polynucleotide,

In another aspect, the invention features a method of treating a subject having a cancer. The method involves administering to the subject a composition containing a pseudotyped viral particle, thereby delivering the heterologous polynucleotide to the target cell in the subject and treating the subject. The pseudotyped viral particle contains (a) an envelope containing a viral envelope glycoprotein domain or fragment thereof fused to a VHH domain or fragment thereof. The VHH domain or fragment thereof specifically binds a tumor antigen present on a target cancer cell. The viral envelope glycoprotein contains an alteration referenced to a measles virus glycoprotein at an amino acid targeted by a measles virus neutralizing antibody. The pseudotyped viral particle also contains (b) a heterologous polynucleotide.

In another aspect, the invention features a method for generating a pseudotyped viral particle for delivering a heterologous polynucleotide to a target cell. The method involves (a) displaying on the cell membrane of a eukaryotic cell a viral envelope glycoprotein domain or fragment thereof fused to a VHH domain or fragment thereof. The VHH domain or fragment thereof specifically binds an antigen present on the target cell. The viral envelope glycoprotein contains an alteration referenced to a measles virus glycoprotein at an amino acid targeted by a measles virus neutralizing antibody. The method also involves (b) transfecting the eukaryotic cell with a viral transfer vector and one or more additional vectors encoding one or more viral polypeptides, thereby generating the pseudotyped viral particle for delivering a heterologous polynucleotide to the target cell.

In another aspect, the invention features a eukaryotic cell for generating a pseudotyped viral particle. The eukaryotic cell contains (a) a cell membrane containing a viral envelope glycoprotein domain or fragment thereof fused to a VHH domain or fragment thereof. The VHH domain or fragment thereof specifically binds an antigen present on a target cell. The viral envelope glycoprotein contains an alteration referenced to a measles virus glycoprotein at an amino acid targeted by a measles virus neutralizing antibody. The eukaryotic cell also contains (b) a viral transfer vector, and (c) one or more additional vectors encoding one or more viral polypeptides.

In another aspect, the invention features a mammalian expression vector containing a polynucleotide encoding a polypeptide containing a viral envelope glycoprotein domain or fragment thereof fused to a VHH domain or fragment thereof. The VHH domain or fragment thereof specifically binds an antigen present on a target cell. The viral envelope glycoprotein contains an alteration referenced to a measles virus glycoprotein at an amino acid targeted by a measles virus neutralizing antibody.

In another aspect, the invention features a pharmaceutical composition containing the pseudotyped viral particle of any of the above aspects, and a pharmaceutically acceptable excipient.

In another aspect, the invention features a kit for use in the method of any of the above aspects. The kit contains the pseudotyped viral particle of any of the above aspects, the mammalian expression vector of any of the above aspects, or the pharmaceutical composition of any of the above aspects. The pseudotyped viral particle contains a heterologous polynucleotide containing a polypeptide-encoding sequence under the control of a promoter. The kit also contains instructions for the use of the kit in the method of any of the above aspects.

In another aspect, the invention features a fusion protein suitable for pseudotyping a viral particle. The fusion protein contains a viral envelope glycoprotein domain fused to a VHH domain. The VHH domain or fragment thereof specifically binds an antigen present on a target cell. The fusion protein contains a sequence with at least 85% sequence identity to a sequence selected from one or more of the following:

DMV-H-MHCII (N11)
MSSPRDKVDAFYKDIPRPRNNRVLLDNERVIIERPLILVGVLAVMFLSLVGLLAIAGVRLQKAT
TNSIEVNRKLSTNLETTVSIEHHVKDVLTPLFKIIGDEVGLRMPQKLTEIMQFISNKIKFLNPD
REYDFNDLHWCVNPPDQVKIDYAQYCNHIAAEELIVTKFKELMNHSLDMSKGRIFPPKNCSGSV
ITRGQTIKPGLTLVNIYTTRNFEVSFMVTVISGGMYGKTYFLKPPEPDDPFEFQAFRIFEVGLV
RDVGSREPVLQMTNFMVIDEDEGLNFCLLSVGELRLAAVCVRGRPVVTKDIGGYKDEPFKVVTL
GIIGGGLSNQKTEIYPTIDSSIEKLYITSHRGIIRNSKARWSVPAIRSDDKDKMEKCTQALCKS
RPPPSCNSSDWEPLTSNRIPAYAYIALEIKEDSGLELDITSNYGPLIIHGAGMDIYEGPSSNQD
WLAIPPLSQSVLGVINKVDFTAGFDIKPHTLTTAVDYESGKCYVPVELSGAKDQDLKLESNLVV
LPTKDFGYVTATYDTSRSEHAIVYYVYDTARSSSYFFPFRIKARGEPIYLRIECFPWSRQLWCH
HYCMINSTVSNEIVVVDNLVSINMSCSRGGGGSGGGGSGGGGSAAAQVQLVQSGGGLVQPGGSL
GLSCAASGNIGSRDNMGWYRQAPGKQREWVATISGYGIATYRDSVKGRFTVAKDTAKNIVSLQM
NYLTTEDTAVYYCYAYAVDSRNIFWSQGTQVTVS;
DMV-H-CD7 (Humanized VHH10)
MSSPRDKVDAFYKDIPRPRNNRVLLDNERVIIERPLILVGVLAVMFLSLVGLLAIAGVRLQKAT
TNSIEVNRKLSTNLETTVSIEHHVKDVLTPLFKIIGDEVGLRMPQKLTEIMQFISNKIKFLNPD
REYDFNDLHWCVNPPDQVKIDYAQYCNHIAAEELIVTKFKELMNHSLDMSKGRIFPPKNCSGSV
ITRGQTIKPGLTLVNIYTTRNFEVSFMVTVISGGMYGKTYFLKPPEPDDPFEFQAFRIFEVGLV
RDVGSREPVLQMTNFMVIDEDEGLNFCLLSVGELRLAAVCVRGRPVVTKDIGGYKDEPFKVVTL
GIIGGGLSNQKTEIYPTIDSSIEKLYITSHRGIIRNSKARWSVPAIRSDDKDKMEKCTQALCKS
RPPPSCNSSDWEPLTSNRIPAYAYIALEIKEDSGLELDITSNYGPLIIHGAGMDIYEGPSSNQD
WLAIPPLSQSVLGVINKVDFTAGFDIKPHTLTTAVDYESGKCYVPVELSGAKDQDLKLESNLVV
LPTKDFGYVTATYDTSRSEHAIVYYVYDTARSSSYFFPFRIKARGEPIYLRIECFPWSRQLWCH
HYCMINSTVSNEIVVVDNLVSINMSCSRGGGGSGGGGSGGGGSAAADVQLQESGGGSVQAGGSL
RLSCAASGYTHSSYCMAWFRQAPGREREGVASIDSDGTTSYADSVKGRFTISQDNAKNTLYLQM
NSLKPEDTAMYYCAARFGPMGCVDLSTLSFGHWGQGTQVTVSITGGGSGGGSYPYDVPDYA;
DMV-H-CD45 (32)
MSSPRDKVDAFYKDIPRPRNNRVLLDNERVIIERPLILVGVLAVMFLSLVGLLAIAGVRLQKAT
TNSIEVNRKLSTNLETTVSIEHHVKDVLTPLFKIIGDEVGLRMPQKLTEIMQFISNKIKFLNPD
REYDFNDLHWCVNPPDQVKIDYAQYCNHIAAEELIVTKFKELMNHSLDMSKGRIFPPKNCSGSV
ITRGQTIKPGLTLVNIYTTRNFEVSFMVTVISGGMYGKTYFLKPPEPDDPFEFQAFRIFEVGLV
RDVGSREPVLQMTNFMVIDEDEGLNFCLLSVGELRLAAVCVRGRPVVTKDIGGYKDEPFKVVTL
GIIGGGLSNQKTEIYPTIDSSIEKLYITSHRGIIRNSKARWSVPAIRSDDKDKMEKCTQALCKS
RPPPSCNSSDWEPLTSNRIPAYAYIALEIKEDSGLELDITSNYGPLIIHGAGMDIYEGPSSNQD
WLAIPPLSQSVLGVINKVDFTAGFDIKPHTLTTAVDYESGKCYVPVELSGAKDQDLKLESNLVV
LPTKDFGYVTATYDTSRSEHAIVYYVYDTARSSSYFFPFRIKARGEPIYLRIECFPWSRQLWCH
HYCMINSTVSNEIVVVDNLVSINMSCSRGGGGSGGGGSGGGGSAAAQVQLVQSGGGLVQPGGSL
RLSCAASGRAFNSAAMGWYRQAPGSQRELVASISAGTASYADAVKGRFTISRDYAKNIIYLQMN
SLKPDDTAVYFCNYRTTYTSGYSEDYWGQGTQVTVSGGGSGGGSYPYDVPDYA;
CDV-H-MHCII (N11)
MLPYQDKVGAFYKDNARANSTKLSLVTEGHGGRRPPYLLFVLLILLVGILALLAITGVRFHQVS
TSNMEFSRLLKEDMEKSEAVHHQVIDVLTPLFKIIGDEIGLRLPQKLNEIKQFILQKTNFFNPN
REFDFRDLHWCINPPSTVKVNFTNYCESIGIRKAIASAANPILLSALSGGRGDIFPPHRCSGAT
TSVGKVFPLSVSLSMSLISRTSEVINMLTAISDGVYGKTYLLVPDDIEREFDTREIRVFEIGFI
KRWLNDMPLLQTTNYMVLPKNSKAKVCTIAVGELTLASLCVEESTVLLYHDSSGSQDGILVVTL
GIFWATPMDHIEEVIPVAHPSMKKIHITNHRGFIKDSIATWMVPALASEKQEEQKGCLESACQR
KTYPMCNQASWEPFGGRQLPSYGRLTLPLDASVDLQLNISFTYGPVILNGDGMDYYESPLLNSG
WLTIPPKDGTISGLINKAGRGDQFTVLPHVLTFAPRESSGNCYLPIQTSQIRDRDVLIESNIVV
LPTQSIRYVIATYDISRSDHAIVYYVYDPIRTISYTHPFRLTTKGRPDFLRIECFVWDDNLWCH
QFYRFEADIANSTTSVENLVRIRFSCNRGGGGSGGGGSGGGGSAAAQVQLVQSGGGLVQPGGSL
GLSCAASGNIGSRDNMGWYRQAPGKQREWVATISGYGIATYRDSVKGRFTVAKDTAKNIVSLQM
NYLTTEDTAVYYCYAYAVDSRNIFWSQGTQVTVSGGGSGGGSYPYDVPDYA;
CDV-H-CD7 (Humanized VHH10)
MLPYQDKVGAFYKDNARANSTKLSLVTEGHGGRRPPYLLFVLLILLVGILALLAITGVRFHQVS
TSNMEFSRLLKEDMEKSEAVHHQVIDVLTPLFKIIGDEIGLRLPQKLNEIKQFILQKTNFFNPN
REFDFRDLHWCINPPSTVKVNFTNYCESIGIRKAIASAANPILLSALSGGRGDIFPPHRCSGAT
TSVGKVFPLSVSLSMSLISRTSEVINMLTAISDGVYGKTYLLVPDDIEREFDTREIRVFEIGFI
KRWLNDMPLLQTTNYMVLPKNSKAKVCTIAVGELTLASLCVEESTVLLYHDSSGSQDGILVVTL
GIFWATPMDHIEEVIPVAHPSMKKIHITNHRGFIKDSIATWMVPALASEKQEEQKGCLESACQR
KTYPMCNQASWEPFGGRQLPSYGRLTLPLDASVDLQLNISFTYGPVILNGDGMDYYESPLLNSG
WLTIPPKDGTISGLINKAGRGDQFTVLPHVLTFAPRESSGNCYLPIQTSQIRDRDVLIESNIVV
LPTQSIRYVIATYDISRSDHAIVYYVYDPIRTISYTHPFRLTTKGRPDFLRIECFVWDDNLWCH
QFYRFEADIANSTTSVENLVRIRFSCNRGGGGSGGGGSGGGGSAAADVQLQESGGGSVQAGGSL
RLSCAASGYTHSSYCMAWFRQAPGREREGVASIDSDGTTSYADSVKGRFTISQDNAKNTLYLQM
NSLKPEDTAMYYCAARFGPMGCVDLSTLSFGHWGQGTQVTVSITGGGSGGGSYPYDVPDYA;
CDV-H-CD45 VHH (32)
MLPYQDKVGAFYKDNARANSTKLSLVTEGHGGRRPPYLLFVLLILLVGILALLAITGVRFHQVS
TSNMEFSRLLKEDMEKSEAVHHQVIDVLTPLFKIIGDEIGLRLPQKLNEIKQFILQKTNFFNPN
REFDFRDLHWCINPPSTVKVNFTNYCESIGIRKAIASAANPILLSALSGGRGDIFPPHRCSGAT
TSVGKVFPLSVSLSMSLISRTSEVINMLTAISDGVYGKTYLLVPDDIEREFDTREIRVFEIGFI
KRWLNDMPLLQTTNYMVLPKNSKAKVCTIAVGELTLASLCVEESTVLLYHDSSGSQDGILVVTL
GIFWATPMDHIEEVIPVAHPSMKKIHITNHRGFIKDSIATWMVPALASEKQEEQKGCLESACQR
KTYPMCNQASWEPFGGRQLPSYGRLTLPLDASVDLQLNISFTYGPVILNGDGMDYYESPLLNSG
WLTIPPKDGTISGLINKAGRGDQFTVLPHVLTFAPRESSGNCYLPIQTSQIRDRDVLIESNIVV
LPTQSIRYVIATYDISRSDHAIVYYVYDPIRTISYTHPFRLTTKGRPDFLRIECFVWDDNLWCH
QFYRFEADIANSTTSVENLVRIRFSCNRGGGGSGGGGSGGGGSAAAQVQLVQSGGGLVQPGGSL
RLSCAASGRAFNSAAMGWYRQAPGSQRELVASISAGTASYADAVKGRFTISRDYAKNIIYLQMN
SLKPDDTAVYFCNYRTTYTSGYSEDYWGQGTQVTVSAAAYPYDVPDYA;
MeV-Hc18-CDV-MHCII (N11)
MGSRIVINREHLMIDRPYVLLAVLFVMFLSLIGLLAIAGIRLHRAAIYTAEIHKSLSTNLDVTN
SIEHQVKDVLTPLFKIIGDEVGLRTPQRFTDLVKFISDKIKFLNPDREYDERDLTWCINPPERI
KLDYDQYCADVAARKAIASAANPILLSALSGGRGDIFPPHRCSGATTSVGKVFPLSVSLSMSLI
SRTSEVINMLTAISDGVYGKTYLLVPDDIEREFDTREIRVFEIGFIKRWLNDMPLLQTTNYMVL
PKNSKAKVCTIAVGELTLASLCVEESTVLLYHDSSGSQDGILVVTLGIFWATPMDHIEEVIPVA
HPSMKKIHITNHRGFIKDSIATWMVPALASEKQEEQKGCLESACQRKTYPMCNQASWEPFGGRQ
LPSYGRLTLPLDASVDLQLNISFTYGPVILNGDGMDYYESPLLNSGWLTIPPKDGTISGLINKA
GRGDQFTVLPHVLTFAPRESSGNCYLPIQTSQIRDRDVLIESNIVVLPTQSIRYVIATYDISRS
DHAIVYYVYDPIRTISYTHPFRLTTKGRPDFLRIECFVWDDNLWCHQFYRFEADIANSTTSVEN
LVRIRFSCNRGGGGSGGGGSGGGGSAAAQVQLVQSGGGLVQPGGSLGLSCAASGNIGSRDNMGW
YRQAPGKQREWVATISGYGIATYRDSVKGRFTVAKDTAKNIVSLQMNYLTTEDTAVYYCYAYAV
DSRNIFWSQGTQVTVSGGGSGGGSYPYDVPDYA;
MeV-Hc18-DMV-MHCII (N11)
MGSRIVINREHLMIDRPYVLLAVLFVMFLSLIGLLAIAGIRLHRAAIYTAEIHKSLSTNLDVTN
SIEHQVKDVLTPLFKIIGDEVGLRTPQRFTDLVKFISDKIKFLNPDREYDFRDLTWCINPPERI
KLDYDQYCADVAAEELIVTKFKELMNHSLDMSKGRIFPPKNCSGSVITRGQTIKPGLTLVNIYT
TRNFEVSFMVTVISGGMYGKTYFLKPPEPDDPFEFQAFRIFEVGLVRDVGSREPVLQMTNFMVI
DEDEGLNFCLLSVGELRLAAVCVRGRPVVTKDIGGYKDEPFKVVTLGIIGGGLSNQKTEIYPTI
DSSIEKLYITSHRGIIRNSKARWSVPAIRSDDKDKMEKCTQALCKSRPPPSCNSSDWEPLTSNR
IPAYAYIALEIKEDSGLELDITSNYGPLIIHGAGMDIYEGPSSNQDWLAIPPLSQSVLGVINKV
DFTAGFDIKPHTLTTAVDYESGKCYVPVELSGAKDQDLKLESNLVVLPTKDFGYVTATYDTSRS
EHAIVYYVYDTARSSSYFFPFRIKARGEPIYLRIECFPWSRQLWCHHYCMINSTVSNEIVVVDN
LVSINMSCSRGGGGSGGGGSGGGGSAAAQVQLVQSGGGLVQPGGSLGLSCAASGNIGSRDNMGW
YRQAPGKQREWVATISGYGIATYRDSVKGRFTVAKDTAKNIVSLQMNYLTTEDTAVYYCYAYAV
DSRNIFWSQGTQVTVSGGGSGGGSYPYDVPDYA;
FMV-H-CD45 (32) polypeptide
MESNNIKYYKDSSRYFGKILDEHKTINSQLYSLSIKVITIIAIIVSLIATIITIINATSGRTTL
NSNTDILLSQRDEIHNIQEMIFDRIYPLINAMSTELGLHIPTLLDELTKAIDQKIKIMHPPVDT
VTSDLNWCIKPPNGIIIDPKSYCESMELSKTYELLLDQLDVSRKKSLIINRKNINQCQLVDNSK
IIFATVNIQSTPRFLNFGHTVSNQRITFGQGTYSSTYVITIQEDGVTDVQYRVFEIGYISDQFG
VFPSLIVSRVLPIRMLLGMESCTLTSDRLGGYFLCMNTLTRSIYDYVSIRDLKSLYITIPHYGK
VNYTYFNFGKIRSPHEIDKIWLTSDRGQIISGYFAAFVTITIRNYNNYPYKCLNNPCFDNSENY
CRGWYKNITGTDDVPILAYLLVEMYDEEGPLITLVAIPPYNYTAPSHNSLYYDDKINKLIMTTS
HIGYIQINEVHEVIVGDNLKAILLNRLSDEHPNLTACRLNQGIKEQYKSDGTIISNSALIDIQE
RMYITVKAIPPAGNYNFTVELHSRSNTSYVSLPKQFNAKYDKLHLECFSWDKSWWCALIPQFSL
SWNESLSVDTAIFNLISCKGGGGSGGGGSGGGGSAAAQVQLVQSGGGLVQPGGSLRLSCAASGR
AFNSAAMGWYRQAPGSQRELVASISAGTASYADAVKGRFTISRDYAKNIIYLQMNSLKPDDTAV
YFCNYRTTYTSGYSEDYWGQGTQVTVSGGGSGGGSYPYDVPDYA;
PPRV-H-CD45 (32)
MSAQRERINAFYKDNLHNKTHRVILDRERLTIERPYILLGVLLVMFLSLIGLLAIAGIRLHRAT
VGTAEIQSRLNTNIELTESIDHQTKDVLTPLFKIIGDEVGIRIPQKFSDLVKFISDKIKFLNPD
REYDFRDLRWCMNPPERVKINFDQFCEYKAAVKSVEHIFESSLNRSERLRLLTLGPGTGCLGRT
VTRAQFSELTLTLMDLDLEIKHNVSSVFTVVEEGLFGRTYTVWRSDTGKPSTSPGIGHFLRVFE
IGLVRDLELGAPIFHMTNYLTVNMSDDYRSCLLAVGELKLTALCTPSETVTLSESGVPKREPLV
VVILNLAGPTLGGELYSVLPTTDPTVEKLYLSSHRGIIKDNEANWVVPSTDVRDLQNKGECLVE
ACKTRPPSFCNGTGIGPWSEGRIPAYGVIRVSLDLASDPGVVITSVFGPLIPHLSGMDLYNNPF
SRAAWLAVPPYEQSFLGMINTIGFPDRAEVMPHILTTEIRGPRGRCHVPIELSSRIDDDIKIGS
NMVVLPTKDLRYITATYDVSRSEHAIVYYIYDTGRSSSYFYPVRLNFRGNPLSLRIECFPWYHK
VWCYHDCLIYNTITNEEVHTRGLTGIEVTCNPVGGGGSGGGGGGGGSAAAQVQLVQSGGGLVQ
PGGSLRLSCAASGRAFNSAAMGWYRQAPGSQRELVASISAGTASYADAVKGRFTISRDYAKNII
YLQMNSLKPDDTAVYFCNYRTTYTSGYSEDYWGQGTQVTVSGGGSGGGSYPYDVPDYA;
RPV-H-CD45 (32)
MSPPRDRVDAYYKDNFQFKNTRVVLNKEQLLIERPCMLLTVLFVMFLSLVGLLAIAGIRLHRAA
VNTAKINNDLTTSIDITKSIEYQVKDVLTPLFKIIGDEVGLRTPQRFTDLTKFISDKIKFLNPD
KEYDFRDINWCINPPERIKIDYDQYCAHTAAEDLITMLVNSSLTGTTVLRTSLVNLGRNCTGPT
TTKGQFSNISLTLSGIYSGRGYNISSMITITGKGMYGSTYLVGKYNQRARRPSIVWQQDYRVFE
VGIIRELGVGTPVFHMTNYLELPRQPELETCMLALGESKLAALCLADSPVALHYGRVGDDNKIR
FVKLGVWASPADRDTLATLTHEPTLDGLYITTHRGIIAAGTAIWAVPVTRTDDQVKMGKCRLEA
CRDRPPPFCNSTDWEPLEAGRIPAYGVLTIKLGLADEPKVDIISEFGPLITHDSGMDLYTSFDG
TKYWLTTPPLQNSALGTVNTLVLEPSLKISPNILTLPIRSGGGDCYTPTYLSDRADDDVKLSSN
LVILPSRDLQYVSATYDISRVEHAIVYHIYSTGRLSSYYYPFKLPIKGDPVSLQIECFPWDRKL
WCHHFCSVIDSGTGEQVTHIGVVGIEITCNGKGGGGSGGGGSGGGGSAAAQVQLVQSGGGLVQP
GGSLRLSCAASGRAFNSAAMGWYRQAPGSQRELVASISAGTASYADAVKGRFTISRDYAKNIIY
LQMNSLKPDDTAVYFCNYRTTYTSGYSEDYWGQGTQVTVSGGGSGGGSYPYDVPDYA;and
RMV-H-CD45 (32)
MSAQRERINAFYKDNPHNKNHRVILDRERLVIERPYILLGVLLVMFLSLIGLLAIAGIRLHRAT
VGTSEIQSRLNTNIELTESIDHQTKDVLTPLFKIIGDEVGIRIPQKFSDLVKFISDKIKFLNPD
REYDFRDLRWCMNPPERVKINFDQFCEYKAAVKSIEHIFESPLNKSKKLQSLTLGPGTGCLGRT
VTRAHFSELTLTLMDLDLEMKHNVSSVFTVVEEGLFGRTYTVWRSDARDPSTDLGIGHFLRVFE
IGLVRDLGLGPPVFHMTNYLTVNMSDDYRRCLLAVGELKLTALCSSSETVTLGERGVPKREPLV
VVILNLAGPTLGGELYSVLPTSDLMVEKLYLSSHRGIIKDDEANWVVPSTDVRDLQNKGECLVE
ACKTRPPSFCNGTGSGPWSEGRIPAYGVIRVSLDLASDPGVVITSVFGPLIPHLSGMDLYNNPF
SRAVWLAVPPYEQSFLGMINTIGFPNRAEVMPHILTTEIRGPRGRCHVPIELSRRVDDDIKIGS
NMVILPTIDLRYITATYDVSRSEHAIVYYIYDTGRSSSYFYPVRLNFKGNPLSLRIECFPWRHK
VWCYHDCLIYNTITDEEVHTRGLTGIEVTCNPVGGGGSGGGGSGGGGSAAAQVQLVQSGGGLVQ
PGGSLRLSCAASGRAFNSAAMGWYRQAPGSQRELVASISAGTASYADAVKGRFTISRDYAKNII
YLQMNSLKPDDTAVYFCNYRTTYTSGYSEDYWGQGTQVTVSGGGSGGGSYPYDVPDYA.

In another aspect, the invention of the disclosure features a chimeric viral envelope glycoprotein polypeptide or fragment thereof suitable for pseudotyping a viral particle. The chimeric viral envelope glycoprotein polypeptide or fragment thereof contains an amino acid sequence at least about 20 amino acids in length derived from a non-measles virus morbillivirus F protein or H protein extravirion domain N-terminal to an amino acid sequence at least about 20 amino acids in length derived from a measles virus F protein or H protein extravirion domain.

In another aspect, the invention of the disclosure features a pseudotyped viral particle containing the chimeric polypeptide of any of the above aspects, or embodiments thereof.

In any of the above aspects, or embodiments thereof, the viral envelope glycoprotein domain or fragment thereof contains a viral hemagglutinin domain or fragment thereof. In embodiments, the viral hemagglutinin domain or fragment thereof is derived from a hemagglutinin polypeptide of a Paramyxovirus. In embodiments, the Paramyxovirus is a Morbillivirus. In embodiments, the Morbillivirus is selected from one or more of canine distemper virus, dolphin Morbillivirus, feline Morbillivirus, measles virus, Peste des petits ruminant virus, phocine Morbillivirus, Rinderpest virus, and small ruminant virus.

In any of the above aspects, or embodiments thereof, the viral envelope glycoprotein domain or fragment thereof contains a stalk polypeptide sequence derived from a measles virus envelope glycoprotein domain and an extravirion domain derived from a non-measles virus envelope glycoprotein. In embodiments, the stalk polypeptide contains an amino acid sequence with at least about 85% sequence identity to the sequence RLHRAAIYTAEIHKSLSTNLDVTNSIEHQVKDVLTPLFKI IGDEVGLRTPQRFTDLVKFISDKI KFLNPDREYDFRDLTWCINPPERIKLDYDQYCADVAA. In embodiments, the extravirion domain is derived from a dolphin Morbillivirus or a canine distemper virus. In embodiments, the extravirion domain contains an amino acid sequence with at least about 85% sequence identity to one of the following sequences:

Extravirion domain of CDV-H
RKAIASAANPILLSALSGGRGDIFPPHRCSGATTSVGKVFPLSVSLSMSLISRTSEVINMLTAI
SDGVYGKTYLLVPDDIEREFDTREIRVFEIGFIKRWLNDMPLLQTTNYMVLPKNSKAKVCTIAV
GELTLASLCVEESTVLLYHDSSGSQDGILVVTLGIFWATPMDHIEEVIPVAHPSMKKIHITNHR
GFIKDSIATWMVPALASEKQEEQKGCLESACQRKTYPMCNQASWEPFGGRQLPSYGRLTLPLDA
SVDLQLNISFTYGPVILNGDGMDYYESPLLNSGWLTIPPKDGTISGLINKAGRGDQFTVLPHVL
TFAPRESSGNCYLPIQTSQIRDRDVLIESNIVVLPTQSIRYVIATYDISRSDHAIVYYVYDPIR
TISYTHPFRLTTKGRPDFLRIECFVWDDNLWCHQFYRFEADIANSTTSVENLVRIRFSCNR;
and
Extravirion domain of DMV-H
EELIVTKFKELMNHSLDMSKGRIFPPKNCSGSVITRGQTIKPGLTLVNIYTTRNFEVSFMVTVI
SGGMYGKTYFLKPPEPDDPFEFQAFRIFEVGLVRDVGSREPVLQMTNFMVIDEDEGLNFCLLSV
GELRLAAVCVRGRPVVTKDIGGYKDEPFKVVTLGIIGGGLSNQKTEIYPTIDSSIEKLYITSHR
GIIRNSKARWSVPAIRSDDKDKMEKCTQALCKSRPPPSCNSSDWEPLTSNRIPAYAYIALEIKE
DSGLELDITSNYGPLIIHGAGMDIYEGPSSNQDWLAIPPLSQSVLGVINKVDFTAGFDIKPHTL
TTAVDYESGKCYVPVELSGAKDQDLKLESNLVVLPTKDFGYVTATYDTSRSEHAIVYYVYDTAR
SSSYFFPFRIKARGEPIYLRIECFPWSRQLWCHHYCMINSTVSNEIVVVDNLVSINMSCSR.

In any of the above aspects, or embodiments thereof, the viral envelope glycoprotein domain or fragment thereof contains an amino acid sequence with at least about 85% sequence identity to one of the following sequences, a fragment thereof, a cytoplasmic, transmembrane, stalk, or extravirion domain thereof, or to one of the following sequences containing a truncated cytoplasmic domain:

DMV-H
MSSPRDKVDAFYKDIPRPRNNRVLLDNERVIIERPLILVGVLAVMFLSLVGLLAIAGVRLQKAT
TNSIEVNRKLSTNLETTVSIEHHVKDVLTPLFKIIGDEVGLRMPQKLTEIMQFISNKIKFLNPD
REYDFNDLHWCVNPPDQVKIDYAQYCNHIAAEELIVTKFKELMNHSLDMSKGRIFPPKNCSGSV
ITRGQTIKPGLTLVNIYTTRNFEVSFMVTVISGGMYGKTYFLKPPEPDDPFEFQAFRIFEVGLV
RDVGSREPVLQMTNFMVIDEDEGLNFCLLSVGELRLAAVCVRGRPVVTKDIGGYKDEPFKVVTL
GIIGGGLSNQKTEIYPTIDSSIEKLYITSHRGIIRNSKARWSVPAIRSDDKDKMEKCTQALCKS
RPPPSCNSSDWEPLTSNRIPAYAYIALEIKEDSGLELDITSNYGPLIIHGAGMDIYEGPSSNQD
WLAIPPLSQSVLGVINKVDFTAGFDIKPHTLTTAVDYESGKCYVPVELSGAKDQDLKLESNLVV
LPTKDFGYVTATYDTSRSEHAIVYYVYDTARSSSYFFPFRIKARGEPIYLRIECFPWSRQLWCH
HYCMINSTVSNEIVVVDNLVSINMSCSR;
CDV-H
MLPYQDKVGAFYKDNARANSTKLSLVTEGHGGRRPPYLLFVLLILLVGILALLAITGVRFHQVS
TSNMEFSRLLKEDMEKSEAVHHQVIDVLTPLFKIIGDEIGLRLPQKLNEIKQFILQKTNFFNPN
REFDFRDLHWCINPPSTVKVNFTNYCESIGIRKAIASAANPILLSALSGGRGDIFPPHRCSGAT
TSVGKVFPLSVSLSMSLISRTSEVINMLTAISDGVYGKTYLLVPDDIEREFDTREIRVFEIGFI
KRWLNDMPLLQTTNYMVLPKNSKAKVCTIAVGELTLASLCVEESTVLLYHDSSGSQDGILVVTL
GIFWATPMDHIEEVIPVAHPSMKKIHITNHRGFIKDSIATWMVPALASEKQEEQKGCLESACQR
KTYPMCNQASWEPFGGRQLPSYGRLTLPLDASVDLQLNISFTYGPVILNGDGMDYYESPLLNSG
WLTIPPKDGTISGLINKAGRGDQFTVLPHVLTFAPRESSGNCYLPIQTSQIRDRDVLIESNIVV
LPTQSIRYVIATYDISRSDHAIVYYVYDPIRTISYTHPFRLTTKGRPDFLRIECFVWDDNLWCH
QFYRFEADIANSTTSVENLVRIRFSCNR;
MeV-Hc18-CDV

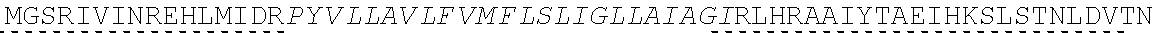

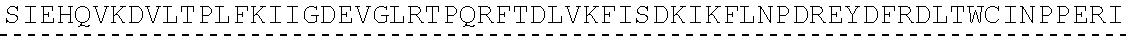

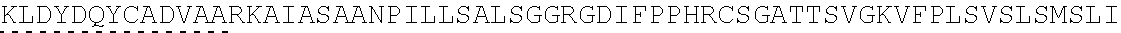

SRTSEVINMLTAISDGVYGKTYLLVPDDIEREFDTREIRVFEIGFIKRWLNDMPLLQTTNYMVL
PKNSKAKVCTIAVGELTLASLCVEESTVLLYHDSSGSQDGILVVTLGIFWATPMDHIEEVIPVA
HPSMKKIHITNHRGFIKDSIATWMVPALASEKQEEQKGCLESACORKTYPMCNQASWEPFGGRQ
LPSYGRLTLPLDASVDLQLNISFTYGPVILNGDGMDYYESPLLNSGWLTIPPKDGTISGLINKA
GRGDQFTVLPHVLTFAPRESSGNCYLPIQTSQIRDRDVLIESNIVVLPTQSIRYVIATYDISRS
DHAIVYYVYDPIRTISYTHPFRLTTKGRPDFLRIECFVWDDNLWCHQFYRFEADIANSTTSVEN
LVRIRFSCNR;
MeV-Hc18-DMV

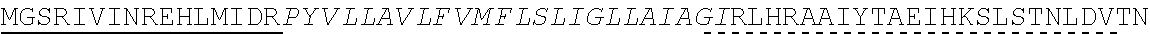

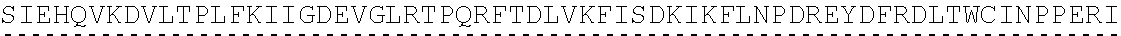

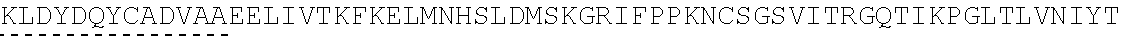

TRNFEVSFMVTVISGGMYGKTYFLKPPEPDDPFEFQAFRIFEVGLVRDVGSREPVLQMTNFMVI
DEDEGLNFCLLSVGELRLAAVCVRGRPVVTKDIGGYKDEPFKVVTLGIIGGGLSNQKTEIYPTI
DSSIEKLYITSHRGIIRNSKARWSVPAIRSDDKDKMEKCTQALCKSRPPPSCNSSDWEPLTSNR
IPAYAYIALEIKEDSGLELDITSNYGPLIIHGAGMDIYEGPSSNQDWLAIPPLSQSVLGVINKV
DFTAGFDIKPHTLTTAVDYESGKCYVPVELSGAKDQDLKLESNLVVLPTKDFGYVTATYDTSRS
EHAIVYYVYDTARSSSYFFPFRIKARGEPIYLRIECFPWSRQLWCHHYCMINSTVSNEIVVVDN
LVSINMSCSR;
FMV-H
MESNNIKYYKDSSRYFGKILDEHKTINSQLYSLSIKVITIIAIIVSLIATIITIINATSGRTTL
NSNTDILLSQRDEIHNIQEMIFDRIYPLINAMSTELGLHIPTLLDELTKAIDQKIKIMHPPVDT
VTSDLNWCIKPPNGIIIDPKSYCESMELSKTYELLLDQLDVSRKKSLIINRKNINQCQLVDNSK
IIFATVNIQSTPRFLNFGHTVSNQRITFGQGTYSSTYVITIQEDGVTDVQYRVFEIGYISDQFG
VFPSLIVSRVLPIRMLLGMESCTLTSDRLGGYFLCMNTLTRSIYDYVSIRDLKSLYITIPHYGK
VNYTYFNFGKIRSPHEIDKIWLTSDRGQIISGYFAAFVTITIRNYNNYPYKCLNNPCFDNSENY
CRGWYKNITGTDDVPILAYLLVEMYDEEGPLITLVAIPPYNYTAPSHNSLYYDDKINKLIMTTS
HIGYIQINEVHEVIVGDNLKAILLNRLSDEHPNLTACRLNQGIKEQYKSDGTIISNSALIDIQE
RMYITVKAIPPAGNYNFTVELHSRSNTSYVSLPKQFNAKYDKLHLECFSWDKSWWCALIPQFSL
SWNESLSVDTAIFNLISCK;
PPRV-H
MSAQRERINAFYKDNLHNKTHRVILDRERLTIERPYILLGVLLVMFLSLIGLLAIAGIRLHRAT
VGTAEIQSRLNTNIELTESIDHQTKDVLTPLFKIIGDEVGIRIPQKFSDLVKFISDKIKFLNPD
REYDFRDLRWCMNPPERVKINFDQFCEYKAAVKSVEHIFESSLNRSERLRLLTLGPGTGCLGRT
VTRAQFSELTLTLMDLDLEIKHNVSSVFTVVEEGLFGRTYTVWRSDTGKPSTSPGIGHFLRVFE
IGLVRDLELGAPIFHMTNYLTVNMSDDYRSCLLAVGELKLTALCTPSETVTLSESGVPKREPLV
VVILNLAGPTLGGELYSVLPTTDPTVEKLYLSSHRGIIKDNEANWVVPSTDVRDLQNKGECLVE
ACKTRPPSFCNGTGIGPWSEGRIPAYGVIRVSLDLASDPGVVITSVFGPLIPHLSGMDLYNNPF
SRAAWLAVPPYEQSFLGMINTIGFPDRAEVMPHILTTEIRGPRGRCHVPIELSSRIDDDIKIGS
NMVVLPTKDLRYITATYDVSRSEHAIVYYIYDTGRSSSYFYPVRLNFRGNPLSLRIECFPWYHK
VWCYHDCLIYNTITNEEVHTRGLTGIEVTCNPV;
RPV-H
MSPPRDRVDAYYKDNFQFKNTRVVLNKEQLLIERPCMLLTVLFVMFLSLVGLLAIAGIRLHRAA
VNTAKINNDLTTSIDITKSIEYQVKDVLTPLFKIIGDEVGLRTPQRFTDLTKFISDKIKFLNPD
KEYDFRDINWCINPPERIKIDYDQYCAHTAAEDLITMLVNSSLTGTTVLRTSLVNLGRNCTGPT
TTKGQFSNISLTLSGIYSGRGYNISSMITITGKGMYGSTYLVGKYNQRARRPSIVWQQDYRVFE
VGIIRELGVGTPVFHMTNYLELPRQPELETCMLALGESKLAALCLADSPVALHYGRVGDDNKIR
FVKLGVWASPADRDTLATLTHEPTLDGLYITTHRGIIAAGTAIWAVPVTRTDDQVKMGKCRLEA
CRDRPPPFCNSTDWEPLEAGRIPAYGVLTIKLGLADEPKVDIISEFGPLITHDSGMDLYTSFDG
TKYWLTTPPLONSALGTVNTLVLEPSLKISPNILTLPIRSGGGDCYTPTYLSDRADDDVKLSSN
LVILPSRDLQYVSATYDISRVEHAIVYHIYSTGRLSSYYYPFKLPIKGDPVSLQIECFPWDRKL
WCHHFCSVIDSGTGEQVTHIGVVGIEITCNGK; and
RMV-H
MSAQRERINAFYKDNPHNKNHRVILDRERLVIERPYILLGVLLVMFLSLIGLLAIAGIRLHRAT
VGTSEIQSRLNTNIELTESIDHQTKDVLTPLFKIIGDEVGIRIPQKFSDLVKFISDKIKFLNPD
REYDFRDLRWCMNPPERVKINFDQFCEYKAAVKSIEHIFESPLNKSKKLQSLTLGPGTGCLGRT
VTRAHFSELTLTLMDLDLEMKHNVSSVFTVVEEGLFGRTYTVWRSDARDPSTDLGIGHFLRVFE
IGLVRDLGLGPPVFHMTNYLTVNMSDDYRRCLLAVGELKLTALCSSSETVTLGERGVPKREPLV
VVILNLAGPTLGGELYSVLPTSDLMVEKLYLSSHRGIIKDDEANWVVPSTDVRDLQNKGECLVE
ACKTRPPSFCNGTGSGPWSEGRIPAYGVIRVSLDLASDPGVVITSVFGPLIPHLSGMDLYNNPF
SRAVWLAVPPYEQSFLGMINTIGFPNRAEVMPHILTTEIRGPRGRCHVPIELSRRVDDDIKIGS
NMVILPTIDLRYITATYDVSRSEHAIVYYIYDTGRSSSYFYPVRLNFKGNPLSLRIECFPWRHK
VWCYHDCLIYNTITDEEVHTRGLTGIEVTCNPV,

where cytoplasmic domains are denoted by underlined text, transmembrane domains are denoted by italicized text, stalks are denoted by text underlined with a dashed line, and extravirion domains are denoted by plain text.

In any of the above aspects, or embodiments thereof, the VHH domain or fragment thereof contains a sequence with at least about 85% sequence identity to a sequence selected from one or more of

anti-major histocompatibility II (MHCII) VHH (N11)
QVQLVQSGGGLVQPGGSLGLSCAASGNIGSRDNMGWYRQAPGKQREWVA
TISGYGIATYRDSVKGRFTVAKDTAKNIVSLQMNYLTTEDTAVYYCYAY
AVDSRNIFWSQGTQVTVS;
anti-CD45 (32) VHH
QVQLVQSGGGLVQPGGSLRLSCAASGRAFNSAAMGWYRQAPGSQRELVA
SISAGTASYADAVKGRFTISRDYAKNIIYLQMNSLKPDDTAVYFCNYRT
TYTSGYSEDYWGQGTQVTVS;
anti-CD7 (VHH10) VHH
DVQLQESGGGSVQAGGSLRLSCAASGYTHSSYCMAWFRQAPGREREGVA
SIDSDGTTSYADSVKGRFTISQDNAKNTLYLQMNSLKPEDTAMYYCAAR
FGPMGCVDLSTLSFGHWGQGTQVTVSIT;
anti-CD4 (03F11) VHH
EVQLVESGGGSVQPGGSLTLSCGTSGRTFNVMGWFRQAPGKEREFVAAV
RWSSTGIYYTQYADSVKSRFTISRDNAKNTVYLEMNSLKPEDTAVYYCA
ADTYNSNPARWDGYDERGQGTQVTVSS;
and
anti-CD8 (R3HCD27) VHH
QVQLQESGGGSVQPGGSLRLSCAASGFTFDDYAMSWVRQVPGKGLEWVS
TINWNGGSAEYAEPVKGRFTISRDNAKNTVYLQMNSLKLEDTAVYYCAK
DADLVWYNLSTGQGTQVTVSS.

In any of the above aspects, or embodiments thereof, the viral envelope glycoprotein domain or fragment thereof and the VHH domain or fragment thereof are separated by a linker. In embodiments, the linker contains the sequence GGGGSGGGGSGGGGS.

In any of the above aspects, or embodiments thereof, where viral envelope glycoprotein domain or fragment thereof fused to the VHH domain or fragment thereof contains a sequence with at least 85% sequence identity to a sequence selected from one or more of:

DMV-H-MHCII (N11)
MSSPRDKVDAFYKDIPRPRNNRVLLDNERVIIERPLILVGVLAVMFLSLVGLLAIAGVRLQKAT
TNSIEVNRKLSTNLETTVSIEHHVKDVLTPLFKIIGDEVGLRMPQKLTEIMQFISNKIKFLNPD
REYDFNDLHWCVNPPDQVKIDYAQYCNHIAAEELIVTKFKELMNHSLDMSKGRIFPPKNCSGSV
ITRGQTIKPGLTLVNIYTTRNFEVSFMVTVISGGMYGKTYFLKPPEPDDPFEFQAFRIFEVGLV
RDVGSREPVLQMTNFMVIDEDEGLNFCLLSVGELRLAAVCVRGRPVVTKDIGGYKDEPFKVVTL
GIIGGGLSNQKTEIYPTIDSSIEKLYITSHRGIIRNSKARWSVPAIRSDDKDKMEKCTQALCKS
RPPPSCNSSDWEPLTSNRIPAYAYIALEIKEDSGLELDITSNYGPLIIHGAGMDIYEGPSSNQD
WLAIPPLSQSVLGVINKVDFTAGFDIKPHTLTTAVDYESGKCYVPVELSGAKDQDLKLESNLVV
LPTKDFGYVTATYDTSRSEHAIVYYVYDTARSSSYFFPFRIKARGEPIYLRIECFPWSRQLWCH
HYCMINSTVSNEIVVVDNLVSINMSCSRGGGGSGGGGSGGGGSAAAQVQLVQSGGGLVQPGGSL
GLSCAASGNIGSRDNMGWYRQAPGKQREWVATISGYGIATYRDSVKGRFTVAKDTAKNIVSLQM
NYLTTEDTAVYYCYAYAVDSRNIFWSQGTQVTVS;
DMV-H-CD7 (Humanized VHH10)
MSSPRDKVDAFYKDIPRPRNNRVLLDNERVIIERPLILVGVLAVMFLSLVGLLAIAGVRLQKAT
TNSIEVNRKLSTNLETTVSIEHHVKDVLTPLFKIIGDEVGLRMPQKLTEIMQFISNKIKFLNPD
REYDFNDLHWCVNPPDQVKIDYAQYCNHIAAEELIVTKFKELMNHSLDMSKGRIFPPKNCSGSV
ITRGQTIKPGLTLVNIYTTRNFEVSFMVTVISGGMYGKTYFLKPPEPDDPFEFQAFRIFEVGLV
RDVGSREPVLQMTNFMVIDEDEGLNFCLLSVGELRLAAVCVRGRPVVTKDIGGYKDEPFKVVTL
GIIGGGLSNQKTEIYPTIDSSIEKLYITSHRGIIRNSKARWSVPAIRSDDKDKMEKCTQALCKS
RPPPSCNSSDWEPLTSNRIPAYAYIALEIKEDSGLELDITSNYGPLIIHGAGMDIYEGPSSNQD
WLAIPPLSQSVLGVINKVDFTAGFDIKPHTLTTAVDYESGKCYVPVELSGAKDQDLKLESNLVV
LPTKDFGYVTATYDTSRSEHAIVYYVYDTARSSSYFFPFRIKARGEPIYLRIECFPWSRQLWCH
HYCMINSTVSNEIVVVDNLVSINMSCSRGGGGSGGGGSGGGGSAAADVQLQESGGGSVQAGGSL
RLSCAASGYTHSSYCMAWFRQAPGREREGVASIDSDGTTSYADSVKGRFTISQDNAKNTLYLQM
NSLKPEDTAMYYCAARFGPMGCVDLSTLSFGHWGQGTQVTVSITGGGSGGGSYPYDVPDYA;
DMV-H-CD45 (32)
MSSPRDKVDAFYKDIPRPRNNRVLLDNERVIIERPLILVGVLAVMFLSLVGLLAIAGVRLQKAT
TNSIEVNRKLSTNLETTVSIEHHVKDVLTPLFKIIGDEVGLRMPQKLTEIMQFISNKIKFLNPD
REYDFNDLHWCVNPPDQVKIDYAQYCNHIAAEELIVTKFKELMNHSLDMSKGRIFPPKNCSGSV
ITRGQTIKPGLTLVNIYTTRNFEVSFMVTVISGGMYGKTYFLKPPEPDDPFEFQAFRIFEVGLV
RDVGSREPVLQMTNFMVIDEDEGLNFCLLSVGELRLAAVCVRGRPVVTKDIGGYKDEPFKVVTL
GIIGGGLSNQKTEIYPTIDSSIEKLYITSHRGIIRNSKARWSVPAIRSDDKDKMEKCTQALCKS
RPPPSCNSSDWEPLTSNRIPAYAYIALEIKEDSGLELDITSNYGPLIIHGAGMDIYEGPSSNQD
WLAIPPLSQSVLGVINKVDFTAGFDIKPHTLTTAVDYESGKCYVPVELSGAKDQDLKLESNLVV
LPTKDFGYVTATYDTSRSEHAIVYYVYDTARSSSYFFPFRIKARGEPIYLRIECFPWSRQLWCH
HYCMINSTVSNEIVVVDNLVSINMSCSRGGGGSGGGGSGGGGSAAAQVQLVQSGGGLVQPGGSL
RLSCAASGRAFNSAAMGWYRQAPGSQRELVASISAGTASYADAVKGRFTISRDYAKNIIYLQMN
SLKPDDTAVYFCNYRTTYTSGYSEDYWGQGTQVTVSGGGSGGGSYPYDVPDYA;
CDV-H-MHCII (N11)
MLPYQDKVGAFYKDNARANSTKLSLVTEGHGGRRPPYLLFVLLILLVGILALLAITGVRFHQVS
TSNMEFSRLLKEDMEKSEAVHHQVIDVLTPLFKIIGDEIGLRLPQKLNEIKQFILQKTNFFNPN
REFDFRDLHWCINPPSTVKVNFTNYCESIGIRKAIASAANPILLSALSGGRGDIFPPHRCSGAT
TSVGKVFPLSVSLSMSLISRTSEVINMLTAISDGVYGKTYLLVPDDIEREFDTREIRVFEIGFI
KRWLNDMPLLQTTNYMVLPKNSKAKVCTIAVGELTLASLCVEESTVLLYHDSSGSQDGILVVTL
GIFWATPMDHIEEVIPVAHPSMKKIHITNHRGFIKDSIATWMVPALASEKQEEQKGCLESACQR
KTYPMCNQASWEPFGGRQLPSYGRLTLPLDASVDLQLNISFTYGPVILNGDGMDYYESPLLNSG
WLTIPPKDGTISGLINKAGRGDQFTVLPHVLTFAPRESSGNCYLPIQTSQIRDRDVLIESNIVV
LPTQSIRYVIATYDISRSDHAIVYYVYDPIRTISYTHPFRLTTKGRPDFLRIECFVWDDNLWCH
QFYRFEADIANSTTSVENLVRIRFSCNRGGGGSGGGGSGGGGSAAAQVQLVQSGGGLVQPGGSL
GLSCAASGNIGSRDNMGWYRQAPGKQREWVATISGYGIATYRDSVKGRFTVAKDTAKNIVSLQM
NYLTTEDTAVYYCYAYAVDSRNIFWSQGTQVTVSGGGSGGGSYPYDVPDYA;
CDV-H-CD7 (Humanized VHH10)
MLPYQDKVGAFYKDNARANSTKLSLVTEGHGGRRPPYLLFVLLILLVGILALLAITGVRFHQVS
TSNMEFSRLLKEDMEKSEAVHHQVIDVLTPLFKIIGDEIGLRLPQKLNEIKQFILQKTNFFNPN
REFDFRDLHWCINPPSTVKVNFTNYCESIGIRKAIASAANPILLSALSGGRGDIFPPHRCSGAT
TSVGKVFPLSVSLSMSLISRTSEVINMLTAISDGVYGKTYLLVPDDIEREFDTREIRVFEIGFI
KRWLNDMPLLQTTNYMVLPKNSKAKVCTIAVGELTLASLCVEESTVLLYHDSSGSQDGILVVTL
GIFWATPMDHIEEVIPVAHPSMKKIHITNHRGFIKDSIATWMVPALASEKQEEQKGCLESACQR
KTYPMCNQASWEPFGGRQLPSYGRLTLPLDASVDLQLNISFTYGPVILNGDGMDYYESPLLNSG
WLTIPPKDGTISGLINKAGRGDQFTVLPHVLTFAPRESSGNCYLPIQTSQIRDRDVLIESNIVV
LPTQSIRYVIATYDISRSDHAIVYYVYDPIRTISYTHPFRLTTKGRPDFLRIECFVWDDNLWCH
QFYRFEADIANSTTSVENLVRIRFSCNRGGGGSGGGGSGGGGSAAADVQLQESGGGSVQAGGSL
RLSCAASGYTHSSYCMAWFRQAPGREREGVASIDSDGTTSYADSVKGRFTISQDNAKNTLYLQM
NSLKPEDTAMYYCAARFGPMGCVDLSTLSFGHWGQGTQVTVSITGGGSGGGSYPYDVPDYA;
CDV-H-CD45 VHH (32)
MLPYQDKVGAFYKDNARANSTKLSLVTEGHGGRRPPYLLFVLLILLVGILALLAITGVRFHQVS
TSNMEFSRLLKEDMEKSEAVHHQVIDVLTPLFKIIGDEIGLRLPQKLNEIKQFILQKTNFFNPN
REFDFRDLHWCINPPSTVKVNFTNYCESIGIRKAIASAANPILLSALSGGRGDIFPPHRCSGAT
TSVGKVFPLSVSLSMSLISRTSEVINMLTAISDGVYGKTYLLVPDDIEREFDTREIRVFEIGFI
KRWLNDMPLLQTTNYMVLPKNSKAKVCTIAVGELTLASLCVEESTVLLYHDSSGSQDGILVVTL
GIFWATPMDHIEEVIPVAHPSMKKIHITNHRGFIKDSIATWMVPALASEKQEEQKGCLESACQR
KTYPMCNQASWEPFGGRQLPSYGRLTLPLDASVDLQLNISFTYGPVILNGDGMDYYESPLLNSG
WLTIPPKDGTISGLINKAGRGDQFTVLPHVLTFAPRESSGNCYLPIQTSQIRDRDVLIESNIVV
LPTQSIRYVIATYDISRSDHAIVYYVYDPIRTISYTHPFRLTTKGRPDFLRIECFVWDDNLWCH
QFYRFEADIANSTTSVENLVRIRFSCNRGGGGSGGGGSGGGGSAAAQVQLVQSGGGLVQPGGSL
RLSCAASGRAFNSAAMGWYRQAPGSQRELVASISAGTASYADAVKGRFTISRDYAKNIIYLQMN
SLKPDDTAVYFCNYRTTYTSGYSEDYWGQGTQVTVSAAAYPYDVPDYA;
MeV-Hc18-CDV-MHCII (N11)
MGSRIVINREHLMIDRPYVLLAVLFVMFLSLIGLLAIAGIRLHRAAIYTAEIHKSLSTNLDVTN
SIEHQVKDVLTPLFKIIGDEVGLRTPQRFTDLVKFISDKIKFLNPDREYDFRDLTWCINPPERI
KLDYDQYCADVAARKAIASAANPILLSALSGGRGDIFPPHRCSGATTSVGKVFPLSVSLSMSLI
SRTSEVINMLTAISDGVYGKTYLLVPDDIEREFDTREIRVFEIGFIKRWLNDMPLLQTTNYMVL
PKNSKAKVCTIAVGELTLASLCVEESTVLLYHDSSGSQDGILVVTLGIFWATPMDHIEEVIPVA
HPSMKKIHITNHRGFIKDSIATWMVPALASEKQEEQKGCLESACQRKTYPMCNQASWEPFGGRQ
LPSYGRLTLPLDASVDLQLNISFTYGPVILNGDGMDYYESPLLNSGWLTIPPKDGTISGLINKA
GRGDQFTVLPHVLTFAPRESSGNCYLPIQTSQIRDRDVLIESNIVVLPTQSIRYVIATYDISRS
DHAIVYYVYDPIRTISYTHPFRLTTKGRPDFLRIECFVWDDNLWCHQFYRFEADIANSTTSVEN
LVRIRFSCNRGGGGSGGGGSGGGGSAAAQVQLVQSGGGLVQPGGSLGLSCAASGNIGSRDNMGW
YRQAPGKQREWVATISGYGIATYRDSVKGRFTVAKDTAKNIVSLQMNYLTTEDTAVYYCYAYAV
DSRNIFWSQGTQVTVSGGGSGGGSYPYDVPDYA;
MeV-Hc18-DMV-MHCII (N11)
MGSRIVINREHLMIDRPYVLLAVLFVMFLSLIGLLAIAGIRLHRAAIYTAEIHKSLSTNLDVTN
SIEHQVKDVLTPLFKIIGDEVGLRTPQRFTDLVKFISDKIKFLNPDREYDFRDLTWCINPPERI
KLDYDQYCADVAAEELIVTKFKELMNHSLDMSKGRIFPPKNCSGSVITRGQTIKPGLTLVNIYT
TRNFEVSFMVTVISGGMYGKTYFLKPPEPDDPFEFQAFRIFEVGLVRDVGSREPVLQMTNFMVI
DEDEGLNFCLLSVGELRLAAVCVRGRPVVTKDIGGYKDEPFKVVTLGIIGGGLSNQKTEIYPTI
DSSIEKLYITSHRGIIRNSKARWSVPAIRSDDKDKMEKCTQALCKSRPPPSCNSSDWEPLTSNR
IPAYAYIALEIKEDSGLELDITSNYGPLIIHGAGMDIYEGPSSNQDWLAIPPLSQSVLGVINKV
DFTAGFDIKPHTLTTAVDYESGKCYVPVELSGAKDQDLKLESNLVVLPTKDFGYVTATYDTSRS
EHAIVYYVYDTARSSSYFFPFRIKARGEPIYLRIECFPWSRQLWCHHYCMINSTVSNEIVVVDN
LVSINMSCSRGGGGSGGGGSGGGGSAAAQVQLVQSGGGLVQPGGSLGLSCAASGNIGSRDNMGW
YRQAPGKQREWVATISGYGIATYRDSVKGRFTVAKDTAKNIVSLQMNYLTTEDTAVYYCYAYAV
DSRNIFWSQGTQVTVSGGGSGGGSYPYDVPDYA;
FMV-H-CD45 (32) polypeptide
MESNNIKYYKDSSRYFGKILDEHKTINSQLYSLSIKVITIIAIIVSLIATIITIINATSGRTTL
NSNTDILLSQRDEIHNIQEMIFDRIYPLINAMSTELGLHIPTLLDELTKAIDQKIKIMHPPVDT
VTSDLNWCIKPPNGIIIDPKSYCESMELSKTYELLLDQLDVSRKKSLIINRKNINQCQLVDNSK
IIFATVNIQSTPRFLNFGHTVSNQRITFGQGTYSSTYVITIQEDGVTDVQYRVFEIGYISDQFG
VFPSLIVSRVLPIRMLLGMESCTLTSDRLGGYFLCMNTLTRSIYDYVSIRDLKSLYITIPHYGK
VNYTYFNFGKIRSPHEIDKIWLTSDRGQIISGYFAAFVTITIRNYNNYPYKCLNNPCFDNSENY
CRGWYKNITGTDDVPILAYLLVEMYDEEGPLITLVAIPPYNYTAPSHNSLYYDDKINKLIMTTS
HIGYIQINEVHEVIVGDNLKAILLNRLSDEHPNLTACRLNQGIKEQYKSDGTIISNSALIDIQE
RMYITVKAIPPAGNYNFTVELHSRSNTSYVSLPKQFNAKYDKLHLECFSWDKSWWCALIPQFSL
SWNESLSVDTAIFNLISCKGGGGSGGGGSGGGGSAAAQVQLVQSGGGLVQPGGSLRLSCAASGR
AFNSAAMGWYRQAPGSQRELVASISAGTASYADAVKGRFTISRDYAKNIIYLQMNSLKPDDTAV
YFCNYRTTYTSGYSEDYWGQGTQVTVSGGGSGGGSYPYDVPDYA;
PPRV-H-CD45 (32)
MSAQRERINAFYKDNLHNKTHRVILDRERLTIERPYILLGVLLVMFLSLIGLLAIAGIRLHRAT
VGTAEIQSRLNTNIELTESIDHQTKDVLTPLFKIIGDEVGIRIPQKFSDLVKFISDKIKFLNPD
REYDFRDLRWCMNPPERVKINFDQFCEYKAAVKSVEHIFESSLNRSERLRLLTLGPGTGCLGRT
VTRAQFSELTLTLMDLDLEIKHNVSSVFTVVEEGLFGRTYTVWRSDTGKPSTSPGIGHFLRVFE
IGLVRDLELGAPIFHMTNYLTVNMSDDYRSCLLAVGELKLTALCTPSETVTLSESGVPKREPLV
VVILNLAGPTLGGELYSVLPTTDPTVEKLYLSSHRGIIKDNEANWVVPSTDVRDLQNKGECLVE
ACKTRPPSFCNGTGIGPWSEGRIPAYGVIRVSLDLASDPGVVITSVFGPLIPHLSGMDLYNNPF
SRAAWLAVPPYEQSFLGMINTIGFPDRAEVMPHILTTEIRGPRGRCHVPIELSSRIDDDIKIGS
NMVVLPTKDLRYITATYDVSRSEHAIVYYIYDTGRSSSYFYPVRLNFRGNPLSLRIECFPWYHK
VWCYHDCLIYNTITNEEVHTRGLTGIEVTCNPVGGGGSGGGGSGGGGSAAAQVQLVQSGGGLVQ
PGGSLRLSCAASGRAFNSAAMGWYRQAPGSQRELVASISAGTASYADAVKGRFTISRDYAKNII
YLQMNSLKPDDTAVYFCNYRTTYTSGYSEDYWGQGTQVTVSGGGSGGGSYPYDVPDYA;
RPV-H-CD45 (32)
MSPPRDRVDAYYKDNFQFKNTRVVLNKEQLLIERPCMLLTVLFVMFLSLVGLLAIAGIRLHRAA
VNTAKINNDLTTSIDITKSIEYQVKDVLTPLFKIIGDEVGLRTPQRFTDLTKFISDKIKFLNPD
KEYDFRDINWCINPPERIKIDYDQYCAHTAAEDLITMLVNSSLTGTTVLRTSLVNLGRNCTGPT
TTKGQFSNISLTLSGIYSGRGYNISSMITITGKGMYGSTYLVGKYNQRARRPSIVWQQDYRVFE
VGIIRELGVGTPVFHMTNYLELPRQPELETCMLALGESKLAALCLADSPVALHYGRVGDDNKIR
FVKLGVWASPADRDTLATLTHEPTLDGLYITTHRGIIAAGTAIWAVPVTRTDDQVKMGKCRLEA
CRDRPPPFCNSTDWEPLEAGRIPAYGVLTIKLGLADEPKVDIISEFGPLITHDSGMDLYTSFDG
TKYWLTTPPLQNSALGTVNTLVLEPSLKISPNILTLPIRSGGGDCYTPTYLSDRADDDVKLSSN
LVILPSRDLQYVSATYDISRVEHAIVYHIYSTGRLSSYYYPFKLPIKGDPVSLQIECFPWDRKL
WCHHFCSVIDSGTGEQVTHIGVVGIEITCNGKGGGGSGGGGSGGGGSAAAQVQLVQSGGGLVQP
GGSLRLSCAASGRAFNSAAMGWYRQAPGSQRELVASISAGTASYADAVKGRFTISRDYAKNIIY
LQMNSLKPDDTAVYFCNYRTTYTSGYSEDYWGQGTQVTVSGGGSGGGSYPYDVPDYA;
and
RMV-H-CD45 (32)
MSAQRERINAFYKDNPHNKNHRVILDRERLVIERPYILLGVLLVMFLSLIGLLAIAGIRLHRAT
VGTSEIQSRLNTNIELTESIDHQTKDVLTPLFKIIGDEVGIRIPQKFSDLVKFISDKIKFLNPD
REYDFRDLRWCMNPPERVKINFDQFCEYKAAVKSIEHIFESPLNKSKKLQSLTLGPGTGCLGRT
VTRAHFSELTLTLMDLDLEMKHNVSSVFTVVEEGLFGRTYTVWRSDARDPSTDLGIGHFLRVFE
IGLVRDLGLGPPVFHMTNYLTVNMSDDYRRCLLAVGELKLTALCSSSETVTLGERGVPKREPLV
VVILNLAGPTLGGELYSVLPTSDLMVEKLYLSSHRGIIKDDEANWVVPSTDVRDLQNKGECLVE
ACKTRPPSFCNGTGSGPWSEGRIPAYGVIRVSLDLASDPGVVITSVFGPLIPHLSGMDLYNNPF
SRAVWLAVPPYEQSFLGMINTIGFPNRAEVMPHILTTEIRGPRGRCHVPIELSRRVDDDIKIGS
NMVILPTIDLRYITATYDVSRSEHAIVYYIYDTGRSSSYFYPVRLNFKGNPLSLRIECFPWRHK
VWCYHDCLIYNTITDEEVHTRGLTGIEVTCNPVGGGGSGGGGSGGGGSAAAQVQLVQSGGGLVQ
PGGSLRLSCAASGRAFNSAAMGWYRQAPGSQRELVASISAGTASYADAVKGRFTISRDYAKNII
YLQMNSLKPDDTAVYFCNYRTTYTSGYSEDYWGQGTQVTVSGGGSGGGSYPYDVPDYA.

In any of the above aspects, or embodiments thereof, the viral particle, cell, expression vector, or method further contains, encodes, or involves a chimeric viral envelope glycoprotein polypeptide or fragment thereof suitable for pseudotyping a viral particle. The chimeric envelope protein polypeptide or fragment thereof contains an amino acid sequence at least about amino acids in length derived from a non-measles virus morbillivirus F protein or H protein extravirion domain N-terminal to an amino acid sequence at least about 20 amino acids in length derived from a measles virus F protein or H protein extravirion domain. In embodiments, the chimeric viral envelope glycoprotein polypeptide contains the amino acid sequence RLDVGTNLGNAIAKLEDAKELLESSDQILRSMKGLSSTS (MeV-F extravirion stalk domain). In any of the above aspects, or embodiments thereof, the non-measles virus morbillivirus is selected from one or more of canine distemper virus, dolphin Morbillivirus, feline Morbillivirus, measles virus, Peste des petits ruminant virus, phocine Morbillivirus, Rinderpest virus, and small ruminant virus. In embodiments, the non-measles virus morbillivirus is dolphin Morbillivirus. In any of the above aspects, or embodiments thereof, the chimeric viral envelope glycoprotein contains at least about 80 amino acids derived from the non-measles virus morbillivirus F protein or H protein extravirion domain.

In any of the above aspects, or embodiments thereof, the envelope further contains a viral envelope glycoprotein containing an amino acid sequence with at least about 85% sequence identity to one or more of the following sequences, or to one or more of the following sequences

MeV-Fc30
MGLKVNVSAIFMAVLLTLQTPTGQIHWGNLSKIGVVGIGSASYKVMTRSSHQSLVIKLMPNITL
LNNCTRVEIAEYRRLLRTVLEPIRDALNAMTQNIRPVQSVASSRRHKRFAGVVLAGAALGVATA
AQITAGIALHQSMLNSQAIDNLRASLETTNQAIEAIRQAGQEMILAVQGVQDYINNELIPSMNQ
LSCDLIGQKLGLKLLRYYTEILSLFGPSLRDPISAEISIQALSYALGGDINKVLEKLGYSGGDL
LGILESRGIKARITHVDTESYFIVLSIAYPTLSEIKGVIVHRLEGVSYNIGSQEWYTTVPKYVA
TQGYLISNFDESSCTFMPEGTVCSQNALYPMSPLLQECLRGSTKSCARTLVSGSFGNRFILSQG
NLIANCASILCKCYTTGTIINQDPDKILTYIAADHCPVVEVNGVTIQVGSRRYPDAVYLHRIDL
GPPISLERLDVGTNLGNAIAKLEDAKELLESSDQILRSMKGLSSTSIVYILIAVCLGGLIGIPA
LICCCRGR;
DMV-F
MAASNGGVMYQSFLTIIILVIMTEGQIHWGNLSKIGIVGTGSASYKVMTRPNHQYLVIKLMPNV
TMIDNCTRTEVTEYRKLLKTVLEPVKNALTVITKNIKPIQSLTTSRRSKRFAGVVLAGVALGVA
TAAQITAGVALHQSIMNSQSIDNLRTSLEKSNQAIEEIRQASQETVLAVQGVQDFINNELIPSM
HQLSCEMLGQKLGLKLLRYYTEILSIFGPSLRDPVSAEISIQALSYALGGDINKILEKLGYSGA
DLLAILESRGIKAKVTHVDLEGYFIVLSIAYPTLSEVKGVIVHKLEAVSYNLGSQEWYTTLPKY
VATNGYLISNFDESSCAFMSEVTICSQNALYPMSPLLQQCLRGSTASCARSLVSGTIGNRFILS
KGNLIANCASVLCKCYSTGTIISQDPDKLLTFVAADKCPLVEVDGITIQVGSREYPDSVYVSRI
DLGPAISLEKLDVGTNLGSALTKLDNAKDLLDSSNQILENVRRSSFGGAMYIGILVCAGALVIL
CVLVYCCRRHCRKRVQTPPKATPGLKPDLTGTTKSYVRSL;
CDV-F
MHRGIPKSSKTQTHTQQDRPPQPSTELEETRTSRARHSTTSAQRSTHYDPRTSDRPVSYTMNRT
RSRKQTSHRLKNIPVHGNHEATIQHIPESVSKGARSQIERRQPNAINSGSHCTWLVLWCLGMAS
LFLCSKAQIHWDNLSTIGIIGTDNVHYKIMTRPSHQYLVIKLIPNASLIENCTKAELGEYEKLL
NSVLEPINQALTLMTKNVKPLQSLGSGRRQRRFAGVVLAGVALGVATAAQITAGIALHQSNLNA
QAIQSLRTSLEQSNKAIEEIREATQETVIAVQGVQDYVNNELVPAMQHMSCELVGQRLGLRLLR
YYTELLSIFGPSLRDPISAEISIQALIYALGGEIHKILEKLGYSGSDMIAILESRGIKTKITHV
DLPGKFIILSISYPTLSEVKGVIVHRLEAVSYNIGSQEWYTTVPRYIATNGYLISNFDESSCVF
VSESAICSQNSLYPMSPLLQQCIRGDTSSCARTLVSGTMGNKFILSKGNIVANCASILCKCYST
STIINQSPDKLLTFIASDTCPLVEIDGATIQVGGRQYPDMVYEGKVALGPAISLDRLDVGTNLG
NALKKLDDAKVLIDSSNQILETVRRSSFNFGSLLSVPILSCTALALLLLIYCCKRRYQQTLKQH
TKVDPAFKPDLTGTSKSYVRSL;
F2 DMV Fusion
MAASNGGVMYQSFLTIIILVIMTEGQIHWGNLSKIGIVGTGSASYKVMTRPNHQYLVIKLMPNV
TMIDNCTRTEVTEYRKLLKTVLEPVKNALTVITKNIKPIQSLYPYDVPDYATTSRRSKRFAGVV
LAGAALGVATAAQITAGIALHQSMLNSQAIDNLRASLETTNQAIEAIRQAGQEMILAVQGVQDY
INNELIPSMNQLSCDLIGQKLGLKLLRYYTEILSLFGPSLRDPISAEISIQALSYALGGDINKV
LEKLGYSGGDLLGILESRGIKARITHVDTESYFIVLSIAYPTLSEIKGVIVHRLEGVSYNIGSQ
EWYTTVPKYVATQGYLISNFDESSCTFMPEGTVCSQNALYPMSPLLQECLRGSTKSCARTLVSG
SFGNRFILSQGNLIANCASILCKCYTTGTIINQDPDKILTYIAADHCPVVEVNGVTIQVGSRRY
PDAVYLHRIDLGPPISLERLDVGTNLGNAIAKLEDAKELLESSDQILRSMKGLSSTSIVYILIA
VCLGGLIGIPALICCCRGR;
S-S DMV Fusion
MAASNGGVMYQSFLTIIILVIMTEGQIHWGNLSKIGIVGTGSASYKVMTRPNHQYLVIKLMPNV
TMIDNCTRTEVTEYRKLLKTVLEPVKNALTVITKNIKPIQSLYPYDVPDYATTSRRSKRFAGVV
LAGVALGVATAAQITAGVALHQSIMNSQSIDNLRTSLEKSNQAIEEIRQASQETVLAVQGVQDF
INNELIPSMHQLSCEMLGQKLGLKLLRYYTEILSIFGPSLRDPISAEISIQALSYALGGDINKV
LEKLGYSGGDLLGILESRGIKARITHVDTESYFIVLSIAYPTLSEIKGVIVHRLEGVSYNIGSQ
EWYTTVPKYVATQGYLISNFDESSCTFMPEGTVCSQNALYPMSPLLQECLRGSTKSCARTLVSG
SFGNRFILSQGNLIANCASILCKCYTTGTIINQDPDKILTYIAADHCPVVEVNGVTIQVGSRRY
PDAVYLHRIDLGPPISLERLDVGTNLGNAIAKLEDAKELLESSDQILRSMKGLSSTSSIVYILI
AVCLGGLIGIPALICCCRGR;
Intermediate DMV Fusion
MAASNGGVMYQSFLTIIILVIMTEGQIHWGNLSKIGIVGTGSASYKVMTRPNHQYLVIKLMPNV
TMIDNCTRTEVTEYRKLLKTVLEPVKNALTVITKNIKPIQSLYPYDVPDYATTSRRSKRFAGVV
LAGVALGVATAAQITAGVALHQSIMNSQSIDNLRTSLEKSNQAIEEIRQASQETVLAVQGVQDF
INNELIPSMHQLSCEMLGQKLGLKLLRYYTEILSIFGPSLRDPVSAEISIQALSYALGGDINKI
LEKLGYSGADLLAILESRGIKAKVTHVDLEGYFIVLSIAYPTLSEVKGVIVHKLEGVSYNIGSQ
EWYTTVPKYVATQGYLISNFDESSCTFMPEGTVCSQNALYPMSPLLQECLRGSTKSCARTLVSG
SFGNRFILSQGNLIANCASILCKCYTTGTIINQDPDKILTYIAADHCPVVEVNGVTIQVGSRRY
PDAVYLHRIDLGPPISLERLDVGTNLGNAIAKLEDAKELLESSDQILRSMKGLSSTSSIVYILI
AVCLGGLIGIPALICCCRGR;
H Interacting Domain DMV Fusion
MAASNGGVMYQSFLTIIILVIMTEGQIHWGNLSKIGIVGTGSASYKVMTRPNHQYLVIKLMPNV
TMIDNCTRTEVTEYRKLLKTVLEPVKNALTVITKNIKPIQSLYPYDVPDYATTSRRSKRFAGVV
LAGVALGVATAAQITAGVALHQSIMNSQSIDNLRTSLEKSNQAIEEIRQASQETVLAVQGVQDE
INNELIPSMHQLSCEMLGQKLGLKLLRYYTEILSIFGPSLRDPVSAEISIQALSYALGGDINKI
LEKLGYSGADLLAILESRGIKAKVTHVDLEGYFIVLSIAYPTLSEVKGVIVHKLEAVSYNLGSQ
EWYTTLPKYVATNGYLISNFDESSCAFMSEVTICSQNALYPMSPLLQQCLRGSTASCARSLVSG
TIGNRFILSKGNLIANCASVLCKCYTTGTIINQDPDKILTYIAADHCPVVEVNGVTIQVGSRRY
PDAVYLHRIDLGPPISLERLDVGTNLGNAIAKLEDAKELLESSDQILRSMKGLSSTSSIVYILI
AVCLGGLIGIPALICCCRGR;
and
Stalk DMV Fusion
MAASNGGVMYQSFLTIIILVIMTEGQIHWGNLSKIGIVGTGSASYKVMTRPNHQYLVIKLMPNV
TMIDNCTRTEVTEYRKLLKTVLEPVKNALTVITKNIKPIQSLYPYDVPDYATTSRRSKRFAGVV
LAGVALGVATAAQITAGVALHQSIMNSQSIDNLRTSLEKSNQAIEEIRQASQETVLAVQGVQDF
INNELIPSMHQLSCEMLGQKLGLKLLRYYTEILSIFGPSLRDPVSAEISIQALSYALGGDINKI
LEKLGYSGADLLAILESRGIKAKVTHVDLEGYFIVLSIAYPTLSEVKGVIVHKLEAVSYNLGSQ
EWYTTLPKYVATNGYLISNFDESSCAFMSEVTICSQNALYPMSPLLQQCLRGSTASCARSLVSG
TIGNRFILSKGNLIANCASVLCKCYSTGTIISQDPDKLLTFVAADKCPLVEVDGITIQVGSREY
PDSVYVSRIDLGPAISLERLDVGTNLGNAIAKLEDAKELLESSDQILRSMKGLSSTSSIVYILI
AVCLGGLIGIPALICCCRGR.

In any of the above aspects, or embodiments thereof, the pseudotyped viral particle is a pseudotyped retroviral viral particle. In embodiments, the pseudotyped retroviral viral particle is a pseudotyped lentiviral viral particle. In embodiments, the pseudotyped retroviral viral particle is a pseudotyped Gammaretrovirus viral particle. In embodiments, the Gammaretrovirus viral particle is a pseudotyped murine leukemia virus particle. In embodiments, the pseudotyped viral particle is self-replicating. In embodiments, the pseudotyped viral particle is not self-replicating. In embodiments, the pseudotyped viral particle is resistant to neutralization by measles-immune human serum.

In any of the above aspects, or embodiments thereof, the target cell is an immune cell. In embodiments, the immune cell is a professional antigen-presenting cell. In embodiments, the target cell is a splenocyte or a thymocyte. In any of the above aspects, or embodiments thereof, the target cell is selected from one or more of a B cell, a dendritic cell, an eosinophil, a granulocyte, an iNKT cell, a macrophage, a monocyte, a natural killer cell, a neutrophil, a lymphoma cell, a regulatory T cell, and a T cell. In any of the above aspects, or embodiments thereof, the target cell is CD4⁺ and/or CD8⁺.

In any of the above aspects, or embodiments thereof, the antigen is selected one or more of BCR/Ig, CD3, CD4, CD7, CD8, CD11, CD19, CD20, CD30, CD34, CD38, CD45, CD133, CD103, CD105, CD110, CD117, CTLA-4, CXCR4, DC-SIGN, EGFR, Emrl, EpCAM, GluA4, Her2/neu, IL3R, IL7R, Mac, MHCII, Mucin 4, NK1.1, P-glycoprotein, TIM3, Thy1, and Thy1.2. In any of the above aspects, or embodiments thereof, the antigen is MHCII or CD7.

In any of the above aspects, or embodiments thereof, the VHH or fragment thereof is derived from a VHH selected from one or more 03F11, 6QRM, aCD8 VHH, aCD11b VHH, Anti-CD3 VHH, DC1, DC1.8, DC2.1, DC8, DC14, DC15, hH6, 281F12, mH2, MU375, MU551, MU1053, R2HCD26, R3HCD27, R3HCD129, VHH4, VHH6, VHH6 Humanized 1, VHH6 Humanized 2, VHH7, VHH10, VHH10 Humanized 1, VHH10 Humanized 2, VHH32, VHH49, VHH51, VHH81, VHHDC13, VHHG7, VHHN11, and VHHV36. In any ofthe above aspects, or embodiments thereof, the VHH or fragment thereof is derived from VHHN11 or VHH10.

In any of the above aspects, or embodiments thereof, the envelope contains a viral fusion polypeptide. In any of the above aspects, or embodiments thereof, the envelope contains a phagocytosis inhibitor. In embodiments, the phagocytosis inhibitor is CD47. In any of the above aspects, or embodiments thereof, the envelope contains a complement regulatory polypeptide. In embodiments, the complement regulatory polypeptide is selected from one or more of CD46, CD55, and CD59.

In any of the above aspects, or embodiments thereof, the viral transfer vector contains a polynucleotide sequence encoding, or the cell membrane further contains, a heterologous polypeptide to be delivered to the target cell. In any of the above aspects, or embodiments thereof, the heterologous polynucleotide encodes or the pseudotyped viral particle further contains a heterologous polypeptide to be delivered to the target cell.

In any of the above aspects, or embodiments thereof, the cell membrane contains the heterologous polypeptide.

In any of the above aspects, or embodiments thereof, the envelope contains the heterologous polypeptide.

In any of the above aspects, or embodiments thereof, the heterologous polypeptide is a chemokine or a cytokine. In any of the above aspects, or embodiments thereof, the heterologous polypeptide is selected from one or more of aCD3, Ccl14, CD28, CD40L, Cxcl10, IL-2, and IL-12. In any of the above aspects, or embodiments thereof, the heterologous polypeptide is a gene-editing polypeptide. In any of the above aspects, or embodiments thereof, the heterologous polypeptide is a cytomegalovirus antigen, a flu virus antigen, or a coronavirus antigen. In embodiments, the coronavirus antigen is a SARS-CoV2 antigen.

In any of the above aspects, or embodiments thereof, the method further involves integrating the heterologous polynucleotide into the genome of the target cell.

In any of the above aspects, or embodiments thereof, the heterologous polynucleotide encodes a chimeric antigen receptor.

In any of the above aspects, or embodiments thereof, the pseudotyped viral particle is administered systemically. In any of the above aspects, or embodiments thereof, the pseudotyped viral particle is administered locally.

In any of the above aspects, or embodiments thereof, the subject is measles-immune. In any of the above aspects, or embodiments thereof, the target cell is a mammalian cell. In any of the above aspects, or embodiments thereof, the target cell is a human cell. In any of the above aspects, or embodiments thereof, the subject is a mammal. In any of the above aspects, or embodiments thereof, the subject is a human.

In any of the above aspects, or embodiments thereof, the cancer is a leukemia or a lymphoma.

In any of the above aspects, or embodiments thereof, the pseudotyped viral particle is a pseudotyped retroviral viral particle and/or the viral transfer vector is a retroviral transfer vector. In embodiments, the pseudotyped retroviral viral particle is a pseudotyped lentiviral viral particle and/or the viral transfer vector is lentiviral transfer vector. In embodiments, the pseudotyped retroviral viral particle is a pseudotyped Gammaretrovirus viral particle and/or the viral transfer vector is a Gammaretrovirus transfer vector. In embodiments, the Gammaretrovirus viral particle is a pseudotyped murine leukemia virus particle and/or the Gammaretrovirus transfer vector is a murine leukemia virus transfer vector.

In any of the above aspects, or embodiments thereof, eukaryotic cell is selected from one or more of a 293T cell, a pan T cell, a Jurkat T cell, a primary human T cell, a SupTI cell, a CHO cell, a HepG2 cell, an MCF-7 cell, and an MEF cell.

In any of the above aspects, or embodiments thereof, expression of the polypeptide is under the control of a promoter.

In any of the above aspects, or embodiments thereof, the expression vector further contains a polynucleotide encoding viral envelope glycoprotein polypeptide containing an amino acid sequence with at least about 85% sequence identity to one or more of the following sequences, or to one or more of the following sequences containing a truncated cytoplasmic domain:

MeV-Fc30
MGLKVNVSAIFMAVLLTLQTPTGQIHWGNLSKIGVVGIGSASYKVMTRSSHQSLVIKLMPNITL
LNNCTRVEIAEYRRLLRTVLEPIRDALNAMTQNIRPVQSVASSRRHKRFAGVVLAGAALGVATA
AQITAGIALHQSMLNSQAIDNLRASLETTNQAIEAIRQAGQEMILAVQGVQDYINNELIPSMNQ
LSCDLIGQKLGLKLLRYYTEILSLFGPSLRDPISAEISIQALSYALGGDINKVLEKLGYSGGDL
LGILESRGIKARITHVDTESYFIVLSIAYPTLSEIKGVIVHRLEGVSYNIGSQEWYTTVPKYVA
TQGYLISNFDESSCTFMPEGTVCSQNALYPMSPLLQECLRGSTKSCARTLVSGSFGNRFILSQG
NLIANCASILCKCYTTGTIINQDPDKILTYIAADHCPVVEVNGVTIQVGSRRYPDAVYLHRIDL
GPPISLERLDVGTNLGNAIAKLEDAKELLESSDQILRSMKGLSSTSIVYILIAVCLGGLIGIPA
LICCCRGR;
DMV-F
MAASNGGVMYQSFLTIIILVIMTEGQIHWGNLSKIGIVGTGSASYKVMTRPNHQYLVIKLMPNV
TMIDNCTRTEVTEYRKLLKTVLEPVKNALTVITKNIKPIQSLTTSRRSKRFAGVVLAGVALGVA
TAAQITAGVALHQSIMNSQSIDNLRTSLEKSNQAIEEIRQASQETVLAVQGVQDFINNELIPSM
HQLSCEMLGQKLGLKLLRYYTEILSIFGPSLRDPVSAEISIQALSYALGGDINKILEKLGYSGA
DLLAILESRGIKAKVTHVDLEGYFIVLSIAYPTLSEVKGVIVHKLEAVSYNLGSQEWYTTLPKY
VATNGYLISNFDESSCAFMSEVTICSQNALYPMSPLLQQCLRGSTASCARSLVSGTIGNRFILS
KGNLIANCASVLCKCYSTGTIISQDPDKLLTFVAADKCPLVEVDGITIQVGSREYPDSVYVSRI
DLGPAISLEKLDVGTNLGSALTKLDNAKDLLDSSNQILENVRRSSFGGAMYIGILVCAGALVIL
CVLVYCCRRHCRKRVQTPPKATPGLKPDLTGTTKSYVRSL;
CDV-F
MHRGIPKSSKTQTHTQQDRPPQPSTELEETRTSRARHSTTSAQRSTHYDPRTSDRPVSYTMNRT
RSRKQTSHRLKNIPVHGNHEATIQHIPESVSKGARSQIERRQPNAINSGSHCTWLVLWCLGMAS
LFLCSKAQIHWDNLSTIGIIGTDNVHYKIMTRPSHQYLVIKLIPNASLIENCTKAELGEYEKLL
NSVLEPINQALTLMTKNVKPLQSLGSGRRQRRFAGVVLAGVALGVATAAQITAGIALHQSNLNA
QAIQSLRTSLEQSNKAIEEIREATQETVIAVQGVQDYVNNELVPAMQHMSCELVGQRLGLRLLR
YYTELLSIFGPSLRDPISAEISIQALIYALGGEIHKILEKLGYSGSDMIAILESRGIKTKITHV
DLPGKFIILSISYPTLSEVKGVIVHRLEAVSYNIGSQEWYTTVPRYIATNGYLISNFDESSCVF
VSESAICSQNSLYPMSPLLQQCIRGDTSSCARTLVSGTMGNKFILSKGNIVANCASILCKCYST
STIINQSPDKLLTFIASDTCPLVEIDGATIQVGGRQYPDMVYEGKVALGPAISLDRLDVGTNLG
NALKKLDDAKVLIDSSNQILETVRRSSFNFGSLLSVPILSCTALALLLLIYCCKRRYQQTLKQH
TKVDPAFKPDLTGTSKSYVRSL;
F2 DMV Fusion
MAASNGGVMYQSFLTIIILVIMTEGQIHWGNLSKIGIVGTGSASYKVMTRPNHQYLVIKLMPNV
TMIDNCTRTEVTEYRKLLKTVLEPVKNALTVITKNIKPIQSLYPYDVPDYATTSRRSKRFAGVV
LAGAALGVATAAQITAGIALHQSMLNSQAIDNLRASLETTNQAIEAIRQAGQEMILAVQGVQDY
INNELIPSMNQLSCDLIGQKLGLKLLRYYTEILSLFGPSLRDPISAEISIQALSYALGGDINKV
LEKLGYSGGDLLGILESRGIKARITHVDTESYFIVLSIAYPTLSEIKGVIVHRLEGVSYNIGSQ
EWYTTVPKYVATQGYLISNFDESSCTFMPEGTVCSQNALYPMSPLLQECLRGSTKSCARTLVSG
SFGNRFILSQGNLIANCASILCKCYTTGTIINQDPDKILTYIAADHCPVVEVNGVTIQVGSRRY
PDAVYLHRIDLGPPISLERLDVGTNLGNAIAKLEDAKELLESSDQILRSMKGLSSTSIVYILIA
VCLGGLIGIPALICCCRGR;
S-S DMV Fusion
MAASNGGVMYQSFLTIIILVIMTEGQIHWGNLSKIGIVGTGSASYKVMTRPNHQYLVIKLMPNV
TMIDNCTRTEVTEYRKLLKTVLEPVKNALTVITKNIKPIQSLYPYDVPDYATTSRRSKRFAGVV
LAGVALGVATAAQITAGVALHQSIMNSQSIDNLRTSLEKSNQAIEEIRQASQETVLAVQGVQDF
INNELIPSMHQLSCEMLGQKLGLKLLRYYTEILSIFGPSLRDPISAEISIQALSYALGGDINKV
LEKLGYSGGDLLGILESRGIKARITHVDTESYFIVLSIAYPTLSEIKGVIVHRLEGVSYNIGSQ
EWYTTVPKYVATQGYLISNFDESSCTFMPEGTVCSQNALYPMSPLLQECLRGSTKSCARTLVSG
SFGNRFILSQGNLIANCASILCKCYTTGTIINQDPDKILTYIAADHCPVVEVNGVTIQVGSRRY
PDAVYLHRIDLGPPISLERLDVGTNLGNAIAKLEDAKELLESSDQILRSMKGLSSTSSIVYILI
AVCLGGLIGIPALICCCRGR;
Intermediate DMV Fusion
MAASNGGVMYQSFLTIIILVIMTEGQIHWGNLSKIGIVGTGSASYKVMTRPNHQYLVIKLMPNV
TMIDNCTRTEVTEYRKLLKTVLEPVKNALTVITKNIKPIQSLYPYDVPDYATTSRRSKRFAGVV
LAGVALGVATAAQITAGVALHQSIMNSQSIDNLRTSLEKSNQAIEEIRQASQETVLAVQGVQDF
INNELIPSMHQLSCEMLGQKLGLKLLRYYTEILSIFGPSLRDPVSAEISIQALSYALGGDINKI
LEKLGYSGADLLAILESRGIKAKVTHVDLEGYFIVLSIAYPTLSEVKGVIVHKLEGVSYNIGSQ
EWYTTVPKYVATQGYLISNFDESSCTFMPEGTVCSQNALYPMSPLLQECLRGSTKSCARTLVSG
SFGNRFILSQGNLIANCASILCKCYTTGTIINQDPDKILTYIAADHCPVVEVNGVTIQVGSRRY
PDAVYLHRIDLGPPISLERLDVGTNLGNAIAKLEDAKELLESSDQILRSMKGLSSTSSIVYILI
AVCLGGLIGIPALICCCRGR;
H Interacting Domain DMV Fusion
MAASNGGVMYQSFLTIIILVIMTEGQIHWGNLSKIGIVGTGSASYKVMTRPNHQYLVIKLMPNV
TMIDNCTRTEVTEYRKLLKTVLEPVKNALTVITKNIKPIQSLYPYDVPDYATTSRRSKRFAGVV
LAGVALGVATAAQITAGVALHQSIMNSQSIDNLRTSLEKSNQAIEEIRQASQETVLAVQGVQDF
INNELIPSMHQLSCEMLGQKLGLKLLRYYTEILSIFGPSLRDPVSAEISIQALSYALGGDINKI
LEKLGYSGADLLAILESRGIKAKVTHVDLEGYFIVLSIAYPTLSEVKGVIVHKLEAVSYNLGSQ
EWYTTLPKYVATNGYLISNFDESSCAFMSEVTICSQNALYPMSPLLQQCLRGSTASCARSLVSG
TIGNRFILSKGNLIANCASVLCKCYTTGTIINQDPDKILTYIAADHCPVVEVNGVTIQVGSRRY
PDAVYLHRIDLGPPISLERLDVGTNLGNAIAKLEDAKELLESSDQILRSMKGLSSTSSIVYILI
AVCLGGLIGIPALICCCRGR;
and
Stalk DMV Fusion
MAASNGGVMYQSFLTIIILVIMTEGQIHWGNLSKIGIVGTGSASYKVMTRPNHQYLVIKLMPNV
TMIDNCTRTEVTEYRKLLKTVLEPVKNALTVITKNIKPIQSLYPYDVPDYATTSRRSKRFAGVV
LAGVALGVATAAQITAGVALHQSIMNSQSIDNLRTSLEKSNQAIEEIRQASQETVLAVQGVQDF
INNELIPSMHQLSCEMLGQKLGLKLLRYYTEILSIFGPSLRDPVSAEISIQALSYALGGDINKI
LEKLGYSGADLLAILESRGIKAKVTHVDLEGYFIVLSIAYPTLSEVKGVIVHKLEAVSYNLGSQ
EWYTTLPKYVATNGYLISNFDESSCAFMSEVTICSQNALYPMSPLLQQCLRGSTASCARSLVSG
TIGNRFILSKGNLIANCASVLCKCYSTGTIISQDPDKLLTFVAADKCPLVEVDGITIQVGSREY
PDSVYVSRIDLGPAISLERLDVGTNLGNAIAKLEDAKELLESSDQILRSMKGLSSTSSIVYILI
AVCLGGLIGIPALICCCRGR.

The invention provides pseudotyped viral particles (e.g., lentiviral or gammaretroviral particles) and compositions and methods of use thereof, where the viral particles contain a VHH domain. Compositions and articles defined by the invention were isolated or otherwise manufactured in connection with the examples provided below. Other features and advantages of the invention will be apparent from the detailed description, and from the claims.

In any of the above aspects, or embodiments thereof, the chimeric viral envelope glycoprotein polypeptide contains the amino acid sequence RLDVGTNLGNAIAKLEDAKELLESSDQILRSMKGLSSTS (MeV-F extravirion stalk domain). In any of the above aspects, or embodiments thereof, the non-measles virus morbillivirus is selected from one or more of canine distemper virus, dolphin Morbillivirus, feline Morbillivirus, measles virus, Peste des petits ruminant virus, phocine Morbillivirus, Rinderpest virus, and small ruminant virus. In embodiments, the non-measles virus morbillivirus is dolphin Morbillivirus. In any of the above aspects, or embodiments thereof, the comeric viral envelope glycoprotein polypeptide contains at least about 80 amino acids derived from the non-measles virus morbillivirus F protein or H protein extravirion domain. In any of the above aspects, or embodiments thereof, the chimeric protein contains a sequence with at least 85% sequence identity to a sequence selected from one or more of

F2 DMV Fusion
MAASNGGVMYQSFLTIIILVIMTEGQIHWGNLSKIGIVGTGSASYKVMTRPNHQYLVIKLMPNV
TMIDNCTRTEVTEYRKLLKTVLEPVKNALTVITKNIKPIQSLYPYDVPDYATTSRRSKRFAGVV
LAGAALGVATAAQITAGIALHQSMLNSQAIDNLRASLETTNQAIEAIRQAGQEMILAVQGVQDY
INNELIPSMNQLSCDLIGQKLGLKLLRYYTEILSLFGPSLRDPISAEISIQALSYALGGDINKV
LEKLGYSGGDLLGILESRGIKARITHVDTESYFIVLSIAYPTLSEIKGVIVHRLEGVSYNIGSQ
EWYTTVPKYVATQGYLISNFDESSCTFMPEGTVCSQNALYPMSPLLQECLRGSTKSCARTLVSG
SFGNRFILSQGNLIANCASILCKCYTTGTIINQDPDKILTYIAADHCPVVEVNGVTIQVGSRRY
PDAVYLHRIDLGPPISLERLDVGTNLGNAIAKLEDAKELLESSDQILRSMKGLSSTSIVYILIA
VCLGGLIGIPALICCCRGR;
S-S DMV Fusion
MAASNGGVMYQSFLTIIILVIMTEGQIHWGNLSKIGIVGTGSASYKVMTRPNHQYLVIKLMPNV
TMIDNCTRTEVTEYRKLLKTVLEPVKNALTVITKNIKPIQSLYPYDVPDYATTSRRSKRFAGVV
LAGVALGVATAAQITAGVALHQSIMNSQSIDNLRTSLEKSNQAIEEIRQASQETVLAVQGVQDF
INNELIPSMHQLSCEMLGQKLGLKLLRYYTEILSIFGPSLRDPISAEISIQALSYALGGDINKV
LEKLGYSGGDLLGILESRGIKARITHVDTESYFIVLSIAYPTLSEIKGVIVHRLEGVSYNIGSQ
EWYTTVPKYVATQGYLISNFDESSCTFMPEGTVCSQNALYPMSPLLQECLRGSTKSCARTLVSG
SFGNRFILSQGNLIANCASILCKCYTTGTIINQDPDKILTYIAADHCPVVEVNGVTIQVGSRRY
PDAVYLHRIDLGPPISLERLDVGTNLGNAIAKLEDAKELLESSDQILRSMKGLSSTSSIVYILI
AVCLGGLIGIPALICCCRGR;
Intermediate DMV Fusion
MAASNGGVMYQSFLTIIILVIMTEGQIHWGNLSKIGIVGTGSASYKVMTRPNHQYLVIKLMPNV
TMIDNCTRTEVTEYRKLLKTVLEPVKNALTVITKNIKPIQSLYPYDVPDYATTSRRSKRFAGVV
LAGVALGVATAAQITAGVALHQSIMNSQSIDNLRTSLEKSNQAIEEIRQASQETVLAVQGVQDF
INNELIPSMHQLSCEMLGQKLGLKLLRYYTEILSIFGPSLRDPVSAEISIQALSYALGGDINKI
LEKLGYSGADLLAILESRGIKAKVTHVDLEGYFIVLSIAYPTLSEVKGVIVHKLEGVSYNIGSQ
EWYTTVPKYVATQGYLISNFDESSCTFMPEGTVCSQNALYPMSPLLQECLRGSTKSCARTLVSG
SFGNRFILSQGNLIANCASILCKCYTTGTIINQDPDKILTYIAADHCPVVEVNGVTIQVGSRRY
PDAVYLHRIDLGPPISLERLDVGTNLGNAIAKLEDAKELLESSDQILRSMKGLSSTSSIVYILI
AVCLGGLIGIPALICCCRGR;
H Interacting Domain DMV Fusion
MAASNGGVMYQSFLTIIILVIMTEGQIHWGNLSKIGIVGTGSASYKVMTRPNHQYLVIKLMPNV
TMIDNCTRTEVTEYRKLLKTVLEPVKNALTVITKNIKPIQSLYPYDVPDYATTSRRSKRFAGVV
LAGVALGVATAAQITAGVALHQSIMNSQSIDNLRTSLEKSNQAIEEIRQASQETVLAVQGVQDF
INNELIPSMHQLSCEMLGQKLGLKLLRYYTEILSIFGPSLRDPVSAEISIQALSYALGGDINKI
LEKLGYSGADLLAILESRGIKAKVTHVDLEGYFIVLSIAYPTLSEVKGVIVHKLEAVSYNLGSQ
EWYTTLPKYVATNGYLISNFDESSCAFMSEVTICSQNALYPMSPLLQQCLRGSTASCARSLVSG
TIGNRFILSKGNLIANCASVLCKCYTTGTIINQDPDKILTYIAADHCPVVEVNGVTIQVGSRRY
PDAVYLHRIDLGPPISLERLDVGTNLGNAIAKLEDAKELLESSDQILRSMKGLSSTSSIVYILI
AVCLGGLIGIPALICCCRGR;
and
Stalk DMV Fusion
MAASNGGVMYQSFLTIIILVIMTEGQIHWGNLSKIGIVGTGSASYKVMTRPNHQYLVIKLMPNV
TMIDNCTRTEVTEYRKLLKTVLEPVKNALTVITKNIKPIQSLYPYDVPDYATTSRRSKRFAGVV
LAGVALGVATAAQITAGVALHQSIMNSQSIDNLRTSLEKSNQAIEEIRQASQETVLAVQGVQDF
INNELIPSMHQLSCEMLGQKLGLKLLRYYTEILSIFGPSLRDPVSAEISIQALSYALGGDINKI
LEKLGYSGADLLAILESRGIKAKVTHVDLEGYFIVLSIAYPTLSEVKGVIVHKLEAVSYNLGSQ
EWYTTLPKYVATNGYLISNFDESSCAFMSEVTICSQNALYPMSPLLQQCLRGSTASCARSLVSG
TIGNRFILSKGNLIANCASVLCKCYSTGTIISQDPDKLLTFVAADKCPLVEVDGITIQVGSREY
PDSVYVSRIDLGPAISLERLDVGTNLGNAIAKLEDAKELLESSDQILRSMKGLSSTSSIVYILI
AVCLGGLIGIPALICCCRGR.

In any of the above aspects, or embodiments thereof, the pseudotyped viral particle resists neutralization by a measles virus neutralizing antibody relative to a reference viral particle pseudotyped with a glycoprotein polypeptide containing a measles virus F protein (MeV-Fc) extravirion domain.

In any of the above aspects, or embodiments thereof, the membrane of the eukaryotic cell displays an anti-cluster of differentiation 3 (CD3) polypeptide and a cluster of differentiation 80 (CD80) polypeptide. In any of the above aspects, or embodiments thereof, the envelope further comprises an anti-CD3 polypeptide and a CD80 polypeptide.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “viral envelope glycoprotein domain” or “glycoprotein domain” is meant a domain that binds a receptor site on the surface of a target cell and/or mediates insertion into a target cell. In embodiments, the viral envelope glycoprotein domain or fragment thereof is fused to a VHH domain or fragment thereof. Exemplary glycoprotein domains include MeV-Hwtc18, CDV-F, CDF-Fc30, DMV-F, DMV-Fc30, DMV-H, DMV-Hc18 (MeV-Hc18-DMV), CDV-H, CDV-Hc18 (MeV-Hc18-CDV), FMV-H, PPRV-H, RPV-H, RMV-H, DMV1-123-MeV122-529 (F2), DMV1-123-MeV122-529 (S-S), DMV1-311-MeV309-529 (Intermediate), DMV1-407-MeV405-529 (H interacting domain), and DMV1-465-MeV463-529 (stalk).

By “VHH domain” is meant an antigen binding domain of a heavy chain only antibody or an antigen binding fragment thereof. Exemplary VHH domains and their respective targets are provided at Table 1.

TABLE 1
Exemplary VHH domains and their respective targets.

| Target | VHH domains |
|---|---|
| CD8a (subset of T cells) | aCD8 VHH, R2HCD26, |
| | R3HCD27, R3HCD129 |
| CD11b (Monocyte) | 51, 81, V36, VHHDC13, |
| | aCD11b VHH |
| CD45 (immune cells) | 32, G7 |
| MHCII | VHH7, 49, N11, DC1, DC8, |
| | DC14, DC15, VHH4 |
| TIM3 | mH2 |
| Dendritic cell | DC1.8, DC2.1 (antigen unknown) |
| CD7 | VHH6, VHH10, VHH6 |
| | Humanized 1, VHH10 |
| | Humanized 1, VHH6 |
| | Humanized 2, VHH10 |
| | Humanized 2 |
| CD3 | Anti-CD3 VHH |
| CD4 | 03F11 |
| CD38 | MU375, MU551, MU1053 |
| TIM3 | hH6 |
| CXCR4 | 281F12 |
| CTLA-4 | 6RQM |

By “anti-cluster of differentiation 3 (CD3) scFv polypeptide” is meant a polypeptide having at least about 85% amino acid sequence identity to the below CD3 scFv polypeptide sequence or comprising VH and/or VL CDRs1-3 of the CD3 scFv polypeptide or antigen binding fragments thereof, wherein each of the scFv, CDRs, and antigen binding fragments specifically bind to a CD3 polypeptide. In embodiments, the scFv or antigen binding fragment thereof has at least 90%, 93%, 95%, 98%, 99% or 100% amino acid sequence identity to the below scFv polypeptide sequence, or a functional fragment thereof.

An exemplary scFv polypeptide sequence is provided below, where a linker is indicated in bold:

QVQLQQSGAELARPGASVKMSCKASGYTFTRYTMHWVKQRPGQGLEWIG
YINPSRGYTNYNQKFKDKATLTTDKSSSTAYMQLSSLTSEDSAVYYCAR
YYDDHYCLDYWGQGTTLTVSSGGGGSGGGGSGGGGSDIVLTQSPAIMSA
SPGEKVTMTCSASSSVSYMNWYQQKSGTSPKRWIYDTSKLASGVPAHFR
GSGSGTSYSLTISGMEAEDAATYYCQQWSSNPFTFGSGTKLEINRGS

A further exemplary scFv polypeptide is provided below, where the scFv polypeptide contains a signal peptide and a polypeptide sequence containing a transmembrane domain, where the signal peptide is in bold italics, and the polypeptide sequence containing a transmembrane domain is underlined:

aCD3 scFv (QKT3 clone)
MGWSCIILFLVATATGVHSQVQLQQSGAELARPGASVKMSCKASGYTFT
RYTMHWVKQRPGQGLEWIGYINPSRGYTNYNQKFKDKATLTTDKSSSTA
YMQLSSLTSEDSAVYYCARYYDDHYCLDYWGQGTTLTVSSGGGGSGGGG
SGGGGSDIVLTQSPAIMSASPGEKVTMTCSASSSVSYMNWYQQKSGTSP
KRWIYDTSKLASGVPAHFRGSGSGTSYSLTISGMEAEDAATYYCQQWSS
NPFTFGSGTKLEINRGSEQKLISEEDLNAVGQDTQEVIVVPHSLPFKVV
VISAILALVVLTIISLIILIMLWQKKPRGSIPNPLLGLD.

Exemplary variable region sequences for an anti-CD3 polypeptide are provided below:

Anti-CD3 scFv heavy chain variable region (VH)
QVQLQQSGAELARPGASVKMSCKASGYTFTRYTMHWVKQRPGQGLEWIG
YINPSRGYTNYNQKFKDKATLTTDKSSSTAYMQLSSLTSEDSAVYYCAR
YYDDHYCLDYWGQGTTLTV
Anti-CD3 scFv light chain variable region (VL)
DIVLTQSPAIMSASPGEKVTMTCSASSSVSYMNWYQQKSGTSPKRWIYD
TSKLASGVPAHFRGSGSGTSYSLTISGMEAEDAATYYCQQWSSNPFTFG
SGTKLEINRGSEQKLISEEDLNAVGQDTQEVIVVPHSLPFKVV

The three complementarity determining regions (CDRs), i.e., CDR1, CDR2 and CDR3, are underlined in the anti-CD3 scFv VH and VL region sequences shown supra. In particular, the three CDRs of the anti-CD3 scFv VH region are as follows:

VH CDR1:
RYTMH
VH CDR2:
YINPSRGYTNYNQKFKD
VH CDR3:
YYDDHYCLDY

The three CDRs of the anti-CD3 scFv antibody VL region are as follows:

VL CDR1:
SASSSVSYMN
VL CDR2:
DTSKLAS
VL CDR3:
QQWSSNPFT

The four framework (FR) regions, i.e., FR1, FR2, FR3, and FR4, of the anti-CD3 scFv are located on either side of each of the CDRs in VH and VL region sequences shown supra, In particular, the four FRs of the anti-CD3 scFv VH region are as follows:

VH FR1:
QVQLQQSGAELARPGASVKMSCKASGYTFT
VH FR2:
WVKQRPGQGLEWIG
VH FR3:
KATLTTDKSSSTAYMQLSSLTSEDSAVYYCAR
VH FR4:
WGQGTTLTV

The four FRs of the anti-CD3 scFv VL region are as follows:

VL FR1:
DIVLTQSPAIMSASPGEKVTMTC
VL FR2:
WYQQKSGTSPKRWIY
VL FR3:
GVPAHFRGSGSGTSYSLTISGMEAEDAATYYC
VL FR4:
FGSGTKLEINRGS

By “anti-cluster of differentiation 3 (CD3) scFv polynucleotide” is meant a polynucleotide encoding an anti-CD3 scFv polypeptide. An exemplary anti-CD3 scFv polynucleotide is provided below.

ATGGGATGGTCATGTATCATACTGTTCCTGGTGGCGACAGCTACCGGCGTGCATAGCCAAGTGC
AGCTCCAGCAGTCCGGCGCCGAGCTTGCTCGCCCGGGCGCAAGTGTAAAGATGTCCTGCAAGGC
TAGCGGCTACACTTTCACCCGTTATACCATGCACTGGGTGAAACAGCGACCTGGTCAGGGTTTA
GAGTGGATCGGCTACATAAACCCCAGCCGGGGGTACACTAATTATAATCAAAAATTTAAAGACA
AGGCCACACTGACGACAGATAAGTCCTCATCTACCGCCTATATGCAGCTTAGCTCTCTTACCTC
CGAAGACAGCGCTGTGTATTACTGCGCAAGGTACTATGATGACCATTACTGTCTAGACTACTGG
GGGCAAGGTACCACACTGACAGTGAGCTCTGGAGGCGGGGGGAGTGGAGGTGGGGGCAGCGGCG
GTGGCGGAAGTGATATCGTTCTGACCCAGAGCCCAGCTATTATGTCTGCCTCTCCCGGCGAGAA
GGTGACTATGACGTGTTCCGCATCATCCAGCGTTAGTTACATGAATTGGTATCAGCAAAAGAGT
GGGACCTCTCCTAAGCGGTGGATCTACGATACGTCAAAGCTCGCCTCCGGCGTACCAGCGCACT
TCAGAGGGTCGGGTTCCGGGACAAGCTATAGTCTGACTATTTCAGGGATGGAAGCCGAAGATGC
CGCAACCTATTACTGCCAGCAGTGGTCCTCAAACCCATTTACTTTCGGAAGCGGAACAAAACTC
GAAATCAACAGGGGATCCGAGCAGAAACTGATTTCTGAGGAAGACCTGAACGCTGTCGGCCAAG
ACACTCAGGAGGTCATAGTTGTCCCCCACTCTCTCCCTTTTAAAGTGGTGGTCATTTCGGCCAT
CCTGGCACTGGTCGTTTTGACTATTATTAGTCTGATCATTTTGATCATGTTGTGGCAGAAGAAA
CCAAGAGGATCAATCCCCAATCCTTTACTCGGACTAGAT.

By “anti-major histocompatibility complex II (MHCII) VHH (N11) polypeptide” is meant a polypeptide or fragment thereof with at least about 85% amino acid sequence identity to the sequence provided below, and that is capable of binding a MHCII polypeptide.

QVQLVQSGGGLVQPGGSLGLSCAASGNIGSRDNMGWYRQAPGKQREWVA
TISGYGIATYRDSVKGRFTVAKDTAKNIVSLQMNYLTTEDTAVYYCYAY
AVDSRNIFWSQGTQVTVS.

By “anti-major histocompatibility II (MHCII) VHH (N11) polynucleotide” is meant a polynucleotide encoding an anti-major histocompatibility complex II (MHCII) VHH (N11) polypeptide. An exemplary anti-MHCII VHH polynucleotide is provided below.

CAGGTTCAACTGGTGCAGAGCGGAGGCGGCCTGGTCCAGCCCGGCGGAA
GCCTGGGCCTGAGCTGTGCCGCTTCTGGCAACATCGGCAGCAGAGATAA
TATGGGCTGGTATAGACAGGCCCCTGGAAAACAGAGAGAGTGGGTGGCT
ACAATCAGCGGCTACGGCATCGCCACATACCGGGACAGCGTGAAGGGCA
GATTCACCGTGGCCAAGGACACCGCCAAGAACATCGTGTCCCTGCAGAT
GAACTACCTGACCACCGAAGATACAGCCGTGTACTACTGCTACGCCTAC
GCCGTGGACAGCCGGAACATCTTCTGGTCCCAGGGCACCCAGGTGACCG
TGTCT.

By “anti-CD45 (32) VHH polypeptide” is meant a polypeptide or fragment thereof with at least about 85% amino acid sequence identity to the sequence provided below, and that is capable of binding a CD45 polypeptide.

QVQLVQSGGGLVQPGGSLRLSCAASGRAFNSAAMGWYRQAPGSQRELVA
SISAGTASYADAVKGRFTISRDYAKNIIYLQMNSLKPDDTAVYFCNYRT
TYTSGYSEDYWGQGTQVTVS.

By “anti-CD45 (32) VHH polynucleotide” is meant a polynucleotide encoding an anti-CD45 (32) VHH polypeptide. An exemplary anti-CD45 VHH polynucleotide is provided below.

CAGGTGCAGCTGGTGCAGAGCGGCGGAGGACTGGTGCAACCTGGAGGCA
GCCTGAGACTGAGCTGCGCCGCTAGCGGCAGAGCCTTTAACAGCGCCGC
CATGGGCTGGTACAGACAGGCCCCTGGCTCTCAGAGAGAGCTGGTCGCC
TCTATCTCCGCCGGCACCGCCTCTTATGCTGATGCCGTGAAAGGCAGAT
TCACCATCAGCCGGGACTACGCCAAGAACATCATCTACCTGCAGATGAA
CAGCCTGAAGCCCGACGACACAGCTGTGTACTTCTGTAATTACCGGACA
ACCTACACCAGCGGCTACAGCGAAGATTACTGGGGCCAGGGCACACAGG
TGACCGTGTCC.

By “anti-CD7 (VHH10) VHH polypeptide” is meant a polypeptide or fragment thereof with at least about 85% amino acid sequence identity to the sequence provided below, and that is capable of binding a CD7 polypeptide.

DVQLQESGGGSVQAGGSLRLSCAASGYTHSSYCMAWFRQAPGREREGVA
SIDSDGTTSYADSVKGRFTISQDNAKNTLYLQMNSLKPEDTAMYYCAAR
FGPMGCVDLSTLSFGHWGQGTQVTVSIT.

By “anti-CD7 (VHH10) VHH polynucleotide” is meant a polynucleotide encoding an anti-CD7 (VHH10) VHH polypeptide. An exemplary anti-CD7 VHH polynucleotide is provided below.

GATGTGCAGCTGCAGGAGAGCGGCGGCGGATCTGTGCAAGCTGGCGGAA
GCCTGAGACTGTCTTGCGCCGCTTCTGGCTACACCCACAGCAGCTATTG
CATGGCCTGGTTCCGGCAGGCCCCTGGCAGAGAACGGGAAGGCGTGGCC
AGCATCGACAGCGACGGCACCACCAGCTACGCCGATAGCGTCAAGGGCA
GATTCACCATCAGCCAGGACAATGCCAAAAACACCCTGTACCTGCAGAT
GAACAGCCTGAAGCCCGAGGACACAGCCATGTACTACTGTGCCGCCAGA
TTTGGCCCTATGGGCTGCGTGGACCTGTCCACCCTCTCCTTCGGCCACT
GGGGACAGGGCACACAGGTGACAGTGTCCATCACC.

By “anti-CD4 (03F11) VHH polypeptide” is meant a polypeptide or fragment thereof with at least about 85% amino acid sequence identity to the sequence provided below, and that is capable of binding a CD4 polypeptide.

EVQLVESGGGSVQPGGSLTLSCGTSGRTFNVMGWFRQAPGKEREFVAAV
RWSSTGIYYTQYADSVKSRFTISRDNAKNTVYLEMNSLKPEDTAVYYCA
ADTYNSNPARWDGYDFRGQGTQVTVSS.

By “anti-CD4 (03F11) VHH polynucleotide” is meant a polynucleotide encoding an anti-CD4 (03F11) VHH polypeptide. An exemplary anti-CD4 VHH polynucleotide is provided below.

GAGGTGCAGCTGGTCGAGAGCGGAGGAGGCAGCGTGCAGCCCGGCGGAT
CTCTGACCCTGAGCTGTGGCACAAGCGGCAGAACCTTTAACGTGATGGG
CTGGTTCAGACAGGCCCCTGGCAAGGAAAGAGAGTTCGTGGCCGCTGTG
CGGTGGTCCTCTACAGGCATCTACTATACACAGTACGCCGACAGCGTTA
AAAGCAGATTCACCATCAGCCGGGACAACGCCAAGAACACCGTGTACCT
GGAAATGAACAGCCTGAAGCCAGAGGATACAGCCGTGTACTACTGCGCC
GCCGATACCTACAACTCTAATCCTGCTAGATGGGACGGCTACGACTTCC
GGGGCCAGGGCACCCAAGTGACCGTGTCCAGC.

By “anti-CD8 (R3HCD27) VHH polypeptide” is meant a polypeptide or fragment thereof with at least about 85% amino acid sequence identity to the sequence provided below, and that is capable of binding a CD8 polypeptide.

QVQLQESGGGSVQPGGSLRLSCAASGFTFDDYAMSWVRQVPGKGLEWVS
TINWNGGSAEYAEPVKGRFTISRDNAKNTVYLQMNSLKLEDTAVYYCAK
DADLVWYNLSTGQGTQVTVSS.

By “anti-CD8 (R3HCD27) VHH polynucleotide” is meant a polynucleotide encoding an anti-CD8 (R3HCD27) VHH polypeptide. An exemplary anti-CD8 VHH polynucleotide is provided below.

CAAGTGCAGCTGCAGGAGAGCGGCGGAGGCAGCGTGCAGCCTGGCGGAA
GCCTGAGACTGTCCTGCGCCGCTTCTGGCTTTACCTTCGACGACTACGC
CATGAGCTGGGTGCGGCAGGTTCCAGGCAAGGGCCTGGAATGGGTCAGC
ACAATCAACTGGAACGGCGGATCTGCTGAGTACGCCGAGCCTGTGAAGG
GCAGATTCACAATCAGCAGAGATAATGCCAAAAACACCGTGTATCTGCA
GATGAACAGCCTCAAGCTGGAAGATACAGCCGTGTACTACTGTGCCAAG
GACGCCGACCTGGTGTGGTACAACCTGAGCACCGGCCAGGGCACCCAGG
TGACCGTGTCCAGCGCCGCTGCCTACCCCTACGACGTGCCTGATTACGG
CTCT.

By “human cluster of differentiation 80 (hCD80; CD80) polypeptide” is meant a polypeptide or fragment thereof with at least about 85% amino acid sequence identity to NCBI Ref. Seq. Accession No. NP_005182.1, provided below, and that has immunomodulatory activity.

>NP_005182.1 T-lymphocyte activation antigen CD80
precursor [Homo sapiens] (hCD80)
MGHTRRQGTSPSKCPYLNFFQLLVLAGLSHFCSGVIHVTKEVKEVATLS
CGHNVSVEELAQTRIYWQKEKKMVLTMMSGDMNIWPEYKNRTIFDITNN
LSIVILALRPSDEGTYECVVLKYEKDAFKREHLAEVTLSVKADFPTPSI
SDFEIPTSNIRRIICSTSGGFPEPHLSWLENGEELNAINTTVSQDPETE
LYAVSSKLDFNMTTNHSFMCLIKYGHLRVNQTFNWNTTKQEHFPDNLLP
SWAITLISVNGIFVICCLTYCFAPRCRERRRNERLRRESVRPV.

By “human cluster of differentiation 80 (hCD80; CD80) polynucleotide” is meant a polynucleotide encoding an hCD80 polypeptide. An exemplary hCD80 polynucleotide is provided below. A further exemplary hCD80 polynucleotide sequence is provided at NCBI Ref. Seq. Accession No. NM_005191.4, which is provided below.

ATGGGCCACACGCGGCGGCAGGGCACCAGCCCTTCCAAGTGTCCATATT
TGAACTTCTTCCAGCTTTTAGTGCTCGCTGGCTTGTCCCATTTCTGCAG
TGGCGTTATTCATGTGACCAAAGAGGTCAAAGAGGTCGCCACCCTTAGT
TGCGGACATAATGTCTCCGTGGAGGAACTGGCGCAGACCAGGATTTACT
GGCAGAAGGAGAAAAAGATGGTGCTTACAATGATGTCCGGGGACATGAA
TATTTGGCCTGAATATAAAAACCGAACCATTTTCGACATCACTAATAAT
CTCAGCATCGTGATTCTGGCTCTAAGGCCCTCAGACGAGGGTACATACG
AGTGTGTAGTCCTGAAATATGAAAAGGATGCATTCAAGCGTGAGCACCT
GGCAGAGGTGACTCTATCAGTTAAGGCCGACTTTCCCACGCCCAGTATC
TCTGATTTCGAGATCCCTACTTCCAACATTAGACGCATCATCTGTAGCA
CTAGCGGGGGGTTTCCGGAACCCCATCTGTCTTGGCTCGAAAATGGAGA
AGAGTTGAATGCCATAAACACAACAGTGAGCCAAGATCCGGAGACCGAG
CTGTACGCCGTATCTTCTAAACTGGATTTCAACATGACGACAAACCACT
CATTCATGTGCTTAATCAAGTACGGTCACCTGCGGGTCAACCAGACATT
TAATTGGAACACTACTAAGCAAGAACACTTTCCAGACAATCTGCTGCCA
TCATGGGCAATAACCCTCATTAGCGTGAACGGAATCTTTGTTATATGTT
GCCTGACCTATTGTTTTGCTCCACGGTGCAGAGAACGCAGGAGAAACGA
AAGGCTCAGACGAGAGTCGGTGCGCCCTGTT.
>NM_005191.4:376-1242 Homo sapiens CD80 molecule
(CD80), mRNA
ATGGGCCACACACGGAGGCAGGGAACATCACCATCCAAGTGTCCATACC
TCAATTTCTTTCAGCTCTTGGTGCTGGCTGGTCTTTCTCACTTCTGTTC
AGGTGTTATCCACGTGACCAAGGAAGTGAAAGAAGTGGCAACGCTGTCC
TGTGGTCACAATGTTTCTGTTGAAGAGCTGGCACAAACTCGCATCTACT
GGCAAAAGGAGAAGAAAATGGTGCTGACTATGATGTCTGGGGACATGAA
TATATGGCCCGAGTACAAGAACCGGACCATCTTTGATATCACTAATAAC
CTCTCCATTGTGATCCTGGCTCTGCGCCCATCTGACGAGGGCACATACG
AGTGTGTTGTTCTGAAGTATGAAAAAGACGCTTTCAAGCGGGAACACCT
GGCTGAAGTGACGTTATCAGTCAAAGCTGACTTCCCTACACCTAGTATA
TCTGACTTTGAAATTCCAACTTCTAATATTAGAAGGATAATTTGCTCAA
CCTCTGGAGGTTTTCCAGAGCCTCACCTCTCCTGGTTGGAAAATGGAGA
AGAATTAAATGCCATCAACACAACAGTTTCCCAAGATCCTGAAACTGAG
CTCTATGCTGTTAGCAGCAAACTGGATTTCAATATGACAACCAACCACA
GCTTCATGTGTCTCATCAAGTATGGACATTTAAGAGTGAATCAGACCTT
CAACTGGAATACAACCAAGCAAGAGCATTTTCCTGATAACCTGCTCCCA
TCCTGGGCCATTACCTTAATCTCAGTAAATGGAATTTTTGTGATATGCT
GCCTGACCTACTGCTTTGCCCCAAGATGCAGAGAGAGAAGGAGGAATGA
GAGATTGAGAAGGGAAAGTGTACGCCCTGTATAA.

By “MeV-Fc30 polypeptide” or “MeV-Fwt polynucleotide” is meant a polypeptide or fragment thereof with at least about 85% amino acid sequence identity to the sequence provided below, and that functions in lentivirus envelope fusion with the membrane of a target cell. In embodiments, the Mev-Fc30 polypeptide functions in combination with MeV-Hwtc18, DMV—H-N11, DHV-H-hCD105, DMV—H-CD7, DMV-H-32, CDV—H-N11, CDV-H-hCD105, or CDV—H-CD7 polypeptide to fuse a lentivirus lipid envelope with a membrane (e.g., a cell membrane). An exemplary MeV-Fc30 polypeptide sequence is provided below.

MGLKVNVSAIFMAVLLTLQTPTGQIHWGNLSKIGVVGIGSASYKVMTRS
SHQSLVIKLMPNITLLNNCTRVEIAEYRRLLRTVLEPIRDALNAMTQNI
RPVQSVASSRRHKRFAGVVLAGAALGVATAAQITAGIALHQSMLNSQAI
DNLRASLETTNQAIEAIRQAGQEMILAVQGVQDYINNELIPSMNQLSCD
LIGQKLGLKLLRYYTEILSLFGPSLRDPISAEISIQALSYALGGDINKV
LEKLGYSGGDLLGILESRGIKARITHVDTESYFIVLSIAYPTLSEIKGV
IVHRLEGVSYNIGSQEWYTTVPKYVATQGYLISNFDESSCTFMPEGTVC
SQNALYPMSPLLQECLRGSTKSCARTLVSGSFGNRFILSQGNLIANCAS
ILCKCYTTGTIINQDPDKILTYIAADHCPVVEVNGVTIQVGSRRYPDAV
YLHRIDLGPPISLERLDVGTNLGNAIAKLEDAKELLESSDQILRSMKGL
SSTSIVYILIAVCLGGLIGIPALICCCRGR.

In the above polypeptide sequence, the signal peptide sequence is underlined, the transmembrane domain is in italics, the intracellular domain (which includes a 30 amino acid truncation) is in bold, and the extravirion domain is in plain text.

By “MeV-Fc30 polynucleotide” or “MeV-Fwt polynucleotide” is meant a polynucleotide encoding an MeV-Fc30 polypeptide. An exemplary MeV-Fc30 polynucleotide sequence is provided below.

ATGGGCCTGAAGGTGAACGTGAGCGCCATCTTCATGGCCGTGCTGCTGA
CCCTGCAGACCCCAACAGGCCAGATCCACTGGGGCAATCTGTCTAAGAT
CGGAGTGGTGGGAATCGGATCCGCCTCTTATAAAGTGATGACAAGGAGC
TCCCACCAGAGCCTGGTCATCAAGCTGATGCCTAACATCACCCTGCTGA
ACAATTGTACACGCGTGGAGATCGCCGAGTACCGGAGACTGCTGAGGAC
CGTGCTGGAGCCAATCAGGGACGCCCTGAACGCCATGACACAGAATATC
AGGCCAGTGCAGAGCGTGGCATCTAGCAGGCGCCACAAGAGATTTGCCG
GCGTGGTGCTGGCAGGAGCCGCCCTGGGAGTGGCAACCGCCGCCCAGAT
CACAGCCGGCATCGCCCTGCACCAGTCCATGCTGAACTCTCAGGCCATC
GATAATCTGAGGGCCTCCCTGGAGACAACAAACCAGGCAATCGAGGCAA
TCAGGCAGGCAGGACAGGAGATGATCCTGGCCGTGCAGGGCGTGCAGGA
CTATATCAACAATGAGCTGATCCCTTCTATGAATCAGCTGAGCTGCGAT
CTGATCGGCCAGAAGCTGGGCCTGAAGCTGCTGCGGTACTATACCGAGA
TCCTGTCCCTGTTCGGACCTTCTCTGCGGGACCCCATCAGCGCCGAGAT
CTCCATCCAGGCCCTGTCCTATGCACTGGGAGGCGACATCAACAAGGTG
CTGGAGAAGCTGGGCTACTCTGGCGGCGATCTGCTGGGCATCCTGGAGA
GCCGGGGCATCAAGGCCAGAATCACCCACGTGGATACAGAGTCTTACTT
CATCGTGCTGAGCATCGCCTACCCCACACTGTCCGAGATCAAGGGCGTG
ATCGTGCACAGGCTACCCAGGGCTACCTGATCTCTAACTTCGACGAGTC
CTCTTGTACCTTTATGCCAGAGGGCACAGTGTGCTCCCAGAATGCCCTG
TACCCAATGTCTCCCCTGCTGCAGGAGTGTCTGCGGGGCTCTACCAAGA
GCTGCGCCAGAACACTGGTGAGCGGCTCCTTCGGCAATCGGTTCATCCT
GTCCCAGGGCAACCTGATCGCCAATTGTGCCTCTATCCTGTGCAAGTGC
TATACCACAGGCACCATCATCAACCAGGACCCCGATAAGATCCTGACAT
ACATCGCAGCAGACCACTGCCCTGTGGTGGAAGTGAATGGCGTGACCAT
CCAAGTGGGCAGCCGGAGATACCCAGACGCCGTGTATCTGCACAGGATC
GATCTGGGACCACCTATCTCCCTGGAGCGCCTGGATGTGGGCACAAACC
TGGGCAATGCCATCGCCAAGCTGGAGGACGCCAAGGAGCTGCTGGAGAG
CTCCGATCAGATCCTGAGGAGCATGAAGGGCCTGTCTAGCACCTCCATC
GTGTACATCCTGATCGCCGTGTGCCTGGGAGGCCTGATCGGAATCCCCG
CCCTGATCTGCTGTTGCAGGGGCCGCTGA.

By “DMV-F polypeptide” is meant a polypeptide or fragment thereof with at least about 85% amino acid sequence identity to the sequence provided below, and that functions in lentivirus envelope fusion with the membrane of a target cell. In embodiments, the DMV-F polypeptide functions in combination with a MeV-Hwtc18, DMV—H-N11, DHV-H-hCD105, DMV—H-CD7, DMV-H-32, CDV—H-N11, CDV-H-hCD105, or CDV—H-CD7 polypeptide to fuse a lentivirus lipid envelope with a membrane (e.g., a cell membrane). An exemplary DMV-F polypeptide sequence is provided below.

MAASNGGVMYQSFLTIIILVIMTEGQIHWGNLSKIGIVGTGSASYKVMT
RPNHQYLVIKLMPNVTMIDNCTRTEVTEYRKLLKTVLEPVKNALTVITK
NIKPIQSLTTSRRSKRFAGVVLAGVALGVATAAQITAGVALHQSIMNSQ
SIDNLRTSLEKSNQAIEEIRQASQETVLAVQGVQDFINNELIPSMHQLS
CEMLGQKLGLKLLRYYTEILSIFGPSLRDPVSAEISIQALSYALGGDIN
KILEKLGYSGADLLAILESRGIKAKVTHVDLEGYFIVLSIAYPTLSEVK
GVIVHKLEAVSYNLGSQEWYTTLPKYVATNGYLISNFDESSCAFMSEVT
ICSQNALYPMSPLLQQCLRGSTASCARSLVSGTIGNRFILSKGNLIANC
ASVLCKCYSTGTIISQDPDKLLTFVAADKCPLVEVDGITIQVGSREYPD
SVYVSRIDLGPAISLEKLDVGTNLGSALTKLDNAKDLLDSSNQILENVR
RSSFGGAMYIGILVCAGALVILCVLVYCCRRHCRKRVQTPPKATPGLKP
DLTGTTKSYVRSL.

In the above polypeptide sequence, the signal peptide sequence is underlined, the DMV-F F2 domain is double-underlined, the transmembrane domain is in italics, the cytoplasmic domain is in bold, and the extravirion domain is in plain text.

By “DMV-Fc30 polypeptide” is meant a DMV-F polypeptide where the cytoplasmic domain has been truncated by the C-terminal 30 amino acids. In an embodiment, the DMV-Fc30 polypeptide comprises a cytoplasmic domain with a sequence about or at least about 80% sequence identity to the amino acid sequence CCRRH.

By “DMV-F polynucleotide” is meant a polynucleotide encoding a DMV-F polypeptide. An exemplary DMV-F polynucleotide sequence is provided below.

ATGGCCGCCAGCAACGGCGGAGTGATGTACCAGTCATTCCTGACCATCA
TCATCCTGGTGATCATGACCGAGGGCCAAATCCATTGGGGCAACCTGAG
CAAGATCGGCATCGTGGGAACAGGCAGCGCTAGCTATAAGGTGATGACC
AGACCTAATCACCAATACCTGGTGATCAAACTGATGCCTAACGTGACCA
TGATTGACAACTGTACACGGACCGAGGTGACCGAGTACAGAAAGCTGCT
GAAAACCGTGCTTGAGCCTGTGAAAAATGCCCTGACCGTGATCACCAAG
AACATCAAGCCTATCCAGAGCCTGACCACAAGCAGACGGAGCAAGAGGT
TCGCCGGAGTGGTGCTGGCCGGCGTCGCCCTCGGTGTCGCAACCGCCGC
CCAGATCACAGCCGGCGTGGCTCTGCACCAGAGCATCATGAACAGCCAG
AGCATTGACAACCTGAGAACCAGCCTGGAGAAAAGCAACCAGGCTATCG
AGGAAATCAGACAGGCCAGCCAGGAGACAGTGCTGGCTGTGCAGGGCGT
GCAGGACTTCATCAACAACGAGCTGATCCCAAGCATGCACCAGCTGTCT
TGTGAAATGCTGGGCCAGAAGCTGGGCCTGAAGCTGCTTAGATACTACA
CCGAGATCCTGAGTATCTTCGGCCCTTCTCTGCGCGACCCCGTGTCCGC
CGAGATAAGCATCCAGGCCCTCAGCTACGCCCTGGGAGGCGACATCAAC
AAGATCCTGGAAAAGCTGGGATATAGCGGCGCCGATCTGCTGGCCATCC
TGGAATCTCGGGGCATCAAGGCCAAGGTGACCCACGTGGATCTGGAAGG
CTACTTCATCGTGCTGTCCATCGCCTACCCGACCCTGTCCGAGGTTAAG
GGCGTTATTGTGCACAAGCTGGAGGCCGTGTCCTACAACCTTGGCTCTC
AAGAGTGGTACACCACCCTGCCTAAGTACGTGGCCACCAATGGCTATCT
GATCAGCAACTTCGACGAGAGCAGCTGTGCTTTTATGTCTGAGGTGACC
ATCTGCAGCCAGAACGCTCTGTACCCAATGAGCCCTCTGCTGCAGCAGT
GCCTGCGGGGCTCCACAGCCTCTTGCGCCAGATCTCTGGTTTCCGGCAC
AATTGGAAATAGATTCATCCTCAGCAAAGGCAACCTGATCGCCAACTGC
GCCAGCGTGCTGTGCAAGTGCTACAGCACCGGAACAATCATCAGCCAGG
ACCCCGACAAGCTGCTGACCTTCGTGGCCGCTGATAAGTGTCCTCTGGT
CGAAGTGGACGGCATCACCATCCAAGTGGGCAGCCGAGAATACCCTGAC
AGCGTATACGTGTCCAGAATCGACCTGGGTCCTGCCATCTCTCTGGAAA
AACTGGACGTGGGCACCAACCTGGGATCTGCCCTGACAAAGTTGGATAA
TGCCAAGGACCTGCTGGATAGCAGCAATCAGATCCTGGAAAACGTGCGG
CGGTCTAGTTTTGGCGGCGCTATGTACATCGGAATCCTGGTCTGCGCCG
GCGCCCTGGTGATCCTGTGCGTGCTGGTGTACTGCTGCAGAAGACACTG
CAGAAAGAGAGTGCAAACACCCCCCAAAGCCACCCCTGGCCTGAAGCCC
GATCTGACAGGCACAACCAAGAGCTACGTGCGGAGCCTGTGA.

By “CDV-F polypeptide” is meant a polypeptide or fragment thereof with at least about 85% amino acid sequence identity to the sequence provided below, and that functions in lentivirus envelope fusion with the membrane of a target cell. In embodiments, the CDV-F polypeptide functions in combination with a MeV-Hwtc18, DMV—H-N11, DHV-H-hCD105, DMV—H-CD7, DMV-H-32, CDV—H-N11, CDV-H-hCD105, or CDV—H-CD7 polypeptide to fuse a lentivirus lipid envelope with a membrane (e.g., a cell membrane). An exemplary DMV-F PGP60, DNV-F polypeptide sequence is provided below.

MHRGIPKSSKTQTHTQQDRPPQPSTELEETRTSRARHSTTSAQRSTHYD
PRTSDRPVSYTMNRTRSRKQTSHRLKNIPVHGNHEATIQHIPESVSKGA
RSQIERRQPNAINSGSHCTWLVLWCLGMASLFLCSKAQIHWDNLSTIGI
IGTDNVHYKIMTRPSHQYLVIKLIPNASLIENCTKAELGEYEKLLNSVL
EPINQALTLMTKNVKPLQSLGSGRRQRRFAGVVLAGVALGVATAAQITA
GIALHQSNLNAQAIQSLRTSLEQSNKAIEEIREATQETVIAVQGVQDYV
NNELVPAMQHMSCELVGQRLGLRLLRYYTELLSIFGPSLRDPISAEISI
QALIYALGGEIHKILEKLGYSGSDMIAILESRGIKTKITHVDLPGKFII
LSISYPTLSEVKGVIVHRLEAVSYNIGSQEWYTTVPRYIATNGYLISNF
DESSCVFVSESAICSQNSLYPMSPLLQQCIRGDTSSCARTLVSGTMGNK
FILSKGNIVANCASILCKCYSTSTIINQSPDKLLTFIASDTCPLVEIDG
ATIQVGGRQYPDMVYEGKVALGPAISLDRLDVGTNLGNALKKLDDAKVL
IDSSNQILETVRRSSFNFGSLLSVPILSCTALALLLLIYCCKRRYQQTL
KQHTKVDPAFKPDLTGTSKSYVRSL.

In the above polypeptide sequence, the signal peptide sequence is underlined, the transmembrane domain is in italics, the cytoplasmic domain is in bold, and the extravirion domain is in plain text.

By “CDV-Fc30 polypeptide” is meant a CDV-F polypeptide where the cytoplasmic domain has been truncated by the C-terminal 30 amino acids. In an embodiment, the CDV-Fc30 polypeptide comprises a cytoplasmic domain comprising the sequence KRR.

By “CDV-F polynucleotide” is meant a polynucleotide encoding a CDV-F polypeptide.

An exemplary CDV-F polynucleotide sequence is provided below.

ATGCACCGGGGCATCCCCAAGAGCTCCAAGACCCAGACCCACACTCAGCAGGACCGGCCTCCTC
AGCCTAGCACAGAGCTGGAAGAAACCCGGACAAGCCGGGCCAGACACTCTACCACAAGCGCTCA
AAGAAGCACACACTACGACCCCAGAACCTCCGATAGACCTGTGTCCTACACCATGAACAGAACA
AGATCTAGAAAGCAGACAAGCCACCGGCTGAAGAACATCCCTGTTCACGGCAATCACGAGGCCA
CAATTCAGCACATCCCGGAAAGCGTGAGCAAGGGCGCAAGAAGCCAGATCGAGCGGAGGCAACC
TAACGCCATTAACAGCGGCTCCCACTGTACCTGGCTGGTCCTGTGGTGCCTGGGCATGGCCAGC
CTGTTTCTGTGCAGCAAGGCCCAGATCCACTGGGACAACCTGTCCACAATCGGCATCATCGGCA
CCGACAACGTGCATTATAAGATCATGACAAGACCATCTCACCAGTACCTGGTGATCAAGCTGAT
CCCCAACGCCAGCCTCATCGAGAACTGCACGAAGGCCGAGCTGGGAGAGTACGAGAAACTGCTT
AATTCTGTGCTGGAGCCTATCAACCAGGCCCTGACCCTGATGACCAAGAATGTGAAACCTCTGC
AAAGCCTCGGGTCTGGCAGAAGACAGAGAAGATTCGCCGGAGTGGTGCTGGCCGGCGTGGCCCT
GGGCGTAGCCACCGCCGCCCAGATCACCGCCGGCATCGCCCTGCACCAGTCTAATCTGAACGCC
CAGGCCATTCAGTCCCTCCGTACCAGCCTGGAACAGAGCAACAAAGCTATCGAGGAAATCAGGG
AAGCTACCCAGGAGACAGTGATCGCCGTGCAGGGCGTGCAAGACTACGTGAACAACGAGCTGGT
CCCCGCCATGCAGCATATGAGCTGTGAACTGGTTGGCCAGAGACTGGGCCTGAGACTGCTGAGA
TACTACACGGAACTGCTTTCTATTTTCGGCCCTAGCCTGCGGGACCCCATCTCTGCTGAAATCT
CCATCCAAGCCCTGATCTACGCTCTGGGAGGCGAGATTCACAAGATCCTGGAAAAGCTGGGCTA
CAGCGGCAGCGATATGATCGCAATCCTGGAGAGCCGGGGCATCAAGACAAAAATCACCCACGTG
GATCTGCCTGGCAAATTCATCATCCTGAGCATCTCTTATCCTACCCTGTCTGAGGTGAAGGGCG
TGATCGTGCACCGGCTGGAAGCCGTGAGCTACAACATCGGATCCCAGGAGTGGTATACAACCGT
GCCTAGATACATCGCCACCAACGGCTACCTGATCAGCAATTTCGACGAGAGTAGCTGTGTGTTC
GTGAGCGAGAGCGCCATCTGCAGCCAGAACAGCCTGTACCCCATGTCTCCACTGCTGCAGCAGT
GCATCAGAGGCGACACCTCTAGCTGCGCCAGAACCCTGGTGTCTGGCACCATGGGCAACAAGTT
TATCCTGTCTAAGGGAAATATCGTGGCCAATTGCGCCAGCATCCTGTGTAAATGCTACAGCACC
AGCACAATCATCAACCAGAGCCCTGATAAGCTCCTGACCTTTATAGCTTCTGATACCTGTCCTC
TGGTCGAGATCGACGGCGCCACCATCCAAGTGGGCGGAAGGCAGTACCCCGATATGGTGTACGA
GGGCAAGGTGGCCCTGGGACCTGCCATCAGCCTGGACAGACTGGATGTGGGCACAAACCTGGGC
AACGCCCTGAAAAAGCTCGACGACGCCAAGGTGCTGATCGACAGCAGCAACCAGATCCTGGAAA
CCGTCAGACGGTCCAGCTTCAACTTCGGAAGCCTGCTGTCCGTGCCCATCCTGAGTTGCACCGC
CTTGGCTCTGCTGCTGCTGATCTACTGCTGCAAGAGACGGTACCAGCAGACCCTGAAGCAGCAC
ACTAAGGTGGACCCTGCCTTCAAGCCTGACCTGACCGGCACAAGCAAAAGCTACGTGAGAAGCC
TGTGA.

By “MeV-Hwtc18 polypeptide” or “MeV-Hwt polypeptide” is meant a polypeptide or fragment thereof having at least about 85% amino acid sequence identity to the sequence provided below, and that functions in lentivirus envelope fusion with the membrane of a target cell. In embodiments, the MeV-Hwtc18 polypeptide functions in combination with an MeV-Fc30 polypeptide to fuse a lentivirus lipid envelope with a membrane (e.g., a cell membrane). In an embodiment, the MeV-Hwtc18 polypeptide contains an N481A alteration. Not intending to be bound by theory, the N481A alteration prevents TLR2 activation. An exemplary MeV-Hwtc18 polypeptide sequence is provided below

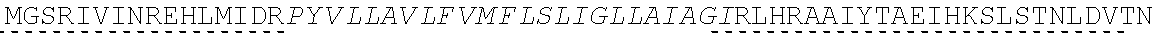

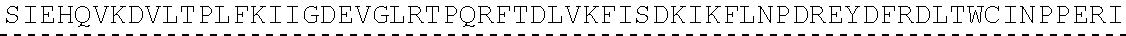

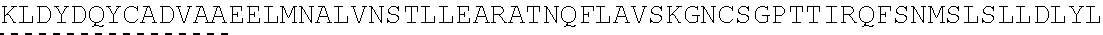

SRGYNVSSIVTMTSQGMYGGTYLVGKPNLSSKGSELSQLSMHRVFEVGVIRNPGLGAPVFHMTN
YFEQPVSNDFSNCMVALGELKFAALCHREDSITIPYQGSGKGVSFQLVKLGVWKSPTDMRSWVP
LSTDDPVIDRLYLSSHRGVIADNQAKWAVPTTRTDDKLRMETCFQQACKGKNQALCENPEWAPL
KDNRIPSYGVLSVNLSLTVELKIKIASGFGPLITHGSGMDLYKTNHNNVYWLTIPPMKNLALGV
INTLEWIPRFKVSPALFTVPIKEAGEDCHAPTYLPAEVDGDVKLSSNLVILPGQDLQYVLATYD
TSRVEHAVVYYVYSPSRSFSYFYPFRLPIKGVPIELQVECFTWDKKLWCRHFCVLADSESGGHI
THSGMVGMGVSCTVTREDGT.

In the above polypeptide sequence, the cytoplasmic domain (which includes an 18 amino acid truncation) is underlined, the transmembrane domain is in italics, the MeV-H stalk is underlined with a dashed line, and the extravirion domain is in plain text.

By “MeV-Hwtc18 polynucleotide” or “MeV-Hwt polynucleotide” is meant a polynucleotide encoding an MeV-Hwtc18 polypeptide. An exemplary MeV-Hwtc18 polynucleotide sequence is provided below.

ATGGGCTCTAGGATCGTGATCAACCGCGAGCACCTGATGATCGACAGGCCATACGTGCTGCTGG
CCGTGCTGTTCGTGATGTTTCTGAGCCTGATCGGCCTGCTGGCAATCGCAGGAATCAGGCTGCA
CAGAGCCGCCATCTATACCGCCGAGATCCACAAGAGCCTGTCCACCAACCTGGACGTGACAAAT
TCCATCGAGCACCAGGTGAAGGATGTGCTGACACCACTGTTCAAGATCATCGGCGACGAAGTGG
GCCTGAGGACCCCACAGCGGTTCACCGACCTGGTGAAGTTCATCAGCGATAAGATCAAGTTTCT
GAACCCTGATCGGGAGTACGACTTCAGAGATCTGACCTGGTGCATCAATCCCCCTGAGCGGATC
AAGCTGGACTACGATCAGTATTGTGCCGACGTGGCCGCCGAGGAGCTGATGAACGCCCTGGTGA
ATTCCACCCTGCTGGAGGCCAGAGCCACAAACCAGTTTCTGGCCGTGAGCAAGGGCAATTGCTC
CGGCCCAACCACAATCCGGGGCCAGTTCTCTAACATGTCTCTGAGCCTGCTGGATCTGTACCTG
AGCAGAGGCTATAATGTGAGCTCCATCGTGACCATGACAAGCCAGGGCATGTACGGCGGCACCT
ATCTGGTGGGCAAGCCCAACCTGTCTAGCAAGGGCTCCGAGCTGTCTCAGCTGAGCATGCACAG
GGTGTTTGAAGTGGGCGTGATCAGGAACCCAGGCCTGGGAGCCCCCGTGTTTCACATGACCAAT
TACTTCGAGCAGCCCGTGAGCAACGATTTTTCTAATTGCATGGTGGCCCTGGGAGAGCTGAAGT
TCGCCGCCCTGTGCCACAGGGAGGACAGCATCACCATCCCATATCAGGGCTCTGGCAAGGGCGT
GAGCTTCCAGCTGGTGAAGCTGGGCGTGTGGAAGTCCCCCACCGACATGAGATCCTGGGTGCCT
CTGTCTACAGACGATCCAGTGATCGATCGGCTGTACCTGTCCTCTCACAGAGGCGTGATCGCCG
ACAATCAGGCAAAGTGGGCAGTGCCAACCACAAGGACCGACGATAAGCTGAGAATGGAGACATG
CTTTCAGCAGGCCTGTAAGGGCAAGAACCAGGCCCTGTGCGAGAATCCAGAGTGGGCACCTCTG
AAGGATAACAGAATCCCTAGCTACGGCGTGCTGAGCGTGAATCTGTCCCTGACCGTGGAGCTGA
AGATCAAGATCGCCAGCGGCTTCGGCCCACTGATCACACACGGCTCCGGCATGGACCTGTACAA
GACCAACCACAACAACGTGTACTGGCTGACAATCCCACCCATGAAGAACCTGGCCCTGGGCGTG
ATCAATACCCTGGAGTGGATCCCCAGGTTTAAGGTGTCCCCTGCCCTGTTCACCGTGCCAATCA
AGGAGGCCGGCGAGGATTGTCACGCCCCAACATACCTGCCCGCCGAGGTGGACGGCGATGTGAA
GCTGAGCTCCAACCTGGTCATCCTGCCTGGCCAGGACCTGCAGTACGTGCTGGCCACCTATGAT
ACAAGCCGCGTGGAGCACGCCGTGGTGTACTACGTGTACTCCCCCTCTCGGAGCTTCTCCTACT
TTTATCCTTTCAGACTGCCAATCAAGGGCGTGCCTATCGAGCTGCAGGTGGAGTGCTTTACCTG
GGATAAGAAGCTGTGGTGCAGGCACTTCTGCGTGCTGGCAGACTCTGAGAGCGGAGGACACATC
ACCCACTCCGGAATGGTGGGCATGGGCGTGTCTTGTACCGTGACACGCGAGGACGGCACATGA.

By “DMV-H polypeptide” is meant a polypeptide or fragment thereof having at least about 85% amino acid sequence identity to the sequence provided below, and that functions in lentivirus envelope fusion with the membrane of a target cell. An exemplary DMV-H polypeptide sequence is provided below.

MSSPRDKVDAFYKDIPRPRNNRVLLDNERVIIERPLILVGVLAVMFLSL
VGLLAIAGVRLQKATTNSIEVNRKLSTNLETTVSIEHHVKDVLTPLFKI
IGDEVGLRMPQKLTEIMQFISNKIKFLNPDREYDFNDLHWCVNPPDQVK
IDYAQYCNHIAAEELIVTKFKELMNHSLDMSKGRIFPPKNCSGSVITRG
QTIKPGLTLVNIYTTRNFEVSFMVTVISGGMYGKTYFLKPPEPDDPFEF
QAFRIFEVGLVRDVGSREPVLQMTNFMVIDEDEGLNFCLLSVGELRLAA
VCVRGRPVVTKDIGGYKDEPFKVVTLGIIGGGLSNQKTEIYPTIDSSIE
KLYITSHRGIIRNSKARWSVPAIRSDDKDKMEKCTQALCKSRPPPSCNS
SDWEPLTSNRIPAYAYIALEIKEDSGLELDITSNYGPLIIHGAGMDIYE
GPSSNQDWLAIPPLSQSVLGVINKVDFTAGFDIKPHTLTTAVDYESGKC
YVPVELSGAKDQDLKLESNLVVLPTKDFGYVTATYDTSRSEHAIVYYVY
DTARSSSYFFPFRIKARGEPIYLRIECFPWSRQLWCHHYCMINSTVSNE
IVVVDNLVSINMSCSR.

In the above polypeptide sequence, the cytoplasmic domain is underlined, the transmembrane domain is in italics, and the extravirion domain is in plain text.

By “DMV-H polynucleotide” is meant a polynucleotide encoding a DMV-H polypeptide.

By “CDV-H polypeptide” is meant a polypeptide or fragment thereof having at least about 85% amino acid sequence identity to the sequence provided below, and that functions in lentivirus envelope fusion with the membrane of a target cell. An exemplary CDV-H polypeptide sequence is provided below.

MLPYQDKVGAFYKDNARANSTKLSLVTEGHGGRRPPYLLFVLLILLVGI
LALLAITGVRFHQVSTSNMEFSRLLKEDMEKSEAVHHQVIDVLTPLFKI
IGDEIGLRLPQKLNEIKQFILQKTNFFNPNREFDFRDLHWCINPPSTVK
VNFTNYCESIGIRKAIASAANPILLSALSGGRGDIFPPHRCSGATTSVG
KVFPLSVSLSMSLISRTSEVINMLTAISDGVYGKTYLLVPDDIEREFDT
REIRVFEIGFIKRWLNDMPLLQTTNYMVLPKNSKAKVCTIAVGELTLAS
LCVEESTVLLYHDSSGSQDGILVVTLGIFWATPMDHIEEVIPVAHPSMK
KIHITNHRGFIKDSIATWMVPALASEKQEEQKGCLESACQRKTYPMCNQ
ASWEPFGGRQLPSYGRLTLPLDASVDLQLNISFTYGPVILNGDGMDYYE
SPLLNSGWLTIPPKDGTISGLINKAGRGDQFTVLPHVLTFAPRESSGNC
YLPIQTSQIRDRDVLIESNIVVLPTQSIRYVIATYDISRSDHAIVYYVY
DPIRTISYTHPFRLTTKGRPDFLRIECFVWDDNLWCHQFYRFEADIANS
TTSVENLVRIRFSCNR.

In the above polypeptide sequence, the cytoplasmic domain is underlined, the transmembrane domain is in italics, and the extravirion domain is in plain text.

By “CDV-H polynucleotide” is meant a polynucleotide encoding a CDV-H polypeptide.

By “MeV-Hc18-CDV polypeptide” is meant a polypeptide or fragment thereof having at least about 85% amino acid sequence identity to the sequence provided below, and that functions in lentivirus envelope fusion with the membrane of a target cell.

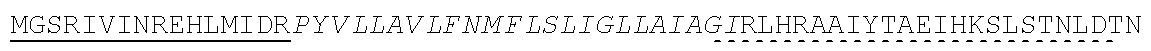

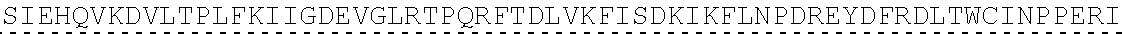

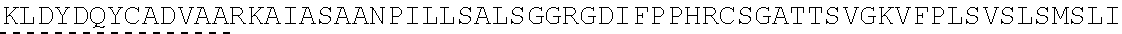

SRTSEVINMLTAISDGVYGKTYLLVPDDIEREFDTREIRVFEIGFIKRWLNDMPLLQTTNYMVL
PKNSKAKVCTIAVGELTLASLCVEESTVLLYHDSSGSQDGILVVTLGIFWATPMDHIEEVIPVA
HPSMKKIHITNHRGFIKDSIATWMVPALASEKQEEQKGCLESACQRKTYPMCNQASWEPFGGRQ
LPSYGRLTLPLDASVDLQLNISFTYGPVILNGDGMDYYESPLLNSGWLTIPPKDGTISGLINKA
GRGDQFTVLPHVLTFAPRESSGNCYLPIQTSQIRDRDVLIESNIVVLPTQSIRYVIATYDISRS
DHAIVYYVYDPIRTISYTHPFRLTTKGRPDFLRIECFVWDDNLWCHQFYRFEADIANSTTSVEN
LVRIRFSCNR.

In the above polypeptide sequence, the cytoplasmic domain is underlined, the transmembrane domain is in italics, the MeV-H stalk is underlined with a dashed line, and the extravirion domain is in plain text.

By “MeV-Hc18-CDV polynucleotide” is meant a polynucleotide encoding an MeV-He18-CDV polypeptide.

By “MeV-Hc18-DMV polypeptide” is meant a polypeptide or fragment thereof having at least about 85% amino acid sequence identity to the sequence provided below, and that functions in lentivirus envelope fusion with the membrane of a target cell

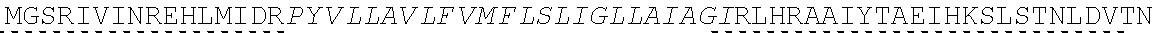

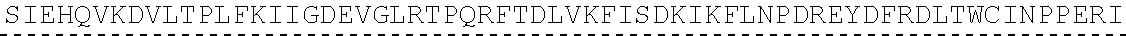

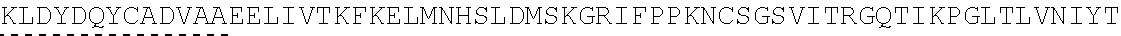

TRNFEVSFMVTVISGGMYGKTYFLKPPEPDDPFEFQAFRIFEVGLVRDVGSREPVLQMTNFMVI
DEDEGLNFCLLSVGELRLAAVCVRGRPVVTKDIGGYKDEPFKVVTLGIIGGGLSNQKTEIYPTI
DSSIEKLYITSHRGIIRNSKARWSVPAIRSDDKDKMEKCTQALCKSRPPPSCNSSDWEPLTSNR
IPAYAYIALEIKEDSGLELDITSNYGPLIIHGAGMDIYEGPSSNQDWLAIPPLSQSVLGVINKV
DFTAGFDIKPHTLTTAVDYESGKCYVPVELSGAKDQDLKLESNLVVLPTKDFGYVTATYDTSRS
EHAIVYYVYDTARSSSYFFPFRIKARGEPIYLRIECFPWSRQLWCHHYCMINSTVSNEIVVVDN
LVSINMSCSR.

In the above polypeptide sequence, the cytoplasmic domain is underlined, the transmembrane domain is in italics, the MeV-H stalk is underlined with a dashed line, and the extravirion domain is in plain text.

By “MeV-Hc18-DMV polynucleotide” is meant a polynucleotide encoding a MeV-Hc18-DMV polypeptide.

By “FMV-H polypeptide” is meant a polypeptide or fragment thereof having at least about 85% amino acid sequence identity to the sequence provided below, and that functions in lentivirus envelope fusion with the membrane of a target cell.

MESNNIKYYKDSSRYFGKILDEHKTINSQLYSLSIKVITIIAIIVSLIAT
IITIINATSGRTTLNSNTDILLSQRDEIHNIQEMIFDRIYPLINAMSTEL
GLHIPTLLDELTKAIDQKIKIMHPPVDTVTSDLNWCIKPPNGIIIDPKSY
CESMELSKTYELLLDQLDVSRKKSLIINRKNINQCQLVDNSKIIFATVNI
QSTPRFLNFGHTVSNQRITFGQGTYSSTYVITIQEDGVTDVQYRVFEIGY
ISDQFGVFPSLIVSRVLPIRMLLGMESCTLTSDRLGGYFLCMNTLTRSIY
DYVSIRDLKSLYITIPHYGKVNYTYFNFGKIRSPHEIDKIWLTSDRGQII
SGYFAAFVTITIRNYNNYPYKCLNNPCFDNSENYCRGWYKNITGTDDVPI
LAYLLVEMYDEEGPLITLVAIPPYNYTAPSHNSLYYDDKINKLIMTTSHI
GYIQINEVHEVIVGDNLKAILLNRLSDEHPNLTACRLNQGIKEQYKSDGT
IISNSALIDIQERMYITVKAIPPAGNYNFTVELHSRSNTSYVSLPKQFNA
KYDKLHLECFSWDKSWWCALIPQFSLSWNESLSVDTAIFNLISCK.

In the above polypeptide sequence, the cytoplasmic domain is underlined, the transmembrane domain is in italics, and the extravirion domain is in plain text.

By “FMV-H polynucleotide” is meant a polynucleotide encoding a FMV-H polypeptide.

By “PPRV-H polypeptide” is meant a polypeptide or fragment thereof having at least about 85% amino acid sequence identity to the sequence provided below, and that functions in lentivirus envelope fusion with the membrane of a target cell.

MSAQRERINAFYKDNLHNKTHRVILDRERLTIERPYILLGVLLVMFLSLI
GLLAIAGIRLHRATVGTAEIQSRLNTNIELTESIDHQTKDVLTPLFKIIG
DEVGIRIPQKFSDLVKFISDKIKFLNPDREYDFRDLRWCMNPPERVKINF
DQFCEYKAAVKSVEHIFESSLNRSERLRLLTLGPGTGCLGRTVTRAQFSE
LTLTLMDLDLEIKHNVSSVFTVVEEGLFGRTYTVWRSDTGKPSTSPGIGH
FLRVFEIGLVRDLELGAPIFHMTNYLTVNMSDDYRSCLLAVGELKLTALC
TPSETVTLSESGVPKREPLVVVILNLAGPTLGGELYSVLPTTDPTVEKLY
LSSHRGIIKDNEANWVVPSTDVRDLQNKGECLVEACKTRPPSFCNGTGIG
PWSEGRIPAYGVIRVSLDLASDPGVVITSVFGPLIPHLSGMDLYNNPFSR
AAWLAVPPYEQSFLGMINTIGFPDRAEVMPHILTTEIRGPRGRCHVPIEL
SSRIDDDIKIGSNMVVLPTKDLRYITATYDVSRSEHAIVYYIYDTGRSSS
YFYPVRLNFRGNPLSLRIECFPWYHKVWCYHDCLIYNTITNEEVHTRGLT
GIEVTCNPV.

In the above polypeptide sequence, the cytoplasmic domain is underlined, the transmembrane domain is in italics, and the extravirion domain is in plain text.

By “PPRV-H polynucleotide” is meant a polynucleotide encoding a PPRV-H polypeptide.

By “RPV-H polypeptide” is meant a polypeptide or fragment thereof having at least about 85% amino acid sequence identity to the sequence provided below, and that functions in lentivirus envelope fusion with the membrane of a target cell.

MSPPRDRVDAYYKDNFQFKNTRVVLNKEQLLIERPCMLLTVLFVMFLSLV
GLLAIAGIRLHRAAVNTAKINNDLTTSIDITKSIEYQVKDVLTPLFKIIG
DEVGLRTPQRFTDLTKFISDKIKFLNPDKEYDFRDINWCINPPERIKIDY
DQYCAHTAAEDLITMLVNSSLTGTTVLRTSLVNLGRNCTGPTTTKGQFSN
ISLTLSGIYSGRGYNISSMITITGKGMYGSTYLVGKYNQRARRPSIVWQQ
DYRVFEVGIIRELGVGTPVFHMTNYLELPRQPELETCMLALGESKLAALC
LADSPVALHYGRVGDDNKIRFVKLGVWASPADRDTLATLSAIDPTLDGLY
ITTHRGIIAAGTAIWAVPVTRTDDQVKMGKCRLEACRDRPPPFCNSTDWE
PLEAGRIPAYGVLTIKLGLADEPKVDIISEFGPLITHDSGMDLYTSFDGT
KYWLTTPPLQNSALGTVNTLVLEPSLKISPNILTLPIRSGGGDCYTPTYL
SDRADDDVKLSSNLVILPSRDLQYVSATYDISRVEHAIVYHIYSTGRLSS
YYYPFKLPIKGDPVSLQIECFPWDRKLWCHHFCSVIDSGTGEQVTHIGVV
GIEITCNGK.

In the above polypeptide sequence, the cytoplasmic domain is underlined, the transmembrane domain is in italics, and the extravirion domain is in plain text.

By “RPV-H polynucleotide” is meant a polynucleotide encoding a RPV-H polypeptide.

By “RMV-H polypeptide” is meant a polypeptide or fragment thereof having at least about 85% amino acid sequence identity to the sequence provided below, and that functions in lentivirus envelope fusion with the membrane of a target cell.

MSAQRERINAFYKDNPHNKNHRVILDRERLVIERPYILLGVLLVMFLSLI
GLLAIAGIRLHRATVGTSEIQSRLNTNIELTESIDHQTKDVLTPLFKIIG
DEVGIRIPQKFSDLVKFISDKIKFLNPDREYDFRDLRWCMNPPERVKINF
DQFCEYKAAVKSIEHIFESPLNKSKKLQSLTLGPGTGCLGRTVTRAHFSE
LTLTLMDLDLEMKHNVSSVFTVVEEGLFGRTYTVWRSDARDPSTDLGIGH
FLRVFEIGLVRDLGLGPPVFHMTNYLTVNMSDDYRRCLLAVGELKLTALC
SSSETVTLGERGVPKREPLVVVILNLAGPTLGGELYSVLPTSDLMVEKLY
LSSHRGIIKDDEANWVVPSTDVRDLQNKGECLVEACKTRPPSFCNGTGSG
PWSEGRIPAYGVIRVSLDLASDPGVVITSVFGPLIPHLSGMDLYNNPFSR
AVWLAVPPYEQSFLGMINTIGFPNRAEVMPHILTTEIRGPRGRCHVPIEL
SRRVDDDIKIGSNMVILPTIDLRYITATYDVSRSEHAIVYYIYDTGRSSS
YFYPVRLNFKGNPLSLRIECFPWRHKVWCYHDCLIYNTITDEEVHTRGLT
GIEVTCNPV.

In the above polypeptide sequence, the cytoplasmic domain is underlined, the transmembrane domain is in italics, and the extravirion domain is in plain text.

By “RMV-H polynucleotide” is meant a polynucleotide encoding a RMV-H polypeptide.

By “DMV-H-MHCII (N11) polypeptide” is meant a polypeptide or fragment thereof having at least about 85% amino acid sequence identity to the sequence provided below, and that functions in lentivirus envelope fusion with the membrane of a target cell expressing an MHCII polypeptide. In some instances the MHCII polypeptide is a murine or human MHCII polypeptide. In embodiments, the DMV-H-MHCII (N11) polypeptide functions in combination with an MeV-Fc30, DMV-F, DMV-Fc30, CDV-F, or CDV-Fc30 polypeptide to fuse a lentivirus lipid envelope with a membrane (e.g., a cell membrane). An exemplary DMV-H-MHCII (N11) polypeptide sequence is provided below.

MSSPRDKVDAFYKDIPRPRNNRVLLDNERVIIERPLILVGVLAVMFLSLV
GLLAIAGVRLQKATTNSIEVNRKLSTNLETTVSIEHHVKDVLTPLFKIIG
DEVGLRMPQKLTEIMQFISNKIKFLNPDREYDFNDLHWCVNPPDQVKIDY
AQYCNHIAAEELIVTKFKELMNHSLDMSKGRIFPPKNCSGSVITRGQTIK
PGLTLVNIYTTRNFEVSFMVTVISGGMYGKTYFLKPPEPDDPFEFQAFRI
FEVGLVRDVGSREPVLQMTNFMVIDEDEGLNFCLLSVGELRLAAVCVRGR
PVVTKDIGGYKDEPFKVVTLGIIGGGLSNQKTEIYPTIDSSIEKLYITSH
RGIIRNSKARWSVPAIRSDDKDKMEKCTQALCKSRPPPSCNSSDWEPLTS
NRIPAYAYIALEIKEDSGLELDITSNYGPLIIHGAGMDIYEGPSSNQDWL
AIPPLSQSVLGVINKVDFTAGFDIKPHTLTTAVDYESGKCYVPVELSGAK
DQDLKLESNLVVLPTKDFGYVTATYDTSRSEHAIVYYVYDTARSSSYFFP
FRIKARGEPIYLRIECFPWSRQLWCHHYCMINSTVSNEIVVVDNLVSINM
SCSRGGGGSGGGGSGGGGSAAAQVQLVQSGGGLVQPGGSLGLSCAASGNI
GSRDNMGWYRQAPGKQREWVATISGYGIATYRDSVKGRFTVAKDTAKNIV
SLQMNYLTTEDTAVYYCYAYAVDSRNIFWSQGTQVTVS.

In the above polypeptide sequence, the cytoplasmic domain is underlined, the transmembrane domain is in italics, the extravirion domain is in plain text, the (G4S)3 linker is in bold, the NotI site is in bold underline, and the anti-MHCII VHH domain is in bold italic.

In an embodiment, the DMV-H-MHCII (N11) polypeptide sequence comprises a C-terminal sequence comprising, from N-terminus to C-terminus, a (G3S)2 linker with the sequence GGGSGGGS and an HA tag with the amino acid sequence YPYDVPDYA.

By “DMV-H-MHCII (N11) polynucleotide” is meant a polynucleotide encoding a DMV-H-MHCII (N11) polypeptide. An exemplary DMV-H-MHCII (N11) polynucleotide sequence is

ATGAGCAGCCCTAGGGACAAGGTGGACGCCTTCTACAAGGACATCCCTCG
GCCCAGAAACAACCGCGTGCTGCTGGACAATGAGCGGGTGATCATCGAGA
GACCACTGATCCTGGTGGGGGTGCTGGCCGTGATGTTCCTGTCTCTGGTG
GGCCTGCTGGCCATCGCCGGCGTGCGGCTGCAAAAGGCCACAACCAACAG
CATCGAGGTCAACAGAAAGCTGTCTACCAATCTGGAAACCACCGTGTCTA
TCGAGCACCACGTGAAGGACGTGCTGACCCCTCTGTTCAAGATCATTGGA
GACGAAGTGGGACTCAGAATGCCACAGAAGCTGACAGAGATCATGCAGTT
CATCAGCAACAAGATCAAGTTTCTCAACCCCGACAGAGAGTACGACTTCA
ACGACCTGCACTGGTGTGTGAACCCTCCTGACCAGGTGAAGATCGACTAC
GCCCAGTACTGTAATCATATCGCTGCTGAGGAACTGATCGTGACCAAGTT
CAAAGAGCTGATGAACCACAGCCTGGACATGTCTAAGGGCAGAATCTTCC
CCCCCAAGAACTGCAGCGGAAGCGTGATCACTCGGGGCCAGACCATCAAA
CCTGGCCTGACCCTGGTGAACATTTACACCACAAGAAACTTCGAGGTGTC
ATTCATGGTCACAGTGATCAGTGGCGGCATGTACGGCAAGACCTACTTCC
TGAAACCTCCGGAACCCGATGATCCTTTCGAGTTCCAGGCTTTTAGAATC
TTTGAGGTGGGACTGGTGCGGGATGTGGGCAGCAGGGAACCTGTGCTGCA
GATGACCAACTTCATGGTGATCGACGAGGACGAGGGCCTGAATTTTTGTC
TGCTGAGCGTAGGCGAGCTGAGACTGGCCGCCGTGTGCGTGCGGGGTAGA
CCTGTTGTGACAAAAGATATCGGCGGCTACAAGGATGAGCCTTTCAAGGT
GGTCACACTGGGCATCATCGGCGGCGGACTTTCTAATCAAAAAACCGAAA
TCTACCCCACCATTGACAGCAGCATCGAAAAGCTGTACATCACCAGCCAC
CGGGGCATCATCCGGAACTCCAAGGCCAGATGGTCCGTGCCCGCTATCAG
ATCTGACGACAAGGACAAGATGGAAAAGTGCACCCAGGCCCTGTGTAAAA
GCAGACCTCCACCTTCCTGCAACAGCTCTGACTGGGAGCCTCTGACAAGC
AATAGAATCCCTGCCTACGCCTATATCGCCCTGGAAATCAAGGAAGATAG
CGGCCTTGAGCTGGATATCACCTCGAACTACGGCCCTCTGATCATCCACG
GCGCCGGCATGGACATCTATGAAGGCCCCTCCAGCAACCAGGATTGGCTG
GCTATTCCCCCCCTGTCTCAGAGCGTTCTGGGCGTGATCAACAAAGTGGA
TTTCACCGCCGGATTCGACATCAAGCCCCACACACTGACCACGGCCGTTG
ATTACGAGAGCGGCAAATGCTACGTGCCTGTGGAACTGAGCGGAGCCAAG
GACCAGGACCTGAAGCTGGAAAGCAATCTGGTGGTGTTGCCTACAAAGGA
TTTTGGCTATGTGACAGCAACCTACGATACATCTAGAAGCGAGCACGCCA
TCGTGTACTACGTGTACGACACCGETAGGTCCAGCAGCTACTTCTTCCCT
TTCCGGATCAAGGCCAGAGGAGAGCCAATCTACCTGCGCATTGAGTGCTT
CCCATGGAGCAGACAGCTGTGGTGCCACCACTACTGCATGATCAACAGCA
CCGTGTCCAACGAAATCGTGGTGGTGGATAACCTGGTCAGCATCAACATG
AGCTGCAGCAGAGGGGGAGGCGGCTCTGGAGGTGGGGGGTCCGGTGGCGG
CGGAAGCGCGGCCGCGCAGGTTCAACTGGTGCAGAGCGGAGGCGGCCTGG
TCCAGCCCGGCGGAAGCCTGGGCCTGAGCTGTGCCGCTTCTGGCAACATC
GGCAGCAGAGATAATATGGGCTGGTATAGACAGGCCCCTGGAAAACAGAG
AGAGTGGGTGGCTACAATCAGCGGCTACGGCATCGCCACATACCGGGACA
GCGTGAAGGGCAGATTCACCGTGGCCAAGGACACCGCCAAGAACATCGTG
TCCCTGCAGATGAACTACCTGACCACCGAAGATACAGCCGTGTACTACTG
CTACGCCTACGCCGTGGACAGCCGGAACATCTTCTGGTCCCAGGGCACCC
AGGTGACCGTGTCT.

By “DMV-H-hCD105 polypeptide” is meant a polypeptide or fragment thereof having at least about 85% amino acid sequence identity to the sequence provided below, and that functions in lentivirus envelope fusion with the membrane of a target cell expressing a CD105 polypeptide. In some instances the CD105 polypeptide is a murine or human CD105 polypeptide. In embodiments, the DMV-H-hCD105 polypeptide functions in combination with an MeV-Fc30, DMV-F, DMV-Fc30, CDV-F, or CDV-Fc30 polypeptide to fuse a lentivirus lipid envelope with a membrane (e.g., a cell membrane). An exemplary DMV-H-hCD105 polypeptide sequence is provided below.

MSSPRDKVDAFYKDIPRPRNNRVLLDNERVIIERPLILVGVLAVMFLSLV
GLLAIAGVRLQKATTNSIEVNRKLSTNLETTVSIEHHVKDVLTPLFKIIG
DEVGLRMPQKLTEIMQFISNKIKFLNPDREYDFNDLHWCVNPPDQVKIDY
AQYCNHIAAEELIVTKFKELMNHSLDMSKGRIFPPKNCSGSVITRGQTIK
PGLTLVNIYTTRNFEVSFMVTVISGGMYGKTYFLKPPEPDDPFEFQAFRI
FEVGLVRDVGSREPVLQMTNFMVIDEDEGLNFCLLSVGELRLAAVCVRGR
PVVTKDIGGYKDEPFKVVTLGIIGGGLSNQKTEIYPTIDSSIEKLYITSH
RGIIRNSKARWSVPAIRSDDKDKMEKCTQALCKSRPPPSCNSSDWEPLTS
NRIPAYAYIALEIKEDSGLELDITSNYGPLIIHGAGMDIYEGPSSNQDWL
AIPPLSQSVLGVINKVDFTAGFDIKPHTLTTAVDYESGKCYVPVELSGAK
DQDLKLESNLVVLPTKDFGYVTATYDTSRSEHAIVYYVYDTARSSSYFFP
FRIKARGEPIYLRIECFPWSRQLWCHHYCMINSTVSNEIVVVDNLVSINM
SCSRGGGGSGGGGSGGGGSAAAMAEVQLLESGGGLVQPGGSLRLSCAASG
FTFSSYAMSWVRQAPGKGLEWVSAIYGSDGDTTYADSVKGRFTISRDNSK
NTLYLQMNSLRAEDTAVYYCARVFYTAGFDYWGQGTLVTVSSGSDSNAGH
ASAGNTSDIELTQSPSSLSASVGDRVTITCRASQSISSSLNWYQQKPGKA
PKLLIYAASSLQSGVPSRFSGSGSGTDFTLTISSLQPEDFATYYCQQAPA
KPPTFGQGTKLEIKRAAAYPYDVPDYA.

In the above polypeptide sequence, the cytoplasmic domain is underlined, the transmembrane domain is in italics, the extravirion domain is in plain text, the (G4S)3 linker is in bold, the NotI site is in bold underline, the anti-hCD105 scFv domain is in bold italic, and the HA tag is double underlined.

By “DMV-H-hCD105 polynucleotide” is meant a polynucleotide encoding a DMV-H-hCD105 polypeptide. An exemplary polynucleotide sequence encoding the first 622 amino acids of a DMV-H-hCD105 polynucleotide is provided below.

ATGAGCAGCCCTAGGGACAAGGTGGACGCCTTCTACAAGGACATCCCTCG
GCCCAGAAACAACCGCGTGCTGCTGGACAATGAGCGGGTGATCATCGAGA
GACCACTGATCCTGGTGGGGGTGCTGGCCGTGATGTTCCTGTCTCTGGTG
GGCCTGCTGGCCATCGCCGGCGTGCGGCTGCAAAAGGCCACAACCAACAG
CATCGAGGTCAACAGAAAGCTGTCTACCAATCTGGAAACCACCGTGTCTA
TCGAGCACCACGTGAAGGACGTGCTGACCCCTCTGTTCAAGATCATTGGA
GACGAAGTGGGACTCAGAATGCCACAGAAGCTGACAGAGATCATGCAGTT
CATCAGCAACAAGATCAAGTTTCTCAACCCCGACAGAGAGTACGACTTCA
ACGACCTGCACTGGTGTGTGAACCCTCCTGACCAGGTGAAGATCGACTAC
GCCCAGTACTGTAATCATATCGCTGCTGAGGAACTGATCGTGACCAAGTT
CAAAGAGCTGATGAACCACAGCCTGGACATGTCTAAGGGCAGAATCTTCC
CCCCCAAGAACTGCAGCGGAAGCGTGATCACTCGGGGCCAGACCATCAAA
CCTGGCCTGACCCTGGTGAACATTTACACCACAAGAAACTTCGAGGTGTC
ATTCATGGTCACAGTGATCAGTGGCGGCATGTACGGCAAGACCTACTTCC
TGAAACCTCCGGAACCCGATGATCCTTTCGAGTTCCAGGCTTTTAGAATC
TTTGAGGTGGGACTGGTGCGGGATGTGGGCAGCAGGGAACCTGTGCTGCA
GATGACCAACTTCATGGTGATCGACGAGGACGAGGGCCTGAATTTTTGTC
TGCTGAGCGTAGGCGAGCTGAGACTGGCCGCCGTGTGCGTGCGGGGTAGA
CCTGTTGTGACAAAAGATATCGGCGGCTACAAGGATGAGCCTTTCAAGGT
GGTCACACTGGGCATCATCGGCGGCGGACTTTCTAATCAAAAAACCGAAA
TCTACCCCACCATTGACAGCAGCATCGAAAAGCTGTACATCACCAGCCAC
CGGGGCATCATCCGGAACTCCAAGGCCAGATGGTCCGTGCCCGCTATCAG
ATCTGACGACAAGGACAAGATGGAAAAGTGCACCCAGGCCCTGTGTAAAA
GCAGACCTCCACCTTCCTGCAACAGCTCTGACTGGGAGCCTCTGACAAGC
AATAGAATCCCTGCCTACGCCTATATCGCCCTGGAAATCAAGGAAGATAG
CGGCCTTGAGCTGGATATCACCTCGAACTACGGCCCTCTGATCATCCACG
GCGCCGGCATGGACATCTATGAAGGCCCCTCCAGCAACCAGGATTGGCTG
GCTATTCCCCCCCTGTCTCAGAGCGTTCTGGGCGTGATCAACAAAGTGGA
TTTCACCGCCGGATTCGACATCAAGCCCCACACACTGACCACGGCCGTTG
ATTACGAGAGCGGCAAATGCTACGTGCCTGTGGAACTGAGCGGAGCCAAG
GACCAGGACCTGAAGCTGGAAAGCAATCTGGTGGTGTTGCCTACAAAGGA
TTTTGGCTATGTGACAGCAACCTACGATACATCTAGAAGCGAGCACGCCA
TCGTGTACTACGTGTACGACACCGCTAGGTCCAGCAGCTACTTCTTCCCT
TTCCGGATCAAGGCCAGAGGAGAGCCAATCTACCTGCGCATTGAGTGCTT
CCCATGGAGCAGACAGCTGTGGTGCCACCACTACTGCATGATCAACAGCA
CCGTGTCCAACGAAATCGTGGTGGTGGATAACCTGGTCAGCATCAACATG
AGCTGCAGCAGAGGGGGAGGCGGCTCTGGAGGTGGGGGGTCCGGTGGCGG
CGGAAGCGCGGCCGCGATGGCCGAGGTGCAGCTGCTGGAGTCCGGAGGAG
GCCTGGTGCAGCCAGGAGGCTCTCTGAGGCTGAGCTGCGCAGCATCCGGC
TTCACCTTTTCTAGCTACGCCATGAGCTGGGTGCGCCAGGCACCAGGCAA
GGGCCTGGAGTGGGTGAGCGCCATCTACGGATCCGACGGCGATACCACAT
ATGCCGATTCCGTGAAGGGCAGGTTCACCATCTCTCGCGACAACAGCAAG
AATACACTGTATCTGCAGATGAACTCTCTGAGGGCCGAGGATACCGCCGT
GTACTATTGCGCCCGCGTGTTTTACACAGCCGGCTTCGACTATTGGGGCC
AGGGCACCCTGGTGACAGTGTCCTCTGGCTCCGATTCTAACGCAGGACAC
GCATCTGCCGGAAATACCAGCGACATCGAGCTGACACAGTCCCCAAGCTC
CCTGAGCGCCTCCGTGGGCGACAGGGTGACCATCACATGTCGCGCCTCTC
AGAGCATCTCTAGCTCCCTGAATTGGTACCAGCAGAAGCCCGGCAAGGCC
CCTAAGCTGCTGATCTATGCAGCATCTAGCCTGCAGTCCGGAGTGCCATC
TAGGTTTTCCGGCTCTGGCAGCGGCACCGATTTCACCCTGACAATCTCCT
CTCTGCAGCCCGAGGACTTTGCCACATACTATTGTCAGCAGGCCCCTGCC
AAGCCTCCAACCTTCGGCCAGGGCACAAAGCTGGAGATCAAGAGG.

By “DMV—H-CD7 (Humanized VHH10) polypeptide” is meant a polypeptide or fragment thereof having at least about 85% amino acid sequence identity to the sequence provided below, and that functions in lentivirus envelope fusion with the membrane of a target cell expressing a CD7 polypeptide. In some instances the CD7 polypeptide is a murine or human CD7 polypeptide. In embodiments, the DMV—H-CD7 (Humanized VHH10) polypeptide functions in combination with an MeV-Fc30, DMV-F, DMV-Fc30, CDV-F, or CDV-Fc30 polypeptide to fuse a lentivirus lipid envelope with a membrane (e.g., a cell membrane). An exemplary DMV—H-CD7 (Humanized VHH10) polypeptide sequence is provided below.

MSSPRDKVDAFYKDIPRPRNNRVLLDNERVIIERPLILVGVLAVMFLSLV
GLLAIAGVRLQKATTNSIEVNRKLSTNLETTVSIEHHVKDVLTPLFKIIG
DEVGLRMPQKLTEIMQFISNKIKFLNPDREYDFNDLHWCVNPPDQVKIDY
AQYCNHIAAEELIVTKFKELMNHSLDMSKGRIFPPKNCSGSVITRGQTIK
PGLTLVNIYTTRNFEVSFMVTVISGGMYGKTYFLKPPEPDDPFEFQAFRI
FEVGLVRDVGSREPVLQMTNFMVIDEDEGLNFCLLSVGELRLAAVCVRGR
PVVTKDIGGYKDEPFKVVTLGIIGGGLSNQKTEIYPTIDSSIEKLYITSH
RGIIRNSKARWSVPAIRSDDKDKMEKCTQALCKSRPPPSCNSSDWEPLTS
NRIPAYAYIALEIKEDSGLELDITSNYGPLIIHGAGMDIYEGPSSNQDWL
AIPPLSQSVLGVINKVDFTAGFDIKPHTLTTAVDYESGKCYVPVELSGAK
DQDLKLESNLVVLPTKDFGYVTATYDTSRSEHAIVYYVYDTARSSSYFFP
FRIKARGEPIYLRIECFPWSRQLWCHHYCMINSTVSNEIVVVDNLVSINM
SCSRGGGGSGGGGSGGGGSAAADVQLQESGGGSVQAGGSLRLSCAASGYT
HSSYCMAWFRQAPGREREGVASIDSDGTTSYADSVKGRFTISQDNAKNTL
YLQMNSLKPEDTAMYYCAARFGPMGCVDLSTLSFGHWGQGTQVTVSIT
GGGSGGGSYPYDVPDYA.

In the above polypeptide sequence, the cytoplasmic domain is underlined, the transmembrane domain is in italics, the extravirion domain is in plain text, the (G4S)3 linker is in bold, the NotI site is in bold underline, the anti-CD7 VHH domain is in bold italic, the (G3S)2 linker is in italic underline, and the HA tag is double-underlined.

By “DMV—H-CD7 (Humanized VHH10) polynucleotide” is meant a polynucleotide encoding a DMV—H-CD7 (Humanized VHH10) polypeptide. An exemplary DMV—H-CD7 (Humanized VHH10) polynucleotide sequence is provided below.

ATGAGCAGCCCTAGGGACAAGGTGGACGCCTTCTACAAGGACATCCCTCG
GCCCAGAAACAACCGCGTGCTGCTGGACAATGAGCGGGTGATCATCGAGA
GACCACTGATCCTGGTGGGGGTGCTGGCCGTGATGTTCCTGTCTCTGGTG
GGCCTGCTGGCCATCGCCGGCGTGCGGCTGCAAAAGGCCACAACCAACAG
CATCGAGGTCAACAGAAAGCTGTCTACCAATCTGGAAACCACCGTGTCTA
TCGAGCACCACGTGAAGGACGTGCTGACCCCTCTGTTCAAGATCATTGGA
GACGAAGTGGGACTCAGAATGCCACAGAAGCTGACAGAGATCATGCAGTT
CATCAGCAACAAGATCAAGTTTCTCAACCCCGACAGAGAGTACGACTTCA
ACGACCTGCACTGGTGTGTGAACCCTCCTGACCAGGTGAAGATCGACTAC
GCCCAGTACTGTAATCATATCGCTGCTGAGGAACTGATCGTGACCAAGTT
CAAAGAGCTGATGAACCACAGCCTGGACATGTCTAAGGGCAGAATCTTCC
CCCCCAAGAACTGCAGCGGAAGCGTGATCACTCGGGGCCAGACCATCAAA
CCTGGCCTGACCCTGGTGAACATTTACACCACAAGAAACTTCGAGGTGTC
ATTCATGGTCACAGTGATCAGTGGCGGCATGTACGGCAAGACCTACTTCC
TGAAACCTCCGGAACCCGATGATCCTTTCGAGTTCCAGGCTTTTAGAATC
TTTGAGGTGGGACTGGTGCGGGATGTGGGCAGCAGGGAACCTGTGCTGCA
GATGACCAACTTCATGGTGATCGACGAGGACGAGGGCCTGAATTTTTGTC
TGCTGAGCGTAGGCGAGCTGAGACTGGCCGCCGTGTGCGTGCGGGGTAGA
CCTGTTGTGACAAAAGATATCGGCGGCTACAAGGATGAGCCTTTCAAGGT
GGTCACACTGGGCATCATCGGCGGCGGACTTTCTAATCAAAAAACCGAAA
TCTACCCCACCATTGACAGCAGCATCGAAAAGCTGTACATCACCAGCCAC
CGGGGCATCATCCGGAACTCCAAGGCCAGATGGTCCGTGCCCGCTATCAG
ATCTGACGACAAGGACAAGATGGAAAAGTGCACCCAGGCCCTGTGTAAAA
GCAGACCTCCACCTTCCTGCAACAGCTCTGACTGGGAGCCTCTGACAAGC
AATAGAATCCCTGCCTACGCCTATATCGCCCTGGAAATCAAGGAAGATAG
CGGCCTTGAGCTGGATATCACCTCGAACTACGGCCCTCTGATCATCCACG
GCGCCGGCATGGACATCTATGAAGGCCCCTCCAGCAACCAGGATTGGCTG
GCTATTCCCCCCCTGTCTCAGAGCGTTCTGGGCGTGATCAACAAAGTGGA
TTTCACCGCCGGATTCGACATCAAGCCCCACACACTGACCACGGCCGTTG
ATTACGAGAGCGGCAAATGCTACGTGCCTGTGGAACTGAGCGGAGCCAAG
GACCAGGACCTGAAGCTGGAAAGCAATCTGGTGGTGTTGCCTACAAAGGA
TTTTGGCTATGTGACAGCAACCTACGATACATCTAGAAGCGAGCACGCCA
TCGTGTACTACGTGTACGACACCGCTAGGTCCAGCAGCTACTTCTTCCCT
TTCCGGATCAAGGCCAGAGGAGAGCCAATCTACCTGCGCATTGAGTGCTT
CCCATGGAGCAGACAGCTGTGGTGCCACCACTACTGCATGATCAACAGCA
CCGTGTCCAACGAAATCGTGGTGGTGGATAACCTGGTCAGCATCAACATG
AGCTGCAGCAGAGGGGGAGGCGGCTCTGGAGGTGGGGGGTCCGGTGGCGG
CGGAAGCGCGGCCGCGGATGTGCAGCTGCAGGAGAGCGGCGGCGGATCTG
TGCAAGCTGGCGGAAGCCTGAGACTGTCTTGCGCCGCTTCTGGCTACACC
CACAGCAGCTATTGCATGGCCTGGTTCCGGCAGGCCCCTGGCAGAGAACG
GGAAGGCGTGGCCAGCATCGACAGCGACGGCACCACCAGCTACGCCGATA
GCGTCAAGGGCAGATTCACCATCAGCCAGGACAATGCCAAAAACACCCTG
TACCTGCAGATGAACAGCCTGAAGCCCGAGGACACAGCCATGTACTACTG
TGCCGCCAGATTTGGCCCTATGGGCTGCGTGGACCTGTCCACCCTCTCCT
TCGGCCACTGGGGACAGGGCACACAGGTGACAGTGTCCATCACCGGCGGC
GGCTCCGGCGGCGGCAGCTATCCCTATGATGTGCCCGACTATGCGTGA.

By “DMV—H-CD45 (32) polypeptide” is meant a polypeptide or fragment thereof having at least about 85% amino acid sequence identity to the sequence provided below, and that functions in lentivirus envelope fusion with the membrane of a target cell expressing a CD45 polypeptide. In some instances the CD45 polypeptide is a murine or human CD45 polypeptide. In embodiments, the DMV—H-CD45 (32) polypeptide functions in combination with an MeV-Fc30, DMV-F, DMV-Fc30, CDV-F, or CDV-Fc30 polypeptide to fuse a lentivirus lipid envelope with a membrane (e.g., a cell membrane). An exemplary DMV—H-CD45 (32) polypeptide sequence is provided below.

MSSPRDKVDAFYKDIPRPRNNRVLLDNERVIIERPLILVGVLAVMFLSLV
GLLAIAGVRLQKATTNSIEVNRKLSTNLETTVSIEHHVKDVLTPLFKIIG
DEVGLRMPQKLTEIMQFISNKIKFLNPDREYDFNDLHWCVNPPDQVKIDY
AQYCNHIAAEELIVTKFKELMNHSLDMSKGRIFPPKNCSGSVITRGQTIK
PGLTLVNIYTTRNFEVSFMVTVISGGMYGKTYFLKPPEPDDPFEFQAFRI
FEVGLVRDVGSREPVLQMTNFMVIDEDEGLNFCLLSVGELRLAAVCVRGR
PVVTKDIGGYKDEPFKVVTLGIIGGGLSNQKTEIYPTIDSSIEKLYITSH
RGIIRNSKARWSVPAIRSDDKDKMEKCTQALCKSRPPPSCNSSDWEPLTS
NRIPAYAYIALEIKEDSGLELDITSNYGPLIIHGAGMDIYEGPSSNQDWL
AIPPLSQSVLGVINKVDFTAGEDIKPHTLTTAVDYESGKCYVPVELSGAK
DQDLKLESNLVVLPTKDFGYVTATYDTSRSEHAIVYYVYDTARSSSYFFP
FRIKARGEPIYLRIECFPWSRQLWCHHYCMINSTVSNEIVVVDNLVSINM
SCSRGGGGSGGGGSGGGGSAAAQVQLVQSGGGLVQPGGSLRLSCAASGRA
FNSAAMGWYRQAPGSQRELVASISAGTASYADAVKGRFTISRDYAKNIIY
LQMNSLKPDDTAVYFCNYRTTYTSGYSEDYWGQGTQVTVSGGGSGGGS
YPYDVPDYA.

In the above polypeptide sequence, the cytoplasmic domain is underlined, the transmembrane domain is in italics, the extravirion domain is in plain text, the (G4S)3 linker is in bold, the NotI site is in bold underline, the anti-CD45 VHH domain is in bold italic, the (G3S)2 linker is in italic underline, and the HA tag is double-underlined.

By “DMV—H-CD45 (32) polynucleotide” is meant a polynucleotide encoding a DMV—H-CD45 (32) polypeptide. An exemplary DMV—H-CD45 (32) polynucleotide sequence is provided below.

ATGAGCAGCCCTAGGGACAAGGTGGACGCCTTCTACAAGGACATCCCTCG
GCCCAGAAACAACCGCGTGCTGCTGGACAATGAGCGGGTGATCATCGAGA
GACCACTGATCCTGGTGGGGGTGCTGGCCGTGATGTTCCTGTCTCTGGTG
GGCCTGCTGGCCATCGCCGGCGTGCGGCTGCAAAAGGCCACAACCAACAG
CATCGAGGTCAACAGAAAGCTGTCTACCAATCTGGAAACCACCGTGTCTA
TCGAGCACCACGTGAAGGACGTGCTGACCCCTCTGTTCAAGATCATTGGA
GACGAAGTGGGACTCAGAATGCCACAGAAGCTGACAGAGATCATGCAGTT
CATCAGCAACAAGATCAAGTTTCTCAACCCCGACAGAGAGTACGACTTCA
ACGACCTGCACTGGTGTGTGAACCCTCCTGACCAGGTGAAGATCGACTAC
GCCCAGTACTGTAATCATATCGCTGCTGAGGAACTGATCGTGACCAAGTT
CAAAGAGCTGATGAACCACAGCCTGGACATGTCTAAGGGCAGAATCTTCC
CCCCCAAGAACTGCAGCGGAAGCGTGATCACTCGGGGCCAGACCATCAAA
CCTGGCCTGACCCTGGTGAACATTTACACCACAAGAAACTTCGAGGTGTC
ATTCATGGTCACAGTGATCAGTGGCGGCATGTACGGCAAGACCTACTTCC
TGAAACCTCCGGAACCCGATGATCCTTTCGAGTTCCAGGCTTTTAGAATC
TTTGAGGTGGGACTGGTGCGGGATGTGGGCAGCAGGGAACCTGTGCTGCA
GATGACCAACTTCATGGTGATCGACGAGGACGAGGGCCTGAATTTTTGTC
TGCTGAGCGTAGGCGAGCTGAGACTGGCCGCCGTGTGCGTGCGGGGTAGA
CCTGTTGTGACAAAAGATATCGGCGGCTACAAGGATGAGCCTTTCAAGGT
GGTCACACTGGGCATCATCGGCGGCGGACTTTCTAATCAAAAAACCGAAA
TCTACCCCACCATTGACAGCAGCATCGAAAAGCTGTACATCACCAGCCAC
CGGGGCATCATCCGGAACTCCAAGGCCAGATGGTCCGTGCCCGCTATCAG
ATCTGACGACAAGGACAAGATGGAAAAGTGCACCCAGGCCCTGTGTAAAA
GCAGACCTCCACCTTCCTGCAACAGCTCTGACTGGGAGCCTCTGACAAGC
AATAGAATCCCTGCCTACGCCTATATCGCCCTGGAAATCAAGGAAGATAG
CGGCCTTGAGCTGGATATCACCTCGAACTACGGCCCTCTGATCATCCACG
GCGCCGGCATGGACATCTATGAAGGCCCCTCCAGCAACCAGGATTGGCTG
GCTATTCCCCCCCTGTCTCAGAGCGTTCTGGGCGTGATCAACAAAGTGGA
TTTCACCGCCGGATTCGACATCAAGCCCCACACACTGACCACGGCCGTTG
ATTACGAGAGCGGCAAATGCTACGTGCCTGTGGAACTGAGCGGAGCCAAG
GACCAGGACCTGAAGCTGGAAAGCAATCTGGTGGTGTTGCCTACAAAGGA
TTTTGGCTATGTGACAGCAACCTACGATACATCTAGAAGCGAGCACGCCA
TCGTGTACTACGTGTACGACACCGETAGGTCCAGCAGCTACTTCTTCCCT
TTCCGGATCAAGGCCAGAGGAGAGCCAATCTACCTGCGCATTGAGTGCTT
CCCATGGAGCAGACAGCTGTGGTGCCACCACTACTGCATGATCAACAGCA
CCGTGTCCAACGAAATCGTGGTGGTGGATAACCTGGTCAGCATCAACATG
AGCTGCAGCAGAGGGGGAGGCGGCTCTGGAGGTGGGGGGTCCGGTGGCGG
CGGAAGCGCGGCCGCGCAGGTGCAGCTGGTGCAGAGCGGCGGAGGACTGG
TGCAACCTGGAGGCAGCCTGAGACTGAGCTGCGCCGCTAGCGGCAGAGCC
TTTAACAGCGCCGCCATGGGCTGGTACAGACAGGCCCCTGGCTCTCAGAG
AGAGCTGGTCGCCTCTATCTCCGCCGGCACCGCCTCTTATGCTGATGCCG
TGAAAGGCAGATTCACCATCAGCCGGGACTACGCCAAGAACATCATCTAC
CTGCAGATGAACAGCCTGAAGCCCGACGACACAGCTGTGTACTTCTGTAA
TTACCGGACAACCTACACCAGCGGCTACAGCGAAGATTACTGGGGCCAGG
GCACACAGGTGACCGTGTCCGGCGGCGGCTCCGGCGGCGGCAGCTATCCC
TATGATGTGCCCGACTATGCGTGA.

By “CDV-H-MHCII (N11) polypeptide” is meant a polypeptide or fragment thereof having at least about 85% amino acid sequence identity to the sequence provided below, and that functions in lentivirus envelope fusion with the membrane of a target cell expressing an MHCII polypeptide. In some instances the MHCII polypeptide is a murine or human MHCII polypeptide. In embodiments, the CDV-H-MHCII (N11) polypeptide functions in combination with an MeV-Fc30, DMV-F, DMV-Fc30, CDV-F, or CDV-Fc30 polypeptide to fuse a lentivirus lipid envelope with a membrane (e.g., a cell membrane). An exemplary CDV-H-MHCII (N11) polypeptide sequence is provided below.

MLPYQDKVGAFYKDNARANSTKLSLVTEGHGGRRPPYLLFVLLILLVGIL
ALLAITGVRFHQVSTSNMEFSRLLKEDMEKSEAVHHQVIDVLTPLFKIIG
DEIGLRLPQKLNEIKQFILQKTNFFNPNREFDFRDLHWCINPPSTVKVNF
TNYCESIGIRKAIASAANPILLSALSGGRGDIFPPHRCSGATTSVGKVFP
LSVSLSMSLISRTSEVINMLTAISDGVYGKTYLLVPDDIEREFDTREIRV
FEIGFIKRWLNDMPLLQTTNYMVLPKNSKAKVCTIAVGELTLASLCVEES
TVLLYHDSSGSQDGILVVTLGIFWATPMDHIEEVIPVAHPSMKKIHITNH
RGFIKDSIATWMVPALASEKQEEQKGCLESACQRKTYPMCNQASWEPFGG
RQLPSYGRLTLPLDASVDLQLNISFTYGPVILNGDGMDYYESPLLNSGWL
TIPPKDGTISGLINKAGRGDQFTVLPHVLTFAPRESSGNCYLPIQTSQIR
DRDVLIESNIVVLPTQSIRYVIATYDISRSDHAIVYYVYDPIRTISYTHP
FRLTTKGRPDFLRIECFVWDDNLWCHQFYRFEADIANSTTSVENLVRIRF
SCNRGGGGSGGGGSGGGGSAAAQVQLVQSGGGLVQPGGSLGLSCAASGNI
GSRDNMGWYRQAPGKQREWVATISGYGIATYRDSVKGRFTVAKDTAKNIV
SLQMNYLTTEDTAVYYCYAYAVDSRNIFWSQGTQVTVSGGGSGGGS
YPYDVPDYA.

In the above polypeptide sequence, the cytoplasmic domain is underlined, the transmembrane domain is in italics, the extravirion domain is in plain text, the (G4S)3 linker is in bold, the NotI site is in bold underline, the anti-MHCII VHH domain is in bold italic, the (G3S)2 linker is in italic underline, and the HA tag is double-underlined.

By “CDV-H-MHCII (N11) polynucleotide” is meant a polynucleotide encoding a CDV-H-MHCII (N11) polypeptide. An exemplary CDV-H-MHCII (N11) polynucleotide sequence is provided below.

ATGCTGCCCTATCAGGATAAGGTGGGCGCTTTTTACAAGGATAACGCTAG
AGCCAACAGCACAAAGCTGAGCCTGGTCACCGAGGGCCACGGCGGCAGAA
GACCTCCATACCTGCTGTTTGTGCTGCTAATTCTGCTGGTGGGCATCCTG
GCACTGCTGGCCATTACAGGCGTGCGGTTTCACCAGGTGTCCACCAGCAA
CATGGAATTCAGCAGACTGCTGAAGGAAGATATGGAAAAGAGCGAGGCCG
TGCACCACCAAGTGATCGACGTGCTCACCCCCCTGTTCAAGATCATCGGC
GACGAGATCGGCCTGAGGTTGCCTCAGAAACTGAACGAGATCAAGCAGTT
TATCCTGCAGAAGACCAATTTCTTCAACCCTAACAGAGAGTTTGACTTCA
GAGATCTGCACTGGTGCATCAACCCCCCCAGCACAGTAAAGGTGAATTTC
ACGAATTACTGCGAGAGCATCGGAATCAGAAAGGCCATCGCCAGCGCCGC
CAACCCCATCCTGCTGTCTGCTCTGAGTGGAGGCAGAGGCGATATATTTC
CTCCTCATAGATGCAGCGGAGCCACAACCAGCGTGGGAAAAGTTTTCCCA
CTGTCCGTCAGCCTGAGCATGAGCCTGATCTCCAGAACCAGCGAGGTGAT
CAACATGCTGACCGCCATTAGCGACGGCGTGTACGGCAAAACCTACCTGC
TCGTTCCTGATGACATCGAAAGAGAGTTCGACACCAGAGAGATCCGGGTG
TTCGAGATCGGCTTCATTAAAAGATGGCTGAACGACATGCCTCTGCTTCA
AACAACCAACTACATGGTCCTGCCTAAGAACAGCAAGGCCAAGGTGTGCA
CCATCGCTGTGGGCGAGCTGACACTGGCTTCCCTGTGCGTGGAAGAAAGC
ACCGTGCTGCTGTACCACGACAGCAGCGGCAGCCAGGACGGAATTCTGGT
CGTGACCCTGGGCATCTTCTGGGCCACCCCTATGGATCACATCGAGGAAG
TGATTCCCGTGGCCCACCCTTCTATGAAGAAGATCCACATCACCAACCAC
AGAGGATTCATCAAGGACAGCATCGCCACCTGGATGGTGCCTGCCCTGGC
TTCTGAGAAACAGGAGGAACAGAAAGGCTGTCTGGAAAGCGCCTGTCAGA
GAAAGACATATCCTATGTGCAACCAGGCCAGCTGGGAGCCTTTCGGCGGA
AGACAGCTGCCCAGCTACGGCCGCCTGACCCTGCCTCTGGACGCCAGCGT
GGACCTGCAACTGAACATCTCTTTCACCTACGGTCCTGTGATTCTGAATG
GCGATGGCATGGACTACTACGAGTCCCCTCTGCTGAACAGCGGCTGGCTG
ACCATCCCCCCTAAGGACGGCACCATCAGCGGCCTGATCAACAAGGCCGG
CAGAGGCGACCAGTTCACCGTGCTGCCACATGTGCTGACATTCGCCCCTA
GAGAATCCTCTGGCAACTGCTACCTGCCTATCCAGACATCTCAGATCCGG
GATAGAGACGTGCTGATCGAATCTAATATCGTGGTGCTGCCAACACAGAG
CATCAGATACGTGATCGCCACTTACGACATCAGCCGGAGCGATCACGCCA
TCGTGTACTACGTGTACGACCCTATCAGGACCATCAGCTATACACACCCC
TTCAGACTGACCACGAAGGGCCGGCCCGATTTCCTGCGGATCGAGTGCTT
CGTGTGGGACGACAACCTGTGGTGTCACCAGTTCTACCGGTTCGAGGCCG
ACATCGCCAATTCTACCACATCTGTGGAAAACCTGGTGCGGATCCGGTTC
TCTTGTAACCGGGGAGGAGGAGGAAGCGGAGGAGGAGGATCCGGCGGCGG
CGGCTCTGCGGCCGCGCAGGTTCAACTGGTGCAGAGCGGAGGCGGCCTGG
TCCAGCCCGGCGGAAGCCTGGGCCTGAGCTGTGCCGCTTCTGGCAACATC
GGCAGCAGAGATAATATGGGCTGGTATAGACAGGCCCCTGGAAAACAGAG
AGAGTGGGTGGCTACAATCAGCGGCTACGGCATCGCCACATACCGGGACA
GCGTGAAGGGCAGATTCACCGTGGCCAAGGACACCGCCAAGAACATCGTG
TCCCTGCAGATGAACTACCTGACCACCGAAGATACAGCCGTGTACTACTG
CTACGCCTACGCCGTGGACAGCCGGAACATCTTCTGGTCCCAGGGCACCC
AGGTGACCGTGTCTGGCGGCGGCTCCGGCGGCGGCAGCTATCCCTATGAT
GTGCCCGACTATGCGTGA.

By “CDV-H-hCD105 scFv polypeptide” is meant a polypeptide or fragment thereof having at least about 85% amino acid sequence identity to the sequence provided below, and that functions in lentivirus envelope fusion with the membrane of a target cell expressing a CD105 polypeptide. In some instances the CD105 polypeptide is a murine or human CD105 polypeptide. In embodiments, the CDV-H-hCD105 scFv polypeptide functions in combination with an MeV-Fc30, DMV-F, DMV-Fc30, CDV-F, or CDV-Fc30 polypeptide to fuse a lentivirus lipid envelope with a membrane (e.g., a cell membrane). An exemplary CDV-H-hCD105 scFv polypeptide sequence is provided below.

MLPYQDKVGAFYKDNARANSTKLSLVTEGHGGRRPPYLLFVLLILLVGIL
ALLAITGVRFHQVSTSNMEFSRLLKEDMEKSEAVHHQVIDVLTPLFKIIG
DEIGLRLPQKLNEIKQFILQKTNFFNPNREFDFRDLHWCINPPSTVKVNF
TNYCESIGIRKAIASAANPILLSALSGGRGDIFPPHRCSGATTSVGKVFP
LSVSLSMSLISRTSEVINMLTAISDGVYGKTYLLVPDDIEREFDTREIRV
FEIGFIKRWLNDMPLLQTTNYMVLPKNSKAKVCTIAVGELTLASLCVEES
TVLLYHDSSGSQDGILVVTLGIFWATPMDHIEEVIPVAHPSMKKIHITNH
RGFIKDSIATWMVPALASEKQEEQKGCLESACQRKTYPMCNQASWEPFGG
RQLPSYGRLTLPLDASVDLQLNISFTYGPVILNGDGMDYYESPLLNSGWL
TIPPKDGTISGLINKAGRGDQFTVLPHVLTFAPRESSGNCYLPIQTSQIR
DRDVLIESNIVVLPTQSIRYVIATYDISRSDHAIVYYVYDPIRTISYTHP
FRLTTKGRPDFLRIECFVWDDNLWCHQFYRFEADIANSTTSVENLVRIRF
SCNRGGGGSGGGGSGGGGSAAAMAEVQLLESGGGLVQPGGSLRLSCAASG
FTFSSYAMSWVRQAPGKGLEWVSAIYGSDGDTTYADSVKGRFTISRDNSK
NTLYLQMNSLRAEDTAVYYCARVFYTAGFDYWGQGTLVTVSSGSDSNAGH
ASAGNTSDIELTQSPSSLSASVGDRVTITCRASQSISSSLNWYQQKPGKA
PKLLIYAASSLQSGVPSRFSGSGSGTDFTLTISSLQPEDFATYYCQQAPA
KPPTFGQGTKLEIKRAAAYPYDVPDYA.

In the above polypeptide sequence, the cytoplasmic domain is underlined, the transmembrane domain is in italics, the extravirion domain is in plain text, the (G4S)3 linker is in bold, the NotI site is in bold underline, the anti-hCD105 scFv domain is in bold italic, and the HA tag is double-underlined.

By “CDV-H-hCD105 scFv polynucleotide” is meant a polynucleotide encoding a CDV-H-hCD105 scFv polypeptide. An exemplary CDV-H-hCD105 scFv polynucleotide sequence is provided below.

ATGCTGCCCTATCAGGATAAGGTGGGCGCTTTTTACAAGGATAACGCTAG
AGCCAACAGCACAAAGCTGAGCCTGGTCACCGAGGGCCACGGCGGCAGAA
GACCTCCATACCTGCTGTTTGTGCTGCTAATTCTGCTGGTGGGCATCCTG
GCACTGCTGGCCATTACAGGCGTGCGGTTTCACCAGGTGTCCACCAGCAA
CATGGAATTCAGCAGACTGCTGAAGGAAGATATGGAAAAGAGCGAGGCCG
TGCACCACCAAGTGATCGACGTGCTCACCCCCCTGTTCAAGATCATCGGC
GACGAGATCGGCCTGAGGTTGCCTCAGAAACTGAACGAGATCAAGCAGTT
TATCCTGCAGAAGACCAATTTCTTCAACCCTAACAGAGAGTTTGACTTCA
GAGATCTGCACTGGTGCATCAACCCCCCCAGCACAGTAAAGGTGAATTTC
ACGAATTACTGCGAGAGCATCGGAATCAGAAAGGCCATCGCCAGCGCCGC
CAACCCCATCCTGCTGTCTGCTCTGAGTGGAGGCAGAGGCGATATATTTC
CTCCTCATAGATGCAGCGGAGCCACAACCAGCGTGGGAAAAGTTTTCCCA
CTGTCCGTCAGCCTGAGCATGAGCCTGATCTCCAGAACCAGCGAGGTGAT
CAACATGCTGACCGCCATTAGCGACGGCGTGTACGGCAAAACCTACCTGC
TCGTTCCTGATGACATCGAAAGAGAGTTCGACACCAGAGAGATCCGGGTG
TTCGAGATCGGCTTCATTAAAAGATGGCTGAACGACATGCCTCTGCTTCA
AACAACCAACTACATGGTCCTGCCTAAGAACAGCAAGGCCAAGGTGTGCA
CCATCGCTGTGGGCGAGCTGACACTGGCTTCCCTGTGCGTGGAAGAAAGC
ACCGTGCTGCTGTACCACGACAGCAGCGGCAGCCAGGACGGAATTCTGGT
CGTGACCCTGGGCATCTTCTGGGCCACCCCTATGGATCACATCGAGGAAG
TGATTCCCGTGGCCCACCCTTCTATGAAGAAGATCCACATCACCAACCAC
AGAGGATTCATCAAGGACAGCATCGCCACCTGGATGGTGCCTGCCCTGGC
TTCTGAGAAACAGGAGGAACAGAAAGGCTGTCTGGAAAGCGCCTGTCAGA
GAAAGACATATCCTATGTGCAACCAGGCCAGCTGGGAGCCTTTCGGCGGA
AGACAGCTGCCCAGCTACGGCCGCCTGACCCTGCCTCTGGACGCCAGCGT
GGACCTGCAACTGAACATCTCTTTCACCTACGGTCCTGTGATTCTGAATG
GCGATGGCATGGACTACTACGAGTCCCCTCTGCTGAACAGCGGCTGGCTG
ACCATCCCCCCTAAGGACGGCACCATCAGCGGCCTGATCAACAAGGCCGG
CAGAGGCGACCAGTTCACCGTGCTGCCACATGTGCTGACATTCGCCCCTA
GAGAATCCTCTGGCAACTGCTACCTGCCTATCCAGACATCTCAGATCCGG
GATAGAGACGTGCTGATCGAATCTAATATCGTGGTGCTGCCAACACAGAG
CATCAGATACGTGATCGCCACTTACGACATCAGCCGGAGCGATCACGCCA
TCGTGTACTACGTGTACGACCCTATCAGGACCATCAGCTATACACACCCC
TTCAGACTGACCACGAAGGGCCGGCCCGATTTCCTGCGGATCGAGTGCTT
CGTGTGGGACGACAACCTGTGGTGTCACCAGTTCTACCGGTTCGAGGCCG
ACATCGCCAATTCTACCACATCTGTGGAAAACCTGGTGCGGATCCGGTTC
TCTTGTAACCGGGGAGGAGGAGGAAGCGGAGGAGGAGGATCCGGCGGCGG
CGGCTCTGCGGCCGCCATGGCGGAGGTGCAGCTGCTGGAGTCCGGAGGAG
GCCTGGTGCAGCCAGGAGGCTCTCTGAGGCTGAGCTGCGCAGCATCCGGC
TTCACCTTTTCTAGCTACGCCATGAGCTGGGTGCGCCAGGCACCAGGCAA
GGGCCTGGAGTGGGTGAGCGCCATCTACGGATCCGACGGCGATACCACAT
ATGCCGATTCCGTGAAGGGCAGGTTCACCATCTCTCGCGACAACAGCAAG
AATACACTGTATCTGCAGATGAACTCTCTGAGGGCCGAGGATACCGCCGT
GTACTATTGCGCCCGCGTGTTTTACACAGCCGGCTTCGACTATTGGGGCC
AGGGCACCCTGGTGACAGTGTCCTCTGGCTCCGATTCTAACGCAGGACAC
GCATCTGCCGGAAATACCAGCGACATCGAGCTGACACAGTCCCCAAGCTC
CCTGAGCGCCTCCGTGGGCGACAGGGTGACCATCACATGTCGCGCCTCTC
AGAGCATCTCTAGCTCCCTGAATTGGTACCAGCAGAAGCCCGGCAAGGCC
CCTAAGCTGCTGATCTATGCAGCATCTAGCCTGCAGTCCGGAGTGCCATC
TAGGTTTTCCGGCTCTGGCAGCGGCACCGATTTCACCCTGACAATCTCCT
CTCTGCAGCCCGAGGACTTTGCCACATACTATTGTCAGCAGGCCCCTGCC
AAGCCTCCAACCTTCGGCCAGGGCACAAAGCTGGAGATCAAGAGGGCCGC
CGCCTACCCATACGATGTTCCAGATTACGCTTGA.

By “CDV—H-CD7 (Humanized VHH10) polypeptide” is meant a polypeptide or fragment thereof having at least about 85% amino acid sequence identity to the sequence provided below, and that functions in lentivirus envelope fusion with the membrane of a target cell expressing a CD7 polypeptide. In some instances the CD7 polypeptide is a murine or human CD7 polypeptide. In embodiments, the CDV—H-CD7 (Humanized VHH10) polypeptide functions in combination with an MeV-Fc30, DMV-F, DMV-Fc30, CDV-F, or CDV-Fc30 polypeptide to fuse a lentivirus lipid envelope with a membrane (e.g., a cell membrane). An exemplary CDV-H-CD7 (Humanized VHH10) polypeptide sequence is provided below.

MLPYQDKVGAFYKDNARANSTKLSLVTEGHGGRRPPYLLFVLLILLVGIL
ALLAITGVRFHQVSTSNMEFSRLLKEDMEKSEAVHHQVIDVLTPLFKIIG
DEIGLRLPQKLNEIKQFILQKTNFFNPNREFDFRDLHWCINPPSTVKVNF
TNYCESIGIRKAIASAANPILLSALSGGRGDIFPPHRCSGATTSVGKVFP
LSVSLSMSLISRTSEVINMLTAISDGVYGKTYLLVPDDIEREFDTREIRV
FEIGFIKRWLNDMPLLQTTNYMVLPKNSKAKVCTIAVGELTLASLCVEES
TVLLYHDSSGSQDGILVVTLGIFWATPMDHIEEVIPVAHPSMKKIHITNH
RGFIKDSIATWMVPALASEKQEEQKGCLESACQRKTYPMCNQASWEPFGG
RQLPSYGRLTLPLDASVDLQLNISFTYGPVILNGDGMDYYESPLLNSGWL
TIPPKDGTISGLINKAGRGDQFTVLPHVLTFAPRESSGNCYLPIQTSQIR
DRDVLIESNIVVLPTQSIRYVIATYDISRSDHAIVYYVYDPIRTISYTHP
FRLTTKGRPDFLRIECFVWDDNLWCHQFYRFEADIANSTTSVENLVRIRF
SCNRGGGGSGGGGSGGGGSAAADVQLQESGGGSVQAGGSLRLSCAASGYT
HSSYCMAWFRQAPGREREGVASIDSDGTTSYADSVKGRFTISQDNAKNTL
YLQMNSLKPEDTAMYYCAARFGPMGCVDLSTLSFGHWGQGTQVTVSITGG
GSGGGSYPYDVPDYA.

In the above polypeptide sequence, the cytoplasmic domain is underlined, the transmembrane domain is in italics, the extravirion domain is in plain text, the (G4S)3 linker is in bold, the NotI site is in bold underline, the anti-CD7 VHH domain is in bold italic, the (G3S)2 linker is in italic underline, and the HA tag is double-underlined.

By “CDV—H-CD7 (Humanized VHH10) polynucleotide” is meant a polynucleotide encoding a CDV—H-CD7 (Humanized VHH10) polypeptide. An exemplary CDV—H-CD7 (Humanized VHH10) polynucleotide sequence is provided below.

ATGCTGCCCTATCAGGATAAGGTGGGCGCTTTTTACAAGGATAACGCTAG
AGCCAACAGCACAAAGCTGAGCCTGGTCACCGAGGGCCACGGCGGCAGAA
GACCTCCATACCTGCTGTTTGTGCTGCTAATTCTGCTGGTGGGCATCCTG
GCACTGCTGGCCATTACAGGCGTGCGGTTTCACCAGGTGTCCACCAGCAA
CATGGAATTCAGCAGACTGCTGAAGGAAGATATGGAAAAGAGCGAGGCCG
TGCACCACCAAGTGATCGACGTGCTCACCCCCCTGTTCAAGATCATCGGC
GACGAGATCGGCCTGAGGTTGCCTCAGAAACTGAACGAGATCAAGCAGTT
TATCCTGCAGAAGACCAATTTCTTCAACCCTAACAGAGAGTTTGACTTCA
GAGATCTGCACTGGTGCATCAACCCCCCCAGCACAGTAAAGGTGAATTTC
ACGAATTACTGCGAGAGCATCGGAATCAGAAAGGCCATCGCCAGCGCCGC
CAACCCCATCCTGCTGTCTGCTCTGAGTGGAGGCAGAGGCGATATATTTC
CTCCTCATAGATGCAGCGGAGCCACAACCAGCGTGGGAAAAGTTTTCCCA
CTGTCCGTCAGCCTGAGCATGAGCCTGATCTCCAGAACCAGCGAGGTGAT
CAACATGCTGACCGCCATTAGCGACGGCGTGTACGGCAAAACCTACCTGC
TCGTTCCTGATGACATCGAAAGAGAGTTCGACACCAGAGAGATCCGGGTG
TTCGAGATCGGCTTCATTAAAAGATGGCTGAACGACATGCCTCTGCTTCA
AACAACCAACTACATGGTCCTGCCTAAGAACAGCAAGGCCAAGGTGTGCA
CCATCGCTGTGGGCGAGCTGACACTGGCTTCCCTGTGCGTGGAAGAAAGC
ACCGTGCTGCTGTACCACGACAGCAGCGGCAGCCAGGACGGAATTCTGGT
CGTGACCCTGGGCATCTTCTGGGCCACCCCTATGGATCACATCGAGGAAG
TGATTCCCGTGGCCCACCCTTCTATGAAGAAGATCCACATCACCAACCAC
AGAGGATTCATCAAGGACAGCATCGCCACCTGGATGGTGCCTGCCCTGGC
TTCTGAGAAACAGGAGGAACAGAAAGGCTGTCTGGAAAGCGCCTGTCAGA
GAAAGACATATCCTATGTGCAACCAGGCCAGCTGGGAGCCTTTCGGCGGA
AGACAGCTGCCCAGCTACGGCCGCCTGACCCTGCCTCTGGACGCCAGCGT
GGACCTGCAACTGAACATCTCTTTCACCTACGGTCCTGTGATTCTGAATG
GCGATGGCATGGACTACTACGAGTCCCCTCTGCTGAACAGCGGCTGGCTG
ACCATCCCCCCTAAGGACGGCACCATCAGCGGCCTGATCAACAAGGCCGG
CAGAGGCGACCAGTTCACCGTGCTGCCACATGTGCTGACATTCGCCCCTA
GAGAATCCTCTGGCAACTGCTACCTGCCTATCCAGACATCTCAGATCCGG
GATAGAGACGTGCTGATCGAATCTAATATCGTGGTGCTGCCAACACAGAG
CATCAGATACGTGATCGCCACTTACGACATCAGCCGGAGCGATCACGCCA
TCGTGTACTACGTGTACGACCCTATCAGGACCATCAGCTATACACACCCC
TTCAGACTGACCACGAAGGGCCGGCCCGATTTCCTGCGGATCGAGTGCTT
CGTGTGGGACGACAACCTGTGGTGTCACCAGTTCTACCGGTTCGAGGCCG
ACATCGCCAATTCTACCACATCTGTGGAAAACCTGGTGCGGATCCGGTTC
TCTTGTAACCGGGGAGGAGGAGGAAGCGGAGGAGGAGGATCCGGCGGCGG
CGGCTCTGCGGCCGCGGATGTGCAGCTGCAGGAGAGCGGCGGCGGATCTG
TGCAAGCTGGCGGAAGCCTGAGACTGTCTTGCGCCGCTTCTGGCTACACC
CACAGCAGCTATTGCATGGCCTGGTTCCGGCAGGCCCCTGGCAGAGAACG
GGAAGGCGTGGCCAGCATCGACAGCGACGGCACCACCAGCTACGCCGATA
GCGTCAAGGGCAGATTCACCATCAGCCAGGACAATGCCAAAAACACCCTG
TACCTGCAGATGAACAGCCTGAAGCCCGAGGACACAGCCATGTACTACTG
TGCCGCCAGATTTGGCCCTATGGGCTGCGTGGACCTGTCCACCCTCTCCT
TCGGCCACTGGGGACAGGGCACACAGGTGACAGTGTCCATCACCGGCGGC
GGCTCCGGCGGCGGCAGCTATCCCTATGATGTGCCCGACTATGCGTGA.

By “CDV—H-CD45 VHH (32) polypeptide” is meant a polypeptide or fragment thereof having at least about 85% amino acid sequence identity to the sequence provided below, and that functions in lentivirus envelope fusion with the membrane of a target cell expressing a CD45 polypeptide. In some instances the CD45 polypeptide is a murine or human CD45 polypeptide. In embodiments, the CDV—H-CD45 VHH (32) polypeptide functions in combination with an MeV-Fc30, DMV-F, DMV-Fc30, CDV-F, or CDV-Fc30 polypeptide to fuse a lentivirus lipid envelope with a membrane (e.g., a cell membrane). An exemplary CDV—H-CD45 VHH (32) polypeptide sequence is provided below.

MLPYQDKVGAFYKDNARANSTKLSLVTEGHGGRRPPYLLFVLLILLVGIL
ALLAITGVRFHQVSTSNMEFSRLLKEDMEKSEAVHHQVIDVLTPLFKIIG
DEIGLRLPQKLNEIKQFILQKTNFFNPNREFDFRDLHWCINPPSTVKVNF
TNYCESIGIRKAIASAANPILLSALSGGRGDIFPPHRCSGATTSVGKVFP
LSVSLSMSLISRTSEVINMLTAISDGVYGKTYLLVPDDIEREFDTREIRV
FEIGFIKRWLNDMPLLQTTNYMVLPKNSKAKVCTIAVGELTLASLCVEES
TVLLYHDSSGSQDGILVVTLGIFWATPMDHIEEVIPVAHPSMKKIHITNH
RGFIKDSIATWMVPALASEKQEEQKGCLESACQRKTYPMCNQASWEPFGG
RQLPSYGRLTLPLDASVDLQLNISFTYGPVILNGDGMDYYESPLLNSGWL
TIPPKDGTISGLINKAGRGDQFTVLPHVLTFAPRESSGNCYLPIQTSQIR
DRDVLIESNIVVLPTQSIRYVIATYDISRSDHAIVYYVYDPIRTISYTHP
FRLTTKGRPDFLRIECFVWDDNLWCHQFYRFEADIANSTTSVENLVRIRF
SCNRGGGGSGGGGSGGGGSAAAQVQLVQSGGGLVQPGGSLRLSCAASGRA
FNSAAMGWYRQAPGSQRELVASISAGTASYADAVKGRFTISRDYAKNIIY
LQMNSLKPDDTAVYFCNYRTTYTSGYSEDYWGQGTQVTVSAAAYPYDVPD
YA.

In the above polypeptide sequence, the cytoplasmic domain is underlined, the transmembrane domain is in italics, the extravirion domain is in plain text, the (G4S)3 linker is in bold, the NotI site is in bold underline, the anti-CD45 VHH domain is in bold italic, the NotI site is in italic underline, and the HA tag is double-underlined.

By “CDV—H-CD45 VHH (32) polynucleotide” is meant a polynucleotide encoding a CDV—H-CD45 VHH (32) polypeptide.

By “MeV-Hc18-CDV-MHCII (N11) polypeptide” is meant a polypeptide or fragment thereof having at least about 85% amino acid sequence identity to the sequence provided below, and that functions in lentivirus envelope fusion with the membrane of a target cell expressing an MHCII polypeptide. In some instances the MHCII polypeptide is a murine or human MHCII polypeptide. In embodiments, the MeV-Hcl8-CDV-MHCII (N11) polypeptide functions in combination with an MeV-Fc30, DMV-F, DMV-Fc30, CDV-F, or CDV-Fc30 polypeptide to fuse a lentivirus lipid envelope with a membrane (e.g., a cell membrane).

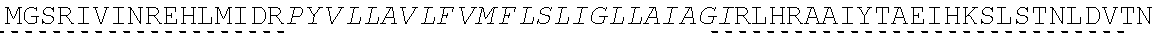

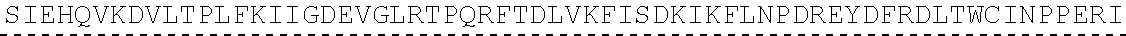

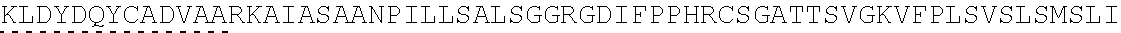

SRTSEVINMLTAISDGVYGKTYLLVPDDIEREFDTREIRVFEIGFIKRWLNDMPLLQTTNYMVL
PKNSKAKVCTIAVGELTLASLCVEESTVLLYHDSSGSQDGILVVTLGIFWATPMDHIEEVIPVA
HPSMKKIHITNHRGFIKDSIATWMVPALASEKQEEQKGCLESACORKTYPMCNQASWEPFGGRQ
LPSYGRLTLPLDASVDLQLNISFTYGPVILNGDGMDYYESPLLNSGWLTIPPKDGTISGLINKA
GRGDQFTVLPHVLTFAPRESSGNCYLPIQTSQIRDRDVLIESNIVVLPTQSIRYVIATYDISRS
DHAIVYYVYDPIRTISYTHPFRLTTKGRPDFLRIECFVWDDNLWCHQFYRFEADIANSTTSVEN
LVRIRFSCNRGGGGSGGGGSGGGGSAAAQVQLVQSGGGLVQPGGSLGLSCAASGNIGSRDNMGW
YRQAPGKQREWVATISGYGIATYRDSVKGRFTVAKDTAKNIVSLOMNYLTTEDTAVYYCYAYAV
DSRNIFWSQGTQVTVSGGGSGGGSYPYDVPDYA.

In the above polypeptide sequence, the cytoplasmic domain is underlined, the transmembrane domain is in italics, the MeV-H stalk is underlined with a dashed line, the extravirion domain is in plain text, the (G4S)3 linker is in bold, the NotI site is in bold underline, the anti-MHCII VHH domain is in bold italic, the (G3S)2 linker is in italic underline, and the HA tag is double-underlined.

By “MeV-Hc18-CDV-MHCII (N11) polynucleotide” is meant a polynucleotide encoding a MeV-Hc18-CDV-MHCII (N11) polypeptide.

By “MeV-Hc18-DMV-MHCII (N11) polypeptide” is meant a polypeptide or fragment thereof having at least about 85% amino acid sequence identity to the sequence provided below, and that functions in lentivirus envelope fusion with the membrane of a target cell expressing an MHCII polypeptide. In some instances the MHCII polypeptide is a murine or human MHCII polypeptide. In embodiments, the MeV-Hc18-DMV-MHCII (N11) polypeptide functions in combination with an MeV-Fc30, DMV-F, DMV-Fc30, CDV-F, or CDV-Fc30 polypeptide to fuse a lentivirus lipid envelope with a membrane (e.g., a cell membrane).

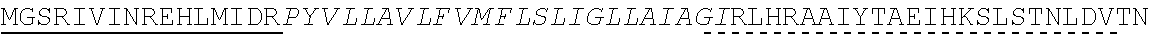

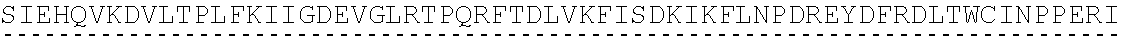

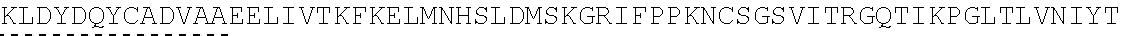

TRNFEVSFMVTVISGGMYGKTYFLKPPEPDDPFEFQAFRIFEVGLVRDVGSREPVLQMTNFMVI
DEDEGLNFCLLSVGELRLAAVCVRGRPVVTKDIGGYKDEPFKVVTLGIIGGGLSNQKTEIYPTI
DSSIEKLYITSHRGIIRNSKARWSVPAIRSDDKDKMEKCTQALCKSRPPPSCNSSDWEPLTSNR
IPAYAYIALEIKEDSGLELDITSNYGPLIIHGAGMDIYEGPSSNQDWLAIPPLSQSVLGVINKV
DFTAGFDIKPHTLTTAVDYESGKCYVPVELSGAKDQDLKLESNLVVLPTKDFGYVTATYDTSRS
EHAIVYYVYDTARSSSYFFPFRIKARGEPIYLRIECFPWSRQLWCHHYCMINSTVSNEIVVVDN
LVSINMSCSRGGGGSGGGGSGGGGSAAAQVOLVOSGGGLVQPGGSLGLSCAASGNIGSRDNMGW
YRQAPGKQREWVATISGYGIATYRDSVKGRFTVAKDTAKNIVSLOMNYLTTEDTAVYYCYAYAV
DSRNIFWSQGTQVTVSGGGSGGGSYPYDVPDYA.

In the above polypeptide sequence, the cytoplasmic domain is underlined, the transmembrane domain is in italics, the MeV-H stalk is underlined with a dashed line, the extravirion domain is in plain text, the (G4S)3 linker is in bold, the NotI site is in bold underline, the anti-MHCII VHH domain is in bold italic, the (G3S)2 linker is in italic underline, and the HA tag is double-underlined.

By “MeV-Hc18-DMV-MHCII (N11) polynucleotide” is meant a polynucleotide encoding a MeV-Hc18-DMV-MHCII (N11) polypeptide.

By “FMV-H-CD45 (32) polypeptide” is meant a polypeptide or fragment thereof having at least about 85% amino acid sequence identity to the sequence provided below, and that functions in lentivirus envelope fusion with the membrane of a target cell expressing a CD45 polypeptide. In some instances the CD45 polypeptide is a murine or human CD45 polypeptide. In embodiments, the FMV-H-CD45 (32) polypeptide functions in combination with an MeV-Fc30, DMV-F, DMV-Fc30, CDV-F, or CDV-Fc30 polypeptide to fuse a lentivirus lipid envelope with a membrane (e.g., a cell membrane). An exemplary FMV-H-CD45 (32)

MESNNIKYYKDSSRYFGKILDEHKTINSQLYSLSIKVITIIAIIVSLIAT
IITIINATSGRTTLNSNTDILLSQRDEIHNIQEMIFDRIYPLINAMSTEL
GLHIPTLLDELTKAIDQKIKIMHPPVDTVTSDLNWCIKPPNGIIIDPKSY
CESMELSKTYELLLDQLDVSRKKSLIINRKNINQCQLVDNSKIIFATVNI
QSTPRFLNFGHTVSNQRITFGQGTYSSTYVITIQEDGVTDVQYRVFEIGY
ISDQFGVFPSLIVSRVLPIRMLLGMESCTLTSDRLGGYFLCMNTLTRSIY
DYVSIRDLKSLYITIPHYGKVNYTYFNFGKIRSPHEIDKIWLTSDRGQII
SGYFAAFVTITIRNYNNYPYKCLNNPCFDNSENYCRGWYKNITGTDDVPI
LAYLLVEMYDEEGPLITLVAIPPYNYTAPSHNSLYYDDKINKLIMTTSHI
GYIQINEVHEVIVGDNLKAILLNRLSDEHPNLTACRLNQGIKEQYKSDGT
IISNSALIDIQERMYITVKAIPPAGNYNFTVELHSRSNTSYVSLPKQFNA
KYDKLHLECFSWDKSWWCALIPQFSLSWNESLSVDTAIFNLISCKGGGGS
GGGGSGGGGSAAAQVQLVQSGGGLVQPGGSLRLSCAASGRAFNSAAMGWY
RQAPGSQRELVASISAGTASYADAVKGRFTISRDYAKNIIYLQMNSLKPD
DTAVYFCNYRTTYTSGYSEDYWGQGTQVTVSGGGSGGGSYPYDVPDYA.

In the above polypeptide sequence, the cytoplasmic domain is underlined, the transmembrane domain is in italics, the extravirion domain is in plain text, the (G4S)3 linker is in bold, the NotI site is in bold underline, the anti-CD45 VHH domain is in bold italic, the (G3S)2 linker is in italic underline, and the HA tag is double-underlined.

By “FMV-H-CD45 (32) polynucleotide” is meant a polynucleotide encoding a FMV-H-CD45 (32) polypeptide.

By “PPRV—H-CD45 (32) polypeptide” is meant a polypeptide or fragment thereof having at least about 85% amino acid sequence identity to the sequence provided below, and that functions in lentivirus envelope fusion with the membrane of a target cell expressing a CD45 polypeptide. In some instances the CD45 polypeptide is a murine or human CD45 polypeptide. In embodiments, the PPRV—H-CD45 (32) polypeptide functions in combination with an MeV-Fc30, DMV-F, DMV-Fc30, CDV-F, or CDV-Fc30 polypeptide to fuse a lentivirus lipid envelope with a membrane (e.g., a cell membrane). An exemplary PPRV—H-CD45 (32) polypeptide sequence is provided below.

MSAQRERINAFYKDNLHNKTHRVILDRERLTIERPYILLGVLLVMFLSL
IGLLAIAGIRLHRATVGTAEIQSRLNTNIELTESIDHQTKDVLTPLFKI
IGDEVGIRIPQKFSDLVKFISDKIKFLNPDREYDFRDLRWCMNPPERVK
INFDQFCEYKAAVKSVEHIFESSLNRSERLRLLTLGPGTGCLGRTVTRA
QFSELTLTLMDLDLEIKHNVSSVFTVVEEGLFGRTYTVWRSDTGKPSTS
PGIGHFLRVFEIGLVRDLELGAPIFHMTNYLTVNMSDDYRSCLLAVGEL
KLTALCTPSETVTLSESGVPKREPLVVVILNLAGPTLGGELYSVLPTTD
PTVEKLYLSSHRGIIKDNEANWVVPSTDVRDLQNKGECLVEACKTRPPS
FCNGTGIGPWSEGRIPAYGVIRVSLDLASDPGVVITSVFGPLIPHLSGM
DLYNNPFSRAAWLAVPPYEQSFLGMINTIGFPDRAEVMPHILTTEIRGP
RGRCHVPIELSSRIDDDIKIGSNMVVLPTKDLRYITATYDVSRSEHAIV
YYIYDTGRSSSYFYPVRLNFRGNPLSLRIECFPWYHKVWCYHDCLIYNT
ITNEEVHTRGLTGIEVTCNPVGGGGSGGGGSGGGGSAAAQVQLVQSGGG
LVQPGGSLRLSCAASGRAFNSAAMGWYRQAPGSQRELVASISAGTASYA
DAVKGRFTISRDYAKNIIYLQMNSLKPDDTAVYFCNYRTTYTSGYSEDY
WGQGTQVTVSGGGSGGGSYPYDVPDYA.

In the above polypeptide sequence, the cytoplasmic domain is underlined, the transmembrane domain is in italics, the extravirion domain is in plain text, the (G4S)3 linker is in bold, the NotI site is in bold underline, the anti-CD45 VHH domain is in bold italic, the (G3S)2 linker is in italic underline, and the HA tag is double-underlined.

By “PPRV—H-CD45 (32) polynucleotide” is meant a polynucleotide encoding a PPRV—H-CD45 (32) polypeptide.

By “RPV—H-CD45 (32) polypeptide” is meant a polypeptide or fragment thereof having at least about 85% amino acid sequence identity to the sequence provided below, and that functions in lentivirus envelope fusion with the membrane of a target cell expressing a CD45 polypeptide. In some instances the CD45 polypeptide is a murine or human CD45 polypeptide. In embodiments, the RPV—H-CD45 (32) polypeptide functions in combination with an MeV-Fc30, DMV-F, DMV-Fc30, CDV-F, or CDV-Fc30 polypeptide to fuse a lentivirus lipid envelope with a membrane (e.g., a cell membrane). An exemplary RPV—H-CD45 (32) polypeptide sequence is provided below.

MSPPRDRVDAYYKDNFQFKNTRVVLNKEQLLIERPCMLLTVLFVMFLSL
VGLLAIAGIRLHRAAVNTAKINNDLTTSIDITKSIEYQVKDVLTPLFKI
IGDEVGLRTPQRFTDLTKFISDKIKFLNPDKEYDFRDINWCINPPERIK
IDYDQYCAHTAAEDLITMLVNSSLTGTTVLRTSLVNLGRNCTGPTTTKG
QFSNISLTLSGIYSGRGYNISSMITITGKGMYGSTYLVGKYNQRARRPS
IVWQQDYRVFEVGIIRELGVGTPVFHMTNYLELPRQPELETCMLALGES
KLAALCLADSPVALHYGRVGDDNKIRFVKLGVWASPADRDTLATLSAID
PTLDGLYITTHRGIIAAGTAIWAVPVTRTDDQVKMGKCRLEACRDRPPP
FCNSTDWEPLEAGRIPAYGVLTIKLGLADEPKVDIISEFGPLITHDSGM
DLYTSFDGTKYWLTTPPLONSALGTVNTLVLEPSLKISPNILTLPIRSG
GGDCYTPTYLSDRADDDVKLSSNLVILPSRDLQYVSATYDISRVEHAIV
YHIYSTGRLSSYYYPFKLPIKGDPVSLQIECFPWDRKLWCHHFCSVIDS
GTGEQVTHIGVVGIEITCNGKGGGGSGGGGSGGGGSAAAQVQLVQSGGG
LVQPGGSLRLSCAASGRAFNSAAMGWYRQAPGSQRELVASISAGTASYA
DAVKGRFTISRDYAKNIIYLQMNSLKPDDTAVYFCNYRTTYTSGYSEDY
WGQGTQVTVSGGGSGGGSYPYDVPDYA.

In the above polypeptide sequence, the cytoplasmic domain is underlined, the transmembrane domain is in italics, the extravirion domain is in plain text, the (G4S)3 linker is in bold, the NotI site is in bold underline, the anti-hCD45 VHH sequence is in bold italic, the (G3S)2 linker is in italic underline, and the HA tag is double-underlined.

By “RPV—H-CD45 (32) polynucleotide” is meant a polynucleotide encoding a RPV—H-CD45 (32) polypeptide.

By “RMV—H-CD45 (32) polypeptide” is meant a polypeptide or fragment thereof having at least about 85% amino acid sequence identity to the sequence provided below, and that functions in lentivirus envelope fusion with the membrane of a target cell expressing a CD45 polypeptide. In some instances the CD45 polypeptide is a murine or human CD45 polypeptide. In embodiments, the RMV—H-CD45 (32) polypeptide functions in combination with an MeV-Fc30, DMV-F, DMV-Fc30, CDV-F, or CDV-Fc30 polypeptide to fuse a lentivirus lipid envelope with a membrane (e.g., a cell membrane). An exemplary RMV—H-CD45 (32) polypeptide sequence is provided below.

MSAQRERINAFYKDNPHNKNHRVILDRERLVIERPYILLGVLLVMFLSL
IGLLAIAGIRLHRATVGTSEIQSRLNTNIELTESIDHQTKDVLTPLFKI
IGDEVGIRIPQKFSDLVKFISDKIKFLNPDREYDFRDLRWCMNPPERVK
INFDQFCEYKAAVKSIEHIFESPLNKSKKLQSLTLGPGTGCLGRTVTRA
HFSELTLTLMDLDLEMKHNVSSVFTVVEEGLFGRTYTVWRSDARDPSTD
LGIGHFLRVFEIGLVRDLGLGPPVFHMTNYLTVNMSDDYRRCLLAVGEL
KLTALCSSSETVTLGERGVPKREPLVVVILNLAGPTLGGELYSVLPTSD
LMVEKLYLSSHRGIIKDDEANWVVPSTDVRDLQNKGECLVEACKTRPPS
FCNGTGSGPWSEGRIPAYGVIRVSLDLASDPGVVITSVFGPLIPHLSGM
DLYNNPFSRAVWLAVPPYEQSFLGMINTIGFPNRAEVMPHILTTEIRGP
RGRCHVPIELSRRVDDDIKIGSNMVILPTIDLRYITATYDVSRSEHAIV
YYIYDTGRSSSYFYPVRLNFKGNPLSLRIECFPWRHKVWCYHDCLIYNT
ITDEEVHTRGLTGIEVTCNPVGGGGSGGGGSGGGGSAAAQVQLVQSGGG
LVQPGGSLRLSCAASGRAFNSAAMGWYRQAPGSQRELVASISAGTASYA
DAVKGRFTISRDYAKNIIYLQMNSLKPDDTAVYFCNYRTTYTSGYSEDY
WGQGTQVTVSGGGSGGGSYPYDVPDYA.

In the above polypeptide sequence, the cytoplasmic domain is underlined, the transmembrane domain is in italics, the extravirion domain is in plain text, the (G4S)3 linker is in bold, the NotI site is in bold underline, the anti-CD45 VHH domain is in bold italic, the (G3S)2 linker is in italic underline, and the HA tag is double-underlined.

By “RMV—H-CD45 (32) polynucleotide” is meant a polynucleotide encoding a RMV—H-CD45 (32) polypeptide.

By “CD46 polypeptide” is meant a polypeptide or fragment thereof having at least about 85% amino acid sequence identity to NCBI Reference Sequence No. NP_758865.1, and that extends viral half-life in a subject. An exemplary CD46 polypeptide sequence is provided below:

MEPPGRRECPFPSWRFPGLLLAAMVLLLYSFSDACEEPPTFEAMELIGK
PKPYYEIGERVDYKCKKGYFYIPPLATHTICDRNHTWLPVSDDACYRET
CPYIRDPLNGQAVPANGTYEFGYQMHFICNEGYYLIGEEILYCELKGSV
AIWSGKPPICEKVLCTPPPKIKNGKHTFSEVEVFEYLDAVTYSCDPAPG
PDPFSLIGESTIYCGDNSVWSRAAPECKVVKCRFPVVENGKQISGFGKK
FYYKATVMFECDKGFYLDGSDTIVCDSNSTWDPPVPKCLKVSTSSTTKS
PASSASGYPKPEEGILDSLDVWVIAVIVIAIVVGVAVICVVPYRYLQRR
KKKGTYLTDETHREVKFTSL.

By “CD46 polynucleotide” is meant a polynucleotide that encodes a CD46 polypeptide or a fragment thereof. An exemplary CD46 polynucleotide sequence is provided at base pairs 160 to 1251 of NCBI Reference Sequence No.: NM_172355.3. An exemplary CD46 polynucleotide sequence is provided below:

GCGGCTCGGGCCACGCCCACCTGTCCTGCAGCACTGGATGCTTTGTGAG
TTGGGGATTGTTGCGTCCCATATCTGGACCCAGAAGGGACTTCCCTGCT
CGGCTGGCTCTCGGTTTCTCTGCTTTCCTCCGGAGAAATAACAGCGTCT
TCCGCGCCGCGCATGGAGCCTCCCGGCCGCCGCGAGTGTCCCTTTCCTT
CCTGGCGCTTTCCTGGGTTGCTTCTGGCGGCCATGGTGTTGCTGCTGTA
CTCCTTCTCCGATGCCTGTGAGGAGCCACCAACATTTGAAGCTATGGAG
CTCATTGGTAAACCAAAACCCTACTATGAGATTGGTGAACGAGTAGATT
ATAAGTGTAAAAAAGGATACTTCTATATACCTCCTCTTGCCACCCATAC
TATTTGTGATCGGAATCATACATGGCTACCTGTCTCAGATGACGCCTGT
TATAGAGAAACATGTCCATATATACGGGATCCTTTAAATGGCCAAGCAG
TCCCTGCAAATGGGACTTACGAGTTTGGTTATCAGATGCACTTTATTTG
TAATGAGGGTTATTACTTAATTGGTGAAGAAATTCTATATTGTGAACTT
AAAGGATCAGTAGCAATTTGGAGCGGTAAGCCCCCAATATGTGAAAAGG
TTTTGTGTACACCACCTCCAAAAATAAAAAATGGAAAACACACCTTTAG
TGAAGTAGAAGTATTTGAGTATCTTGATGCAGTAACTTATAGTTGTGAT
CCTGCACCTGGACCAGATCCATTTTCACTTATTGGAGAGAGCACGATTT
ATTGTGGTGACAATTCAGTGTGGAGTCGTGCTGCTCCAGAGTGTAAAGT
GGTCAAATGTCGATTTCCAGTAGTCGAAAATGGAAAACAGATATCAGGA
TTTGGAAAAAAATTTTACTACAAAGCAACAGTTATGTTTGAATGCGATA
AGGGTTTTTACCTCGATGGCAGCGACACAATTGTCTGTGACAGTAACAG
TACTTGGGATCCCCCAGTTCCAAAGTGTCTTAAAGTGTCGACTTCTTCC
ACTACAAAATCTCCAGCGTCCAGTGCCTCAGGATATCCTAAACCTGAGG
AAGGAATACTTGACAGTTTGGATGTTTGGGTCATTGCTGTGATTGTTAT
TGCCATAGTTGTTGGAGTTGCAGTAATTTGTGTTGTCCCGTACAGATAT
CTTCAAAGGAGGAAGAAGAAAGGCACATACCTAACTGATGAGACCCACA
GAGAAGTAAAATTTACTTCTCTCTGAGAAGGAGAGATGAGAGAAAGGTT
TGCTTTTATCATTAAAAGGAAAGCAGATGGTGGAGCTGAATATGCCACT
TACCAGACTAAATCAACCACTCCAGCAGAGCAGAGAGGCTGAATAGATT
CCACAACCTGGTTTGCCAGTTCATCTTTTGACTCTATTAAAATCTTCAA
TAGTTGTTATTCTGTAGTTTCACTCTCATGAGTGCAACTGTGGCTTAGC
TAATATTGCAATGTGGCTTGAATGTAGGTAGCATCCTTTGATGCTTCTT
TGAAACTTGTATGAATTTGGGTATGAACAGATTGCCTGCTTTCCCTTAA
ATAACACTTAGATTTATTGGACCAGTCAGCACAGCATGCCTGGTTGTAT
TAAAGCAGGGATATGCTGTATTTTATAAAATTGGCAAAATTAGAGAAAT
ATAGTTCACAATGAAATTATATTTTCTTTGTAAAGAAAGTGGCTTGAAA
TCTTTTTTGTTCAAAGATTAATGCCAACTCTTAAGATTATTCTTTCACC
AACTATAGAATGTATTTTATATATCGTTCATTGTAAAAAGCCCTTAAAA
ATATGTGTATACTACTTTGGCTCTTGTGCATAAAAACAAGAACACTGAA
AATTGGGAATATGCACAAACTTGGCTTCTTTAACCAAGAATATTATTGG
AAAATTCTCTAAAAGTTAATAGGGTAAATTCTCTATTTTTTGTAATGTG
TTCGGTGATTTCAGAAAGCTAGAAAGTGTATGTGTGGCATTTGTTTTCA
CTTTTTAAAACATCCCTAACTGATCGAATATATCAGTAATTTCAGAATC
AGATGCATCCTTTCATAAGAAGTGAGAGGACTCTGACAGCCATAACAGG
AGTGCCACTTCATGGTGCGAAGTGAACACTGTAGTCTTGTTGTTTTCCC
AAAGAGAACTCCGTATGTTCTCTTAGGTTGAGTAACCCACTCTGAATTC
TGGTTACATGTGTTTTTCTCTCCCTCCTTAAATAAAGAGAGGGGTTAAA
CATGCCCTCTAAAAGTAGGTGGTTTTGAAGAGAATAAATTCATCAGATA
ACCTCAAGTCACATGAGAATCTTAGTCCATTTACATTGCCTTGGCTAGT
AAAAGCCATCTATGTATATGTCTTACCTCATCTCCTAAAAGGCAGAGTA
CAAAGTAAGCCATGTATCTCAGGAAGGTAACTTCATTTTGTCTATTTGC
TGTTGATTGTACCAAGGGATGGAAGAAGTAAATATAGCTCAGGTAGCAC
TTTATACTCAGGCAGATCTCAGCCCTCTACTGAGTCCCTTAGCCAAGCA
GTTTCTTTCAAAGAAGCCAGCAGGCGAAAAGCAGGGACTGCCACTGCAT
TTCATATCACACTGTTAAAAGTTGTGTTTTGAAATTTTATGTTTAGTTG
CACAAATTGGGCCAAAGAAACATTGCCTTGAGGAAGATATGATTGGAAA
ATCAAGAGTGTAGAAGAATAAATACTGTTTTACTGTCCAAAGACATGTT
TATAGTGCTCTGTAAATGTTCCTTTCCTTTGTAGTCTCTGGCAAGATGC
TTTAGGAAGATAAAAGTTTGAGGAGAACAAACAGGAATTCTGAATTAAG
CACAGAGTTGAAGTTTATACCCGTTTCACATGCTTTTCAAGAATGTCGC
AATTACTAAGAAGCAGATAATGGTGTTTTTTAGAAACCTAATTGAAGTA
TATTCAACCAAATACTTTAATGTATAAAATAAATATTATACAATATACT
TGTATAGCAGTTTCTGCTTCACATTTGATTTTTTCAAATTTAATATTTA
TATTAGAGATCTATATATGTATAAATATGTATTTTGTCAAATTTGTTAC
TTAAATATATAGAGACCAGTTTTCTCTGGAAGTTTGTTTAAATGACAGA
AGCGTATATGAATTCAAGAAAATTTAAGCTGCAAAAATGTATTTGCTAT
AAAATGAGAAGTCTCACTGATAGAGGTTCTTTATTGCTCATTTTTTAAA
AAATGGACTCTTGAAATCTGTTAAAATAAAATTGTACATTTGGA.

By “CD47 polypeptide” is meant a polypeptide or fragment thereof having at least about 85% amino acid sequence identity to NCBI Reference Sequence No. NP_001768.1, and that that extends viral half-life in a subject. An exemplary CD47 polypeptide sequence is provided below:

MWPLVAALLLGSACCGSAQLLFNKTKSVEFTFCNDTVVIPCFVTNMEAQ
NTTEVYVKWKFKGRDIYTFDGALNKSTVPTDFSSAKIEVSQLLKGDASL
KMDKSDAVSHTGNYTCEVTELTREGETIIELKYRVVSWFSPNENILIVI
FPIFAILLFWGQFGIKTLKYRSGGMDEKTIALLVAGLVITVIVIVGAIL
FVPGEYSLKNATGLGLIVTSTGILILLHYYVFSTAIGLTSFVIAILVIQ
VIAYILAVVGLSLCIAACIPMHGPLLISGLSILALAQLLGLVYMKFVAS
NQKTIQPPRKAVEEPLNAFKESKGMMNDE.

By “CD47 polynucleotide” is meant a polynucleotide that encodes a CD47 polypeptide or a fragment thereof. An exemplary CD47 polynucleotide sequence is provided at base pairs 124 to 1095 of NCBI Reference Sequence No.: NM_001777.4. An exemplary CD47 polynucleotide sequence is provided below:

GCAGCCTGGGCAGTGGGTCCTGCCTGTGACGCGCGGCGGCGGTCGGTCC
TGCCTGTAACGGCGGCGGCGGCTGCTGCTCCGGACACCTGCGGCGGCGG
CGGCGACCCCGCGGCGGGCGCGGAGATGTGGCCCCTGGTAGCGGCGCTG
TTGCTGGGCTCGGCGTGCTGCGGATCAGCTCAGCTACTATTTAATAAAA
CAAAATCTGTAGAATTCACGTTTTGTAATGACACTGTCGTCATTCCATG
CTTTGTTACTAATATGGAGGCACAAAACACTACTGAAGTATACGTAAAG
TGGAAATTTAAAGGAAGAGATATTTACACCTTTGATGGAGCTCTAAACA
AGTCCACTGTCCCCACTGACTTTAGTAGTGCAAAAATTGAAGTCTCACA
ATTACTAAAAGGAGATGCCTCTTTGAAGATGGATAAGAGTGATGCTGTC
TCACACACAGGAAACTACACTTGTGAAGTAACAGAATTAACCAGAGAAG
GTGAAACGATCATCGAGCTAAAATATCGTGTTGTTTCATGGTTTTCTCC
AAATGAAAATATTCTTATTGTTATTTTCCCAATTTTTGCTATACTCCTG
TTCTGGGGACAGTTTGGTATTAAAACACTTAAATATAGATCCGGTGGTA
TGGATGAGAAAACAATTGCTTTACTTGTTGCTGGACTAGTGATCACTGT
CATTGTCATTGTTGGAGCCATTCTTTTCGTCCCAGGTGAATATTCATTA
AAGAATGCTACTGGCCTTGGTTTAATTGTGACTTCTACAGGGATATTAA
TATTACTTCACTACTATGTGTTTAGTACAGCGATTGGATTAACCTCCTT
CGTCATTGCCATATTGGTTATTCAGGTGATAGCCTATATCCTCGCTGTG
GTTGGACTGAGTCTCTGTATTGCGGCGTGTATACCAATGCATGGCCCTC
TTCTGATTTCAGGTTTGAGTATCTTAGCTCTAGCACAATTACTTGGACT
AGTTTATATGAAATTTGTGGCTTCCAATCAGAAGACTATACAACCTCCT
AGGAAAGCTGTAGAGGAACCCCTTAATGCATTCAAAGAATCAAAAGGAA
TGATGAATGATGAATAACTGAAGTGAAGTGATGGACTCCGATTTGGAGA
GTAGTAAGACGTGAAAGGAATACACTTGTGTTTAAGCACCATGGCCTTG
ATGATTCACTGTTGGGGAGAAGAAACAAGAAAAGTAACTGGTTGTCACC
TATGAGACCCTTACGTGATTGTTAGTTAAGTTTTTATTCAAAGCAGCTG
TAATTTAGTTAATAAAATAATTATGATCTATGTTGTTTGCCCAATTGAG
ATCCAGTTTTTTGTTGTTATTTTTAATCAATTAGGGGCAATAGTAGAAT
GGACAATTTCCAAGAATGATGCCTTTCAGGTCCTAGGGCCTCTGGCCTC
TAGGTAACCAGTTTAAATTGGTTCAGGGTGATAACTACTTAGCACTGCC
CTGGTGATTACCCAGAGATATCTATGAAAACCAGTGGCTTCCATCAAAC
CTTTGCCAACTCAGGTTCACAGCAGCTTTGGGCAGTTATGGCAGTATGG
CATTAGCTGAGAGGTGTCTGCCACTTCTGGGTCAATGGAATAATAAATT
AAGTACAGGCAGGAATTTGGTTGGGAGCATCTTGTATGATCTCCGTATG
ATGTGATATTGATGGAGATAGTGGTCCTCATTCTTGGGGGTTGCCATTC
CCACATTCCCCCTTCAACAAACAGTGTAACAGGTCCTTCCCAGATTTAG
GGTACTTTTATTGATGGATATGTTTTCCTTTTATTCACATAACCCCTTG
AAACCCTGTCTTGTCCTCCTGTTACTTGCTTCTGCTGTACAAGATGTAG
CACCTTTTCTCCTCTTTGAACATGGTCTAGTGACACGGTAGCACCAGTT
GCAGGAAGGAGCCAGACTTGTTCTCAGAGCACTGTGTTCACACTTTTCA
GCAAAAATAGCTATGGTTGTAACATATGTATTCCCTTCCTCTGATTTGA
AGGCAAAAATCTACAGTGTTTCTTCACTTCTTTTCTGATCTGGGGCATG
AAAAAAGCAAGATTGAAATTTGAACTATGAGTCTCCTGCATGGCAACAA
AATGTGTGTCACCATCAGGCCAACAGGCCAGCCCTTGAATGGGGATTTA
TTACTGTTGTATCTATGTTGCATGATAAACATTCATCACCTTCCTCCTG
TAGTCCTGCCTCGTACTCCCCTTCCCCTATGATTGAAAAGTAAACAAAA
CCCACATTTCCTATCCTGGTTAGAAGAAAATTAATGTTCTGACAGTTGT
GATCGCCTGGAGTACTTTTAGACTTTTAGCATTCGTTTTTTACCTGTTT
GTGGATGTGTGTTTGTATGTGCATACGTATGAGATAGGCACATGCATCT
TCTGTATGGACAAAGGTGGGGTACCTACAGGAGAGCAAAGGTTAATTTT
GTGCTTTTAGTAAAAACATTTAAATACAAAGTTCTTTATTGGGTGGAAT
TATATTTGATGCAAATATTTGATCACTTAAAACTTTTAAAACTTCTAGG
TAATTTGCCACGCTTTTTGACTGCTCACCAATACCCTGTAAAAATACGT
AATTCTTCCTGTTTGTGTAATAAGATATTCATATTTGTAGTTGCATTAA
TAATAGTTATTTCTTAGTCCATCAGATGTTCCCGTGTGCCTCTTTTATG
CCAAATTGATTGTCATATTTCATGTTGGGACCAAGTAGTTTGCCCATGG
CAAACCTAAATTTATGACCTGCTGAGGCCTCTCAGAAAACTGAGCATAC
TAGCAAGACAGCTCTTCTTGAAAAAAAAAATATGTATACACAAATATAT
ACGTATATCTATATATACGTATGTATATACACACATGTATATTCTTCCT
TGATTGTGTAGCTGTCCAAAATAATAACATATATAGAGGGAGCTGTATT
CCTTTATACAAATCTGATGGCTCCTGCAGCACTTTTTCCTTCTGAAAAT
ATTTACATTTTGCTAACCTAGTTTGTTACTTTAAAAATCAGTTTTGATG
AAAGGAGGGAAAAGCAGATGGACTTGAAAAAGATCCAAGCTCCTATTAG
AAAAGGTATGAAAATCTTTATAGTAAAATTTTTTATAAACTAAAGTTGT
ACCTTTTAATATGTAGTAAACTCTCATTTATTTGGGGTTCGCTCTTGGA
TCTCATCCATCCATTGTGTTCTCTTTAATGCTGCCTGCCTTTTGAGGCA
TTCACTGCCCTAGACAATGCCACCAGAGATAGTGGGGGAAATGCCAGAT
GAAACCAACTCTTGCTCTCACTAGTTGTCAGCTTCTCTGGATAAGTGAC
CACAGAAGCAGGAGTCCTCCTGCTTGGGCATCATTGGGCCAGTTCCTTC
TCTTTAAATCAGATTTGTAATGGCTCCCAAATTCCATCACATCACATTT
AAATTGCAGACAGTGTTTTGCACATCATGTATCTGTTTTGTCCCATAAT
ATGCTTTTTACTCCCTGATCCCAGTTTCTGCTGTTGACTCTTCCATTCA
GTTTTATTTATTGTGTGTTCTCACAGTGACACCATTTGTCCTTTTCTGC
AACAACCTTTCCAGCTACTTTTGCCAAATTCTATTTGTCTTCTCCTTCA
AAACATTCTCCTTTGCAGTTCCTCTTCATCTGTGTAGCTGCTCTTTTGT
CTCTTAACTTACCATTCCTATAGTACTTTATGCATCTCTGCTTAGTTCT
ATTAGTTTTTTGGCCTTGCTCTTCTCCTTGATTTTAAAATTCCTTCTAT
AGCTAGAGCTTTTCTTTCTTTCATTCTCTCTTCCTGCAGTGTTTTGCAT
ACATCAGAAGCTAGGTACATAAGTTAAATGATTGAGAGTTGGCTGTATT
TAGATTTATCACTTTTTAATAGGGTGAGCTTGAGAGTTTTCTTTCTTTC
TGTTTTTTTTTTTTGTTTTTTTTTTTTTTTTTTTTTTTTTTTTTTTTGA
CTAATTTCACATGCTCTAAAAACCTTCAAAGGTGATTATTTTTCTCCTG
GAAACTCCAGGTCCATTCTGTTTAAATCCCTAAGAATGTCAGAATTAAA
ATAACAGGGCTATCCCGTAATTGGAAATATTTCTTTTTTCAGGATGCTA
TAGTCAATTTAGTAAGTGACCACCAAATTGTTATTTGCACTAACAAAGC
TCAAAACACGATAAGTTTACTCCTCCATCTCAGTAATAAAAATTAAGCT
GTAATCAACCTTCTAGGTTTCTCTTGTCTTAAAATGGGTATTCAAAAAT
GGGGATCTGTGGTGTATGTATGGAAACACATACTCCTTAATTTACCTGT
TGTTGGAAACTGGAGAAATGATTGTCGGGCAACCGTTTATTTTTTATTG
TATTTTATTTGGTTGAGGGATTTTTTTATAAACAGTTTTACTTGTGTCA
TATTTTAAAATTACTAACTGCCATCACCTGCTGGGGTCCTTTGTTAGGT
CATTTTCAGTGACTAATAGGGATAATCCAGGTAACTTTGAAGAGATGAG
CAGTGAGTGACCAGGCAGTTTTTCTGCCTTTAGCTTTGACAGTTCTTAA
TTAAGATCATTGAAGACCAGCTTTCTCATAAATTTCTCTTTTTGAAAAA
AAGAAAGCATTTGTACTAAGCTCCTCTGTAAGACAACATCTTAAATCTT
AAAAGTGTTGTTATCATGACTGGTGAGAGAAGAAAACATTTTGTTTTTA
TTAAATGGAGCATTATTTACAAAAAGCCATTGTTGAGAATTAGATCCCA
CATCGTATAAATATCTATTAACCATTCTAAATAAAGAGAACTCCAGTGT
TGCTATGTGCAAGATCCTCTCTTGGAGCTTTTTTGCATAGCAATTAAAG
GTGTGCTATTTGTCAGTAGCCATTTTTTTGCAGTGATTTGAAGACCAAA
GTTGTTTTACAGCTGTGTTACCGTTAAAGGTTTTTTTTTTTATATGTAT
TAAATCAATTTATCACTGTTTAAAGCTTTGAATATCTGCAATCTTTGCC
AAGGTACTTTTTTATTTAAAAAAAAACATAACTTTGTAAATATTACCCT
GTAATATTATATATACTTAATAAAACATTTTAAGCTATTTTGTTGGGCT
ATTTCTATTGCTGCTACAGCAGACCACAAGCACATTTCTGAAAAATTTA
ATTTATTAATGTATTTTTAAGTTGCTTATATTCTAGGTAACAATGTAAA
GAATGATTTAAAATATTAATTATGAATTTTTTGAGTATAATACCCAATA
AGCTTTTAATTAGAGCAGAGTTTTAATTAAAAGTTTTAAATCAGTCCAA.

By “CD55 polypeptide” is meant a polypeptide or fragment thereof having at least about 85% amino acid sequence identity to NCBI Reference Sequence No. NP_001108224.1, and that extends viral half-life in a subject. An exemplary CD55 polypeptide sequence is provided below:

MTVARPSVPAALPLLGELPRLLLLVLLCLPAVWGDCGLPPDVPNAQPAL
EGRTSFPEDTVITYKCEESFVKIPGEKDSVICLKGSQWSDIEEFCNRSC
EVPTRLNSASLKQPYITQNYFPVGTVVEYECRPGYRREPSLSPKLTCLQ
NLKWSTAVEFCKKKSCPNPGEIRNGQIDVPGGILFGATISFSCNTGYKL
FGSTSSFCLISGSSVQWSDPLPECREIYCPAPPQIDNGIIQGERDHYGY
RQSVTYACNKGFTMIGEHSIYCTVNNDEGEWSGPPPECRGKSLTSKVPP
TVQKPTTVNVPTTEVSPTSQKTTTKTTTPNAQATRSTPVSRTTKHFHET
TPNKGSGTTSGTTRLLSGSRPVTQAGMRWCDRSSLQSRTPGFKRSFHFS
LPSSWYYRAHVFHVDRFAWDASNHGLADLAKEELRRKYTQVYRLFLVS.

By “CD55 polynucleotide” is meant a polynucleotide that encodes a CD55 polypeptide or a fragment thereof. An exemplary CD55 polynucleotide sequence is provided at base pairs 89 to 1411 of NCBI Reference Sequence No.: NM_001114752.3. An exemplary CD55 polynucleotide sequence is provided below:

CTGCTTACTGCAACTCGCTCCGGCCGCTGGGCGTAGCTGCGACTCGGCG
GAGTCCCGGCGGCGCGTCCTTGTTCTAACCCGGCGCGCCATGACCGTCG
CGCGGCCGAGCGTGCCCGCGGCGCTGCCCCTCCTCGGGGAGCTGCCCCG
GCTGCTGCTGCTGGTGCTGTTGTGCCTGCCGGCCGTGTGGGGTGACTGT
GGCCTTCCCCCAGATGTACCTAATGCCCAGCCAGCTTTGGAAGGCCGTA
CAAGTTTTCCCGAGGATACTGTAATAACGTACAAATGTGAAGAAAGCTT
TGTGAAAATTCCTGGCGAGAAGGACTCAGTGATCTGCCTTAAGGGCAGT
CAATGGTCAGATATTGAAGAGTTCTGCAATCGTAGCTGCGAGGTGCCAA
CAAGGCTAAATTCTGCATCCCTCAAACAGCCTTATATCACTCAGAATTA
TTTTCCAGTCGGTACTGTTGTGGAATATGAGTGCCGTCCAGGTTACAGA
AGAGAACCTTCTCTATCACCAAAACTAACTTGCCTTCAGAATTTAAAAT
GGTCCACAGCAGTCGAATTTTGTAAAAAGAAATCATGCCCTAATCCGGG
AGAAATACGAAATGGTCAGATTGATGTACCAGGTGGCATATTATTTGGT
GCAACCATCTCCTTCTCATGTAACACAGGGTACAAATTATTTGGCTCGA
CTTCTAGTTTTTGTCTTATTTCAGGCAGCTCTGTCCAGTGGAGTGACCC
GTTGCCAGAGTGCAGAGAAATTTATTGTCCAGCACCACCACAAATTGAC
AATGGAATAATTCAAGGGGAACGTGACCATTATGGATATAGACAGTCTG
TAACGTATGCATGTAATAAAGGATTCACCATGATTGGAGAGCACTCTAT
TTATTGTACTGTGAATAATGATGAAGGAGAGTGGAGTGGCCCACCACCT
GAATGCAGAGGAAAATCTCTAACTTCCAAGGTCCCACCAACAGTTCAGA
AACCTACCACAGTAAATGTTCCAACTACAGAAGTCTCACCAACTTCTCA
GAAAACCACCACAAAAACCACCACACCAAATGCTCAAGCAACACGGAGT
ACACCTGTTTCCAGGACAACCAAGCATTTTCATGAAACAACCCCAAATA
AAGGAAGTGGAACCACTTCAGGTACTACCCGTCTTCTATCTGGTTCTCG
TCCTGTCACCCAGGCTGGTATGCGGTGGTGTGATCGTAGCTCACTGCAG
TCTCGAACTCCTGGGTTCAAGCGATCCTTCCACTTCAGCCTCCCAAGTA
GCTGGTACTACAGGGCACACGTGTTTCACGTTGACAGGTTTGCTTGGGA
CGCTAGTAACCATGGGCTTGCTGACTTAGCCAAAGAAGAGTTAAGAAGA
AAATACACACAAGTATACAGACTGTTCCTAGTTTCTTAGACTTATCTGC
ATATTGGATAAAATAAATGCAATTGTGCTCTTCATTTAGGATGCTTTCA
TTGTCTTTAAGATGTGTTAGGAATGTCAACAGAGCAAGGAGAAAAAAGG
CAGTCCTGGAATCACATTCTTAGCACACCTACACCTCTTGAAAATAGAA
CAACTTGCAGAATTGAGAGTGATTCCTTTCCTAAAAGTGTAAGAAAGCA
TAGAGATTTGTTCGTATTTAGAATGGGATCACGAGGAAAAGAGAAGGAA
AGTGATTTTTTTCCACAAGATCTGTAATGTTATTTCCACTTATAAAGGA
AATAAAAAATGAAAAACATTATTTGGATATCAAAAGCAAATAAAAACCC
AATTCAGTCTCTTCTAAGCAAAATTGCTAAAGAGAGATGAACCACATTA
TAAAGTAATCTTTGGCTGTAAGGCATTTTCATCTTTCCTTCGGGTTGGC
AAAATATTTTAAAGGTAAAACATGCTGGTGAACCAGGGGTGTTGATGGT
GATAAGGGAGGAATATAGAATGAAAGACTGAATCTTCCTTTGTTGCACA
AATAGAGTTTGGAAAAAGCCTGTGAAAGGTGTCTTCTTTGACTTAATGT
CTTTAAAAGTATCCAGAGATACTACAATATTAACATAAGAAAAGATTAT
ATATTATTTCTGAATCGAGATGTCCATAGTCAAATTTGTAAATCTTATT
CTTTTGTAATATTTATTTATATTTATTTATGACAGTGAACATTCTGATT
TTACATGTAAAACAAGAAAAGTTGAAGAAGATATGTGAAGAAAAATGTA
TTTTTCCTAAATAGAAATAAATGATCCCATTTTTTGGTATCATGTAGTA
TGTGAAATTTATTCTTAAACGTGACTACTTTATTTCTAAATAAGAAATT
CCCTACCTGCTTCCTACAAGCAGTTCAGAATGCCATGCCTTGGTTGTCC
TAGTGTGAATAATTTTCAGCTACTTTAAAATTATATTGTACTTTCTCAA
GCATGTCATATCCTTTCCTATTAGAGTATCTATATTACTTGTTACTGAT
TTACCTGAAGGCAATCTGATTAATTTCTAGGTTTTTACCATATTCTTGT
CATCTTGCCAATTACATTTTAAGTGTTAGACTAGACTAAGATGTACTAG
TTGTATAGAATATAACTAGATTTATTATGGCAATGTTTATTTTGTCATT
TTGCTTCATCTGTTTTGTTGTTGAAGTACTTTAAATTTCATACGTTCAT
GGCATTTCACTGTAAAGACTTTAATGTGTATTTCTTAAAATAAAACTTT
TTTTCCTCCTTAA.

By “CD59 polypeptide” is meant a polypeptide or fragment thereof having at least about 85% amino acid sequence identity to NCBI Reference Sequence No. NP_976075.1, and that extends viral half-life in a subject. An exemplary CD59 polypeptide sequence is provided below:

MGIQGGSVLFGLLLVLAVFCHSGHSLQCYNCPNPTADCKTAVNCSSDFD
ACLITKAGLQVYNKCWKFEHCNFNDVTTRLRENELTYYCCKKDLCNFNE
QLENGGTSLSEKTVLLLVTPFLAAAWSLHP.

By “CD59 polynucleotide” is meant a polynucleotide that encodes a CD59 polypeptide or a fragment thereof. An exemplary CD59 polynucleotide sequence is provided at base pairs 278 to 664 of NCBI Reference Sequence No.: NM_203330.2. An exemplary CD59 polynucleotide sequence is provided below:

GGGGCCGGGGGGCGGAGCCTTGCGGGCTGGAGCGAAAGAATGCGGGGGCTGAGCGCAGAAGCGG
CTCGAGGCTGGAAGAGGATCTTGGGCGCCGCCAGTCTCTCTCTGTTGCCCAAGCTGGAGTGCAG
TGGCACAGTCTTGGCTCACTGCAACCTCCACCTCCTGGGTGCAAGCGATTCTCGTGTCTCAGCC
TCTCAAGTAGCTGGGATTACAGTCTTTAGCACCAGTTGGTGTAGGAGTTGAGACCTACTTCACA
GTAGTTCTGTGGACAATCACAATGGGAATCCAAGGAGGGTCTGTCCTGTTCGGGCTGCTGCTCG
TCCTGGCTGTCTTCTGCCATTCAGGTCATAGCCTGCAGTGCTACAACTGTCCTAACCCAACTGC
TGACTGCAAAACAGCCGTCAATTGTTCATCTGATTTTGATGCGTGTCTCATTACCAAAGCTGGG
TTACAAGTGTATAACAAGTGTTGGAAGTTTGAGCATTGCAATTTCAACGACGTCACAACCCGCT
TGAGGGAAAATGAGCTAACGTACTACTGCTGCAAGAAGGACCTGTGTAACTTTAACGAACAGCT
TGAAAATGGTGGGACATCCTTATCAGAGAAAACAGTTCTTCTGCTGGTGACTCCATTTCTGGCA
GCAGCCTGGAGCCTTCATCCCTAAGTCAACACCAGGAGAGCTTCTCCCAAACTCCCCGTTCCTG
CGTAGTCCGCTTTCTCTTGCTGCCACATTCTAAAGGCTTGATATTTTCCAAATGGATCCTGTTG
GGAAAGAATAAAATTAGCTTGAGCAACCTGGCTAAGATAGAGGGGCTCTGGGAGACTTTGAAGA
CCAGTCCTGTTTGCAGGGAAGCCCCACTTGAAGGAAGAAGTCTAAGAGTGAAGTAGGTGTGACT
TGAACTAGATTGCATGCTTCCTCCTTTGCTCTTGGGAAGACCAGCTTTGCAGTGACAGCTTGAG
TGGGTTCTCTGCAGCCCTCAGATTATTTTTCCTCTGGCTCCTTGGATGTAGTCAGTTAGCATCA
TTAGTACATCTTTGGAGGGTGGGGCAGGAGTATATGAGCATCCTCTCTCACATGGAACGCTTTC
ATAAACTTCAGGGATCCCGTGTTGCCATGGAGGCATGCCAAATGTTCCATATGTGGGTGTCAGT
CAGGGACAACAAGATCCTTAATGCAGAGCTAGAGGACTTCTGGCAGGGAAGTGGGGAAGTGTTC
CAGATAGCAGGGCATGAAAACTTAGAGAGGTACAAGTGGCTGAAAATCGAGTTTTTCCTCTGTC
TTTAAATTTTATATGGGCTTTGTTATCTTCCACTGGAAAAGTGTAATAGCATACATCAATGGTG
TGTTAAAGCTATTTCCTTGCCTTTTTTTTATTGGAATGGTAGGATATCTTGGCTTTGCCACACA
CAGTTACAGAGTGAACACTCTACTACATGTGACTGGCAGTATTAAGTGTGCTTATTTTAAATGT
TACTGGTAGAAAGGCAGTTCAGGTATGTGTGTATATAGTATGAATGCAGTGGGGACACCCTTTG
TGGTTACAGTTTGAGACTTCCAAAGGTCATCCTTAATAACAACAGATCTGCAGGGGTATGTTTT
ACCATCTGCATCCAGCCTCCTGCTAACTCCTAGCTGACTCAGCATAGATTGTATAAAATACCTT
TGTAACGGCTCTTAGCACACTCACAGATGTTTGAGGCTTTCAGAAGCTCTTCTAAAAAATGATA
CACACCTTTCACAAGGGCAAACTTTTTCCTTTTCCCTGTGTATTCTAGTGAATGAATCTCAAGA
TTCAGTAGACCTAATGACATTTGTATTTTATGATCTTGGCTGTATTTAATGGCATAGGCTGACT
TTTGCAGATGGAGGAATTTCTTGATTAATGTTGAAAAAAAACCCTTGATTATACTCTGTTGGAC
AAACCGAGTGCAATGAATGATGCTTTTCTGAAAATGAAATATAACAAGTGGGTGAATGTGGTTA
TGGCCGAAAAGGATATGCAGTATGCTTAATGGTAGCAACTGAAAGAAGACATCCTGAGCAGTGC
CAGCTTTCTTCTGTTGATGCCGTTCCCTGAACATAGGAAAATAGAAACTTGCTTATCAAAACTT
AGCATTACCTTGGTGCTCTGTGTTCTCTGTTAGCTCAGTGTCTTTCCTTACATCAATAGGTTTT
TTTTTTTTTTTTTGGCCTGAGGAAGTACTGACCATGCCCACAGCCACCGGCTGAGCAAAGAAGC
TCATTTCATGTGAGTTCTAAGGAATGAGAAACAATTTTGATGAATTTAAGCAGAAAATGAATTT
CTGGGAACTTTTTTGGGGGGGGGGGGGTGGGGAATTCAGCCACACTCCAGAAAGCCAGGAGTCG
ACAGTTTTGGAAGCCTCTCTCAGGATTGAGATTCTAGGATGAGATTGGCTTACTGCTATCTTGT
GTCATGTACCCACTTTTTGGCCAGACTACACTGGGAAGAAGGTAGTCCTCTAAAGCAAAATCTG
AGTGCCACTAAATGGGGAGATGGGGCTGTTAAGCTGTCCAAATCAACAAGGGTCATATAAATGG
CCTTAAACTTTGGGGTTGCTTTCTGCAAAAAGTTGCTGTGACTCATGCCATAGACAAGGTTGAG
TGCCTGGACCCAAAGGCAATACTGTAATGTAAAGACATTTATAGTACTAGGCAAACAGCACCCC
AGGTACTCCAGGCCCTCCTGGCTGGAGAGGGCTGTGGCAATAGAAAATTAGTGCCAACTGCAGT
GAGTCAGCCTAGGTTAAATAGAGAGTGTAAGAGTGCTGGACAGGAACCTCCACCCTCATGTCAC
ATTTCTTCAATGTGACCCTTCTGGCCCCTCTCCTCCTGACAGCGGAACAATGACTGCCCCGATA
GGTGAGGCTGGAGGAAGAATCAGTCCTGTCCTTGGCAAGCTCTTCACTATGACAGTAAAGGCTC
TCTGCCTGCTGCCAAGGCCTGTGACTTTCTAACCTGGCCTCACGCTGGGTAAGCTTAAGGTAGA
GGTGCAGGATTAGCAAGCCCACCTGGCTACCAGGCCGACAGCTACATCCTCCAACTGACCCTGA
TCAACGAAGAGGGATTCATGTGTCTGTCTCAGTTGGTTCCAAATGAAACCAGGGAGCAGGGGAG
TTAGGAATCGAACACCAGTCATGCCTACTGGCTCTCTGCTCGAGAGCCAATACCCTGTGCCCTC
CACTCATCTGGATTTACAGGAACTGTCATAGTGTTCAGTATTGGGTGGTGATAAGCCCATTGGA
TTGTCCCCTTGGGGGGATGAGCTAGGGGTGCAAGGAACACCTGATGAGTAGATAAGTGGAGCTC
ATGGTATTTCCTGAAAGATGCTAATCTATTTGCCAAACTTGGTCTTGAATGTACTGGGGGCTTC
AAGGTATGGGTATATTTTTCTTGTGTCCTTGCAGTTAGCCCCCATGTCTTATGTGTGTCCTGAA
AAAATAAGAGCCTGCCCAAGACTTTGGGCCTCTTGACAGAATTAACCACTTTTATACATCTGAG
TTCTCTTGGTAAGTTCTTTAGCAGTGTTCAAAGTCTACTAGCTCGCATTAGTTTCTGTTGCTGC
CAACAGATCTGAACTAATGCTAACAGATCCCCCTGAGGGATTCTTGATGGGCTGAGCAGCTGGC
TGGAGCTAGTACTGACTGACATTCATTGTGATGAGGGCAGCTTTCTGGTACAGGATTCTAAGCT
CTATGTTTTATATACATTTTCATCTGTACTTGCACCTCACTTTACACAAGAGGAAACTATGCAA
AGTTAGCTGGATCGCTCAAGGTCACTTAGGTAAGTTGGCAAGTCCATGCTTCCCACTCAGCTCC
TCAGGTCAGCAAGTCTACTTCTCTGCCTATTTTGTATACTCTCTTTAATATGTGCCTAGCTTTG
GAAAGTCTAGAATGGGTCCCTGGTGCCTTTTTACTTTGAAGAAATCAGTTTCTGCCTCTTTTTG
GAAAAGAAAACAAAGTGCAATTGTTTTTTACTGGAAAGTTACCCAATAGCATGAGGTGAACAGG
ACGTAGTTAGGCCTTCCTGTAAACAGAAAATCATATCAAAACACTATCTTCCCATCTGTTTCTC
AATGCCTGCTACTTCTTGTAGATATTTCATTTCAGGAGAGCAGCAGTTAAACCCGTGGATTTTG
TAGTTAGGAACCTGGGTTCAAACCCTCTTCCACTAATTGGCTATGTCTCTGGACAAGTTTTTTT
TTTTTTTTTTTTTTAAACCCTTTCTGAACTTTCACTTTCTATGTCTACCTCAAAGAATTGTTGT
GAGGCTTGAGATAATGCATTTGTAAAGGGTCTGCCAGATAGGAAGATGCTAGTTATGGATTTAC
AAGGTTGTTAAGGCTGTAAGAGTCTAAAACCTACAGTGAATCACAATGCATTTACCCCCACTGA
CTTGGACATAAGTGAAAACTAGCCAGAAGTCTCTTTTTCAAATTACTTACAGGTTATTCAATAT
AAAATTTTTGTAATGGATAATCTTATTTATCTAAACTAAAGCTTCCTGTTTATACACACTCCTG
TTATTCTGGGATAAGATAAATGACCACAGTACCTTAATTTCTAGGTGGGTGCCTGTGATGGTTC
ATTGTAGGTAAGGACATTTTCTCTTTTTCAGCAGCTGTGTAGGTCCAGAGCCTCTGGGAGAGGA
GGGGGGTAGCATGCACCCAGCAGGGGACTGAACTGGGAAACTCAAGGTTCTTTTTACTGTGGGG
TAGTGAGCTGCCTTTCTGTGATCGGTTTCCCTAGGGATGTTGCTGTTCCCCTCCTTGCTATTCG
CAGCTACATACAACGTGGCCAACCCCAGTAGGCTGATCCTATATATGATCAGTGCTGGTGCTGA
CTCTCAATAGCCCCACCCAAGCTGGCTATAGGTTTACAGATACATTAATTAGGCAACCTAAAAT
ATTGATGCTGGTGTTGGTGTGACATAATGCTATGGCCAGAACTGAAACTTAGAGTTATAATTCA
TGTATTAGGGTTCTCCAGAGGGACAGAATTAGTAGGATATATGTATATATGAAAGGGAGGTTAT
TAGGGAGAACTGGCTCCCACAGTTAGAAGGCGAAGTCGCACAATAGGCCGTCTGCAAGCTGGGT
TAGAGAGAAGCCAGTAGTGGCTCAGCCTGAGTTCAAAAACCTCAAAACTGGGGAAGCTGACAGT
GCAGCCAGCCTTCAGTCTGTGGCCAAAGGCCCAAGAGCCCCTGGCAACCAACCCACTGGTGCAA
GTCCTAGATTCCAAAGGCTGAAGAACCTGGAGTCTGATGTCCAAGAGCAGGAAGAGTGGAAGAA
AGCCAGAAGACTCAGCAAACAAGGTAGACAGTGTCTACCACCATAGTGGCCATACCAAAGAGGC
TACCGATTCCTTCCTGCTACCTGGATCCCTGAAGTTGCCCTGGTCTCTGCACCTTCTAAACCTA
GTTCTTAAGAGCTTTCCATTACATGAGCTGTCTCAAAGCCCTCCAATAAATTCTCAGTGTAAGC
TTCTGTTGCTTGTGGACAGAAAATTCTGACAGACCTACCCTATAAGTGTTACTGTCAGGATAAC
ATGAGAACGCACAACAGTAAGTGGTCACTAAGTGTTAGCTACGGTTATTTTGCCCAAGGTAGCA
TGGCTAGTTGATGCCGGTTGATGGGGCTTAAACCCAGCTCCCTCATCTTCCAGGCCTCTGTACT
CCCTATTCCACTAAACTACCTCTCAGGTTTATTTTTTTAAATTCTTACTCTGCAAGTACATAGG
ACCACATTTACCTGGGAAAACAAGAATAAAGGCTGCTCTGCATTTTTTAGAAACTTTTTTGAAA
GGGAGATGGGAATGCCTGCACCCCCAAGTCCAGACCAACACAATGGTTAATTGAGATGAATAAT
AAAGGAAAGACTGTTCTGGGCTTCCCAGAATAGCTTGGTCCTTAAATTGTGGCACAAACAACCT
CCTGTCAGAGCCAGCCTCCTGCCAGGAAGAGGGGTAGGAGACTAGAGGCCGTGTGTGCAGCCTT
GCCCTGAAGGCTAGGGTGACAATTTGGAGGCTGTCCAAACACCCTGGCCTCTAGAGCTGGCCTG
TCTATTTGAAATGCCGGCTCTGATGCTAATCGGCGACCCTCAGGCAAGTTACTTAACCTTACAT
GCCTCAGTTTTCTCATCTGGAAAATGAGAACCCTAGGTTTAGGGTTGTTAGAAAAGTTAAATGA
GTTAAGACAAGTGCCTGGGACACAGTAGCCTCTTGTGTGTGTTTATCATTATGTCCTCAGCAGG
TCGTAGAAGCAGCTTCTCAGGTGTGAGGCTGGCGCGATTATCTGGAGTGGGTTGGGTTTTCTAG
GATGGACCCCCTGCTGCATTTTCCTCATTCATCCACCAGGGCTTAATGGGGAATCAAGGAATCC
ATGTGTAACTGTATAATAACTGTAGCCACACTCCAATGACCACCTACTAGTTGTCCCTGGCACT
GCTTATACATATGTCCATCAAATCAATCCTATGAAGTAGATACTGTCTTCATTTTATAGATCAG
AGACAATTGGGGTTCAGAGAGCTGATGTGATTTTCCCAGGGTCACAGAGAGTCCCAGATTCAGG
CACAACTCTTGTATTCCAAGACACAACCACTACATGTCCAAAGGCTGCCCAGAGCCACCGGGCA
CGGCAAATTGTGACATATCCCTAAAGAGGCTGAGCACCTGGTCAGGATCTGATGGCTGACAGTG
TGTCCAGATGCAGAGCTGGAGTGGGGGAGGGGAAGGGGGGCTCCTTGGGACAGAGAAGGCTTTC
TGTGCTTTCTCTGAAGGGAGCAGTCTGAGGACCAAGGGAACCCGGCAAACAGCACCTCAGGTAC
TCCAGGCCCTCCTGGCTGGAGAGGGCTGTGGCAATGGAAAATTAGTGCCAACTGCAATGAGTCA
GCCTCGGTTAAATAGAGAGTGAAGAATGCTGGACAGGAACCTCCACCCTCATGTCACATTTCTT
CAGTGTGACCCTTCTGGCCCCTCTCCTCCTGACAGCGGAACAATGACTGCCCCGATAGGTGAGG
CTGGAGGAAGAATCAGTCCTGTCCTTGGCAAGCTCTTCACTATGACAGTAAAGGCTCTCTGCCT
GCTGCCAAGGCCTGTGACTTTCTAACCTGGCCTCACGCTGGGTAAGCTTAAGGTAGAGGTGCAG
GATTAGCAAGCCCACCTGGCTACCAGGCCGACAGCTACATCTTTCAACTGACCCTGATCAACGA
AGAGGGACTTGTGTCTCTCAGTTGGTTCCAAATGAAACCAGGGAGCAGGGGCGTTAGGAAGCTC
CAACAGGATGGTACTTAATGGGGCATTTGAGTGGAGAGGTAGGTGACATAGTGCTTTGGAGCCC
AGGGAGGGAAAGGTTCTGCTGAAGTTGAATTCAAGACTGTTCTTTCATCACAAACTTGAGTTTC
CTGGACATTTGTTTGCAGAAACAACCGTAGGGTTTTGCCTTAACCTCGTGGGTTTATTATTACC
TCATAGGGACTTTGCCTCCTGACAGCAGTTTATGGGTGTTCATTGTGGCACTTGAGTTTTCTTG
CATACTTGTTAGAGAAACCAAGTTTGTCATCAACTTCTTATTTAACCCCCTGGCTATAACTTCA
TGGATTATGTTATAATTAAGCCATCCAGAGTAAAATCTGTTTAGATTATCTTGGAGTAAGGGGG
AAAAAATCTGTAATTTTTTCTCCTCAACTAGATATATACATAAAAAATGATTGTATTGCTTCAT
TTAAAAAATATAACGCAAAATCTCTTTTCCTTCTAAAAAAAAAAAAAAAAAA.

By “CD4 polypeptide” is meant a polypeptide or fragment thereof having at least about 85% amino acid sequence identity to GenBank accession no. AAV38594.1, provided below, and that functions as a co-receptor for a T-cell receptor (TCR).

MNRGVPFRHLLLVLQLALLPAATQGKKVVLGKKGDTVELTCTASQKKSIQ
FHWKNSNQIKILGNQGSFLTKGPSKLNDRADSRRSLWDQGNFPLIIKNLK
IEDSDTYICEVEDQKEEVQLLVFGLTANSDTHLLQGQSLTLTLESPPGSS
PSVQCRSPRGKNIQGGKTLSVSQLELQDSGTWTCTVLQNQKKVEFKIDIV
VLAFQKASSIVYKKEGEQVEFSFPLAFTVEKLTGSGELWWQAERASSSKS
WITFDLKNKEVSVKRVTQDPKLQMGKKLPLHLTLPQALPQYAGSGNLTLA
LEAKTGKLHQEVNLVVMRATQLQKNLTCEVWGPTSPKLMLSLKLENKEAK
VSKREKAVWVLNPEAGMWQCLLSDSGQVLLESNIKVLPTWSTPVQPMALI
VLGGVAGLLLFIGLGIFFCVRCRHRRRQAERMSQIKRLLSEKKTCQCPHR
FQKTCSPI.

By “CD4 polynucleotide” is meant a polynucleotide that encodes a CD4 polypeptide or a fragment thereof. An exemplary CD4 polynucleotide sequence is provided at GenBank accession no. BT019791.1, provided below.

ATGAACCGGGGAGTCCCTTTTAGGCACTTGCTTCTGGTGCTGCAACTGGC
GCTCCTCCCAGCAGCCACTCAGGGAAAGAAAGTGGTGCTGGGCAAAAAAG
GGGATACAGTGGAACTGACCTGTACAGCTTCCCAGAAGAAGAGCATACAA
TTCCACTGGAAAAACTCCAACCAGATAAAGATTCTGGGAAATCAGGGCTC
CTTCTTAACTAAAGGTCCATCCAAGCTGAATGATCGCGCTGACTCAAGAA
GAAGCCTTTGGGACCAAGGAAACTTTCCCCTGATCATCAAGAATCTTAAG
ATAGAAGACTCAGATACTTACATCTGTGAAGTGGAGGACCAGAAGGAGGA
GGTGCAATTGCTAGTGTTCGGATTGACTGCCAACTCTGACACCCACCTGC
TTCAGGGGCAGAGCCTGACCCTGACCTTGGAGAGCCCCCCTGGTAGTAGC
CCCTCAGTGCAATGTAGGAGTCCAAGGGGTAAAAACATACAGGGGGGGAA
GACCCTCTCCGTGTCTCAGCTGGAGCTCCAGGATAGTGGCACCTGGACAT
GCACTGTCTTGCAGAACCAGAAGAAGGTGGAGTTCAAAATAGACATCGTG
GTGCTAGCTTTCCAGAAGGCCTCCAGCATAGTCTATAAGAAAGAGGGGGA
ACAGGTGGAGTTCTCCTTCCCACTCGCCTTTACAGTTGAAAAGCTGACGG
GCAGTGGCGAGCTGTGGTGGCAGGCGGAGAGGGCTTCCTCCTCCAAGTCT
TGGATCACCTTTGACCTGAAGAACAAGGAAGTGTCTGTAAAACGGGTTAC
CCAGGACCCTAAGCTCCAGATGGGCAAGAAGCTCCCGCTCCACCTCACCC
TGCCCCAGGCCTTGCCTCAGTATGCTGGCTCTGGAAACCTCACCCTGGCC
CTTGAAGCGAAAACAGGAAAGTTGCATCAGGAAGTGAACCTGGTGGTGAT
GAGAGCCACTCAGCTCCAGAAAAATTTGACCTGTGAGGTGTGGGGACCCA
CCTCCCCTAAGCTGATGCTGAGCTTGAAACTGGAGAACAAGGAGGCAAAG
GTCTCGAAGCGGGAGAAGGCGGTGTGGGTGCTGAACCCTGAGGCGGGGAT
GTGGCAGTGTCTGCTGAGTGACTCGGGACAGGTCCTGCTGGAATCCAACA
TCAAGGTTCTGCCCACATGGTCCACCCCGGTGCAGCCAATGGCCCTGATT
GTGCTGGGGGGCGTCGCCGGCCTCCTGCTTTTCATTGGGCTAGGCATCTT
CTTCTGTGTCAGGTGCCGGCACCGAAGGCGCCAAGCAGAGCGGATGTCTC
AGATCAAGAGACTCCTCAGTGAGAAGAAGACCTGCCAGTGCCCTCACCGG
TTTCAGAAGACATGTAGCCCCATTTAG.

By “CD7 polypeptide” is meant a polypeptide or fragment thereof having at least about 85% amino acid sequence identity to GenBank accession no. AAA51953.1, provided below, and that functions in T-cell or B-cell interactions during early lymphoid development.

MAGPPRLLLLPLLLALARGLPGALAAQEVQQSPHCTTVPVGASVNITCST
SGGLRGIYLRQLGPQPQDIIYYEDGVVPTTDRRFRGRIDFSGSQDNLTIT
MHRLQLSDTGTYTCQAITEVNVYGSGTLVLVTEEQSQGWHRCSDAPPRAS
ALPAPPTGSALPDPQTASALPDPPAASALPAALAVISFLLGLGLGVACVL
ARTQIKKLCSWRDKNSAACVVYEDMSHSRCNTLSSPNQYQ.

By “CD7 polynucleotide” is meant a polynucleotide that encodes a CD7 polypeptide or a fragment thereof. An exemplary CD7 polynucleotide sequence is provided at GenBank accession no. M37271.1, provided below.

ATGGCCGGGCCTCCGAGGCTCCTGCTGCTGCCCCTGCTTCTGGCGCTGGC
TCGCGGCCTGCCTGGGGCCCTGGCTGCCCAAGAGGTGCAGCAGTCTCCCC
ACTGCACGACTGTCCCCGTGGGAGCCTCCGTCAACATCACCTGCTCCACC
AGCGGGGGCCTGCGTGGGATCTACCTGAGGCAGCTCGGGCCACAGCCCCA
AGACATCATTTACTACGAGGACGGGGTGGTGCCCACTACGGACAGACGGT
TCCGGGGCCGCATCGACTTCTCAGGGTCCCAGGACAACCTGACTATCACC
ATGCACCGCCTGCAGCTGTCGGACACTGGCACCTACACCTGCCAGGCCAT
CACGGAGGTCAATGTCTACGGCTCCGGCACCCTGGTCCTGGTGACAGAGG
AACAGTCCCAAGGATGGCACAGATGCTCGGACGCCCCACCAAGGGCCTCT
GCCCTCCCTGCCCCACCGACAGGCTCCGCCCTCCCTGACCCGCAGACAGC
CTCTGCCCTCCCTGACCCGCCAGCAGCCTCTGCCCTCCCTGCGGCCCTGG
CGGTGATCTCCTTCCTCCTCGGGCTGGGCCTGGGGGTGGCGTGTGTGCTG
GCGAGGACACAGATAAAGAAACTGTGCTCGTGGCGGGATAAGAATTCGGC
GGCATGTGTGGTGTACGAGGACATGTCGCACAGCCGCTGCAACACGCTGT
CCTCCCCCAACCAGTACCAGTGA.

By “CD8 polypeptide” is meant a polypeptide or fragment thereof having at least about 85% amino acid sequence identity to GenBank accession no. AAA79217.1, provided below, and that functions in T cell signaling and aids with cytotoxic T cell antigen interactions.

MALPVTALLLPLALLLHAARPSQFRVSPLDRTWNLGETVELKCQVLLSNP
TSGCSWLFQPRGAAASPTFLLYLSQNKPKAAEGLDTQRFSGKRLGDTFVL
TLSDFRRENEGYYFCSALSNSIMYFSHFVPVFLPAKPTTTPAPRPPTPAP
TIASQPLSLRPEACRPAAGGAVHTRGLDFACDIYIWAPLAGTCGVLLLSL
VITLYCNHRNRRRVCKCPRPVVKSGDKPSLSARYV.

By “CD8 polynucleotide” is meant a polynucleotide that encodes a CD8 polypeptide or a fragment thereof. An exemplary CD8 polynucleotide sequence is provided at GenBank accession no. AH003215.2, provided below.

ATGGCCTTACCAGTGACCGCCTTGCTCCTGCCGCTGGCCTTGCTGCTCCA
CGCCGCCAGGCCGAGCCAGTTCCGGGTGTCGCCGCTGGATCGGACCTGGA
ACCTGGGCGAGACAGTGGAGCTGAAGTGCCAGGTGCTGCTGTCCAACCCG
ACGTCGGGCTGCTCGTGGCTCTTCCAGCCGCGCGGCGCCGCCGCCAGTCC
CACCTTCCTCCTATACCTCTCCCAAAACAAGCCCAAGGCGGCCGAGGGGC
TGGACACCCAGCGGTTCTCGGGCAAGAGGTTGGGGGACACCTTCGTCCTC
ACCCTGAGCGACTTCCGCCGAGAGAACGAGGGCTACTATTTCTGCTCGGC
CCTGAGCAACTCCATCATGTACTTCAGCCACTTCGTGCCGGTCTTCCTGC
CAGCGAAGCCCACCACGACGCCAGCGCCGCGACCACCAACACCGGCGCCC
ACCATCGCGTCGCAGCCCCTGTCCCTGCGCCCAGAGGCGTGCCGGCCAGC
GGCGGGGGGCGCAGTGCACACGAGGGGGCTGGACTTCGCCTGTGATATCT
ACATCTGGGCGCCCTTGGCCGGGACTTGTGGGGTCCTTCTCCTGTCACTG
GTTATCACCCTTTACTGCAACCACAGGAACCGAAGACGTGTTTGCAAATG
TCCCCGGCCTGTGGTCAAATCGGGAGACAAGCCCAGCCTTTCGGCGAGAT
ACGTCTAA.

By “CD45 polypeptide” is meant a polypeptide or fragment thereof having at least about 85% amino acid sequence identity to NCBI Reference Sequence no. NP_002829.3, provided below, and that functions in T cell signaling and aids with cytotoxic T cell antigen interactions.

MTMYLWLKLLAFGFAFLDTEVFVTGQSPTPSPTGLTTAKMPSVPLSSDPL
PTHTTAFSPASTFERENDFSETTTSLSPDNTSTQVSPDSLDNASAFNTTG
VSSVQTPHLPTHADSQTPSAGTDTQTFSGSAANAKLNPTPGSNAISDVPG
ERSTASTFPTDPVSPLTTTLSLAHHSSAALPARTSNTTITANTSDAYLNA
SETTTLSPSGSAVISTTTIATTPSKPTCDEKYANITVDYLYNKETKLFTA
KLNVNENVECGNNTCTNNEVHNLTECKNASVSISHNSCTAPDKTLILDVP
PGVEKFQLHDCTQVEKADTTICLKWKNIETFTCDTQNITYRFQCGNMIFD
NKEIKLENLEPEHEYKCDSEILYNNHKFTNASKIIKTDFGSPGEPQIIFC
RSEAAHQGVITWNPPQRSFHNFTLCYIKETEKDCLNLDKNLIKYDLQNLK
PYTKYVLSLHAYIIAKVQRNGSAAMCHFTTKSAPPSQVWNMTVSMTSDNS
MHVKCRPPRDRNGPHERYHLEVEAGNTLVRNESHKNCDFRVKDLQYSTDY
TFKAYFHNGDYPGEPFILHHSTSYNSKALIAFLAFLIIVTSIALLVVLYK
IYDLHKKRSCNLDEQQELVERDDEKQLMNVEPIHADILLETYKRKIADEG
RLFLAEFQSIPRVFSKFPIKEARKPFNQNKNRYVDILPYDYNRVELSEIN
GDAGSNYINASYIDGFKEPRKYIAAQGPRDETVDDFWRMIWEQKATVIVM
VTRCEEGNRNKCAEYWPSMEEGTRAFGDVVVKINQHKRCPDYIIQKLNIV
NKKEKATGREVTHIQFTSWPDHGVPEDPHLLLKLRRRVNAFSNFFSGPIV
VHCSAGVGRTGTYIGIDAMLEGLEAENKVDVYGYVVKLRRQRCLMVQVEA
QYILIHQALVEYNQFGETEVNLSELHPYLHNMKKRDPPSEPSPLEAEFQR
LPSYRSWRTQHIGNQEENKSKNRNSNVIPYDYNRVPLKHELEMSKESEHD
SDESSDDDSDSEEPSKYINASFIMSYWKPEVMIAAQGPLKETIGDFWQMI
FQRKVKVIVMLTELKHGDQEICAQYWGEGKQTYGDIEVDLKDTDKSSTYT
LRVFELRHSKRKDSRTVYQYQYTNWSVEQLPAEPKELISMIQVVKQKLPQ
KNSSEGNKHHKSTPLLIHCRDGSQQTGIFCALLNLLESAETEEVVDIFQV
VKALRKARPGMVSTFEQYQFLYDVIASTYPAQNGQVKKNNHQEDKIEFDN
EVDKVKQDANCVNPLGAPEKLPEAKEQAEGSEPTSGTEGPEHSVNGPASP
ALNQGS.

By “CD45 polynucleotide” is meant a polynucleotide that encodes a CD45 polypeptide or a fragment thereof. An exemplary CD45 polynucleotide sequence is provided at NCBI Reference Sequence no. NM_002838.5, provided below.

ATGACCATGTATTTGTGGCTTAAACTCTTGGCATTTGGCTTTGCCTTTCTGGACACAGAAGTAT
TTGTGACAGGGCAAAGCCCAACACCTTCCCCCACTGGATTGACTACAGCAAAGATGCCCAGTGT
TCCACTTTCAAGTGACCCCTTACCTACTCACACCACTGCATTCTCACCCGCAAGCACCTTTGAA
AGAGAAAATGACTTCTCAGAGACCACAACTTCTCTTAGTCCAGACAATACTTCCACCCAAGTAT
CCCCGGACTCTTTGGATAATGCTAGTGCTTTTAATACCACAGGTGTTTCATCAGTACAGACGCC
TCACCTTCCCACGCACGCAGACTCGCAGACGCCCTCTGCTGGAACTGACACGCAGACATTCAGC
GGCTCCGCCGCCAATGCAAAACTCAACCCTACCCCAGGCAGCAATGCTATCTCAGATGTCCCAG
GAGAGAGGAGTACAGCCAGCACCTTTCCTACAGACCCAGTTTCCCCATTGACAACCACCCTCAG
CCTTGCACACCACAGCTCTGCTGCCTTACCTGCACGCACCTCCAACACCACCATCACAGCGAAC
ACCTCAGATGCCTACCTTAATGCCTCTGAAACAACCACTCTGAGCCCTTCTGGAAGCGCTGTCA
TTTCAACCACAACAATAGCTACTACTCCATCTAAGCCAACATGTGATGAAAAATATGCAAACAT
CACTGTGGATTACTTATATAACAAGGAAACTAAATTATTTACAGCAAAGCTAAATGTTAATGAG
AATGTGGAATGTGGAAACAATACTTGCACAAACAATGAGGTGCATAACCTTACAGAATGTAAAA
ATGCGTCTGTTTCCATATCTCATAATTCATGTACTGCTCCTGATAAGACATTAATATTAGATGT
GCCACCAGGGGTTGAAAAGTTTCAGTTACATGATTGTACACAAGTTGAAAAAGCAGATACTACT
ATTTGTTTAAAATGGAAAAATATTGAAACCTTTACTTGTGATACACAGAATATTACCTACAGAT
TTCAGTGTGGTAATATGATATTTGATAATAAAGAAATTAAATTAGAAAACCTTGAACCCGAACA
TGAGTATAAGTGTGACTCAGAAATACTCTATAATAACCACAAGTTTACTAACGCAAGTAAAATT
ATTAAAACAGATTTTGGGAGTCCAGGAGAGCCTCAGATTATTTTTTGTAGAAGTGAAGCTGCAC
ATCAAGGAGTAATTACCTGGAATCCCCCTCAAAGATCATTTCATAATTTTACCCTCTGTTATAT
AAAAGAGACAGAAAAAGATTGCCTCAATCTGGATAAAAACCTGATCAAATATGATTTGCAAAAT
TTAAAACCTTATACGAAATATGTTTTATCATTACATGCCTACATCATTGCAAAAGTGCAACGTA
ATGGAAGTGCTGCAATGTGTCATTTCACAACTAAAAGTGCTCCTCCAAGCCAGGTCTGGAACAT
GACTGTCTCCATGACATCAGATAATAGTATGCATGTCAAGTGTAGGCCTCCCAGGGACCGTAAT
GGCCCCCATGAACGTTACCATTTGGAAGTTGAAGCTGGAAATACTCTGGTTAGAAATGAGTCGC
ATAAGAATTGCGATTTCCGTGTAAAAGATCTTCAATATTCAACAGACTACACTTTTAAGGCCTA
TTTTCACAATGGAGACTATCCTGGAGAACCCTTTATTTTACATCATTCAACATCTTATAATTCT
AAGGCACTGATAGCATTTCTGGCATTTCTGATTATTGTGACATCAATAGCCCTGCTTGTTGTTC
TCTACAAAATCTATGATCTACATAAGAAAAGATCCTGCAATTTAGATGAACAGCAGGAGCTTGT
TGAAAGGGATGATGAAAAACAACTGATGAATGTGGAGCCAATCCATGCAGATATTTTGTTGGAA
ACTTATAAGAGGAAGATTGCTGATGAAGGAAGACTTTTTCTGGCTGAATTTCAGAGCATCCCGC
GGGTGTTCAGCAAGTTTCCTATAAAGGAAGCTCGAAAGCCCTTTAACCAGAATAAAAACCGTTA
TGTTGACATTCTTCCTTATGATTATAACCGTGTTGAACTCTCTGAGATAAACGGAGATGCAGGG
TCAAACTACATAAATGCCAGCTATATTGATGGTTTCAAAGAACCCAGGAAATACATTGCTGCAC
AAGGTCCCAGGGATGAAACTGTTGATGATTTCTGGAGGATGATTTGGGAACAGAAAGCCACAGT
TATTGTCATGGTCACTCGATGTGAAGAAGGAAACAGGAACAAGTGTGCAGAATACTGGCCGTCA
ATGGAAGAGGGCACTCGGGCTTTTGGAGATGTTGTTGTAAAGATCAACCAGCACAAAAGATGTC
CAGATTACATCATTCAGAAATTGAACATTGTAAATAAAAAAGAAAAAGCAACTGGAAGAGAGGT
GACTCACATTCAGTTCACCAGCTGGCCAGACCACGGGGTGCCTGAGGATCCTCACTTGCTCCTC
AAACTGAGAAGGAGAGTGAATGCCTTCAGCAATTTCTTCAGTGGTCCCATTGTGGTGCACTGCA
GTGCTGGTGTTGGGCGCACAGGAACCTATATCGGAATTGATGCCATGCTAGAAGGCCTGGAAGC
CGAGAACAAAGTGGATGTTTATGGTTATGTTGTCAAGCTAAGGCGACAGAGATGCCTGATGGTT
CAAGTAGAGGCCCAGTACATCTTGATCCATCAGGCTTTGGTGGAATACAATCAGTTTGGAGAAA
CAGAAGTGAATTTGTCTGAATTACATCCATATCTACATAACATGAAGAAAAGGGATCCACCCAG
TGAGCCGTCTCCACTAGAGGCTGAATTCCAGAGACTTCCTTCATATAGGAGCTGGAGGACACAG
CACATTGGAAATCAAGAAGAAAATAAAAGTAAAAACAGGAATTCTAATGTCATCCCATATGACT
ATAACAGAGTGCCACTTAAACATGAGCTGGAAATGAGTAAAGAGAGTGAGCATGATTCAGATGA
ATCCTCTGATGATGACAGTGATTCAGAGGAACCAAGCAAATACATCAATGCATCTTTTATAATG
AGCTACTGGAAACCTGAAGTGATGATTGCTGCTCAGGGACCACTGAAGGAGACCATTGGTGACT
TTTGGCAGATGATCTTCCAAAGAAAAGTCAAAGTTATTGTTATGCTGACAGAACTGAAACATGG
AGACCAGGAAATCTGTGCTCAGTACTGGGGAGAAGGAAAGCAAACATATGGAGATATTGAAGTT
GACCTGAAAGACACAGACAAATCTTCAACTTATACCCTTCGTGTCTTTGAACTGAGACATTCCA
AGAGGAAAGACTCTCGAACTGTGTACCAGTACCAATATACAAACTGGAGTGTGGAGCAGCTTCC
TGCAGAACCCAAGGAATTAATCTCTATGATTCAGGTCGTCAAACAAAAACTTCCCCAGAAGAAT
TCCTCTGAAGGGAACAAGCATCACAAGAGTACACCTCTACTCATTCACTGCAGGGATGGATCTC
AGCAAACGGGAATATTTTGTGCTTTGTTAAATCTCTTAGAAAGTGCGGAAACAGAAGAGGTAGT
GGATATTTTTCAAGTGGTAAAAGCTCTACGCAAAGCTAGGCCAGGCATGGTTTCCACATTCGAG
CAATATCAATTCCTATATGACGTCATTGCCAGCACCTACCCTGCTCAGAATGGACAAGTAAAGA
AAAACAACCATCAAGAAGATAAAATTGAATTTGATAATGAAGTGGACAAAGTAAAGCAGGATGC
TAATTGTGTTAATCCACTTGGTGCCCCAGAAAAGCTCCCTGAAGCAAAGGAACAGGCTGAAGGT
TCTGAACCCACGAGTGGCACTGAGGGGCCAGAACATTCTGTCAATGGTCCTGCAAGTCCAGCTT
TAAATCAAGGTTCATAG.

By “major histocompatibility complex II (MHCII) polypeptide” is meant a polypeptide or fragment thereof having at least about 85% amino acid sequence identity to the MHCII alpha chain or MHCII beta chain amino acid sequence provided below, and that is capable of functioning in antigen presentation.

> MHCII alpha chain
EEHVIIQAEFYLNPDQSGEFMFDFDGDEIFHVDMAKKETVWRLEEFGRFA
SFEAQGALANIAVDKANLEIMTKRSNYTPITNVPPEVTVLTNSPVELREP
NVLICFIDKFTPPVVNVTWLRNGKPVTTGVSETVFLPREDHLFRKFHYLP
FLPSTEDVYDCRVEHWGLDEPLLKHWEFDA.
> MHCII beta chain
TRPRFLWQLKFECHFFNGTERVRLLERCIYNQEESVRFDSDVGEYRAVTE
LGRPDAEYWNSQKDLLEQRRAAVDTYCRHNYGVGESFTVQRRVEPKVTVY
PSKTQPLQHHNLLVCSVSGFYPGSIEVRWFRNGQEEKAGVVSTGLIQNGD
WTFQTLVMLETVPRSGEVYTCQVEHPSVTSPLTVEWRA.

By “MHCII polynucleotide” is meant a polynucleotide that encodes a MHCII polypeptide or a fragment thereof.

By “DMV1-123-MeV122-529 (F2 DMV fusion) polypeptide” is meant a polypeptide or fragment thereof having at least about 85% amino acid sequence identity to the sequence provided below, and that functions in lentivirus envelope fusion with the membrane of a target cell.

MAASNGGVMYQSFLTIIILVIMTEGQIHWGNLSKIGIVGTGSASYKVMTR
PNHQYLVIKLMPNVTMIDNCTRTEVTEYRKLLKTVLEPVKNALTVITKNI
KPIQSLYPYDVPDYATTSRRSKRFAGVVLAGAALGVATAAQITAGIALHQ
SMLNSQAIDNLRASLETTNQAIEAIRQAGQEMILAVQGVQDYINNELIPS
MNQLSCDLIGQKLGLKLLRYYTEILSLFGPSLRDPISAEISIQALSYALG
GDINKVLEKLGYSGGDLLGILESRGIKARITHVDTESYFIVLSIAYPTLS
EIKGVIVHRLEGVSYNIGSQEWYTTVPKYVATQGYLISNFDESSCTFMPE
GTVCSQNALYPMSPLLQECLRGSTKSCARTLVSGSFGNRFILSQGNLIAN
CASILCKCYTTGTIINQDPDKILTYIAADHCPVVEVNGVTIQVGSRRYPD
AVYLHRIDLGPPISLERLDVGTNLGNAIAKLEDAKELLESSDQILRSMKG
LSSTSIVYILIAVCLGGLIGIPALICCCRGR.

In the above polypeptide sequence, an DMF-F signal peptide is underlined, an DMV-F F2 domain is in plain text, an HA tag is in bold text, a furin cleavage site is in italic text, an MeV-F extravirion domain is double-underlined, an MeV-F transmembrane domain is in bold italic text, and an MeV-F intravirion domain is in bold underlined text.

By “DMV1-123-MeV122-529 (F2 DMV fusion) polynucleotide” is meant a polynucleotide encoding an F2 DMV fusion polypeptide.

By “DMV1-229-MeV229-529 (S-S DMV fusion) polypeptide” is meant a polypeptide or fragment thereof having at least about 85% amino acid sequence identity to the sequence provided below, and that has activity associated with fusion of a lentivirus envelope containing an H protein domain with a membrane.

MAASNGGVMYQSFLTIIILVIMTEGQIHWGNLSKIGIVGTGSASYKVMTRPNHQYLVIKLMPNV

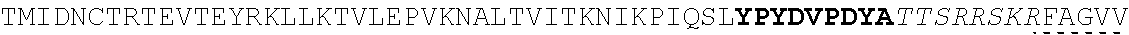

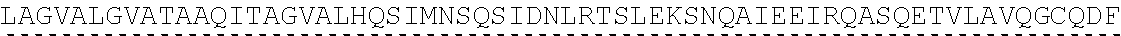

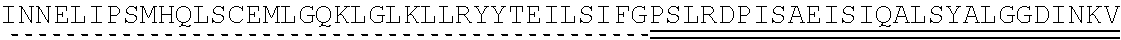

LEKLGYSGGDLLGILESRGIKARITHVDTESYFIVLSIAYPTLSEIKGVIVHRLEGVSYNIGSQ
EWYTTVPKYVATOGYLISNFDESSCTFMPEGTVCSONALYPMSPLLQECLRGSTKSCARTLVSG
SFGNRFILSOGNLIANCASILCKCYTTGTIINQDPDKILTYIAADHCPVVEVNGVTIQVGSRRY
PDAVYLHRIDLGPPISLERLDVGTNLGNAIAKLEDAKELLESSDOILRSMKGLSSTSSIVYILI
AVCLGGLIGIPALICCCRGR.

In the above polypeptide sequence, an DMF-F signal peptide is underlined, an DMV-F F2 domain is in plain text, an HA tag is in bold text, a furin cleavage site is in italic text, a DMV-F extravirion domain is underlined with a dashed line, an MeV-F extravirion domain is double-underlined, an MeV-F transmembrane domain is in bold italic text, and an MeV-F intravirion domain is in bold underlined text.

By “DMV1-229-MeV229-529 (S-S DMV fusion) polynucleotide” is meant a polynucleotide encoding an S-S DMV fusion polypeptide.

By “DMV1-311-MeV309-529 (intermediate DMV fusion) polypeptide” is meant a polypeptide or fragment thereof having at least about 85% amino acid sequence identity to the sequence provided below, and that functions in lentivirus envelope fusion with the membrane of a target cell.

MAASNGGVMYQSFLTIIILVIMTEGQIHWGNLSKIGIVGTGSASYKVMTRPNHQYLVIKLMPNV

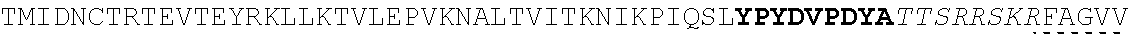

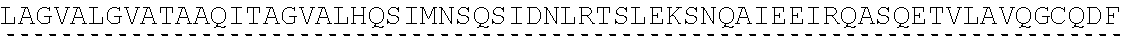

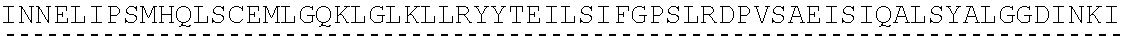

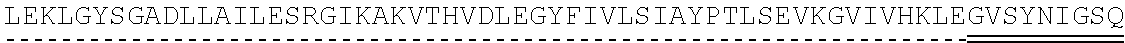

EWYTTVPKYVATQGYLISNFDESSCTFMPEGTVCSONALYPMSPLLOECLRGSTKSCARTLVSG
SFGNRFILSOGNLIANCASILCKCYTTGTIINODPDKILTYIAADHCPVVEVNGVTIQVGSRRY
PDAVYLHRIDLGPPISLERLDVGTNLGNAIAKLEDAKELLESSDOILRSMKGLSSTSSIVYILI
AVCLGGLIGIPALICCCRGR.

In the above polypeptide sequence, an DMF-F signal peptide is underlined, an DMV-F F2 domain is in plain text, an HA tag is in bold text, a furin cleavage site is in italic text, a DMV-F extravirion domain is underlined with a dashed line, an MeV-F extravirion domain is double-underlined, an MeV-F transmembrane domain is in bold italic text, and an MeV-F intravirion domain is in bold underlined text.

By “DMV1-311-MeV309-529 (intermediate DMV fusion) polynucleotide” is meant a polynucleotide encoding an intermediate DMV fusion polypeptide.

By “DMV1-407-MeV405-529 (H interacting domain DMV fusion) polypeptide” is meant a polypeptide or fragment thereof having at least about 85% amino acid sequence identity to the sequence provided below, and that functions in lentivirus envelope fusion with the membrane of a target cell.

MAASNGGVMYQSFLTIIILVIMTEGQIHWGNLSKIGIVGTGSASYKVMTRPNHQYLVIKLMPNV

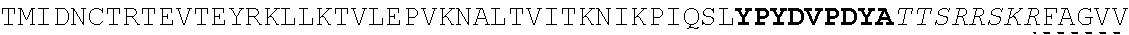

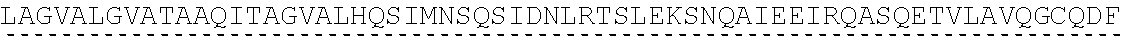

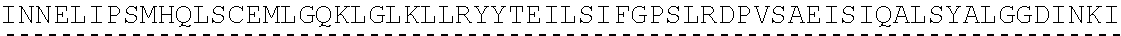

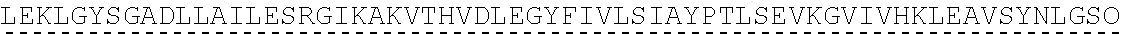

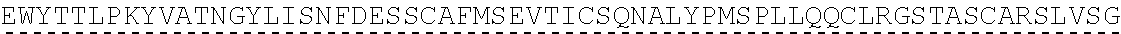

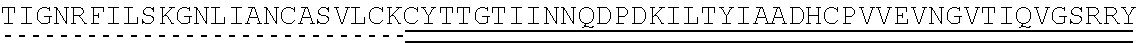

PDAVYLHRIDLGPPISLERLDVGTNLGNAIAKLEDAKELLESSDOILRSMKGLSSTSSIVYILI
AVCLGGLIGIPALICCCRGR.

In the above polypeptide sequence, an DMF-F signal peptide is underlined, an DMV-F F2 domain is in plain text, an HA tag is in bold text, a furin cleavage site is in italic text, a DMV-F extravirion domain is underlined with a dashed line, an MeV-F extravirion domain is double-underlined, an MeV-F transmembrane domain is in bold italic text, and an MeV-F intravirion domain is in bold underlined text.

By “DMV1-407-MeV405-529 (H interacting domain DMV fusion) polynucleotide” is meant a polynucleotide encoding an H interacting domain DMV fusion polypeptide.

By “DMV1-465-MeV463-529 (stalk DMV fusion) polypeptide” is meant a polypeptide or fragment thereof having at least about 85% amino acid sequence identity to the sequence provided below, and that functions in lentivirus envelope fusion with the membrane of a target cell.

MAASNGGVMYQSFLTIIILVIMTEGQIHWGNLSKIGIVGTGSASYKVMTRPNHQYLVIKLMPNV

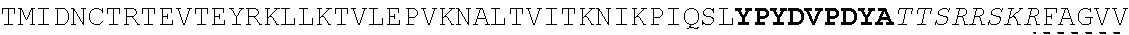

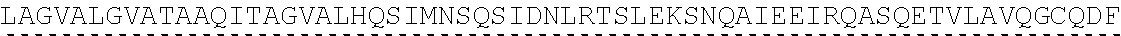

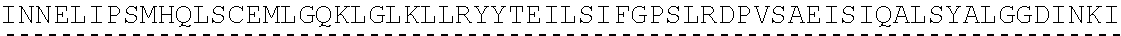

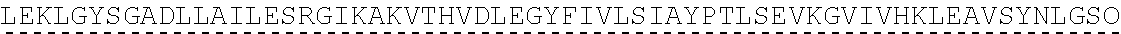

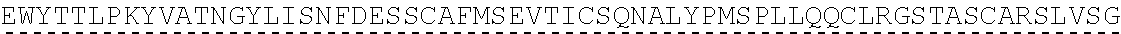

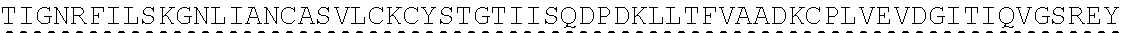

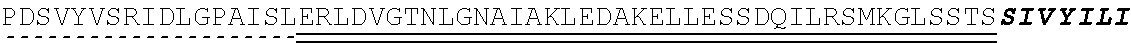

AVCLGGLIGIPALICCCRGR.

In the above polypeptide sequence, an DMF-F signal peptide is underlined, an DMV-F F2 domain is in plain text, an HA tag is in bold text, a furin cleavage site is in italic text, a DMV-F extravirion domain is underlined with a dashed line, an MeV-F extravirion domain is double-underlined, an MeV-F transmembrane domain is in bold italic text, and an MeV-F intravirion domain is in bold underlined text.

By “DMV1-465-MeV463-529 (stalk DMV fusion) polynucleotide” is meant a polynucleotide encoding a stalk DMV fusion polypeptide.

By “administering” is meant giving, supplying, dispensing a composition, agent, therapeutic product, and the like to a subject, or applying or bringing the composition and the like into contact with the subject. Administering or administration may be accomplished by any of a number of routes, such as, for example, without limitation, parenteral or systemic, intravenous (IV), (injection), subcutaneous, intrathecal, intracranial, intramuscular, dermal, intradermal, inhalation, rectal, intravaginal, topical, oral, subcutaneous, intramuscular, or intraocular. In embodiments, administration is systemic, such as by inoculation, injection, or intravenous injection.

By “agent” is meant any small molecule chemical compound, antibody, nucleic acid molecule, or polypeptide, or fragments thereof.

By “alteration” is meant a change (increase or decrease) in the structure, expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.”

By “ameliorate” is meant decrease, suppress, attenuate, diminish, arrest, or stabilize the development or progression of a disease.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

As used herein, the term “antibody” (Ab) refers to an immunoglobulin molecule that specifically binds to, or is immunologically reactive with, a particular antigen, and antigen binding fragments thereof. Exemplary antibodies encompass polyclonal, monoclonal, genetically and molecularly engineered and otherwise modified forms of antibodies, including, but not limited to, chimeric antibodies, humanized antibodies, heteroconjugate antibodies (e.g., bi- tri- and quad-specific antibodies, diabodies, triabodies, and tetrabodies), and antigen-binding fragments of antibodies, including e.g., Fab′, F(ab′)2, Fab, Fv, rlgG, and scFv fragments. Antibodies (immunoglobulins) comprise two heavy chains linked together by disulfide bonds, and two light chains, with each light chain being linked to a respective heavy chain by disulfide bonds in a “Y” shaped configuration. Each heavy chain has at one end a variable domain (VH) followed by a number of constant domains (CH). Each light chain has a variable domain (VL) at one end and a constant domain (CL) at its other end. The variable domain of the light chain (VL) is aligned with the variable domain of the heavy chain (VL), and the light chain constant domain (CL) is aligned with the first constant domain of the heavy chain (CH1). The variable domains of each pair of light and heavy chains form the antigen binding site. The isotype of the heavy chain (gamma, alpha, delta, epsilon or mu) determines the immunoglobulin class (IgG, IgA, IgD, IgE or IgM, respectively). The light chain is either of two isotypes (kappa (κ) or lambda (λ)) found in all antibody classes. The terms “antibody” or “antibodies” include intact antibodies, such as polyclonal antibodies or monoclonal antibodies (mAbs), as well as proteolytic portions or fragments thereof, such as the Fab or F(ab′)₂ fragments, that are capable of specifically binding to a target protein. Antibodies may include chimeric antibodies; recombinant and engineered antibodies, and antigen binding fragments thereof.

By “antigen” is meant an agent to which an antibody or other polypeptide capture molecule specifically binds. Exemplary antigens include small molecules, carbohydrates, proteins, and polynucleotides. In embodiments, the polypeptide capture molecule is a VHH.

By “chimeric polypeptide” or “chimera” is meant a polypeptide derived from two or more original polypeptide sequences. For example, in embodiments, a chimeric polypeptide of the present disclosure comprises amino acid sequences derived from two or more viral envelope glycoproteins (e.g., two or more of canine distemper virus, dolphin Morbillivirus, feline Morbillivirus, measles virus, Peste des petits ruminant virus, phocine Morbillivirus, Rinderpest virus, and small ruminant virus). In some instances, the chimeric polypeptide contains an amino acid sequences derived from two or more hemagglutinin polypeptides. In embodiments, the chimeric polypeptide contains amino acid sequences derived from two or more F proteins.

By “Chimeric Antigen Receptor” or alternatively a “CAR” is meant a polypeptide capable of providing an immune effector cell with specificity for a target cell, typically a cancer cell. In some embodiments, a CAR comprises at least an extracellular antigen binding domain, a transmembrane domain and a cytoplasmic signaling domain comprising a functional signaling domain derived from a stimulatory molecule and/or costimulatory molecule. In embodiments, the stimulatory molecule is the zeta chain associated with the T cell receptor complex. In one embodiment, the cytoplasmic signaling domain further comprises one or more functional signaling domains derived from at least one costimulatory molecule. In one embodiment, the costimulatory molecule is 4-1BB (i.e., CD137), CD27 and/or CD28. In one embodiment, the CAR comprises a chimeric fusion protein comprising an extracellular antigen binding domain, a transmembrane domain and an intracellular signaling domain comprising a functional signaling domain derived from a stimulatory molecule. In one embodiment, the CAR comprises a chimeric fusion protein comprising an extracellular antigen binding domain, a transmembrane domain and an intracellular signaling domain comprising a functional signaling domain derived from a costimulatory molecule and a functional signaling domain derived from a stimulatory molecule. In one embodiment, the CAR comprises a chimeric fusion protein comprising an extracellular antigen binding domain, a transmembrane domain and an intracellular signaling domain comprising two functional signaling domains derived from one or more costimulatory molecule(s) and a functional signaling domain derived from a stimulatory molecule. In one embodiment, the CAR comprises a chimeric fusion protein comprising an extracellular antigen binding domain, a transmembrane domain and an intracellular signaling domain comprising at least two functional signaling domains derived from one or more costimulatory molecule(s) and a functional signaling domain derived from a stimulatory molecule. In one embodiment the CAR comprises an optional leader sequence at the amino-terminus (N-ter) of the CAR fusion protein. In one embodiment, the CAR further comprises a leader sequence at the N-terminus of the extracellular antigen binding domain, wherein the leader sequence is optionally cleaved from the antigen binding domain (e.g., a scFv) during cellular processing and localization of the CAR to the cellular membrane.

As used herein, the term “complementarity determining region” (CDR) refers to a hypervariable region found both in the light chain and the heavy chain variable domains (VL and VH domains, respectively). The more highly conserved portions of variable domains are called the framework regions (FRs). As is appreciated in the art, the amino acid positions that delineate a hypervariable region of an antibody can vary, depending on the context and the various definitions known in the art. Some positions within a variable domain may be viewed as hybrid hypervariable positions in that these positions can be deemed to be within a hypervariable region under one set of criteria while being deemed to be outside a hypervariable region under a different set of criteria. One or more of these positions can also be found in extended hypervariable regions. The variable domains of native heavy and light chains each comprise four framework regions (FR1, FR2, FR3, FR4) that primarily adopt a beta-sheet configuration, connected by three CDRs (CDR1, CDR2, CDR3), which form loops that connect, and in some cases form part of, the beta-sheet structure. The CDRs in each chain are held together in close proximity by the FR regions in the order FR1-CDR1-FR2-CDR2-FR3-CDR3-FR4. and the CDRs in each antibody chain contribute to the formation of the target binding site of antibodies (see Kabat et al, Sequences of Proteins of Immunological Interest (National Institute of Health, Bethesda, Md. 1987; incorporated herein by reference). As used herein, numbering of immunoglobulin amino acid residues is done according to the immunoglobulin amino acid residue numbering system of Kabat et al, unless otherwise indicated.

The term a “costimulatory molecule” refers to a cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that contribute to an efficient immune response. Costimulatory molecules include, but are not limited to an MHC class I molecule, BTLA and a Toll ligand receptor, as well as OX40, CD27, CD28, CDS, ICAM-1, LFA-1 (CD1 1a/CD18), ICOS (CD278), and 4-1BB (CD137). Further examples of such costimulatory molecules include CDS, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11 b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAMI, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments. Any embodiments specified as “comprising” a particular component(s) or element(s) are also contemplated as “consisting of” or “consisting essentially of” the particular component(s) or element(s) in some embodiments.

By “consist essentially” it is meant that the ingredients include only the listed components along with the normal impurities present in commercial materials and with any other additives present at levels which do not affect the operation of the disclosure, for instance at levels less than 5% by weight or less than 1% or even 0.5% by weight.

By “disease” is meant any condition or disorder that damages or interferes with the normal function of a cell, tissue, or organ. Non-limiting examples of diseases include a cancer or tumor. In embodiments, the disease is a cytomegalovirus (CMV) infection, a cancer or tumor, a lymphoma (e.g., a B-cell lymphoma), a neoplasia, an influenza infection, or coronavirus disease of 2019 (COVID-19).

By “effective amount” is meant the amount of an agent required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1,000 nucleotides or amino acids.

As used herein, “heterologous” is used to refer to a gene, polynucleotide, or polypeptide experimentally put into a cell or viral particle that does not normally comprise that polynucleotide or polypeptide. In various embodiments “heterologous” is used to refer to a sequence derived from a different cell or virus from that virus or cell into which the sequence has been introduced.

By “increases” is meant a positive alteration of at least 10%, 25%, 50%, 75%, or 100%.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “measles-immune” is meant a subject comprising antibodies capable of neutralizing the Measles morbillivirus.

As used herein, “obtaining” as in “obtaining an agent” includes synthesizing, purchasing, or otherwise acquiring the agent.

As used herein, the term “pharmaceutically acceptable” refers to molecular entities, biological products and compositions that are physiologically tolerable and do not typically produce an allergic or other adverse reaction, such as gastric upset, dizziness and the like, when administered to a subject.

By “polypeptide” or “amino acid sequence” is meant any chain of amino acids, regardless of length or post-translational modification. In various embodiments, the post-translational modification is glycosylation or phosphorylation. In various embodiments, conservative amino acid substitutions may be made to a polypeptide to provide functionally equivalent variants, or homologs of the polypeptide. In some embodiments the invention embraces sequence alterations that result in conservative amino acid substitutions. In some embodiments, a “conservative amino acid substitution” refers to an amino acid substitution that does not alter the relative charge or size characteristics of the protein in which the conservative amino acid substitution is made. Variants can be prepared according to methods for altering polypeptide sequence known to one of ordinary skill in the art such as are found in references that compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Non-limiting examples of conservative substitutions of amino acids include substitutions made among amino acids within the following groups: (a) M, I, L, V; (b) F, Y, W; (c) K, R, H; (d) A, G; (e) S, T; (f) Q, N; and (g) E, D. In various embodiments, conservative amino acid substitutions can be made to the amino acid sequence of the proteins and polypeptides disclosed herein.

The term “promoter” refers to a DNA sequence recognized by polypeptides required to initiate the transcription of a polynucleotide sequence in a cell.

As used herein, the term “pseudotyped” refers to a viral particle that contains one or more heterologous viral proteins. In embodiments the heterologous viral protein is an envelope glycoprotein. A pseudotyped virus may be one in which the envelope glycoproteins of an enveloped virus or the capsid proteins of a non-enveloped virus originate from a virus that differs from the source of the original virus genome and the genome replication apparatus. (D. A. Sanders, 2002, Curr. Opin. Biotechnol., 13:437-442). The foreign viral envelope proteins of a pseudotyped virus can be utilized to alter host tropism or to increase or decrease the stability of the virus particles. FIG. 1 provides a representative list of envelope glycoproteins. Further non-limiting examples of envelope glycoproteins include MeV-Hwtc18, CDV-F, CDF-Fc30, DMV-F, DMV-Fc30, DMV-H, DMV-Hc18 (MeV-Hc18-DMV), CDV-H, CDV-Hc18 (MeV-Hc18-CDV), FMV-H, PPRV-H, RPV-H, and RMV-H. Examples of pseudotyped viral particles include a virus that contains one or more envelope glycoproteins that do not naturally occur on the exterior of the wild-type virus. Pseudotyped viral particles can infect cells and express and produce proteins or molecules encoded by polynucleotides contained within the viral particles.

By “reduces” is meant a negative alteration of at least 10%, 25%, 50%, 75%, or 100%.

By “reference” is meant a healthy subject or a subject prior to administration of a pseudotyped viral particle of the invention. In embodiments, the reference has never been administered a pseudotyped viral particle of the invention. In embodiments, the reference is a healthy subject prior to a particular instance of administration of a pseudotyped viral particle of the invention. A healthy subject is a subject free from a disease treated using a pseudotyped viral particle of the invention.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

The term “signaling domain” refers to the functional portion of a protein which acts by transmitting information within the cell to regulate cellular activity via defined signaling pathways by generating second messengers or functioning as effectors by responding to such messengers.

An “intracellular signaling domain,” refers to an intracellular portion of a molecule. The intracellular signaling domain generates a signal that promotes an immune effector function of a CAR containing cell, e.g., a CART cell. Examples of immune effector function, e.g., in a CART cell, include cytolytic activity and helper activity, including the secretion of cytokines.

By “specifically binds” is meant in the context of an antibody or other polypeptide capture molecule recognizes and binds a polypeptide of the invention, but which does not substantially recognize and bind other molecules in a sample, for example, a biological sample, which naturally includes a polypeptide of the invention. In embodiments, the capture molecule is a VHH domain or a fragment thereof. A VHH domain or fragment thereof that specifically binds to an antigen will bind to the antigen with a K_{D} of less than 100 nM. For example, a VHH domain or fragment thereof that specifically binds to an antigen will bind to the antigen with a K_{D} of up to 100 nM (e.g., between 1 pM and 100 nM). A VHH domain or fragment thereof that does not exhibit specific binding to a particular antigen or epitope thereof will exhibit a K_{D} of greater than 100 nM (e.g., greater than 500 nm, 1 uM, 100 uM, 500 uM, or 1 mM) for that particular antigen or epitope thereof. A variety of immunoassay formats may be used to select a VHH domain or fragment thereof that specifically immunoreactive with a particular protein or carbohydrate. For example, solid-phase ELISA immunoassays are routinely used to select VHH domains or fragments thereof specifically immunoreactive with a protein or carbohydrate. See, Harlow & Lane, Antibodies, A Laboratory Manual, Cold Spring Harbor Press, New York (1988) and Harlow & Lane, Using Antibodies, A Laboratory Manual, Cold Spring Harbor Press, New York (1999), for a description of immunoassay formats and conditions that can be used to determine specific immunoreactivity.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal, such as a murine, bovine, equine, canine, ovine, or feline.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the term “vector” refers to a polynucleotide suitable for delivery of a gene sequence to a cell, or to a pseudotyped virus particle. Non-limiting examples of vectors include plasmids and cosmids. A “vector” further refers to a nucleic acid (polynucleotide) molecule into which foreign nucleic acid can be inserted without disrupting the ability of the vector to be expressed in, replicate in, and/or integrate into a host cell. A variety of vectors have been developed for the delivery of polynucleotides encoding exogenous proteins into a prokaryotic or eukaryotic cell. A vector may contain a polynucleotide sequence that includes gene of interest (e.g., a heterologous gene, such as a therapeutic gene, or a reporter gene) as well as, for example, additional sequence elements capable of regulating transcription, translation, and/or the integration of these polynucleotide sequences into the genome of a cell. A vector may contain regulatory sequences, such as a promoter, e.g., a subgenomic promoter, region and an enhancer region, which direct gene transcription. A vector may contain polynucleotide sequences (enhancer sequences) that enhance the rate of translation of these genes or improve the stability or nuclear export of the mRNA that results from gene transcription. These sequence elements may include, e.g., 5′ and 3′ untranslated regions, an internal ribosomal entry site (IRES), and/or a polyadenylation signal site in order to direct efficient transcription of a gene carried on the expression vector. Vectors, such as the pseudotyped viral particles described herein, may also be referred to as expression vectors.

By “viral transfer vector” is meant a vector comprising a polynucleotide encoding a heterologous polypeptide and containing viral cis-elements required for packaging into a viral particle and insertion into host genome.

“Transduction” refers to a process by which DNA or polynucleotide, e.g., one or more heterologous genes, contained in a virus or pseudotyped viral particle is introduced or transferred into a cell by the virus or pseudotyped viral particle, wherein the DNA or polynucleotide is expressed. In an embodiment, the DNA or polynucleotide transduced into a cell is stably expressed in the cell. In some cases, the virus or virus vector is said to infect a cell.

As used herein, the term “vehicle” refers to a solvent, diluent, or carrier component of a pharmaceutical composition.

By “viral particle” is meant an agent capable of infecting a cell and that exists as an independent particle containing a core viral genome or polynucleotide, a capsid, which surrounds the genetic material and protects it, and an envelope of lipids surrounding the capsid. A viral particle may refer to the form of a virus before it infects a cell and becomes intracellular, or to the form of the virus that infects a cell.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1 is a chart providing an overview of information relating to different lentiviral vectors. The chart of FIG. 1 is taken from Frank and Bucholz, “Surface-Engineered Lentiviral Vectors for Selective Gene Transfer into Subtypes of Lymphocytes,” Molecular Therapy—Methods & Clinical Development, 12:19-31 (2019), doi: 10.1016/j.omtm.2018.10.006.

FIGS. 2A-2C provide phylogenetic trees, a schematic providing an overview of the domain structure of MeV-H, and a multiple sequence alignment for glycoproteins derived from the indicated viruses. FIGS. 2A and 2C provide phylogenetic tress showing clustering of different viral envelope glycoprotein amino acid sequences. In FIG. 2B the term “TM” designates “transmembrane domain, the term “MeV-H” designates envelope protein H from the measles virus, the term “CDV-H” designates envelope protein H from the canine distemper virus, and “DMV-H” designates the envelope protein H from the dolphin Morbillivirus. In FIG. 2B, stars and medium-grey highlighted text identify residues targeted by MeV-H neutralizing antibodies (e.g., neutralizing antibodies in the serum of a subject(s) vaccinated against the measles virus). Not being bound by theory, MeV-H, CDV-H, and DMV-H each have similar domain structures; however, the globular head domains (alternatively referred to as “extracellular domains” or “extravirion domains”) of CDV-H and DMV-H lack residues found in MeV-H that are targeted by MeV-H neutralizing antibodies. FIG. 2A is adapted from a figure provided in Marsh, Wang, et al., The Role of Animals in Emerging Viral Diseases, 2014, the disclosure of which is incorporated herein in its entirety by reference for all purposes. FIG. 2C is adapted from a figure provided in Pfeffermannat, et al., Advances in Virus Research, 2018, the disclosure of which is incorporated herein in its entirety by reference for all purposes. In FIG. 2B, residues targeted by MeV-H neutralizing antibodies are indicated by arrows.

FIG. 3 presents overlaid flow cytometry histograms demonstrating that alternative MoV-H (Morbillivirus-H) polypeptides fused with VHH32 (anti-aCD45) and an HA tag are highly expressed on the cell surface. In FIG. 3 the black lines correspond to the measles virus envelope protein H (MeV-Hwt), the thick line in dark grey represents the aCD45-VHH MoV (Morbillivirus) fusion protein comprising a MoV-H domain from the indicated virus, and the filled-in grey curve represents unstained cells. In FIG. 3, the term “DMV” designates the dolphin Morbillivirus, the term “RPV” designates the Rinderpest virus, the term “RMV” designates the small ruminant virus, the term “PPRV” designates the Peste des petits ruminant virus, and the term “FMV” designates the feline Morbillivirus. The VHH domain did not impede expression. All of the MoV—H-VHH32 fusions showed surface expression.

FIG. 4 provides stacked sets of flow cytometry histograms demonstrating that anti-hCD7 VHH and anti-MHCII VHH (N11) domains were well tolerated on the surface of producer HEK293T cells when fused to CDV-H, DMV-H, or MeV-H. In FIG. 4 the term “MeV-H” designates envelope protein H from the measles virus, the term “CDV-H” designates envelope protein H from the canine distemper virus, and “DMV-H” designates the envelope protein H from the dolphin Morbillivirus. The terms “CD7 VHH” and “MHCII VHH” at the top of each set of stacked flow cytometry histograms indicate the VHH domain to which the envelope protein H indicated on the far right was fused. Higher surface expression is indicated by a higher rightmost peak in the curve (e.g., the second hump in each of the CDV-H, DMV-H, and MeV-H curves).

FIG. 5 provides overlaid flow cytometry histograms demonstrating that MV-DMV N11 (aMHCII) and MV-CDV N11 (aMHCII) fusion proteins were highly expressed on the surface of producer cells. In FIG. 5, the grey-filled curves correspond to unstained cells, the thin lines in dark grey correspond to MeV-Hwt, and the thick grey lines correspond to MHCII-VHH MoV fusions (alternatively, chimeras). In FIG. 5, the term “DMV” designates fusions comprising the dolphin Morbillivirus envelope protein H, and the term “CDV” designates fusion proteins comprising the canine distemper virus envelope protein H.

FIGS. 6A and 6B provide flow cytometry scatter plots demonstrating the ability of fusion proteins to facilitate selective transduction of MHCII+ cells, and a schematic illustrating the domain structure of the fusion proteins (MeV-DMV-N11 and MeV-CDV-N11) evaluated in the scatter plots. CDV-H (the canine distemper virus envelope protein H) and DMV-H (the dolphin Morbillivirus envelope protein H) fused to a VHH targeting MHC-II efficiently and selectively infected MHC-II+ cells. The lentivirus used to infect the cells contained the fusion proteins and the measles virus fusion protein (MeV-Fc30). As shown in FIG. 6B, The envelope protein H domains contained the cytoplasmic and stalk domains from the measles virus envelope protein H and a globular head domain from either the canine distemper virus (CDV) or the dolphin Morbillivirus (DMV). In FIG. 6B, the term “VHH” designates a VHH domain targeting MHC-II (N11), and the term “HA” designates the HA tag. Infected cells expressed GFP. All of the evaluated fusion proteins specifically infected cells expressing MHCII, as can be seen from there being few cells in Q1 and many cells in Q2. In FIG. 6A, designators Q1, Q2, Q3, and Q4 indicate quadrants one through four, respectively, and the numbers beneath each quadrant designator indicate the percent of total cells counted falling within that quadrant.

FIG. 7 provides two flow cytometry scatter plots demonstrating that viruses pseudotyped with Morbillivirus envelope proteins H and F from the same virus are more effective in infecting cells. In FIG. 7, the plot on the left shows the efficiency of cell transduction by lentivirus pseudotyped with MeV-Hwt-32 (anti-mCD45) and MeV-Fc30 and the plot on the right shows the efficiency of cell transduction by a lentivirus pseudotyped with DMV-Hwt-32 (anti-mCD45) and MeV-Fc30. In FIG. 7, designators Q1, Q2, Q3, and Q4 indicate quadrants one through four, respectively, and the numbers beneath each quadrant designator indicate the percent of total cells counted falling within that quadrant. Successfully infected cells express GFP and fall within Q2.

FIG. 8 provides overlaid flow cytometry histograms demonstrating that DMV-F was well expressed in 293 packaging cells regardless of truncation of the cytoplasmic domain.

FIG. 9 provides flow cytometry scatter plots demonstrating transduction of A20 cells by lentivirus pseudotyped with the indicated combinations of truncated or full-length dolphin Morbillivirus envelope proteins F and H, where the envelope protein H was fused to an anti-MHCII VHH (N11). In the truncations the cytoplasmic domains were truncated. The cytoplasmic domain of the DMV-H envelope protein was truncated by 18 amino acids (DMV-Hc18) and the cytoplasmic domain of the DMV-F envelope protein was truncated by 30 amino acids (DMV-Fc30). Successfully transduced cells expressed GFP and fell within the boxed regions of each figure, where the number above each boxed region indicates the percent of total counted cells falling within the boxed region. The terms “DMV-Hwt” and “DMV-Fwt” indicate the full-length envelope proteins H and F, respectively.

FIG. 10 provides schematics showing the domain structures of the measles virus envelope proteins H (MeV-H) and F (MeV-F). In FIG. 10 “18AA” refers to an 18 amino acid deletion from the N-terminus of the cytoplasmic domain of MeV-H and “30AA” refers to a 30 amino acid deletion from the C-terminus of the cytoplasmic domain of MeV-F.

FIGS. 11A-11C provide charts and a schematic showing the effect or location of different terminal amino acid deletions on the function of morbillivirus envelope glycoproteins. Truncation of the cytoplasmic domain of Morbillivirus H and F proteins is beneficial for the efficacy of lentiviral particles pseudotyped therewith. FIG. 11A shows the effect of C-terminal truncations (24 amino acids or 30 amino acids) of MeV-F on the screening titer of lentivirus particles pseudotyped with the truncated MeV-F polypeptides. The truncations increased screening titer. FIG. 11B shows the effect of N-terminal truncations (21-30 amino acids) and/or N-terminal alanine (A) amino acid additions to MeV-H on the screening titer and fusion helper function of lentivirus particles pseudotyped with the truncated and/or extended MeV-H polypeptides. The truncations increased screening titer and the addition of N-terminal alanine amino acids reduced screening titer. FIG. 11C provides a schematic showing the location within the larger domain architecture of truncations to the morbillivirus envelope proteins G and F that are beneficial to the functionality of lentiviral particles pseudotyped therewith. FIGS. 11A and 11B are adapted from figures provided in Funke, et al., Molecular Therapy, 2008, the disclosure of which is incorporated herein by reference in its entirety for all purposes. FIG. 11C is adapted from a figure provided in Bender, et al., PLOS Pathogens, 2016.

FIGS. 12A-12D provide ribbon diagrams and a schematic summarizing the design of fusion proteins containing amino acid sequences derived from the morbillivirus (MeV) envelope glycoprotein F and the dolphin morbillivirus (DMV) envelope glycoprotein F to optimize the design of the fusion proteins and improve efficacy. Optimization of the fusion proteins improved functional titers. In embodiments, the fusion proteins are suitable for use in combination with MeV-DMV-H fusion proteins (i.e., fusion proteins comprising amino acid sequences derived from the MeV envelope glycoprotein H and the DMV envelope glycoprotein H). In FIGS. 12A-12C, the shaded region of the ribbon diagrams indicates the portion of the fusion protein derived from the DMV envelope glycoprotein F (DMV-F) and the unshaded region indicates the portion of the fusion protein derived from the MeV envelope glycoprotein F (MeV-F). The ribbon diagrams present the protein structure of the extravirion domain of the fusion proteins. FIG. 12D provides a schematic summarizing the domain architecture of the fusion proteins. FIGS. 12A-12C are adapted from figures provided in Jumper, J, et al., Highly accurate protein structure prediction with AlphaFold. Nature (2021); and Varadi, M, et al., AlphaFold Protein Structure Database: massively expanding the structural coverage of protein-sequence space with high-accuracy models. Nucleic Acids Research (2021), the disclosures of which are incorporated herein by reference in their entireties for all purposes.

FIG. 13 provides flow cytometry histograms showing that the MeV-DMV-F fusion proteins were highly expressed on the surface of HEK293 cells. The surface expression of the polypeptides was detected using a fluorescently-labeled antibody specific for the HA tag within each of the fusion proteins.

FIGS. 14A-14F provide flow cytometry scatter plots and ribbon diagrams showing the amount of functional lentiviral particles produced using the indicated fusion proteins. Lentiviral particles pseudotyped with MeV-DMV-F and MeV-DMV-H fusion proteins created functional lentiviral particles. FIG. 14A provides a matrix of flow cytometry scatter plots showing the transduction efficacy, as measured using GFP expression levels in transduced cells, of lentiviruses pesudotyped with the envelope glycoproteins H indicated on the left of the matrix in combination with the envelope glycoproteins F indicated on the top of the matrix. In FIGS. 14B-14F, the upper portion of the figures provides a ribbon diagram showing the structure of the MeV-DMV-F fusion protein (i.e., F2, SS, Intermediate (F Int), H interacting/binding domain (HBD), or stalk) corresponding to the right two plots of each set of flow cytometry scatter plots (see also FIGS. 12A-12C). In FIGS. 14A-14F, the y-axes represent GFP expression and the x-axes represent SSC (side scatter). In FIGS. 14A-14F high GFP expression (boxed regions in the plots) indicates effective transduction of a cell and the numbers within the plots indicate the percent of total counted cells that were effectively transduced using viral particles pseudotyped with the indicated envelope glycoprotein H and envelope glycoprotein F. The fusion proteins MeV-DMV-F Int showed high efficacy in vitro. The combination of the fusion proteins MeV-DMV-H and MeV-DMV-F Stalk avoids neutralization by anti-MeV antibodies (e.g., antibodies produced by a subject administered a measles virus vaccine or having recovered from a measles virus infection).

FIGS. 15A and 15B provide plots showing results from serum neutralization assays to evaluate the impact of serum neutralization on the activity of lentiviral particles pseudotyped with the indicated envelope glycoprotein fusion polypeptides provided herein (e.g., MeV-DMV-H N11, MeV-DMV-F Int, and MeV-DMV-F Stalk). In FIGS. 15A and 15B lentiviral particles pseudotyed with MeV-H N11 and MeV-F were used as a control. In FIG. 15A, “Donor F62” was a 62-year-old female immunized against the measles virus. In FIG. 15B, “Donor M12” was a 12-year-old male immunized against the measles virus. The fusion proteins showed improved resistance to neutralization by the human serum relative to the MeV-H N11 and MeV-F envelope glycoproteins, which did not contain amino acid sequences derived from the dolphin morbillivirus (DMV).

FIG. 16 provides a schematic diagram showing a pseudotyped lentiviral particle capable of activating a T cell. The lipid envelope of the lentiviral particle contains a CD80 polypeptide, a membrane-tethered anti-CD3 scFv polypeptide, and a virus envelope protein (e.g., an envelope glycoprotein) fused to a VHH domain.

FIG. 17 provides a flow cytometry scatter plot demonstrating the surface-expression of a human cluster of differentiation 80 (hCD80) polypeptide on the surface of producer HEK293 T cells transduced with polynucleotides encoding the CD80 polypeptide and an anti-CD3 scFv polypeptide. In FIG. 17, the numbers “0.080” and “99.3” represent the number of total counted cells that surface-expressed hCD80.

FIG. 18 provides a bar graph confirming that producer HEK293 T cells surface-expressing a human cluster of differentiation 80 (hCD80) polypeptide and a membrane-tethered anti-CD3 scFv polypeptide activated T cells in co-culture, as measured by increased expression of CD25 and CD69 in the activated T cells. 100k 293T cells were co-cultured with scaled T-cells. The cells were co-cultured at different effector-to-target cell ratios (E:T), as indicated on the x-axis (i.e., 4:1, 1:1, or 1:4), where effector cells were the T cells and the target cells were the producer HEK293T cells. As a positive control, the T cells were activated using beads, which contained an anti-CD3 antigen-binding polypeptide and an anti-CD28 antiben-binding polypeptide.

FIG. 19 provides a series of plots demonstrating that VSVg-pseudotyped viruses containing a human cluster of differentiation 80 (hCD80) polypeptide and an anti-CD3 scFv polypeptide in their envelopes improved infection of unstimulated T cells. T cell activation was quantified through measuring expression of cluster of differentiation 25 (CD25) in the activated cells, and levels of infection were quantified by measuring eGFP expression in the cells. As a control, cells were activated with the transduction enhancer, LentiBOOST™. 10× concentrated VSVg particles containing a polynucleotide encoding enhanced green fluorescent protein (eGFP) were used to infect 100k T-cells with or without LentiBOOST™. The plots on the left in FIG. 19 correspond to T cells that were not activated prior to administration of the virus particles, and the plots on the right correspond to T cells that were activated prior to administration of the virus particles. T cells were activated using beads, which contained an anti-CD3 antigen-binding polypeptide and an anti-CD28 antigen-binding polypeptide. In FIG. 19, HEK293 aCD3/hCD80 indicates infection with VSVg particles that contained the hCD80 polypeptide and the membrane-tehtered anti-CD3 scFv polypeptide. The x-axis of FIG. 19 represents increasing doses of virus used to infect the T cells.

FIGS. 20A-20D provide bar graphs and schematic diagrams showing levels of infection of cells using viral particles pseudotyped using the indicated chimeric polypeptides of the disclosure. The viral particles contained a polynucleotide encoding enhanced green fluorescent protein (eGFP) and infection levels were quantified by measuring eGFP levels in cells contacted with the viral particles. Each viral envelope protein H polypeptide was fused to an anti-MHCII VHH domain. A20 cells were transduced with 10 μL of a 100× polyethylene glycol (PEG) concentrated virus and expression of eGFP was measured four (4) days post-infection. FIG. 20A provides a schematic diagram showing how the indicated chimeric F proteins (F2, SS, Int, h-dom (HBD), and Stalk) contained progressively higher percentages of amino acids derived from the dolphin morbillivirus and correspondingly smaller percentages derived from the measles virus (MeV). FIG. 20B provides a bar graph and a schematic diagram showing levels of infection of A20 cells using lentiviral particles pseudotyped using an MeV-DMV-H polypeptide fused to an anti-MHCII VHH domain (i.e., MeV-DMV-H N11) and one of the viral envelope F proteins indicated along the X-axis, namely MeV-Fc30, MeV-DMV-F2, MeV-DMV-SS, MeV-DMV-F Int, MeV-DMV-F HBD (h-dom), and MeV-DMV-F Stalk. The schematic diagram of FIG. 20B indicates that both the H protein and the F protein were chimeric. FIG. 20C provides a bar graph and a schematic diagram showing levels of infection of A20 cells using lentiviral particles pseudotyped using an MeV-H polypeptide fused to an anti-MHCII VHH domain (i.e., MeV-H N11) and one of the viral envelope F proteins indicated along the X-axis, namely MeV-Fc30, MeV-DMV-F2, MeV-DMV-SS, MeV-DMV-F Int, MeV-DMV-F HBD (h-dom), and MeV-DMV-F Stalk. The schematic diagram of FIG. 20C indicates the H protein was not chimeric and the F protein was. FIG. 20D provides a bar graph and a schematic diagram showing levels of infection of A20 cells using lentiviral particles pseudotyped using an DMV-H polypeptide fused to an anti-MHCII VHH domain (i.e., DMV-H N11) and one of the viral envelope F proteins indicated along the X-axis, namely MeV-Fc30, MeV-DMV-F2, MeV-DMV-SS, MeV-DMV-F Int, MeV-DMV-F HBD (h-dom), and MeV-DMV-F Stalk. In FIGS. 20B-20D, lentiviral particles pseudotyped using MeV-H N11 and MeV-F (i.e., “Full MeV WT Cntrl”) were used as a positive control.

FIG. 21 provides a plot showing that producer HEK293T cells surface-expressing MeV H or MeV F were bound by antibodies in blood serum from a human expressing anti-measles virus antibodies. HEK293T cells were transfected with polynucleotides encoding MeV H, MeV F, or VSVg HA (i.e., VSVg with an HA tag). Each of MeV H, MeV F, and VSVg contained an HA tag. The next day, the cells were contacted donor samples containing high or low anti-MeV IgG levels. The serum samples were heat neutralized at 56° C. for 20 minutes prior to the contacting. The transfected cells were contacted with the heat-neutralized serum on ice for 1 hour. Then, the cells were stained using anti-Human IgG antibody and an anti-HA antibody, and binding was measured using flow cytometry. The y-axis of FIG. 21 represents the number of cells bound by IgG that surface-expressed MeV H or MeV F. In FIG. 21 “Marker+” indicates cells surface-expressing the HA tag.

FIGS. 22A and 22B provide a flow cytometry histogram and a plot showing that VSVg HA (i.e., VSVg containing an HA tag) had poor surface expression in HEK293T cells. FIG. 22A provides a flow cytometry histogram showing HEK293T cells surface expressed higher levels of MeV H and MeV F than VSVg HA. FIG. 22B provides a plot showing levels of immunoglobulin binding to producer HEK293T cells surface expressing VSVg HA when the cells were contacted with human serum containing high (i.e., “High IgG Sera”) or low (i.e., “Low IgG Sera”) levels of anti-VSVg HA antibodies. Comparing binding levels seen in FIG. 22B to those shown in FIG. 21 indicates that VSVg hemagglutinin (HA) had poor surface expression in HEK293T cells relative to levels observed for MeV H and MeV F. Cells were stained as described for FIG. 21. The y-axis of FIG. 22B represents the number of cells bound by IgG that surface-expressed VSVg. In FIG. 22B, “Marker+” indicates cells surface-expressing the HA tag.

FIGS. 23A and 23B provide plots showing levels of antibody binding to HEK293 T cells surface expressing MeV H or MeV F when the cells were contacted with human serum containing high (“Donor 1”) or low (“Donor 2”) levels of anti-morbillivirus antibodies. Each of MeV H and MeV F contained an HA tag. As a negative control, donor serum was contacted with HEK293 T cells that did not express MeV H or MeV F (i.e., “Untransfected” cells). FIG. 23A provides a plot showing antibody binding from serum from Donor 1 or Donor 2 at different levels of dilution. FIG. 23B provides a plot showing antibody binding from serum from Donor 1 at different levels of dilution. Cells were stained as described for FIG. 21. The y-axis of FIGS. 23A and 23B represents the number of cells counted using flow cytometry that were bound by human IgG.

FIG. 24 provides a plot showing that anti-measles antibodies in human serum bound with a higher affinity to MeV H than to DMV H, CDV H, or to chimeras of MeV H and DMV H (MeV/DMV H; MeV-DMV H) or CDV H (MeV/CDV H; MeV-CDV H). Each polypeptide contained an HA tag. HEK293T cells were transfected with polynucleotides encoding Mev H, DMV H, CDV H, MeV-DMV H, or MeV-CDV H. Transfected cells were then contacted with human serum containing high tigers of anti-MeV IgG antibodies after neutralizing the serum at 56° C. for 20 minutes. The cells were incubated on ice in the presence of the serum for one hour and subsequently stained using an anti-Human IgG antibody and an anti-HA tag antibody. Staining was measured using flow cytometry. The y-axis of FIG. 24 represents the percent of all cells surface expressing MeV H, DMV H, CDV H, MeV-DMV H, or MeV-CDV H that were bound by human IgG.

FIG. 25 provides flow cytometry histograms showing that anti-measles antibodies in human serum bound with a higher affinity to MeV H than to CDV H or MeV-CDV H. Each of MeV H, CDV H, and MeV-CDV H contained an HA tag. Cells were prepared and stained as described for FIG. 24. In FIG. 25, the numbers in each quadrant indicate the total percent of cells counted that fell within the indicated quadrant. Cells falling within Quadrant 2 (Q2) represented cells bound by human anti-measles IgG and surface-expressing MeV H, CDV H, or MeV-CDV H.

FIG. 26 provides a plot showing binding of anti-measles antibodies in human serum to HEK293T cells surface expressing MeV H, CDV H, MeV-DMV H, MeV-CDV H, Rinderpest virus H protein (RPV), small ruminant virus H protein (RMV), and Peste de pestis ruminant virus H protein (PPRV). HEK293T cells were transfected with polynucleotides encoding MeV H, CDV H, MeV-DMV H, MeV-CDV H, RPV, RMV, or PPRV, each containing an HA tag. Cells were stained as described for FIG. 24. The y-axis of FIG. 26 represents the percent of total cells expressing the MeV H, CDV H, MeV-DMV H, MeV-CDV H, RPV, RMV, or PPRV polypeptide that were bound by anti-measles antibodies in the human serum.

FIGS. 27A and 27B provide a schematic diagram and flow cytometry scatter plots showing that infection of unstimulated human pan T cells using lentivirus particles pseudotyped using MeV-DMV-H fused to an anti-CD7 VHH domain and MeV-DMV-F Int and containing a polynucleotide encoding an anti-CD19 chimeric antigen receptor (CAR) led to generation of CAR-expressing T cells and elimination of CD19⁺ human leukemia cells (NALM6) in co-culture. The lentivirus particles contained a polynucleotide encoding enhanced green fluorescent protein (eGFP). As shown in the schematic diagram of FIG. 27A, prior to infection with the lentivirus particles, 5000 NALM6 cells were co-cultured with 5000 unstimulated T cells at day zero (0). While in co-culture, the unstimulated T cells were infected using 10 μL of 100×ultraconcentrated virus. No LentiBOOST™ was used to transduce the cells. The lentivirus particles each contained a human cluster of differentiation 80 (hCD80) polypeptide and a membrane-tethered anti-CD3 scFv polypeptide to activate the T cells. FIG. 27B provides flow cytometry plots showing that at each effector to target cell ratio (E:T) evaluated (i.e., 15:1, 10:1, and 5:1) the infected cells were able to kill all or nearly all of the NALM6 cells in the co-cultures within 6 days of infection. In FIG. 27B, the numbers in each quadrant indicate the total percent of cells counted that fell within the indicated quadrant. As a negative control, uninfected cells were co-cultured with NALM6 cells. The NALM6 cells surface-expressed human cluster of differentiation 19 (hCD19) polypeptides. In the flow cytometry scatter plots of FIG. 27B, NALM6 cells fell within the lower-right quadrant, and uninfected human pan T cells fell within the lower-left quadrant.

FIG. 28 provides a bar graph showing the number of CD19⁺ cells (i.e., NALM6 cells) in the co-cultures of FIGS. 27A and 27B over time. In FIG. 28, “d0” means “day zero,” “d3” means “day 3,” “d6” means “day 6.”

FIGS. 29A and 29B provide schematic diagrams showing an experimental design for evaluating in vivo generation of CAR T cells and clearance of tumors in NSG mice. FIG. 29A provides a schematic diagram showing how mice were treated and how samples were taken and evaluated from the mice. The experimental groups (n=3) were NALM6 cells only, PAN T cells and NALM6 cells, PAN T cells+virus+NALM6 cells, and ex vivo generated CAR T cells and NALM6 cells. The lentivirus used to generate CAR T cells in vivo and ex vivo was pseudotyped using a MeV-DMV-H polypeptide fused to an anti-CD7 VHH antigen binding domain (i.e., MeV-DMV-H-aCD7) and a MeV-DMV-F Int polypeptide. FIG. 29B provides a schematic diagram showing a timeline for the experiment. In FIG. 29B, “D6, 9, 13, 16” indicates “days 6, 9, 13, and 16,” and “D40” indicates “day 40.” In FIG. 29B, “BCS” indicates “body condition score,” where lower BCS indicates emaciation and the highest BCS scores (e.g., higher than 5) indicate obesity, and “TD” indicates “take-down” or “euthanization.”

FIGS. 30A and 30B provide flow cytometry scatter plots and flow cytometry contour plots showing that in vivo generated CAR T cells prepared as described for FIGS. 29A and 29B were detected in mice at day 6 post-infection (FIG. 30A) and persisted through day 13 (FIG. 30B). Blood was collected using submandibular bleeds. Viable cells surface-expressing human cluster of differentiation 45 (hCD45) (i.e., human T cells) were counted to prepare the flow cytometry scatter plots. In FIGS. 30A and 30B, the numbers in the square boxes represent the frequency of total T cells counted that surface expressed the chimeric antigen receptor (CAR).

FIGS. 31A-31C provides a survival curve, a growth curve, and images showing that in vivo generated CAR T cells generated as described for FIGS. 29A and 29B showed improved therapeutic effect over ex vivo generated CAR T cells. FIG. 31A provides a survival curve showing that the in vivo generated CAR T cells were associated with increased survival times.

FIG. 31B provides a growth curve showing that NALM6 cell proliferation was lowest in mice containing in vivo generated CAR T cells. FIG. 31C provides images showing that mice containing in vivo generated CAR T cells survived longer than mice administered ex vivo generated T cells and showed improved reduction in tumor size.

FIG. 32 provides flow cytometry scatter plots showing that lentiviral particles pseudotyped using a MeV-DMV-H polypeptide fused to an anti-CD7 VHH antigen binding domain (i.e., MeV-DMV-H-aCD7) and a DMV-F Int polypeptide, and also containing a human cluster of differentiation 80 (hCD80) polypeptide and an anti-CD3 scFv polypeptide in their lipid envelope, successfully activated pan T cells in vitro. The viral particles contained a polypeptide encoding enhanced green fluorescent protein (eGFP). T cell activation was quantified by measuring expression of human cluster of differentiation 69 (hCD69) in the activated cells, and levels of infection were quantified by measuring eGFP expression in the cells. As a control, VSVg particles containing the hCD80 polypeptide and the anti-CD3 scFv polypeptide, and VSVg particles not expressing hCD80 polypeptide or the anti-CD3 scFv polypeptide, were used to infect the cells. In FIG. 32 “unmod” indicates virus particles that did not contain the hCD80 polypeptide or the anti-CD3 scFv polypeptide, aCD3/hCD80 indicates virus particles that did, and “293s” indicates that pah T cells were contacted with the virus particles. In FIG. 32, the numbers in each quadrant indicate the total percent of cells counted that fell within the indicated quadrant. In FIG. 32, “stimulated” indicates that T cells were activated using beads containing an anti-CD3 antigen-binding polypeptide and an anti-CD28 antigen-binding polypeptide prior to being contacted with the virus particles.

### DETAILED DESCRIPTION OF THE INVENTION

The invention features pseudotyped viral particles (e.g., lentiviral or gammaretroviral particles) and compositions and methods of use thereof, where the viral particles comprise a VHH domain. The pseudotyped viral particles are useful for, among other things, the in vivo delivery of a polynucleotide and/or polypeptide to a cell to treat a disease or condition (e.g., cancer) in a subject (e.g., a measles-immune subject).

The invention is based, at least in part, upon the discovery that viral fusion proteins containing a VHH domain and a non-measles virus Morbillivirus hemagglutinin domain (VHH-MV-HA fusions) showed high levels of surface expression in producer cells. Further, Lentiviral particles pseudotyped with the VHH-MV-HA fusions effectively targeted and transfected cells displaying the VHH antigen. The viral fusion proteins contain amino acid alterations associated with reduced neutralization by measles-virus neutralizing antibodies relative to viral fusion proteins comprising an extracellular domain (e.g., globular head or extravirion domain) from a measles virus envelope glycoprotein (e.g., envelope glycoprotein H or F).

In embodiments, pseudotyped viral particles of the invention can be used in methods for in vivo cellular reprogramming of target cells, optionally where the cells are in a measles-immune subject. In various embodiments, such methods allow for a dramatic reduction ion manufacturing costs and time required for cell therapy and an increase in the number of patients that can benefit from cell therapy. The methods can have the advantage of allowing for in vivo editing of cells that are difficult to expand ex vivo, such as macrophage and NK cells. The lentiviral particles of the present invention have the advantage of having a large packaging unit and, thus, enable delivery of larger payloads than possible using adeno-associated virus (AAV) vectors or some nanoparticle approaches.

### Pseudotyped Viral Particles

The present invention features pseudotyped viral particles. In embodiments, the viral particle is a retroviral particle (e.g., a lentiviral particle or a gammaretroviral particle). In embodiments, the retroviral particle comprises a viral glycoprotein (e.g., a Morbillivirus H or F protein) or fragment thereof fused to a VHH domain or fragment thereof. Retroviral particles comprise an lipid envelope surrounding a viral capsid, where the viral capsid encapsidates (i.e., surrounds) a polynucleotide (e.g., single or double-stranded RNA). A retrovirus is a type of virus that inserts a copy of its genome (i.e., the encapsidated polynucleotide) into the genome of a host cell that it invades/infects. Once inside the host cell's cytoplasm, a retrovirus uses its own reverse transcriptase enzyme to produce DNA from the virus' own RNA genome. The DNA produced by the reverse transcriptase is then incorporated into the host cell genome by an integrase enzyme. Such incorporation results in stable expression of a gene(s) encoded by the polynucleotide in the infected cell and its progeny. There are three basic groups of retroviruses: oncoretroviruses, lentiviruses, and spumaviruses. Human retroviruses include HIV-1, HIV-2, and the human T-lymphotrophic virus. Mouse retroviruses include the murine leukemia virus.

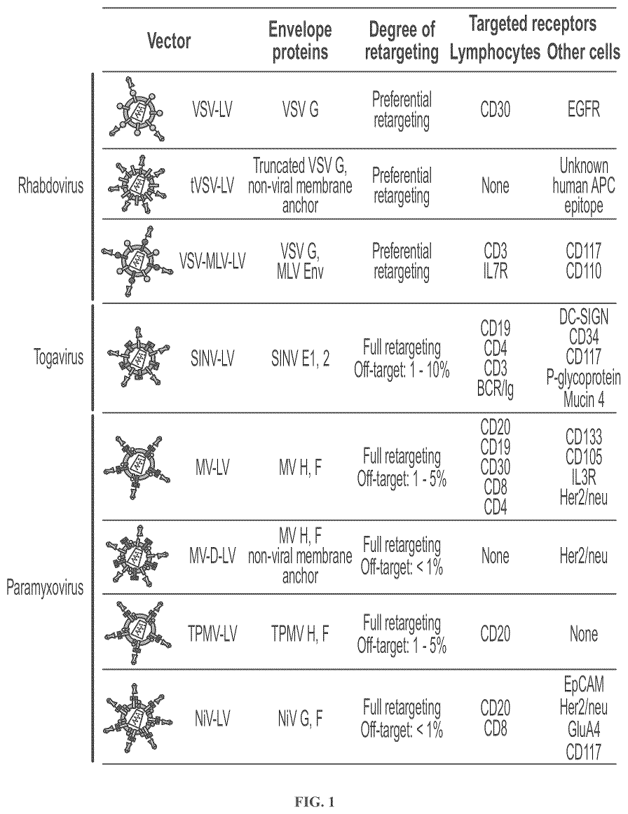

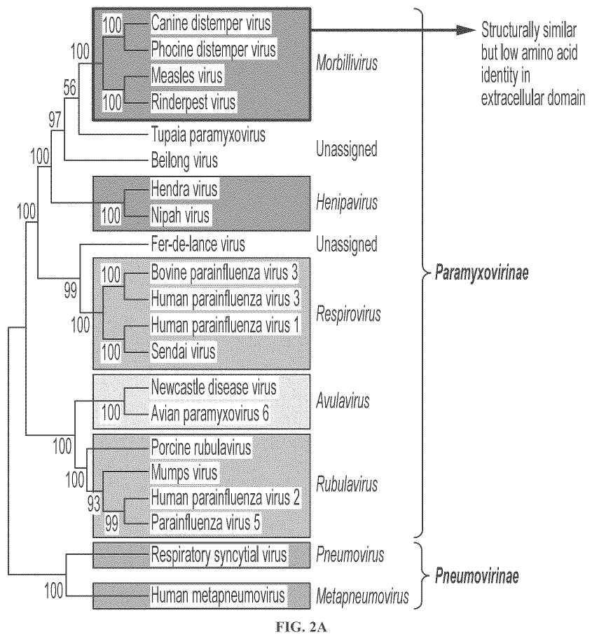

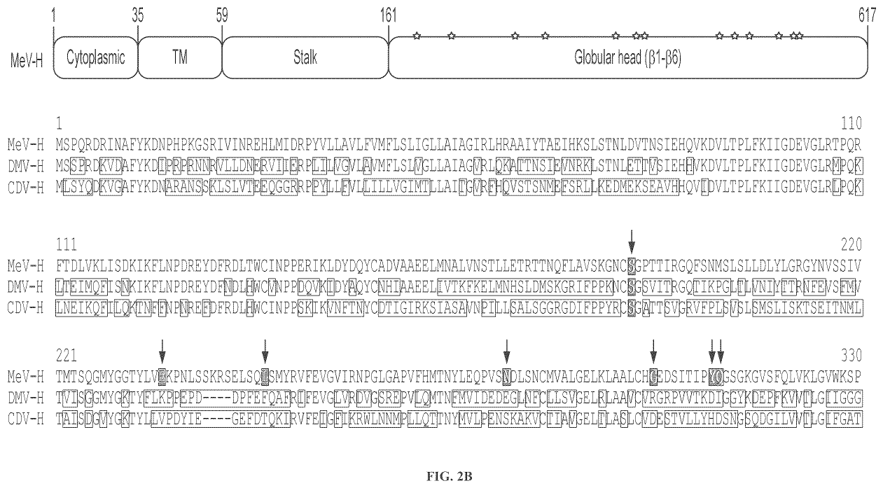

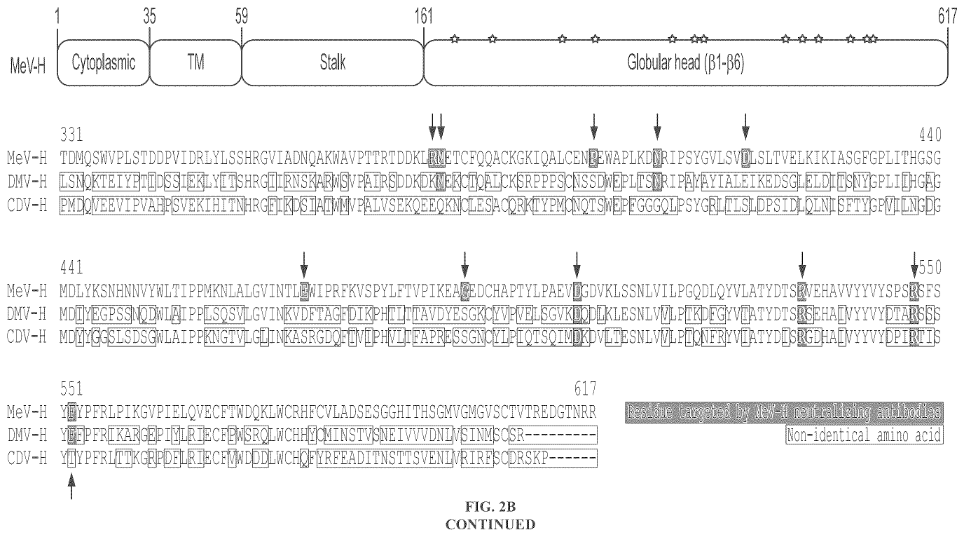

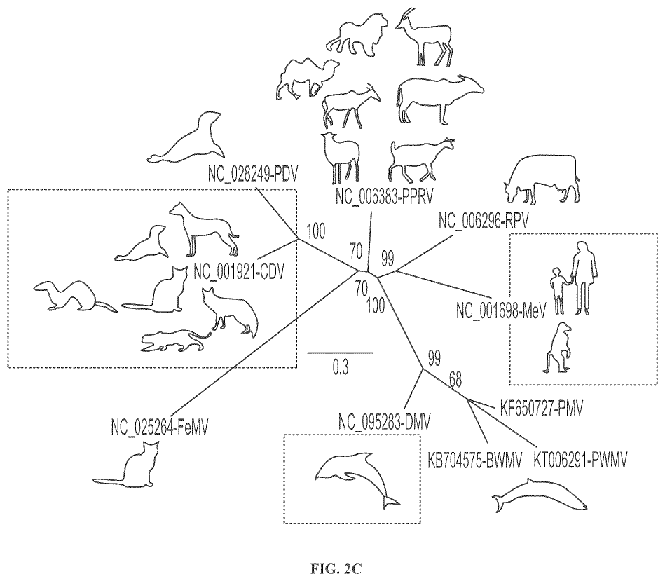

Retrovirus particles comprise a lipid envelope and are about 75-125 nm in diameter. The outer lipid envelope contains glycoprotein. Examples of glycoproteins contained in the lipid envelope of different retroviral particles are provided in FIGS. 1, 2A, and 2C. Further non-limiting examples of glycoproteins contained in the lipid envelope of retroviral particles include MeV-Hwtc18, CDV-F, CDF-Fc30, DMV-F, DMV-Fc30, DMV-H, DMV-Hc18 (MeV-Hc18-DMV), CDV-H, CDV-Hc18 (MeV-Hc18-CDV), FMV-H, PPRV-H, RPV-H, RMV-H, DMV1-123-MeV122-529 (F2), DMV1-123-MeV122-529 (S-S), DMV1-311-MeV309-529 (Intermediate), DMV1-407-MeV405-529 (H interacting domain), and DMV1-465-MeV463-529 (stalk). A retroviral particle can be pseudotyped by replacing the retroviral particle's endemic envelope proteins (e.g., a glycoprotein) with a heterologous envelope protein(s) (e.g., MeV-Hwtc18, CDV-F, CDF-Fc30, DMV-F, DMV-Fc30, DMV-H, DMV-Hc18 (MeV-Hc18-DMV), CDV-H, CDV-Hc18 (MeV-Hc18-CDV), FMV-H, PPRV-H, RPV-H, RMV-H, DMV1-123-MeV122-529 (F2), DMV1-123-MeV122-529 (S-S), DMV1-311-MeV309-529 (Intermediate), DMV1-407-MeV405-529 (H interacting domain), DMV1-465-MeV463-529 (stalk) or those listed in FIGS. 1, 2A, 2B, 2C, 6B, 11A-11C, or 12A-12D). In embodiments, the retroviral particle is pseudotyped with a glycoprotein (e.g., envelope protein H or F) from a Paramyxovirinae virus. In embodiments, the Paramyxovirinae virus is a Morbilllivirus. In embodiments, the Morbillivirus is canine distemper virus, dolphin Morbillivirus, feline Morbillivirus, measles virus, Peste des petits ruminant virus, phocine Morbillivirus, Rinderpest virus, or small ruminant virus. Glycoproteins facilitate targeting of the viral particle to a target cell. In embodiments, the glycoprotein (e.g., envelope protein H) of the invention is fused to a VHH domain. In some instances, the glycoprotein of the invention is fused to the VHH domain by a linker (e.g., a (G3S)₂ linker or a (G4S)₃ linker). In embodiments, the glycoprotein or fragment thereof is mutated so as to no longer target a surface protein of a cell. Retroviruses typically have a genome comprising two single-stranded RNA molecules 7-10 kb in length. The two molecules can exist as a dimer formed through complementary base-pairing. In embodiments, a retrovirus genome encodes group-specific antigen (gag) proteins, protease (pro) proteins, polymerase (pol) proteins, and envelope (env) proteins. Gag proteins in embodiments are a major component of the viral capsid, and a viral capsid can comprise from about 2000 to about 4000 gag proteins. Gag proteins contain nucleic acid binding domains, including matrix (MA) and nucleocapsid (NC), that assist in packaging the polynucleotide into the capsid. Gag proteins are important for many aspects of virion assembly. Protease assists in virion maturation by, for example, assisting in proper gag protein and pol protein processing. Pol proteins are responsible for synthesis of viral DNA and integration into host DNA following infection. Env proteins (e.g., a glycoprotein) facilitate cell targeting and entry of the encapsidated polynucleotide into the target cell.

In some embodiments the cytoplasmic domain of the envelope protein (e.g., MeV-Hwtc18, CDV-F, CDF-Fc30, DMV-F, DMV-Fc30, DMV-H, DMV-Hcl8 (MeV-Hcl8-DMV), CDV-H, CDV-Hcl8 (MeV-Hcl8-CDV), FMV-H, PPRV-H, RPV-H, RMV-H, DMV1-123-MeV122-529 (F2), DMV1-123-MeV122-529 (S-S), DMV1-311-MeV309-529 (Intermediate), DMV1-407-MeV405-529 (H interacting domain), and DMV1-465-MeV463-529 (stalk)) is truncated. For example, in some instances the cytoplasmic domain of the envelope protein is truncated by about or at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 60, 70, or 80 amino acids. In some embodiments, the cytoplasmic domain comprises less than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 amino acids. In some instances the glycoprotein and/or glycoprotein fused to the VHH domain is resistant to neutralization by measles virus neutralizing antibodies relative to measles virus glycoproteins or fusions thereof. In some cases the glycoprotein contains alterations at about or at least about 1, 2, 3, 4, 5, 6, 7, 8, 9, 10, 11, 12, 13, 14, 15, 20, 30, 40, or 50 amino acid positions corresponding to amino acids of a morbillivirus glycoprotein that are targeted by morbillivirus neutralizing antibodies. In some instances, lentivirus particles pesudotyped with the glycoprotein or glycoprotein fusion are associated with higher in vivo transduction rates of target cells than a glycoprotein or glycoprotein fusion comprising an extravirion domain (e.g., a globular head domain) derived from a measles virus envelope protein (e.g., measles virus envelope protein H). In embodiments, lentivirus particles pseudotyped with glycoproteins or glycoprotein fusions of the present disclosure are more effective at transducing a cell in a measles-immune subject than lentivirus particles pseudotyped with a polypeptide comprising an extravirion domain derived from a measles virus envelope protein (e.g., measles virus envelope protein H and/or F).

In embodiments, an envelope glycoprotein F fusion polypeptide or chimeric polypeptide contains an extravirion domain containing an extravirion domain fragment derived from a dolphin morbillivirus envelope glycoprotein F (DMV-F) or an alternative envelope glycoprotein F extravirion domain and an extravirion domain fragment derived from an envelope glycoprotein F (MeV-F). In embodiments, the extravirion domain is about or at least about 25, 50, 75, 100, 125, 150, 175, 200, 225, 250, 275, 300, 225, 350, 375, 400, 425, 450, 475, 500, 525, 550, 575, or 600 amino acids in length. In embodiments, the extravirion domain is no more than about 25, 50, 75, 100, 125, 150, 175, 200, 225, 250, 275, 300, 225, 350, 375, 400, 425, 450, 475, 500, 525, 550, 575, or 600 amino acids in length. In some instances, the extravirion domain is derived from an MeV-F extravirion domain where a C-terminal and/or N-Terminal portion thereof has been replaced by a corresponding portion from a DMV-F extravirion domain or the extravirion domain of an alternative envelope glycoprotein F extravirion domain. In embodiments, In embodiments, the extravirion domain contains a stretch of about or at least about 10, 20, 30, 40, 50, 60, 70, 80, 90, 100, 110, 120, 130, 140, 150, 160, 170, 180, 190, 200, 210, 220, 230, 240, 250, 260, 270 280, 290, 300, 310, 320, 330, 340, 350, 360, 370, 380, 390, 400, 410, 420, 430, 440, 450, 460, 470, 480, 490, 500, or 510 contiguous amino acids derived from a first envelope glycoprotein F domain (e.g., DMV-F) and a stretch of about or at least about 10, 20, 30, 40, 50, 60, 70, 80, 90, 100, 110, 120, 130, 140, 150, 160, 170, 180, 190, 200, 210, 220, 230, 240, 250, 260, 270 280, 290, 300, 310, 320, 330, 340, 350, 360, 370, 380, 390, 400, 410, 420, 430, 440, 450, 460, 470, 480, 490, 500, or 510 contiguous amino acids derived from a second envelope glycoprotein F domain (e.g., MeV-F). In some instances, the extravirion domain comprises a C-terminal stretch of contiguous amino acids derived from the first envelope glycoprotein F domain and an N-terminal stretch of contiguous amino acids derived from the second envelope glycoprotein F domain, where in some embodiments the two stretches of contiguous amino acids make up a full extravirion domain (e.g., an extravirion domain corresponding to that of DMV-F or MeV-F).

In embodiments, an envelope glycoprotein H fusion polypeptide or chimeric polypeptide contains an extravirion domain containing an extravirion domain fragment derived from a dolphin morbillivirus envelope glycoprotein H (DMV-H) or an alternative envelope glycoprotein H extravirion domain and an extravirion domain fragment derived from an envelope glycoprotein H (MeV-H). In embodiments, the extravirion domain is about or at least about 25, 50, 75, 100, 125, 150, 175, 200, 225, 250, 275, 300, 225, 350, 375, 400, 425, 450, 475, 500, 525, 550, 575, or 600 amino acids in length. In embodiments, the extravirion domain is no more than about 25, 50, 75, 100, 125, 150, 175, 200, 225, 250, 275, 300, 225, 350, 375, 400, 425, 450, 475, 500, 525, 550, 575, or 600 amino acids in length. In some instances, the extravirion domain is derived from an MeV-H extravirion domain where a C-terminal and/or N-Terminal portion thereof has been replaced by a corresponding portion from a DMV-H extravirion domain or the extravirion domain of an alternative envelope glycoprotein H extravirion domain. In embodiments, In embodiments, the extravirion domain contains a stretch of about or at least about 10, 20, 30, 40, 50, 60, 70, 80, 90, 100, 110, 120, 130, 140, 150, 160, 170, 180, 190, 200, 210, 220, 230, 240, 250, 260, 270 280, 290, 300, 310, 320, 330, 340, 350, 360, 370, 380, 390, 400, 410, 420, 430, 440, 450, 460, 470, 480, 490, 500, or 510 contiguous amino acids derived from a first envelope glycoprotein H domain (e.g., DMV-H) and a stretch of about or at least about 10, 20, 30, 40, 50, 60, 70, 80, 90, 100, 110, 120, 130, 140, 150, 160, 170, 180, 190, 200, 210, 220, 230, 240, 250, 260, 270 280, 290, 300, 310, 320, 330, 340, 350, 360, 370, 380, 390, 400, 410, 420, 430, 440, 450, 460, 470, 480, 490, 500, or 510 contiguous amino acids derived from a second envelope glycoprotein H domain (e.g., MeV-H). In some instances, the extravirion domain comprises a C-terminal stretch of contiguous amino acids derived from the first envelope glycoprotein H domain and an N-terminal stretch of contiguous amino acids derived from the second envelope glycoprotein H domain, where in some embodiments the two stretches of contiguous amino acids make up a full extravirion domain (e.g., an extravirion domain corresponding to that of DMV-H or MeV-H).

Lentiviruses and gammaretroviruses are genuses of retroviruses. In embodiments, the pseudotyped viral particles of the invention are pseudotyped lentiviral or gammaretroviral particles.

Retroviral particles have the advantage of being comparatively large (e.g., in comparison to adeno-associated virus (AAV) particles) and, therefore, capable of delivering larger polynucleotide sequences and/or a larger number of polypeptide sequences to a target cell than would be possible using alternative viral particles. Retroviral particles have the further advantage of possessing a viral envelope within which may be displayed a variety of polypeptides for delivery to a target cell. Delivering polypeptides to a target cell, as opposed to a polynucleotide, can have the advantage of facilitating the temporal introduction of an activity (e.g., an enzymatic or stimulatory activity) to a cell rather than constitutive activity (e.g., through integration of a polynucleotide sequence encoding a heterologous polypeptide into the genome of the target cell). A further advantage of retroviral particles is that, by virtue of containing a viral envelope, the surface of the viral particles (i.e., the envelope) may be altered to alter targeting of the retroviral particle or to alter interactions between the retroviral particle and the target cell.

The pseudotyped viral particles of the invention contain a polynucleotide. In embodiments, the polynucleotide encodes a heterologous gene. In embodiments, the heterologous gene is a chimeric antigen receptor, or a component thereof.

In embodiments, the viral envelope displays a polypeptide facilitating evasion of a subject's immune system by the viral particle. In embodiments, the viral envelope contains a polypeptide that inhibits phagocytosis. In embodiments, the viral envelope comprises a CD47 polypeptide. In embodiments, the viral envelope contains a complement regulatory polypeptide. Non-limiting examples of complement regulatory polypeptides include CD46, CD55, and CD59.

In embodiments, the viral particle contains (e.g., as displayed on the viral envelope) polypeptides that activate a physiological response (e.g., proliferation, T cell activation, survival, intracellular signaling, changes in gene expression, apoptosis, or differentiation) in the target cell (e.g., through introduction of a cytokine or a chemokine to the target cell). Non-limiting examples of cytokines or chemokines that can be included in the viral envelope include of aCD3, Ccl14, CD28, CD40L, Cxcl10, IL-2, IL-7, IL-12, IL-15, IL-18, and IL-21. In some cases, the target cell is a T cell and the physiological response is T cell activation, which can be measured as an increase in surface expression of CD25 and/or CD69 in the target cell. It can be advantageous for a viral particle to be capable of activating a T cell because T cell activation can increase infection efficiencies of viral particles. In some instances, a viral particle contains a membrane-tethered anti-cluster of differentiation 3 (CD3) polypeptide and a cluster of differentiation 80 (CD80) polypeptide and is capable of activating a T cell with which the viral particle is contacted (see, e.g., Dobson, C. S., et al. Nat Methods 19, 449-460 (2022), the disclosure of which is incorporated herein in its entirety for all purposes).

Methods for displaying polypeptides in a viral envelope are known and are suitable for use in embodiments of the invention. See, for example, Taube, et al., “Lentivirus Display: Stable Expression of Human Antibodies on the Surface of Human Cells and Virus Particles”, PLoS ONE, 3: e3181 (2008).

In embodiments, the viral particle is not capable of self-replication. In embodiments, the viral particle is capable of self-replication.

### VHH Domains

In embodiments, pseudotyped viral particles of the invention comprise VHH domains. In embodiments, the VHH domain binds an antigen selected from, as non-limiting examples, BCR/Ig, CD3, CD4, CD7, CD8, CD11, CD19, CD20, CD30, CD34, CD38, CD45, CD133, CD103, CD105, CD110, CD117, CTLA-4, CXCR4, DC-SIGN, EGFR, Emrl, EpCAM, GluA4, Her2/neu, IL3R, IL7R, Mac, MHCII, Mucin 4, NK1.1, P-glycoprotein, TIM3, Thy1, and Thy1.2. In embodiments, the VHH binds an antigen associated with a target cell. In embodiments, the target cell is an immune cell. As non-liming examples, the target cell can be a B cell, a dendritic cell, an eosinophil, a granulocyte, an iNKT cell, a macrophage, a monocyte, a natural killer cell, a neutrophil, a lymphoma cell, a regulatory T cell, or a T cell. In embodiments, the immune cell is CD4⁺ and/or CD8⁺.

VHH domains are derived from nanobodies. Nanobodies are antibody-derived therapeutic proteins that contain the unique structural and functional properties of naturally-occurring heavy-chain antibodies. These heavy-chain antibodies contain a single variable domain (VHH) and two constant domains (CH2 and CH3). Importantly, the cloned and isolated VHH domain is a stable polypeptide harboring the full antigen-binding capacity of the original heavy-chain antibody. Nanobodies have a high homology with the VH domains of human antibodies and can be further humanized without any loss of activity. Importantly, Nanobodies have a low immunogenic potential, which has been confirmed in primate studies with Nanobody lead compounds.

Nanobodies combine the advantages of conventional antibodies with important features of small molecule drugs. Like conventional antibodies, Nanobodies show high target specificity, high affinity for their target and low inherent toxicity. However, like small molecule drugs they can inhibit enzymes and readily access receptor clefts. Furthermore, Nanobodies are stable, can be administered by means other than injection (see, e.g., WO2004041867A2, which is herein incorporated by reference in its entirety) and are easy to manufacture. Other advantages of Nanobodies include recognizing uncommon or hidden epitopes as a result of their small size, binding into cavities or active sites of protein targets with high affinity and selectivity due to their unique 3-dimensional, drug format flexibility, tailoring of half-life and ease and speed of drug discovery.

Nanobodies are encoded by single genes and are efficiently produced in almost all prokaryotic and eukaryotic hosts, e.g., E. coli (see, e.g., U.S. Pat. No. 6,765,087, which is herein incorporated by reference in its entirety), molds (for example Aspergillus or Trichoderma) and yeast (for example Saccharomyces, Kluyveromyces, Hansenula, or Pichia) (see, e.g., U.S. Pat. No. 6,838,254, which is herein incorporated by reference in its entirety).

Methods known in the art may be used to generate nanobodies. These nanobodies may then serve as the basis for the generation of a library which may be produced and selected from according using methods such as, for example, the Nanoclone method (see, e.g., WO 06/079372, which is herein incorporated by reference in its entirety), which is a proprietary method for generating Nanobodies against a desired target, based on automated high-throughput selection of B-cells and could be used in the context of the invention. The successful selection of nanobodies using the Nanoclone method may provide an initial set of nanobodies, which are then used to discover bispecific molecules comprising nanobodies using the methods described herein.

A variety of VHH domains are commercially available, any of which may be used in embodiments of the present invention. A list of VHH domains that may be used in connection with embodiments of the invention is provided in Table 1 above.

### Method of Producing Pseudotyped Viral Particles

A method of producing a pseudotyped viral (e.g., lentiviral or gammaretroviral) particle described herein will generally involve introducing a viral transfer vector and one or more additional vectors (e.g., a retroviral packaging vector) into a cell. A variety of methods suitable for production of pseudotyped viral vectors of the invention are known, such as those presented in Merten, et al., “Production of lentiviral vectors”, Mol Ther Methods C/in Dev, 3:16017 (2016) and in Nasri, et al., “Production, purification and titration of a lentivirus-based vector for gene delivery purposes”, Cytotechnology, 66:1031-1038 (2014), the disclosures of which are incorporated herein by reference in their entireties for all purposes.

In embodiments, the production of a pseudotyped viral particle involves introducing into a cell (i.e., a producer cell) a viral transfer vector containing a heterologous gene sequence, a packaging vector, and an envelope vector (e.g., a vector encoding a glycoprotein or fragment thereof fused to a VHH or fragment thereof). In embodiments, the viral transfer vector contains a heterologous polynucleotide sequence containing a heterologous gene flanked by long terminal repeat (LTR) sequences, which facilitate integration of the heterologous gene sequence into the genome of a target cell. In embodiments, the transfer vector may contain a deletion in a 3′LTR to render the pseudotyped viral particle self-inactivating (SIN) after integration of the polynucleotide into the genome of the target cell.

The vectors may be introduced into the cell using transfection methods well known in the art. After transfection, the cell may be permitted to express viral proteins encoded by the viral transfer vector and/or the one or more additional vectors (e.g., by incubating the cell under standard conditions known in the art for inducing viral gene expression). In embodiments, the viral genes are expressed under the control of a constitutive or inducible promoter. In the latter case, viral gene expression may be selectively induced by incubating the cell under conditions suitable for activating the inducible promoter. Viral proteins produced by the cell may subsequently form a viral particle, which buds from the cell surface and can be isolated from the solution (e.g., according to methods well known in the art). When the viral particle buds from the cell surface and obtains a viral envelope containing a portion of the lipid membrane of the cell from which it budded as well as associated membrane proteins (e.g., a hemagglutinin) that were contained within the lipid membrane of the cell. During formation of the virus, a polynucleotide encoding a heterologous polypeptide may be incorporated into the viral particle. Thus, this process yields a pseudotyped retroviral particle that includes a polynucleotide encoding a heterologous gene (e.g., a heterologous polypeptide), where the polynucleotide sequence originated from the viral transfer vector.

The heterologous gene may include a gene encoding a polypeptide or a gene for a noncoding RNA that is to be expressed in a target cell. In some instances, the heterologous protein ORF is positioned downstream of a Kozak sequence. In some instances, the polynucleotide of the viral transfer vector will be present in a retroviral particle produced in a cell transfected with the viral transfer vector and, optionally, one or more additional vectors (e.g., packaging vectors). In certain instances, the polynucleotide may be integrated into the genome of a cell infected with the pseudotyped retroviral particle. Integration of the heterologous nucleic acid into the genome of such a cell may permit the cell and its progeny to express the heterologous gene of interest. The gene of interest may be any gene known in the art. Exemplary genes of interest include, without limitation, genes encoding chimeric antigen receptors (CARs), binding moieties (e.g., antibodies and antibody fragments), signaling proteins, cell surface proteins (e.g., T cell receptors), proteins involved in disease (e.g., cancers, autoimmune diseases, neurological disorders, or any other disease known in the art), or any derivative or combination thereof. In embodiments, the heterologous polypeptide is an antigen (e.g., an influenza, coronavirus, cancer, or cytomegalovirus antigen). In embodiments, the heterologous polypeptide is a therapeutic polypeptide (e.g., a chimeric antigen receptor (CAR)).

A viral transfer vector of the invention may be introduced into a cell (producer cell). The viral transfer vector is generally co-transfected into the cell together with one or more additional vectors (e.g., one or more packaging vectors). The one or more additional vectors may encode viral proteins and/or regulatory proteins. Co-transfection of the viral transfer vector and the one or more additional vectors (e.g., a vector encoding a glycoprotein fused to a VHH) enables the host cell to produce a pseudotyped viral particle (e.g., a lentivirus or gammaretrovirus containing a polynucleotide from the lentiviral transfer vector). Pseudotyped retroviral particles produced by a cell as described herein may be used to infect another cell. The polynucleotide containing a heterologous gene sequence (e.g., encoding a polypeptide of interest) and/or one or more additional elements (e.g., promoters and viral elements) may be integrated into the genome of the infected cell, thereby permitting the cell and its progeny to express gene(s) originating from the viral transfer vector.

A producer cell suitable for transfection with the lentiviral transfer vector (and one or more packaging vectors) may be a eukaryotic cell, such as a mammalian cell. The host cell may originate from a cell line (e.g., an immortalized cell line). For example, the host cell may be a HEK 293 cell.

Target cell is the cell that is infected (transduced) with a pseudotyped viral particle containing a polynucleotide encoding a gene of interest. After transduction, the heterologous gene of interest is stably inserted into target cell genome and can be detected by molecular biology methods such as PCR and Southern blot. Transgene can be expressed in target cell and detected by flow cytometry or Western blot. In some instances, target cell is a human cell. In certain instances, the host cell is a particular cell type of interest, e.g., a primary T cell, SupTI cell, Jurkat cell, or 293T cell.

The viral transfer vectors may include one or more of the following: a promoter (e.g., a CMV, RSV, or EF1a promoter) driving expression of one or more viral sequences, long terminal repeat (LTR) regions (e.g., an R region or an U5 region), optionally flanking a heterologous gene sequence, a primer binding site (PBS), a packaging signal (psi) (e.g., a packaging signal including a major splice donor site (SD)), acPPT element, a Kozak sequence positioned upstream (e.g., immediately upstream) of a heterologous gene sequence to be transferred to a cell), a Rev-response element (RRE), a subgenomic promoter (e.g., P-EF1a), a heterologous gene (e.g., a heterologous gene encoding a CAR gene), a post-transcriptional regulatory element (e.g., a WPRE or HPRE), a polyA sequence, a selectable marker (e.g., a kanamycin resistance gene (nptII), ampicilin resistance gene, or a chloramphenicol resistance gene), and an origin of replication (e.g., a pUC origin of replication, an SV40 origin of replication, or an fl origin of replication).

The viral transfer vector may also include elements suitable for driving expression of a heterologous protein in a cell. In certain instances, a Kozak sequence is positioned upstream of the heterologous protein open reading frame. For example, the viral transfer vector may include a promoter (e.g., a CMV, RSV, or EFla promoter) that controls the expression of the heterologous nucleic acid. Other promoters suitable for use in the lentiviral transfer vector include, for example, constitutive promoters or tissue/cell type-specific promoters. In some instances, the lentiviral transfer vector includes a means of selectively marking a gene product (e.g., a polypeptide or RNA) encoded by at least a portion of the polynucleotide (e.g., a polynucleotide encoding a gene product of interest). For example, the viral transfer vector may include a marker gene (e.g., a gene encoding a selectable marker, such as a fluorescent protein (e.g., GFP, YFP, RFP, dsRed, mCherry, or any derivative thereof)). The marker gene may be expressed independently of the gene product of interest. Alternatively, the marker gene may be co-expressed with the gene product of interest. For example, the marker gene may be under the control of the same or different promoter as the gene product of interest. In another example, the marker gene may be fused to the gene product of interest. The elements of the viral transfer vectors of the invention are, in general, in operable association with one another, to enable the transfer vectors together with one or more packaging vectors to participate in the formation of a pesudotyped viral particle in a transfected cell.

The viral transfer vectors of the invention may be co-transfected into a cell together with one or more additional vectors. In some instances, the one or more additional vectors may include lentiviral packaging vectors and/or envelop vectors. In certain instances, the one or more additional vectors may include an envelope vector (e.g., an envelope vector encoding a glycoprotein fused to a VHH). Generally, a packaging vector includes one or more polynucleotide sequences encoding viral proteins (e.g., gag, pol, env, tat, rev, vif, vpu, vpr, and/or nef protein, or a derivative, combination, or portion thereof). A packaging vector to be co-transfected into a cell with a viral transfer vector of the invention may include sequence(s) encoding one or more viral proteins not encoded by the transfer vector. For example, a viral transfer vector may be co-transfected with a first packaging vector encoding gag and pol and a second packaging vector encoding rev. Thus, co-transfection of a viral transfer vector with such packaging vector(s) may result in the introduction of all genes required for viral particle formation into the cell, thereby enabling the cell to produce viral particles that may be isolated. Further, the viral particles produced by the cell lack genes critical for viral particle formation and are, thus, incapable of self-replication. For various safety reasons, it can be advantageous to produce pseudotyped viral particles and are incapable of self-replication. Appropriate packaging vectors for use in the invention can be selected by those of skill in the art based on, for example, consideration of the features selected for a viral transfer vector of the invention. For examples of packaging vectors that can be used or adapted for use in the invention see, e.g., WO 03/064665, WO 2009/153563, U.S. Pat. No. 7,419,829, WO 2004/022761, U.S. Pat. No. 5,817,491, WO 99/41397, U.S. Pat. Nos. 6,924,123, 7,056,699, WO 99/32646, WO 98/51810, and WO 98/17815. In some instances, a packaging vector may encode a gag and/or pol protein, and may optionally include an RRE sequence (e.g., an pMDLgpRRE vector; see, e.g., Dull et al., J. Virol. 72(11):8463-8471, 1998). In certain instances, a packaging vector may encode a rev protein (e.g., a pRSV-Rev vector).

### Genome Editing

Therapeutic gene editing is a major focus of biomedical research, embracing the interface between basic and clinical science. An immune cell may be treated according to the methods of the present invention by knocking out (e.g., by deletion) or inhibiting expression of a target gene(s). The development of novel “gene editing” tools provides the ability to manipulate the DNA sequence of a cell (e.g., to delete a target gene) at a specific chromosomal locus, without introducing mutations at other sites of the genome. This technology effectively enables the researcher to manipulate the genome of a subject's cells in vitro or in vivo.

In one embodiment, gene editing involves targeting an endonuclease (an enzyme that causes DNA breaks internally within a DNA molecule) to a specific site of the genome and thereby triggering formation of a chromosomal double strand break (DSB) at the chosen site. If, concomitant with the introduction of the chromosome breaks, a donor DNA molecule may be introduced (for example, by plasmid or oligonucleotide introduction), interactions between the broken chromosome and the introduced DNA can occur, especially if the two sequences share homology. In this instance, a process termed “gene targeting” can occur, in which the DNA ends of the chromosome invade homologous sequences of the donor DNA by homologous recombination (HR). By using the donor plasmid sequence as a template for HR, a seamless repair of the chromosomal DSB can be accomplished. In some embodiments, no donor DNA molecule is introduced and the double-stranded break is repaired by the error-prone non-homologous end joining NHEJ pathway leading to knock-out or deletion of the target gene (e.g., through the introduction of indels or nonsense mutations). In some embodiments, an endonuclease(s) can be targeted to at least two distinct chosen sites located within a gene sequence so that chromosomal double strand breaks at the distinct sites leads to excision and deletion of a nucleotide sequence flanked by the two distinct sites.

Current genome editing tools use the induction of double strand breaks (DSBs) to enhance gene manipulation of cells, including the deletion or knockout of genes. Such methods include zinc finger nucleases (ZFNs; described for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, and U.S. Pat. Publ. Nos. 20030232410 and US2009020314, which are incorporated herein by reference), Transcription Activator-Like Effector Nucleases (TALENs; described for example in U.S. Pat. Nos. 8,440,431, 8,440,432, 8,450,471, 8,586,363, and 8,697,853, and U.S. Pat. Publ. Nos. 20110145940, 20120178131, 20120178169, 20120214228, 20130122581, 20140335592, and 20140335618, which are incorporated herein by reference), and the CRISPR (Clustered Regularly Interspaced Short Palindromic Repeats)/Cas9 system (described for example in U.S. Pat. Nos. 8,697,359, 8,771,945, 8,795,965, 8,871,445, 8,889,356, 8,906,616, 8,932,814, 8,945,839, 8,993,233, and 8,999,641, and U.S. Pat. Publ. Nos. 20140170753, 20140227787, 20140179006, 20140189896, 20140273231, 20140242664, 20140273232, 20150184139, 20150203872, 20150031134, 20150079681, 20150232882, and 20150247150, which are incorporated herein by reference). In some embodiments a CRISPR/Cas12 system can be used for gene editing. In some embodiments, the Cas12 polypeptide is Cas12b. In some embodiments any Cas polypeptide can be used for gene editing (e.g., CasX). In various embodiments, the Cas polypeptide is selected so that a nucleotide encoding the Cas polypeptide can fit within an adeno-associated virus (AAV) capsid. For example, ZFN DNA sequence recognition capabilities and specificity can be unpredictable. Similarly, TALENs and CRISPR/Cas9 cleave not only at the desired site, but often at other “off-target” sites, as well. These methods have significant issues connected with off-target double-stranded break induction and the potential for deleterious mutations, including indels, genomic rearrangements, and chromosomal rearrangements, associated with these off-target effects. ZFNs and TALENs entail use of modular sequence-specific DNA binding proteins to generate specificity for −18 bp sequences in the genome.

CRISPR/Cas9, TALENs, and ZFNs have all been used in clinical trials (see, e.g., Li., H, et al., “Applications of genome editing technology in the targeted therapy of human diseases: mechanisms, advances and prospects”, Signal Transduct Target Ther., 5:1 (2020), DOI: 10.1038/s41392-019-0089-y).

RNA-guided nucleases-mediated genome editing, based on Type 2 CRISPR (Clustered Regularly Interspaced Short Palindromic Repeat)/Cas (CRISPR Associated) systems, offers a valuable approach to alter the genome. In brief, Cas9, a nuclease guided by single-guide RNA (sgRNA), binds to a targeted genomic locus next to the protospacer adjacent motif (PAM) and generates a double-strand break (DSB). The DSB is then repaired either by non-homologous end joining (NHEJ), which leads to insertion/deletion (indel) mutations, or by homology-directed repair (HDR), which requires an exogenous template and can generate a precise modification at a target locus (Mali et al., Science. 2013 Feb. 15; 339(6121):823-6). Genetic manipulation using engineered nucleases has been demonstrated in tissue culture cells and rodent models of diseases.

CRISPR has been used in a wide range of organisms including baker's yeast (S. cerevisiae), zebra fish, nematodes (C. elegans), plants, mice, and several other organisms. Additionally, CRISPR has been modified to make programmable transcription factors that allow scientists to target and activate or silence specific genes. Libraries of tens of thousands of guide RNAs are now available.

Since 2012, the CRISPR/Cas system has been used for gene editing (silencing, enhancing or changing specific genes) that even works in eukaryotes like mice and primates. By inserting a plasmid containing Cas genes and specifically designed CRISPRs, an organism's genome can be cut at any desired location.

CRISPR repeats range in size from 24 to 48 base pairs. They usually show some dyad symmetry, implying the formation of a secondary structure such as a hairpin, but are not truly palindromic. Repeats are separated by spacers of similar length. Some CRISPR spacer sequences exactly match sequences from plasmids and phages, although some spacers match the prokaryote's genome (self-targeting spacers). New spacers can be added rapidly in response to phage infection.

CRISPR-associated (cas) genes are often associated with CRISPR repeat-spacer arrays. As of 2013, more than forty different Cas protein families had been described. Of these protein families, Cas1 appears to be ubiquitous among different CRISPR/Cas systems. Particular combinations of Cas genes and repeat structures have been used to define 8 CRISPR subtypes (E. coli, Y. pest, Nmeni, Dvulg, Tneap, Hmari, Apern, and Mtube), some of which are associated with an additional gene module encoding repeat-associated mysterious proteins (RAMPs). More than one CRISPR subtype may occur in a single genome. The sporadic distribution of the CRISPR/Cas subtypes suggests that the system is subject to horizontal gene transfer during microbial evolution.

Exogenous DNA is apparently processed by proteins encoded by Cas genes into small elements (about 30 base pairs in length), which are then somehow inserted into the CRISPR locus near the leader sequence. RNAs from the CRISPR loci are constitutively expressed and are processed by Cas proteins to small RNAs composed of individual, exogenously-derived sequence elements with a flanking repeat sequence. The RNAs guide other Cas proteins to silence exogenous genetic elements at the RNA or DNA level. Evidence suggests functional diversity among CRISPR subtypes. The Cse (Cas subtype E. coli) proteins (called CasA-E in E. coli) form a functional complex, Cascade, that processes CRISPR RNA transcripts into spacer-repeat units that Cascade retains. In other prokaryotes, Cas6 processes the CRISPR transcripts. Interestingly, CRISPR-based phage inactivation in E. coli requires Cascade and Cas3, but not Cas1 and Cas2. The Cmr (Cas RAMP module) proteins found in Pyrococcusfuriosus and other prokaryotes form a functional complex with small CRISPR RNAs that recognizes and cleaves complementary target RNAs. RNA-guided CRISPR enzymes are classified as type V restriction enzymes. See also U.S. Patent Publication 2014/0068797, which is incorporated by reference in its entirety.

### Cas9

Cas9 is a nuclease, an enzyme specialized for cutting DNA, with two active cutting sites, one for each strand of the double helix. The team demonstrated that they could disable one or both sites while preserving Cas9's ability to home located its target DNA. Jinek et al. (2012) combined tracrRNA and spacer RNA into a “single-guide RNA” molecule that, mixed with Cas9, could find and cut the correct DNA targets. It has been proposed that such synthetic guide RNAs might be able to be used for gene editing (Jinek et al., Science. 2012 Aug. 17; 337(6096):816-21).

Cas9 proteins are highly enriched in pathogenic and commensal bacteria. CRISPR/Cas-mediated gene regulation may contribute to the regulation of endogenous bacterial genes, particularly during bacterial interaction with eukaryotic hosts. For example, Cas protein Cas9 of Francisella novicida uses a unique, small, CRISPR/Cas-associated RNA (scaRNA) to repress an endogenous transcript encoding a bacterial lipoprotein that is critical for F. novicida to dampen host response and promote virulence. Coinjection of Cas9 mRNA and sgRNAs into the germline (zygotes) generated mice with mutations. Delivery of Cas9 DNA sequences also is contemplated.

### gRNA

As an RNA guided protein, Cas9 requires a short RNA to direct the recognition of DNA targets. Though Cas9 preferentially interrogates DNA sequences containing a PAM sequence NGG it can bind here without a protospacer target. However, the Cas9-gRNA complex requires a close match to the gRNA to create a double strand break. CRISPR sequences in bacteria are expressed in multiple RNAs and then processed to create guide strands for RNA. Because Eukaryotic systems lack some of the proteins required to process CRISPR RNAs the synthetic construct gRNA was created to combine the essential pieces of RNA for Cas9 targeting into a single RNA expressed with the RNA polymerase type 21 promoter U6). Synthetic gRNAs are slightly over 100 bp at the minimum length and contain a portion which is targets the 20 protospacer nucleotides immediately preceding the PAM sequence NGG; gRNAs do not contain a PAM sequence.

### Pharmaceutical Compositions

In some aspects, the present invention provides pharmaceutical compositions. To prepare the pharmaceutical compositions of this invention, an effective amount of an agent (e.g., a pseudotyped viral particle) is combined with a pharmaceutically acceptable carrier, which carrier may take a wide variety of forms depending on the form of preparation desired for administration. In some embodiments, the pharmaceutical composition comprises a cell that can be used to produce pseudotyped viral particles of the invention. These pharmaceutical compositions are desirable in unitary dosage form suitable, particularly, for administration percutaneously, or by parenteral injection. Any of the usual pharmaceutical media may be employed such as, for example, water, glycols, oils, alcohols and the like in the case of oral liquid preparations such as suspensions, syrups, elixirs and solutions; or solid carriers such as starches, sugars, kaolin, lubricants, binders, disintegrating agents and the like in the case of powders, pills, capsules, and tablets. For parenteral compositions, the carrier will usually comprise sterile water, at least in large part, though other ingredients, for example, to aid solubility and cell viability, may be included. Other ingredients may include antioxidants, viscosity stabilizers, chelating agents, buffers, preservatives. If desired, further ingredients may be incorporated in the compositions, e.g. anti-inflammatory agents, antibacterials, antifungals, disinfectants, vitamins, antibiotics.

Agents of the invention may be administered as part of a pharmaceutical composition. The compositions should be sterile and contain a therapeutically effective amount of the polypeptides or nucleic acid molecules in a unit of weight or volume suitable for administration to a subject.

Agents of the invention (e.g., a pseudotyped viral particle) may be administered within a pharmaceutically-acceptable diluent, carrier, or excipient, in unit dosage form. Conventional pharmaceutical practice may be employed to provide suitable formulations or compositions to administer the compounds to patients suffering from a neurological condition. Administration may begin before the patient is symptomatic. Any appropriate route of administration may be employed, for example, administration may be parenteral, intravenous, intraarterial, subcutaneous, intratumoral, intramuscular, intracranial, intraorbital, ophthalmic, intraventricular, intrahepatic, intracapsular, intrathecal, intracisternal, intraperitoneal, intranasal, aerosol, suppository, or oral administration. For example, therapeutic formulations may be in the form of liquid solutions or suspensions; for oral administration, formulations may be in the form of tablets or capsules; and for intranasal formulations, in the form of powders, nasal drops, or aerosols. In some embodiments, the composition is administered locally to a patient (e.g., proximal to a tumor) and not systemically. In some embodiment, the composition is administered systemically.

Methods well known in the art for making formulations are found, for example, in “Remington: The Science and Practice of Pharmacy” Ed. A. R. Gennaro, Lippincourt Williams & Wilkins, Philadelphia, Pa., 2000. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel. The formulations can be administered to human patients in therapeutically effective amounts (e.g., amounts which prevent, eliminate, or reduce a pathological condition) to provide therapy for a neoplastic disease or condition. The preferred dosage of an agent of the invention is likely to depend on such variables as the type and extent of the disorder, the overall health status of the particular subject, the formulation of the compound excipients, and its route of administration.

Generally, doses of pseudotyped viral particles of the present invention can be from about or at least about 1×10e7 transduction units (TU), 1×10e8 TU, 1×10e9 TU, 1×10e10 TU, or 1×10e11 TU. In embodiments, the dose of the pseudotyped viral particle of the present invention is about or at least about 1×10e7 TU/kg, 1×10e8 TU/kg, 1×10e9 TU/kg, 1×10e10 TU/kg, or 1×10e1 1 TU/kg. Lower doses will result from certain forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of an agent of the invention.

A variety of administration routes are available. The methods of the invention, generally speaking, may be practiced using any mode of administration that is medically acceptable, meaning any mode that produces effective levels of the active compounds without causing clinically unacceptable adverse effects. Other modes of administration include oral, rectal, topical, intraocular, buccal, intravaginal, intracisternal, intracerebroventricular, intratracheal, nasal, transdermal, within/on implants, e.g., fibers such as collagen, osmotic pumps, or grafts comprising appropriately transformed cells, etc., or parenteral routes.

### Methods of Treatment

The present invention provides methods of treating disease and/or disorders or symptoms thereof which comprise administering a therapeutically effective amount of a pharmaceutical composition comprising a pseudotyped viral particle (e.g., a pseudotyped lentiviral particle or a psedudotyped gammaretroviral particle). Thus, one embodiment is a method of treating a subject suffering from or susceptible to a cancer or infection (e.g., cytomegalovirus (CMV), influenza, or coronavirus disease of 2019 (COVID-19)). The method includes the step of administering to the mammal a therapeutic amount of a pseudotyped viral particle sufficient to treat the disease or disorder or symptom thereof, under conditions such that the disease or disorder is treated.

The methods herein include administering to the subject (including a subject identified as in need of such treatment) an effective amount of a pesudotyped viral particle described herein, or a composition described herein to produce such effect. Identifying a subject in need of such treatment can be in the judgment of a subject or a health care professional and can be subjective (e.g. opinion) or objective (e.g. measurable by a test or diagnostic method).

The therapeutic methods of the invention (which include prophylactic treatment) in general comprise administration of a therapeutically effective amount of the pseudotyped viral particle herein to a subject (e.g., animal, human) in need thereof, including a mammal, particularly a human. Such treatment will be suitably administered to subjects, particularly humans, suffering from, having, susceptible to, or at risk for a disease (e.g., a cancer, cytomegalovirus (CMV), influenza, or coronavirus disease of 2019 (COVID-19)), disorder, or symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like).

The cancer can be a hematologic cancer, e.g., a cancer chosen from one or more of chronic lymphocytic leukemia (CLL), acute leukemias, acute lymphoid leukemia (ALL), B-cell acute lymphoid leukemia (B-ALL), T-cell acute lymphoid leukemia (T-ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, or pre-leukemia.

The cancer can also be chosen from colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers, combinations of said cancers, and metastatic lesions of said cancers. In one embodiment, the invention provides a method of monitoring treatment progress. The method includes the step of determining a level of diagnostic marker (Marker) or diagnostic measurement (e.g., screen, assay) in a subject suffering from or susceptible to a disorder or symptoms thereof associated with a disease (e.g., a cancer), in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

The pharmaceutical compositions of this invention can be administered by any suitable routes including, by way of illustration, oral, topical, rectal, transdermal, subcutaneous, intravenous, intramuscular, intranasal, intracranial, intracerebral, intraventricular, intrathecal, and the like. In some embodiments, the administration modalities as described in U.S. Pat. Nos. 5,543,158; 5,641,515 and 5,399,363 may be used to deliver compositions of the present invention.

For therapeutic uses, the compositions and agents disclosed herein may be administered by any convenient method; for example, parenterally, conveniently in a pharmaceutically or physiologically acceptable carrier, e.g., phosphate buffered saline, saline, deionized water, or the like. The compositions may be added to a retained physiological fluid such as blood or synovial fluid. For central nervous system (CNS) administration, a variety of techniques are available for promoting transfer of an agent across the blood brain barrier including disruption by surgery or injection, drugs which transiently open adhesion contact between central nervous system (CNS) vasculature endothelial cells, and compounds which facilitate translocation through such cells. As examples, many of the disclosed compositions are amenable to be directly injected or infused or contained within implants e.g. osmotic pumps, grafts comprising appropriately transformed cells. Compositions of the present invention may also be amenable to direct injection or infusion, topical, intratracheal/nasal administration e.g. through aerosol, intraocularly, or within/on implants e.g. fibers e.g. collagen, osmotic pumps, or grafts comprising appropriately transformed cells. Generally, the amount administered will be empirically determined. Other additives may be included, such as stabilizers, bactericides, etc. In various embodiments, these additives can be present in conventional amounts.

In various embodiments, the agents of the present invention are administered in sufficient amounts to provide sufficient levels of the agent in a subject without undue adverse effects. Conventional and pharmaceutically acceptable routes of administration include, but are not limited to, direct delivery to a selected organ or tissue (e.g., the spinal cord or brain), oral, inhalation (including intranasal and intratracheal delivery), intraocular, intravenous, intramuscular, subcutaneous, intradermal, intratumoral, and other parental routes of administration. Routes of administration may be combined, if desired.

The dose of an agent used to achieve a particular “therapeutic effect” will vary based on several factors including, but not limited to: the route of administration, the level of gene or RNA expression used to achieve a therapeutic effect, the specific disease or disorder being treated, and the stability of the agent. One of skill in the art can readily determine a dose range to treat a patient having a particular disease, injury, or condition based on the aforementioned factors, as well as other factors that are well known in the art.

Administration of agents of the present invention to a subject may be by, for example, intramuscular injection or by administration into the bloodstream of the subject. Administration into the bloodstream may be by injection into a vein, an artery, or any other vascular conduit. Agents of the present invention can be inserted into a delivery device which facilitates introduction by injection or implantation into a subject. Such delivery devices include tubes, e.g., catheters, for injecting cells and fluids into the body of a recipient subject. In a preferred embodiment, the tubes additionally have a needle, e.g., a syringe, through which the contents of the invention can be introduced into the subject at a desired location. Agents of the invention can be inserted into such a delivery device, e.g., a syringe, in different forms. For example, an agent can be suspended in a solution or embedded in a support matrix when contained in such a delivery device. As used herein, the term “solution” includes a pharmaceutically acceptable carrier or diluent in which the agent of the invention remain functional and/or viable. Pharmaceutically acceptable carriers and diluents include saline, aqueous buffer solutions, solvents and/or dispersion media. The use of such carriers and diluents is well known in the art. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. In some embodiments, the selection of the carrier is not a limitation of the present invention. The solution is preferably sterile and fluid. Preferably, the solution is stable under the conditions of manufacture and storage and preserved against the contaminating action of microorganisms such as bacteria and fungi through the use of, for example, parabens, chlorobutanol, phenol, ascorbic acid, thimerosal, and the like. Solutions of the invention can be prepared by incorporating recombinant adeno-associated virus particles, nucleotide molecules, and/or vectors as described herein in a pharmaceutically acceptable carrier or diluent and, as other ingredients enumerated herein, followed by filtered sterilization. Optionally, an agent may be administered on support matrices. Support matrices in which an agent can be incorporated or embedded include matrices which are recipient-compatible and which degrade into products which are not harmful to the recipient. Natural and/or synthetic biodegradable matrices are examples of such matrices. Natural biodegradable matrices include plasma clots, e.g., derived from a mammal, and collagen matrices. Synthetic biodegradable matrices include synthetic polymers such as polyanhydrides, polyorthoesters, and polylactic acid. Other examples of synthetic polymers and methods of incorporating or embedding cells into these matrices are known in the art. These matrices provide support and protection for the cells in vivo.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a bioactive factor at a particular target site.

One feature of certain embodiments of an implant can be the linear release of an agent of the present invention, which can be achieved through the manipulation of the polymer composition and form. By choice of monomer composition or polymerization technique, the amount of water, porosity and consequent permeability characteristics can be controlled. The selection of the shape, size, polymer, and method for implantation can be determined on an individual basis according to the disorder, injury, or disease to be treated and the individual patient response. The generation of such implants is generally known in the art.

In another embodiment of an implant an agent of the invention is encapsulated in implantable hollow fibers or the like. Such fibers can be pre-spun and subsequently loaded with the agent, or can be co-extruded with a polymer which acts to form a polymeric coat about the agent.

In addition to the methods of delivery described above, the following techniques are also contemplated as alternative methods of delivering an agent to a subject. Ultrasound has been used as a device for enhancing the rate and efficacy of drug permeation into and through a circulatory system. Other drug delivery alternatives contemplated are intraosseous injection (see, e.g., U.S. Pat. No. 5,779,708), microchip devices (see, e.g., U.S. Pat. No. 5,797,898), ophthalmic formulations, transdermal matrices (see, e.g., U.S. Pat. Nos. 5,770,219 and 5,783,208), and feedback-controlled delivery (see, e.g., U.S. Pat. No. 5,697,899).

### Kits

Also provided are kits for preventing or treating a disease (e.g., a cancer, an influenza infection, a coronavirus disease, or a cytomegalovirus infection), condition, or pathology in a subject in need thereof. In one embodiment, the kit provides a therapeutic or prophylactic composition containing an effective amount of a pseudotyped viral particle as described herein, which contains a glycoprotein domain or fragment thereof fused to a VHH domain or fragment thereof, where the kit is for use in administering the pseudotyped viral particle to a subject. In embodiments, the pseudotyped viral particle targets an immune cell (e.g., a B cell, a dendritic cell, an eosinophil, a granulocyte, an iNKT cell, a macrophage, a monocyte, a natural killer cell, a neutrophil, a lymphoma cell, a regulatory T cell, and a T cell).

In another embodiment, the kit provides a therapeutic or prophylactic composition containing an effective amount of a pseudotyped viral particle as described herein.

In some embodiments, the kit comprises a sterile container which contains the therapeutic or prophylactic composition; such containers can be boxes, ampoules, bottles, vials, tubes, bags, pouches, blister-packs, or other suitable container forms known in the art. The containers can be made of plastic, glass, laminated paper, metal foil, or other materials suitable for holding medicaments.

A composition comprising a viral particle pseudotyped with a glycoprotein domain or fragment thereof fused to a VHH domain or fragment thereof, as described herein, is provided together with instructions for administering the composition to a subject having or at risk of developing a disease. The instructions will generally include information about the use of the composition for the treatment of the disease. In other embodiments, the instructions include at least one of the following: description of the therapeutic agent; dosage schedule and administration for treatment or prevention of a disease (e.g., cancer) or symptoms thereof; precautions; warnings; indications; counter-indications; overdosage information; adverse reactions; animal pharmacology; clinical studies; and/or references. The instructions may be printed directly on the container (when present), or as a label applied to the container, as information stored on a remotely-accessible server, or as a separate sheet, pamphlet, card, or folder supplied in or with the container.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: Surface-Expression in Producer Cells of Non-Measles Virus Morbillivirus Glycoprotein Domains Fused to VHH Domains

Many lentiviral vectors (LVs) are pseudotyped with the vesicular stomatitis virus glycoprotein (VSVg) (FIG. 1), due to its broad tissue tropism. However, LDL-R, the cellular receptor mediating VSVg LV entry, is poorly expressed on immune cells. To overcome this challenge, the effectiveness of retargeting Morbillivirus-pseudotyped lentiviral vectors using nanobodies (VHH's) was evaluated. This was done by fusing a lentivirus envelope glycoprotein to VHH's selectively targeted to specific cell surface antigens. Non-limiting examples of VHH's include the anti-major histocompatibility complex II (MHCII) VHH (N11) polypeptide, the anti-CD45 (32) VHH polypeptide, the anti-CD7 (VHH10) VHH polypeptide, the anti-CD4 (03F11) VHH polypeptide, and the anti-CD8 (R3HCD27) VHH polypeptide. Non-limiting examples of lentiviral envelope glycoproteins are presented in FIG. 1 and further include MeV-Hwtc18, CDV-F, CDF-Fc30, DMV-F, DMV-Fc30, DMV-H, DMV-Hc18 (MeV-Hcl8-DMV), CDV-H, CDV-Hc18 (MeV-Hc18-CDV), FMV-H, PPRV-H, RPV-H, and RMV-H.

Since lentiviral particles pseudotyped with envelope glycoproteins from the measles virus may be neutralized by antibodies present in subjects immunized against the measles virus, experiments were undertaken to evaluate the ability of lentivirus pseudotyped with envelope glycoproteins from alternative Morbilliviruses to transduce cells. As shown in FIGS. 2A-2C Morbillivirus envelope glycoprotein H's (MoV-H's) were identified sharing structural similarity with the measles virus MoV-H polypeptide but with low amino acid sequence identity in the extravirion globular head domain. In particular, it was determined that the identified Morbillivirus glycoproteins included alterations relative to the measles virus glycoprotein at amino acid positions that in the measles virus are targeted by neutralizing antibodies produced by subjects vaccinated against the measles virus. The identified Morbillivirus glycoproteins included DMV-H (dolphin Morbillivirus envelope protein H), CDV-H (canine distemper virus envelope protein H), FMV-H (feline Morbillivirus envelope protein H), PPRV-H (Peste des petits ruminant virus envelope protein H), RPV-H (Rinderpest virus envelope protein H), and RMV-H (small ruminant virus envelope protein H).

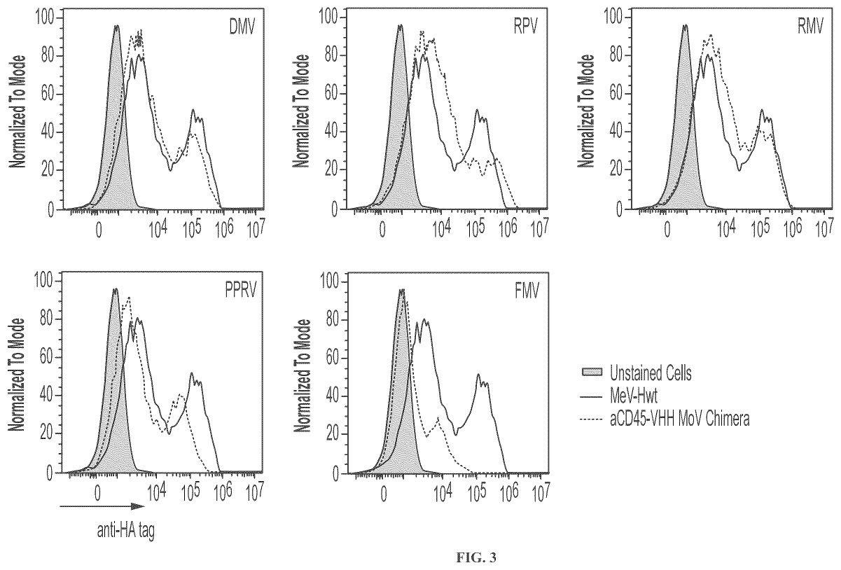

The identified Morbillivirus glycoproteins were fused to an anti-CD45 VHH domain (32) and surface expression was evaluated in producer cells (HEK293T cells). All of the fusions showed high surface expression, see FIG. 3, thereby demonstrating that the identified Morbillivirus glycoproteins can be used to pseudotype lentivirus particles.

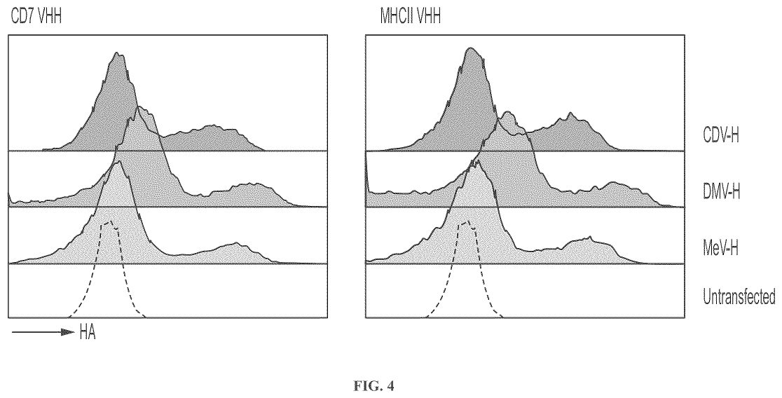

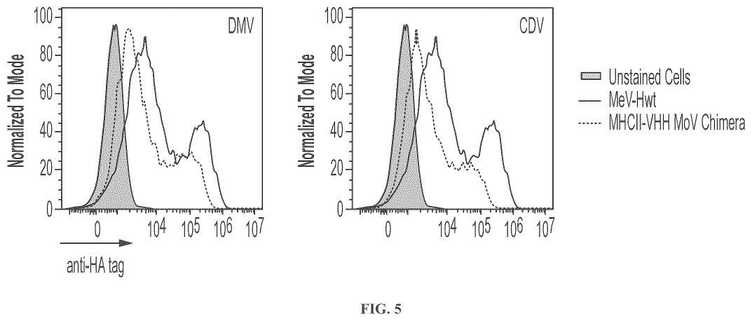

Next, surface-expression of CDV-H and DMV-H fused to an anti-CD7 VHH or an anti MHCII VHH was evaluated, see FIGS. 4 and 5. It was found that both fusion proteins showed high levels of surface expression. The fusion proteins were well tolerated on the surface of producer HEK293T cells. Thus, it was demonstrated that the identified Morbillivirus glycoproteins can be used to pseudotype lentivirus particles. In embodiments, virus particles pseudotyped with the non-measles virus Morbillivirus glycoproteins are not neutralized by measles virus neutralizing antibodies (e.g., those present in a measles-immune subject), or are subject to lower levels of neutralization by such antibodies than virus particles pseudotyped using measles virus glycoproteins.

### Example 2: Transducing Cells Using Lentivirus Particles Targeted Using Non-Measles Virus Morbillivirus Glycoprotein Domains Fused to VHH Domains

Having demonstrated that non-measles virus Morbillivirus glycoproteins fused to the VHH domains show high levels of expression in producer cells (HEK293T cells), experiments were then undertaken to determine the efficacy of lentivirus particles pseudotyped with the fusion proteins in transducing cells.

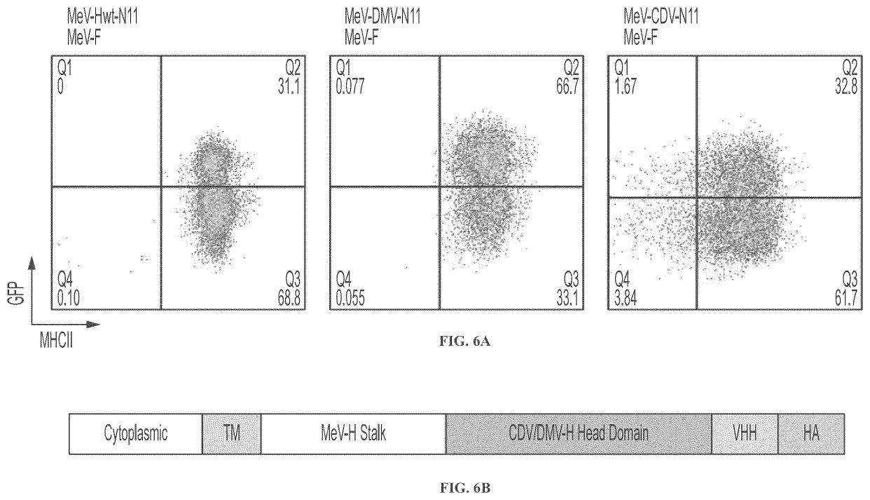

First, given that the CDV-H and DMV-H glycoproteins differ from the measles virus envelope protein H (MeV-Hwt) primarily in the globular head domain, and given that all of the amino acid sites in the measles virus envelope protein H that are target by neutralizing antibodies fall within the globular head domain, fusion proteins were prepared as shown in FIG. 6B. In particular, two fusion proteins were prepared by replacing the globular head domain of MeV-Hwt-N1 1 (i.e., the measles virus envelope protein H fused to an anti-MHCII VHH domain) with the globular head domain from CDV-H or DMV-H. The ability of these fusion proteins to facilitate targeted transduction of cells by lentivirus particles pseudotyped with them was then evaluated, see FIG. 6A. It was determined that lentivirus particles pseudotyped with the fusion proteins and the measles virus envelope protein F were effective in transducing targeted cells. In fact, the fusions showed higher levels of transduction than MeV-Hwt-N11.

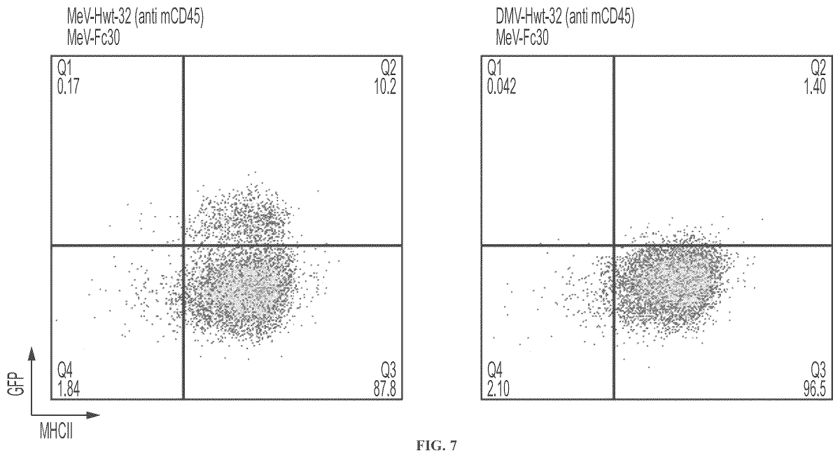

Next, experiments were undertaken to prepare lentivirus particles pseudotyped using only polypeptides derived from non-measles virus glycoproteins. As a first step, it was determined, as shown in FIG. 7, that lentivirus particles pseudotyped with DMV-Hwt-32 (anti-mCD45) and MeV-Fc30 showed transduction.

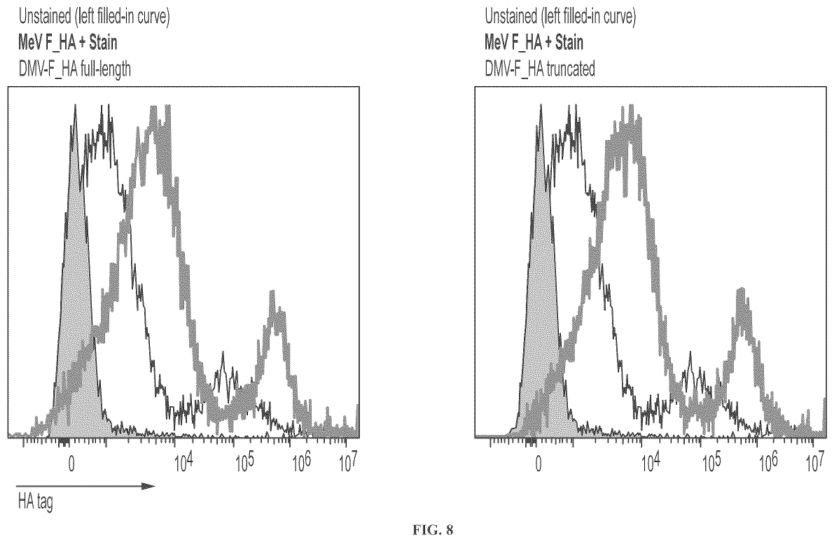

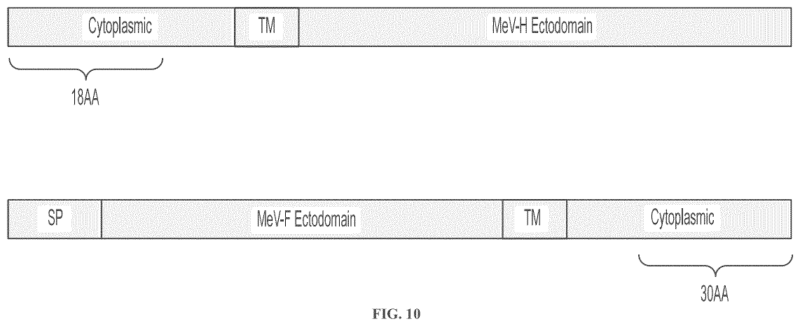

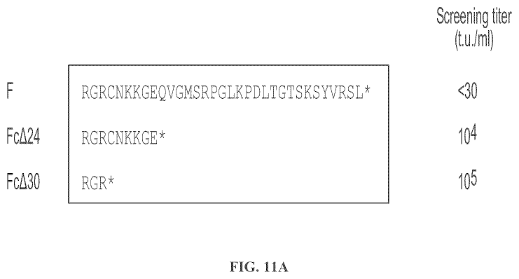

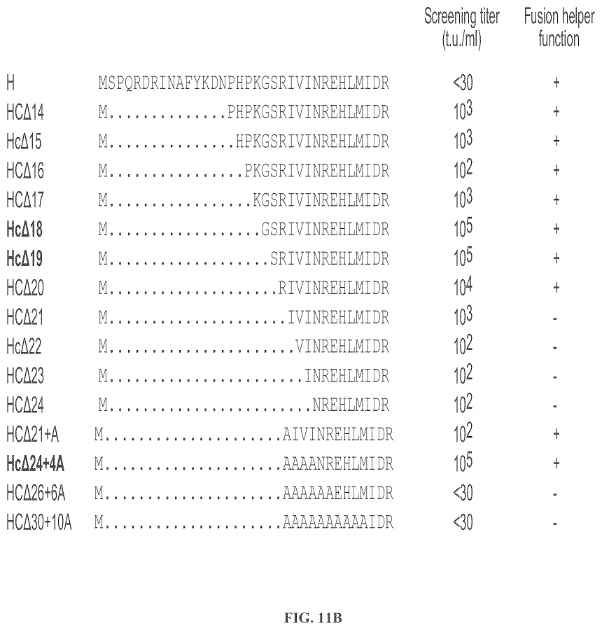

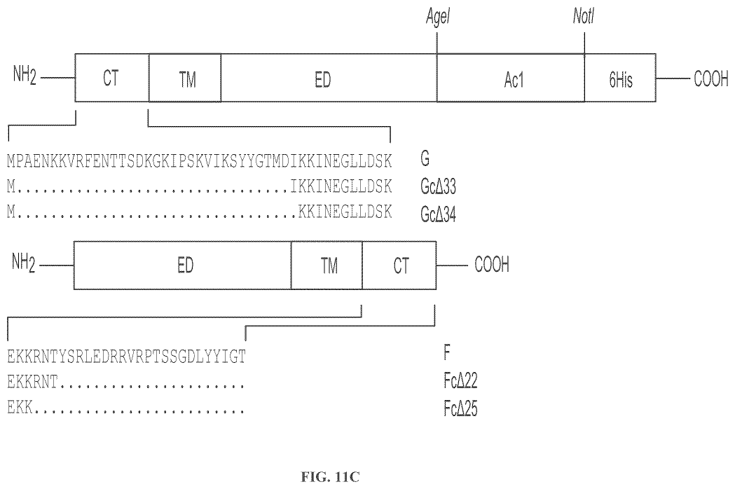

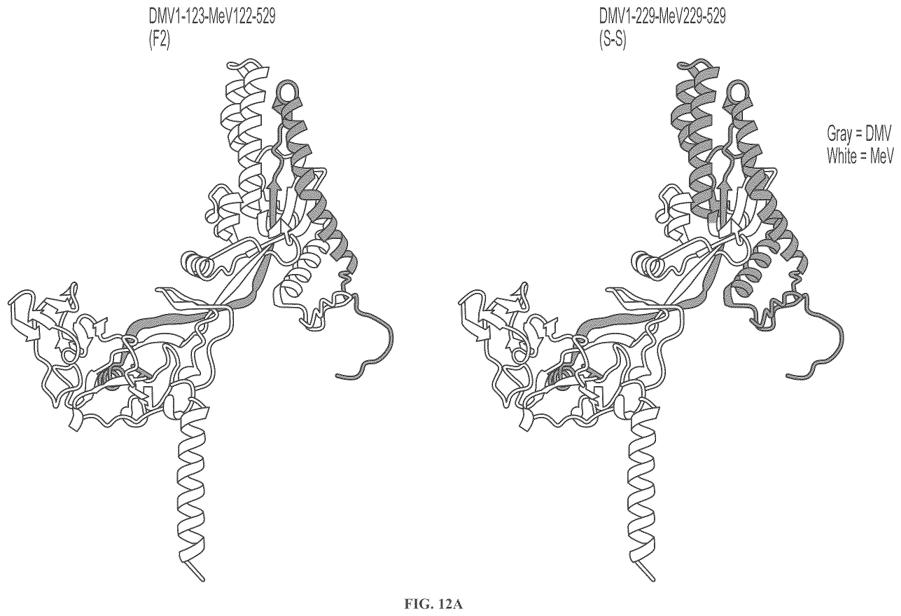

Having demonstrated that the DMV-HWT-32 (anti-mCD45) fusion protein does not function well in combination with MeV-Fc30 (where the number 30 designates a truncation of the cytoplasmic domain by 30 amino acids, as shown in FIG. 10), experiments were undertaken to optimize surface expression of the DMV-F polypeptide in producer cells (HEK293T cells) in truncated and non-truncated forms. A 30-amino acid truncation of the cytoplasmic domain of the DMV-F polypeptide was evaluated to determine whether the truncation improved transduction efficiencies. Such truncations were prepared because truncation of envelope glycoproteins H and F improves the efficacy and titer of lentiviral particles pesutodyped therewith (FIGS. 11A-11C). It was determined, as shown in FIG. 8, that both the truncated and non-truncated forms of the DMV-F polypeptide expressed well in the producer cells. Further, a DMV-Hc18-N11 (anti-MHCII; where the number 18 designates atruncation of the cytoplasmic domain by 18 amino acids, as shown in FIG. 10) polypeptide was prepared by truncating the cytoplasmic domain of the DMV-H protein domain of DMV—H-N11 by 18 amino acids to determine whether the truncation improved transduction efficiencies.

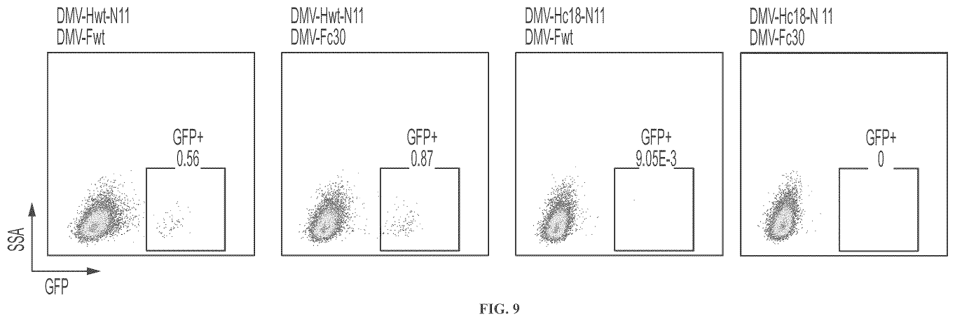

Transduction efficiencies were evaluated for lentivirus particles pseudotyped with fusion proteins comprising truncated or non-truncated DMV-H proteins in combination with truncated or non-truncated DMV-F proteins, see FIG. 9. Virus particles were prepared using a 5:3 ratio DMV-H:DMV-F plasmid. The particles were concentrated 100×and then applied to A20 cells (A20 mouse B cell lymphoma model, which is CD45⁺ and MHCII+) and analyzed by flow cytometry after 6 days. It was determined that lentivirus particles pseudotyped with DMV-Hwt-N11 (anti-MHCII; full-length DMV-H) and DMV-Fc30 (truncated DMV-F) or DMV-Fwt (non-truncated DMV-F) effectively transduced cells. Thus, it was demonstrated that lentivirus particles pseudotyped with proteins derived from dolphin Morbillivirus glycoproteins effectively transduced cells.

### Example 3: Lentiviral Particles Pseudotyped with Combinations of Dolphin Morbillivirus (DMV)—Measles Virus (MeV) Envelope Glycoproteins H and F Fusion Polypeptides were Functional

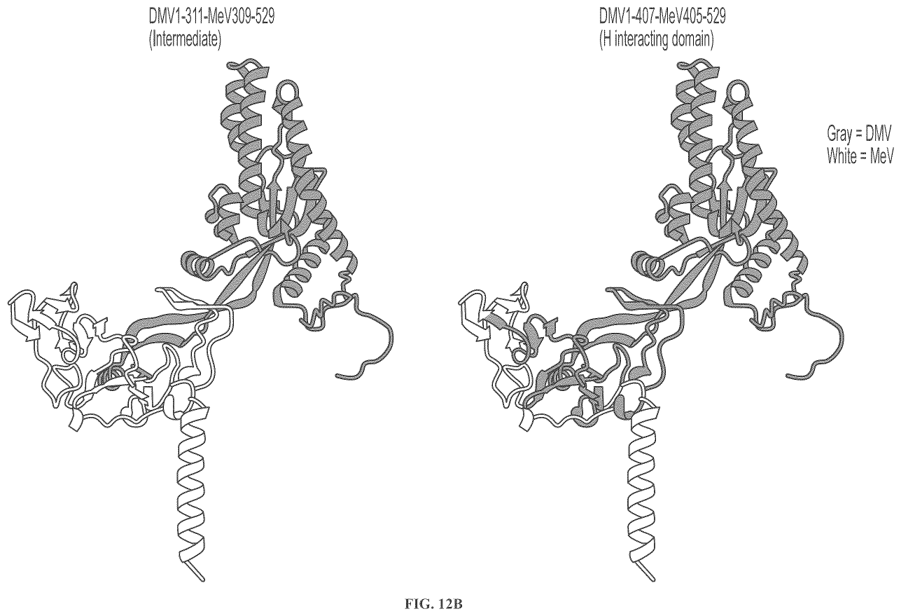

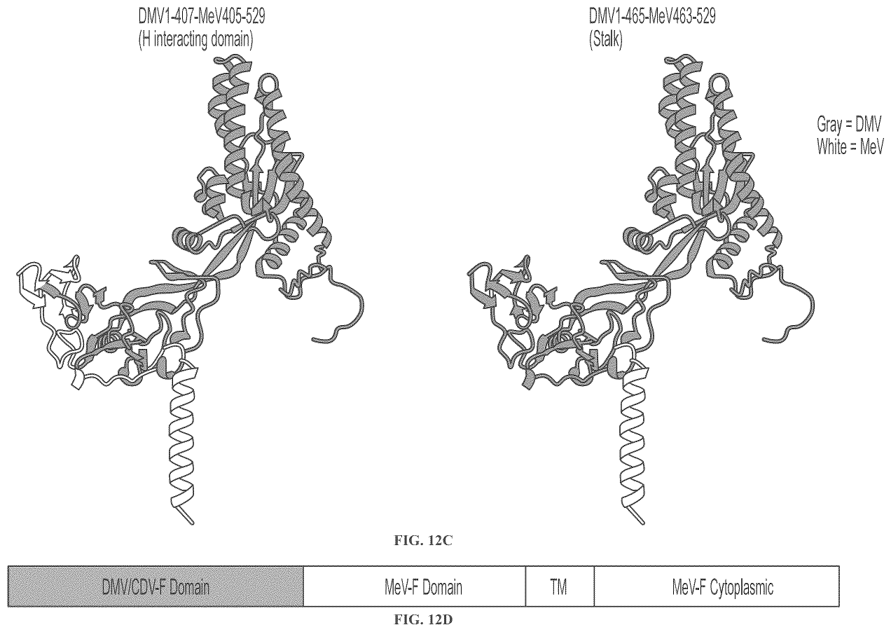

To prepare morbillivirus-pseudotyped lentiviral particles with improved capacity to avoid neutralization by human sera, experiments were undertaken to design envelope glycoprotein F fusion polypeptides (see FIGS. 12A-12D) that would be suitable for use in combination with the envelope glycoprotein H fusion polypeptides of Example 3 containing an MeV-H stalk domain and a DMV-H head domain (i.e., the MeV-DMV—H-N11 polypeptides shown in FIG. 6B).

Envelope glycoprotein F fusion proteins were designed as described in FIGS. 12A-12D that comprised progressively longer N-terminal portions thereof that were derived from the dolphin morbillivirus envelope glycoprotein F (DMV-F) and correspondingly shorter portions derived from the measles virus envelope glycoprotein F (MeV-F) on account of being replaced by the longer DMV-F portions (see FIGS. 12A-12D). The designed fusion proteins were named F2, S-S, H interacting domain, and stalk in order of shortest-to-longest length of the proportion of the fusion polypeptide derived from DMV-F.

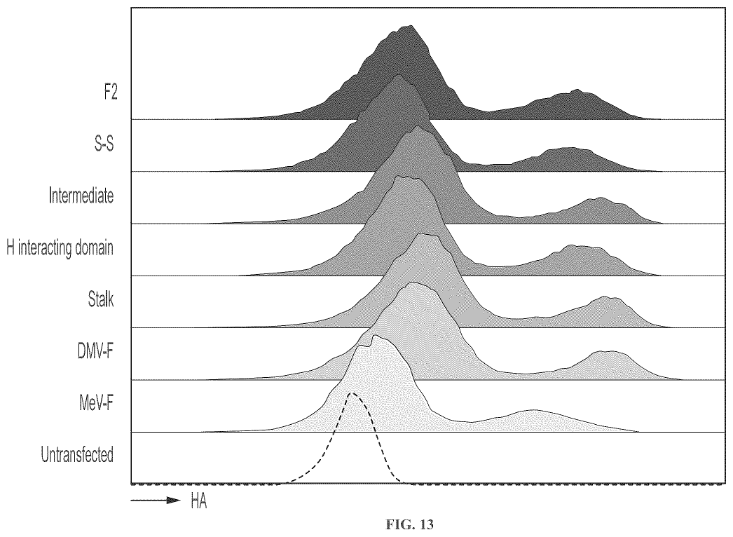

HEK293 cells were transduced with polynucleotides encoding the fusion polypeptides, DMV-F, and MeV-F and surface-expression of the polypeptides was measured using flow cytometry. All of the fusion polypeptides were highly expressed on the surface of the HEK293 cells (see FIG. 13).

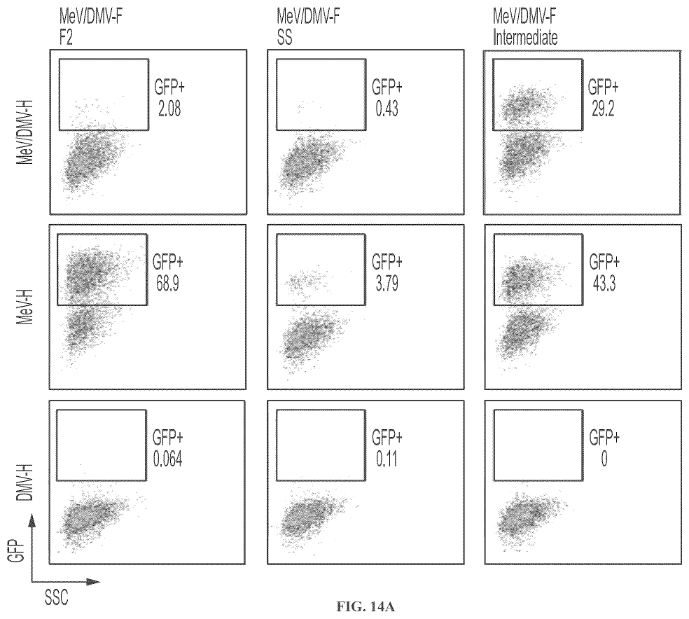

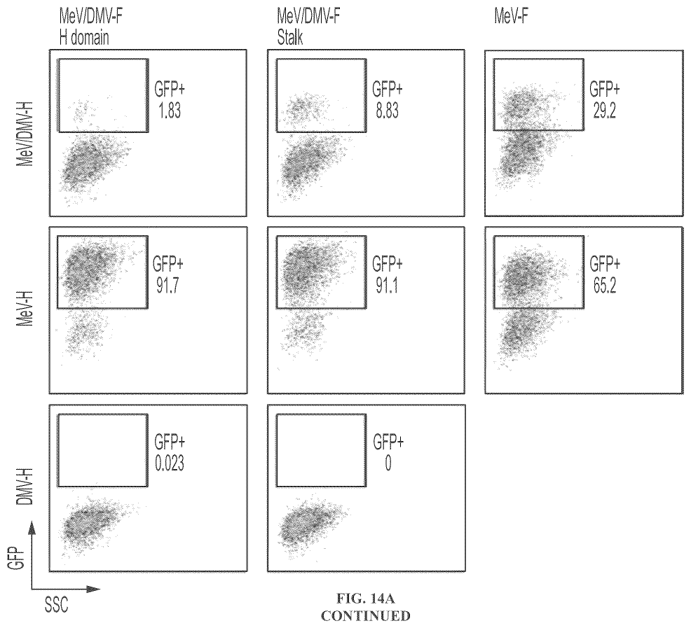

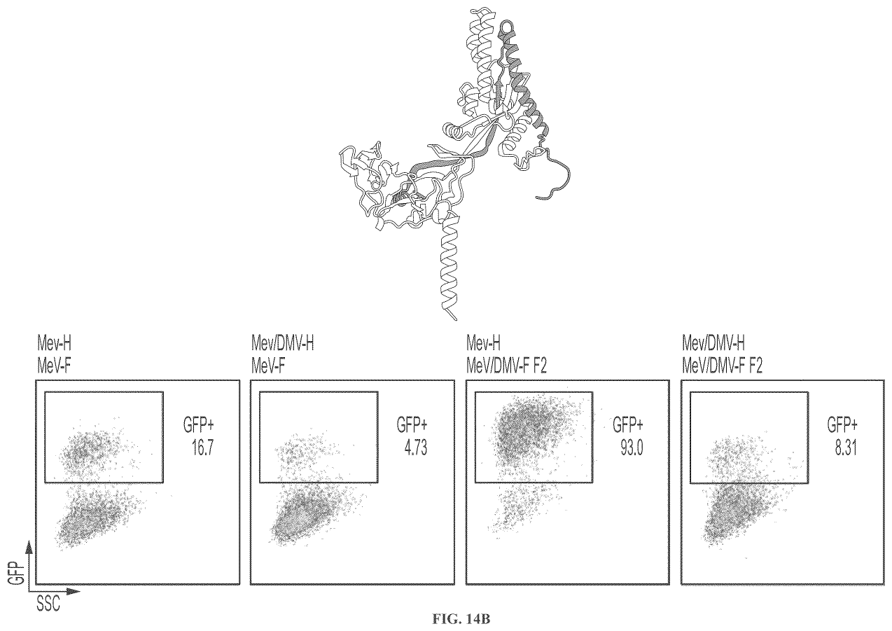

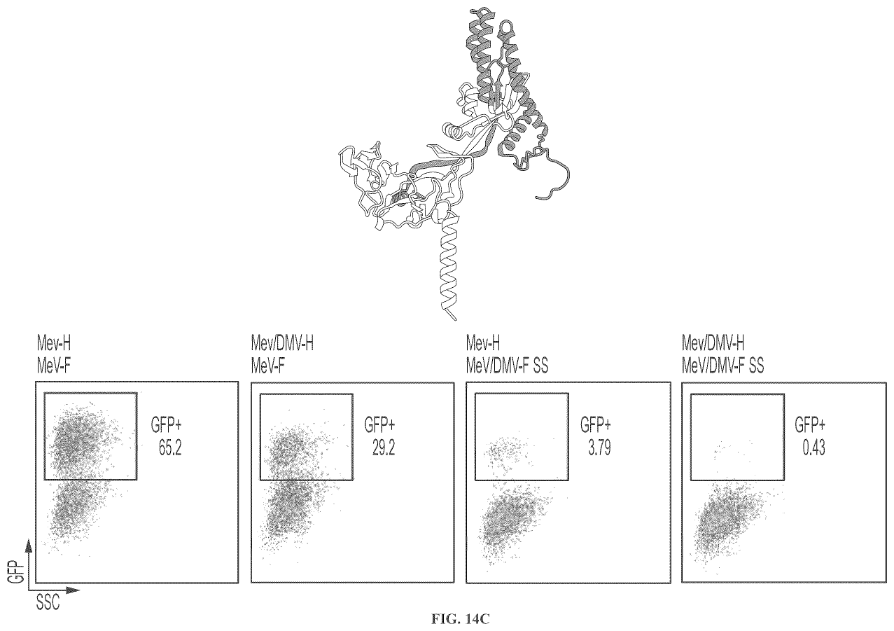

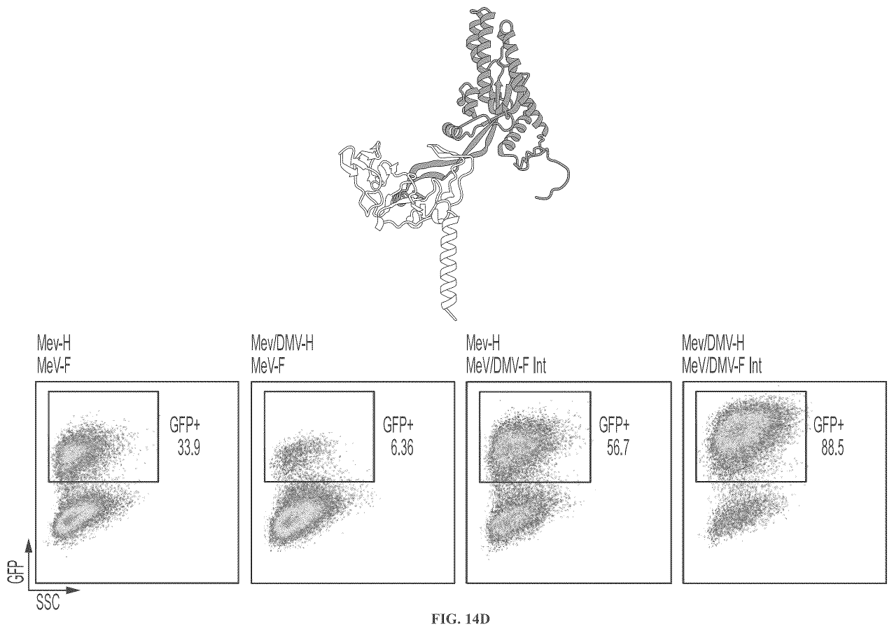

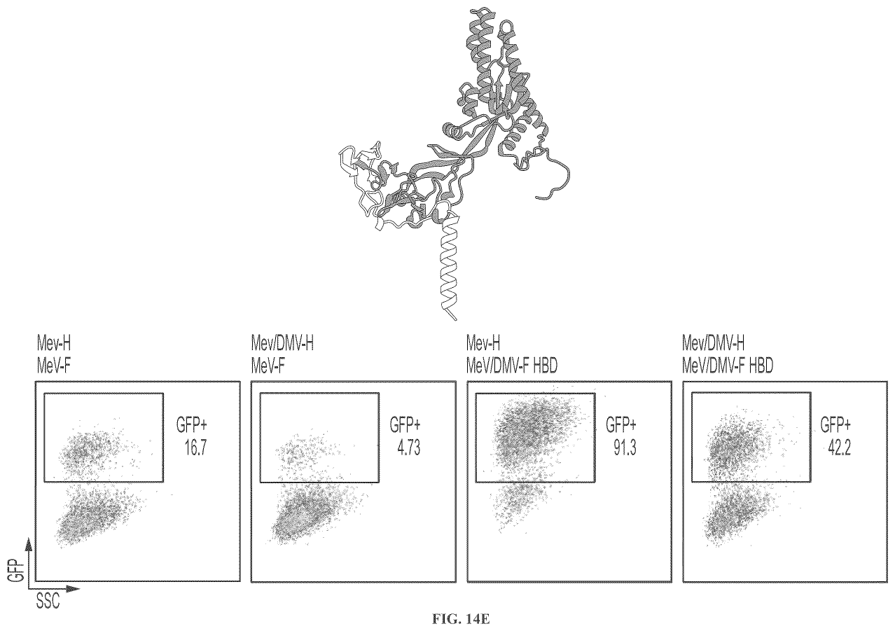

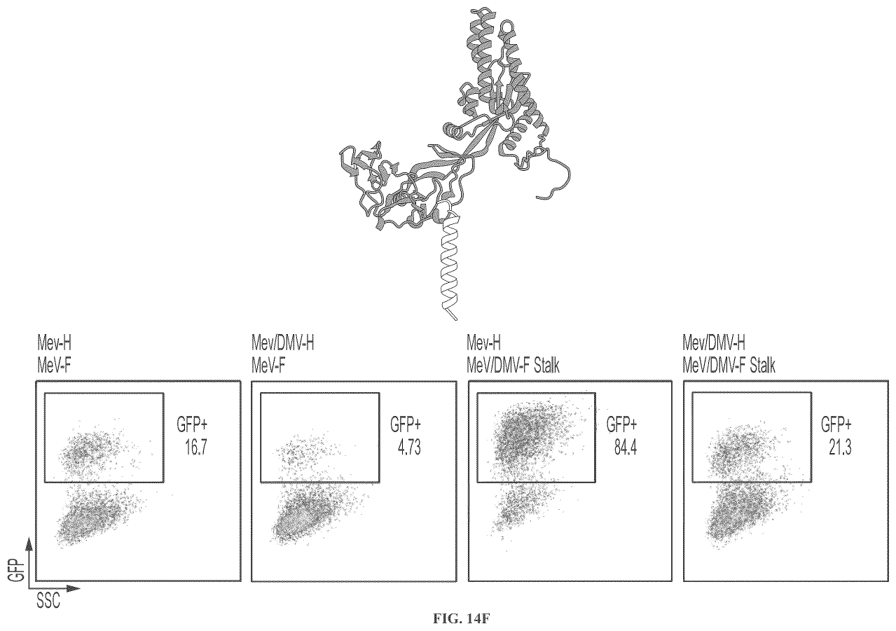

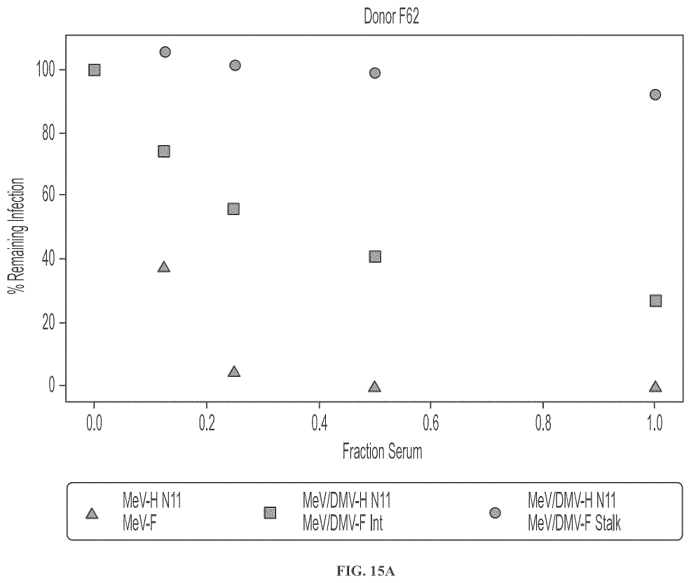

Experiments were then undertaken to evaluate the ability of lentiviruses pseudotyped with different combinations of envelope glycoprotein fusion polypeptides to infect A20 cells (FIGS. 14A-14F). The glycoprotein H fusions contained an N11 VHH domain. Lentiviral particles containing polynucleotides encoding GFP were pseudotyped using the envelope glycoprotein F fusions F2, SS, intermediate, or H domain (i.e., “H interacting/binding domain), or with MeV-F in combination with the envelope glycoprotein H fusion MeV-DMV—H-N11-, or with MeV—H-N11 or DMV—H-N11 (FIGS. 14A-14F). Lentivral particles pseudotyped with the envelope glycoprotein F fusions in combination with the envelope glycoprotein H fusion were capable of infecting the cells. Lentiviral particles pseudotyped with the intermediate fusion protein in combination with the MeV-DMV—H-VHH fusion protein showed the highest infection levels in vitro (FIG. 14D). Pseudotyping with the stalk fusion protein, which represented the greatest potential to avoid MeV-mediated neutralization, in combination with MeV-DMV—H-N11 was able to effectively infect cells (FIG. 14F).

### Example 4: Lentiviral Particles Pseudotyped with Combinations of Dolphin Morbillivirus (DMV)—Measles Virus (MeV) Envelope Glycoproteins H and F Fusion Polypeptides were Resistant to Neutralization by Human Serum Containing Anti-Measles Virus Antibodies

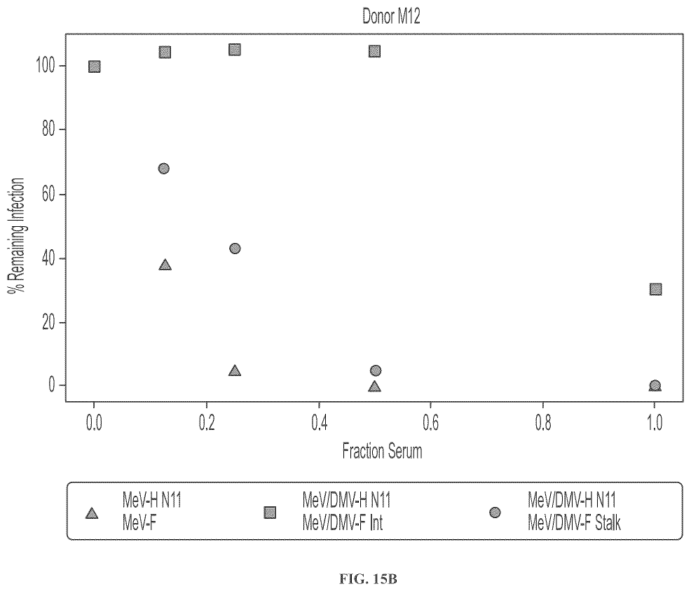

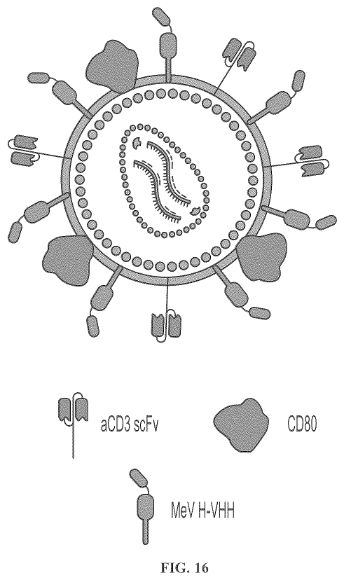

Experiments were undertaken to evaluate the ability of lentivirus particles pseudotyped with the MeV-DMV-H and MeV-DMV-F fusion polypeptides to evade neutralization by human serum obtained from humans previously vaccinated against the measles virus (FIGS. 15A and 15B).

Lentiviral particles containing expression constructs encoding GFP were prepared that were pseudotyped using the following combinations of envelope glycoproteins: MeV-H N11+MeV-F; MeV-DMV-H N11+MeV-DMV-F Int; MeV-DMV-H N11+MeV-DMV-F Stalk. Human serum was heated to 56 Celsius for 1 hour prior to incubation with the pseudotyped lentiviral particles. Concentrated lentiviral particles were diluted in validated measles immune human serum from a 62-year-old female or a 12-year-old male subject. The serum-virus mixture was incubated at 37 Celsius for 1 hour. The incubated serum-virus mixture was applied to A20 cells. The A20 cells were then analyzed for GFP expression (i.e., effective infection) using flow cytometry 2, 4, and 6 days post-infection. Percent remaining infection was calculated as [(GFP expressing cells contacted with virus particles exposed to human serum)/(GFP expressing cells contacted with virus particles never exposed to human serum)]*100% and plotted at each time point (FIGS. 15A and 15B). Lentiviral particles pseudotyped with the stalk or intermediate fusion proteins in combination with MeV-DMV-H N11 showed improved levels of resistance to neutralization by the human serum relative to lentiviral particles pesudotyped with MeV-H N11 and MeV-F.

### Example 5: Lentiviral Particles Capable of Both Activating and Infecting T Cells

Activation of T cells can improve efficiency of infection using lentivirus particles. Therefore, experiments were undertaken to develop lentiviral particles pseudotypes with envelope glycoprotein H fusion polypeptides and envelope glycoprotein F fusion polypeptides described in the preceding examples and capable of activating T cells. To enable the lentiviral particles to activate T cells an anti-CD3 scFv antigen binding polypeptide and a cluster of differentiation 80 (CD80) polypeptide was introduced to the envelope of the lentivirus particles by expressing the two polypeptides on the surface of producer HEK293T cells used to prepare the lentivirus particles (see, e.g., Dobson, C. S., et al. Nat Methods 19, 449-460 (2022), the disclosure of which is incorporated herein in its entirety for all purposes) (FIG. 16).

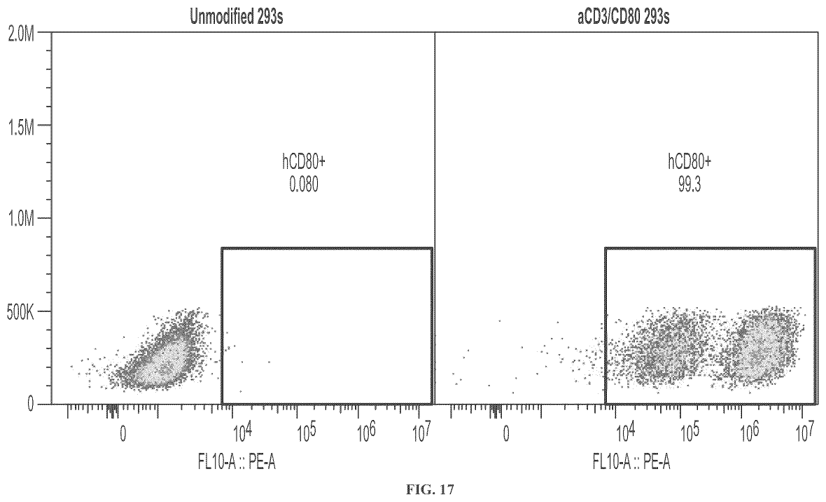

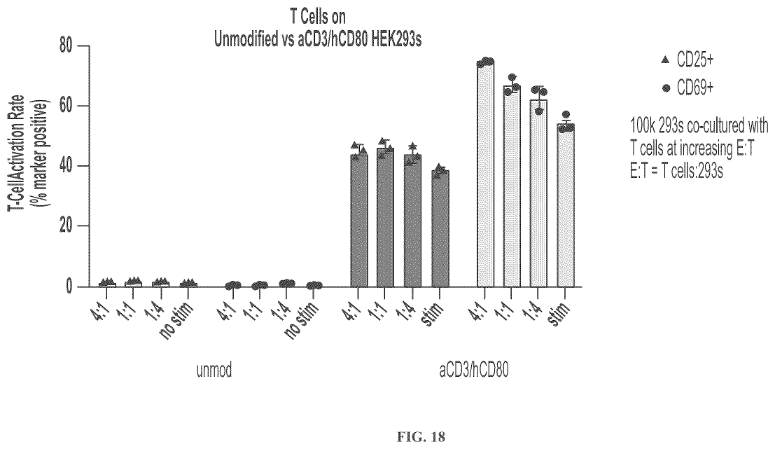

Surface-expression of the anti-CD3 scFv polypeptide and the CD80 polypeptide in the producer HEK293T cells was confirmed using flow cytometry (FIG. 17). To confirm that the anti-CD3 scFv polypeptide and the CD80 polypeptide could activate T cells, the producer HEK293T cells surface-expressing the anti-CD3 scFv polypeptide and the CD80 polypeptide were co-cultured with T cells and activation of the T cells was measured by detecting levels of CD25 and CD69 expression in the T cells using flow cytometry (FIG. 18).

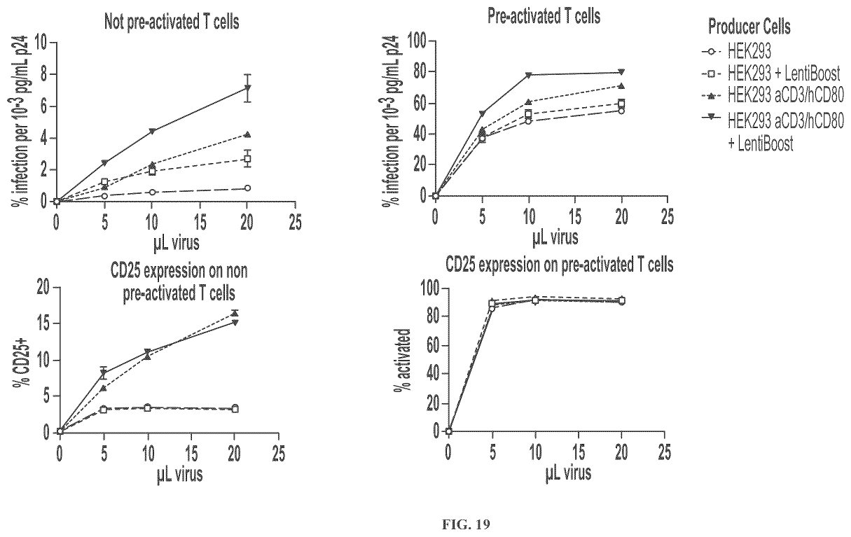

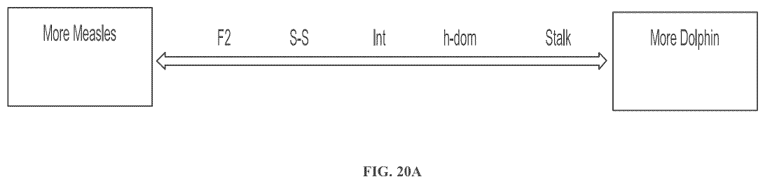

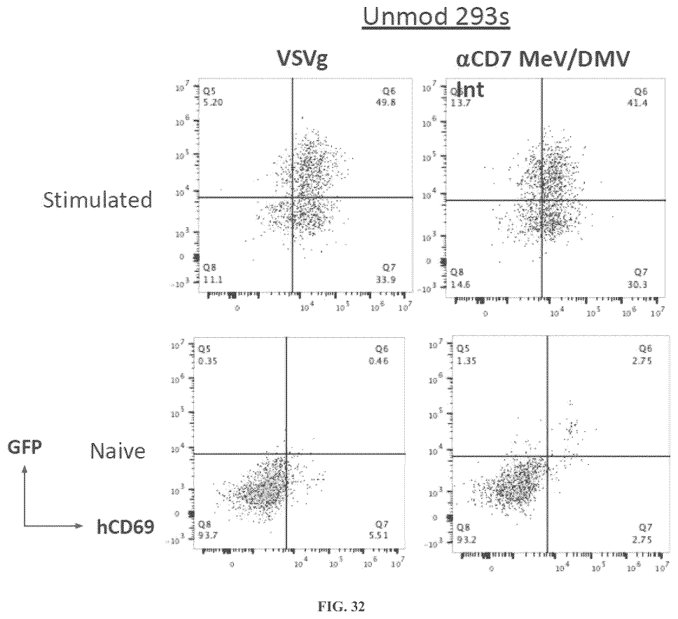

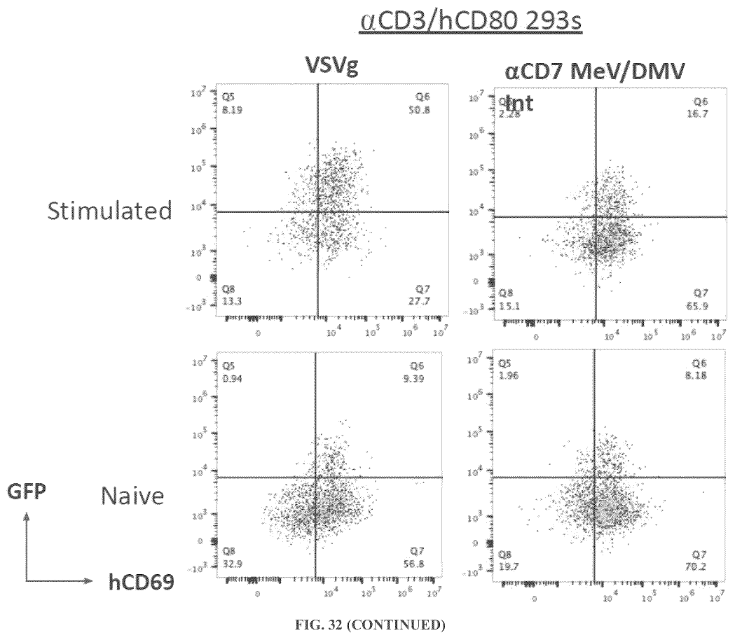

Having established that the combination of the anti-CD3 scFv and CD80 polypeptides was effective in activating T cells, VSVg-pseudotyped lentiviral particles were prepared displaying the two polypeptides to determine if the viral particles also would be effective in activating T cells. It was determined that the anti-CD3 scFv and CD80 polypeptide combination improved infection of unstimulated T cells with the VSVg-pseudotyped lentiviral particles (FIGS. 19 and 32).

Finally, lentiviral particles pseudotyped with an MeV-DMV H fusion protein fused to an anti-CD7 VHH domain and a MeV-DMV-F Int fusion protein (the “chimeric proteins”) and containing the anti-CD3 scFv and CD80 polypeptides in their envelope were prepared and their ability to infect producer HEK293T cells was evaluated in vitro. The lentiviral particles pseudotyped with the chimeric proteins, the anti-CD3 scFv polypeptide, and the CD80 polypeptide showed improved levels of infection of non-stimulated T cells (FIG. 32). Therefore, it was established that infection efficiencies of lentiviral particles pseudotyped with fusion polypeptides of the disclosure was improved by functionalizing the lentiviral particles by introducing to their envelopes the anti-CD3 scFv and CD80 polypeptides.

### Example 6: Infection of Cells Using Lentiviral Particles Containing Combinations of Chimeric and Non-Chimeric Envelope Glycoproteins

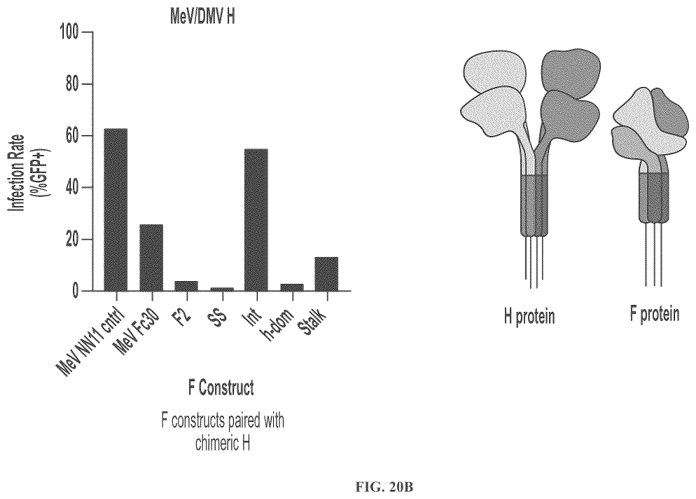

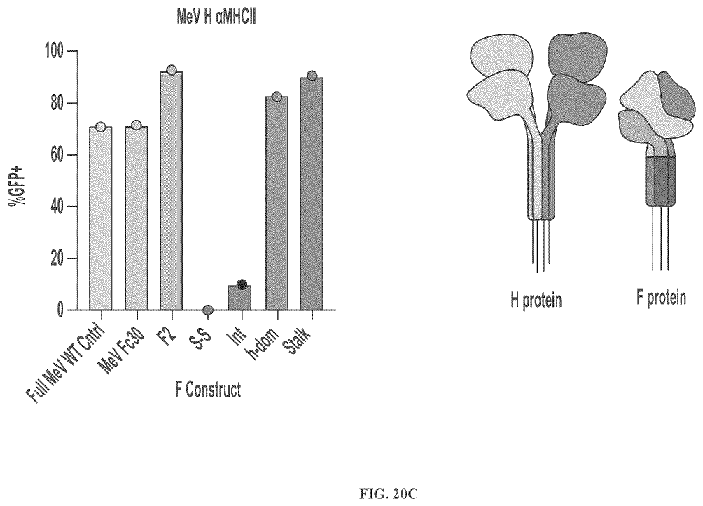

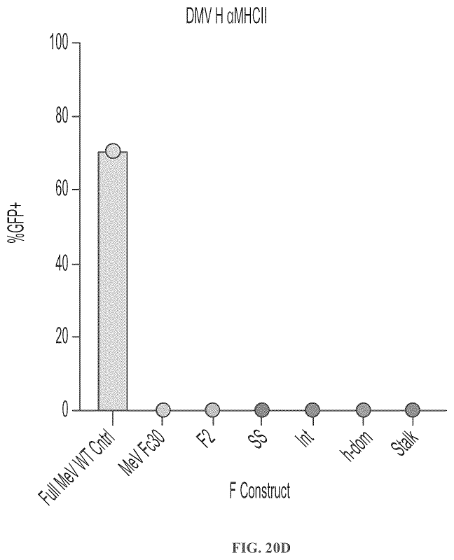

Experiments were undertaken to identify combinations of MeV-DMV-H and MeV-DMV-F envelope glycoprotein fusion (FIG. 20A). Lentiviral particles were pseudotyped with each of MeV-DMV H fused to an anti-MHCII VHH domain, MeV H fused to an anti-MHCII VHH domain, and DMV H fused to an anti-MHCII VHH domain combined with one of MeV-Fc30, MeV-DMV-F2, MeV-DMV-SS, MeV-DMV-F Int, MeV-DMV-F HBD (h-dom), and MeV-DMV-F Stalk. The lentiviral particles containing the different fusion protein combinations were used to infect A20 cells and infection efficiencies were measured (FIGS. 20B-20D). Each lentiviral particle contained encapsidated a polynucleotide encoding an enhanced green fluorescent protein (eGFP) allowing for infection efficiencies to be measured based upon levels of fluorescence in infected cells. It was determined that a number of the combinations had good infection efficiencies (e.g., MeV-DMV-H combined with either MeV-DMV-F int or MeV-DMV-F stalk).

### Example 7: Binding of Human Serum Antibodies to Chimeric and Non-Chimeric Envelope Glycoproteins

Experiments were undertaken to evaluate binding of anti-measles antibodies from the serum of human subjects to the envelope glycoprotein fusion polypeptides.

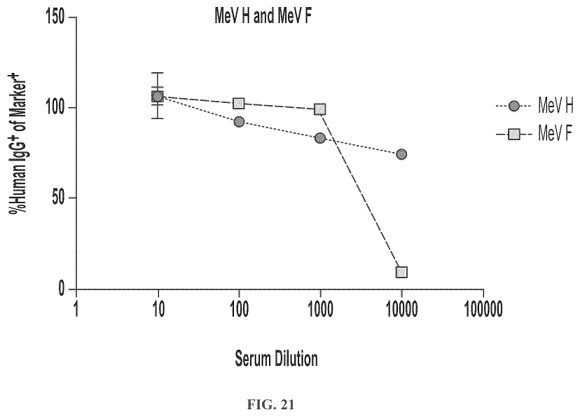

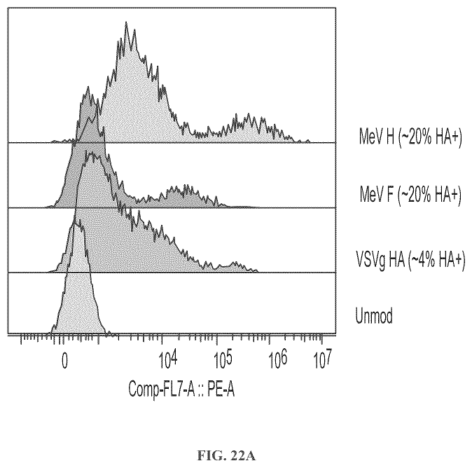

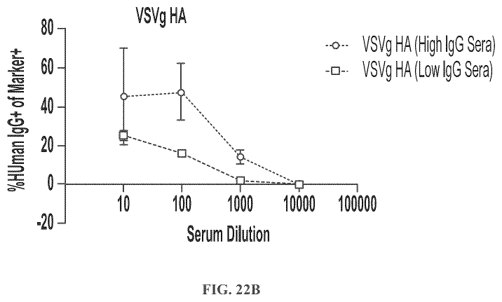

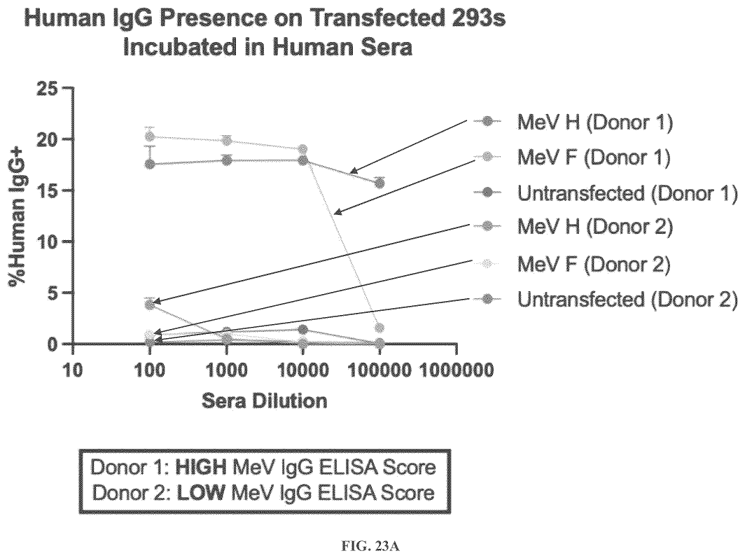

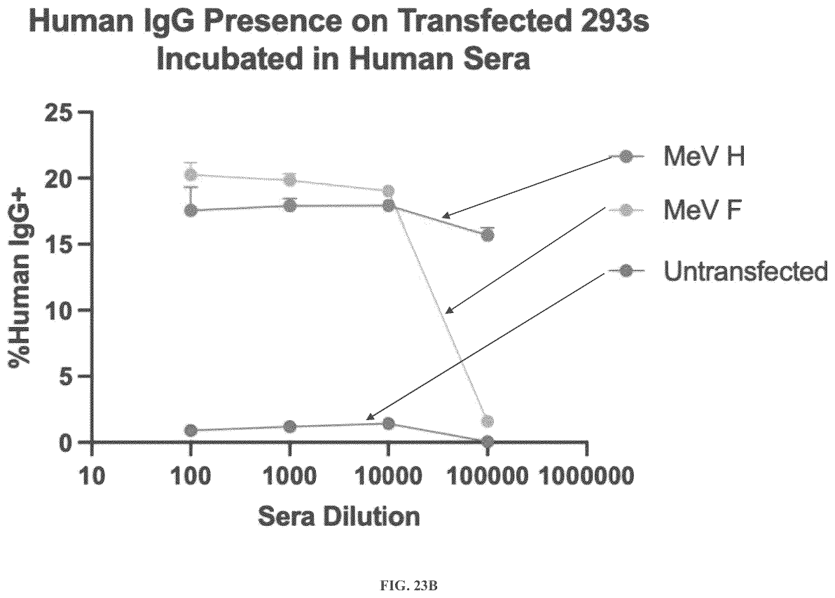

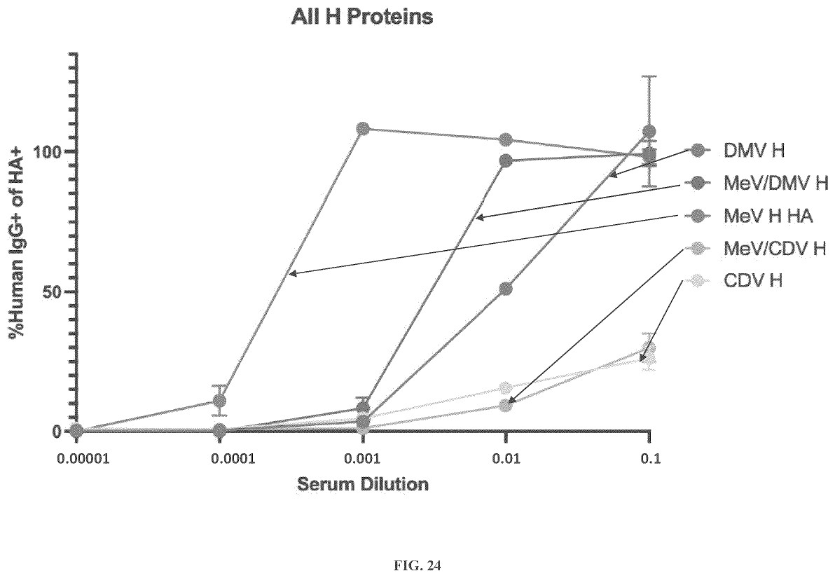

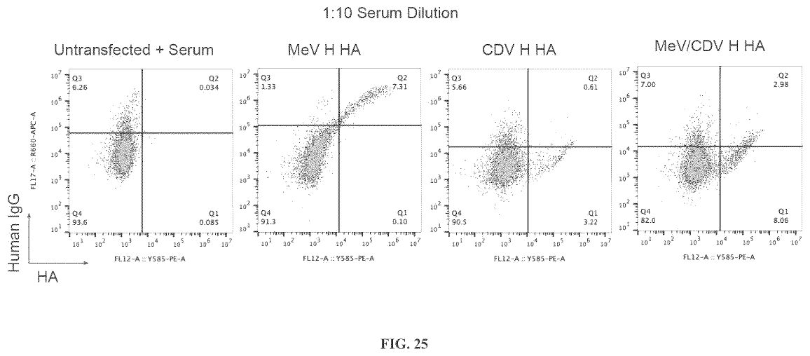

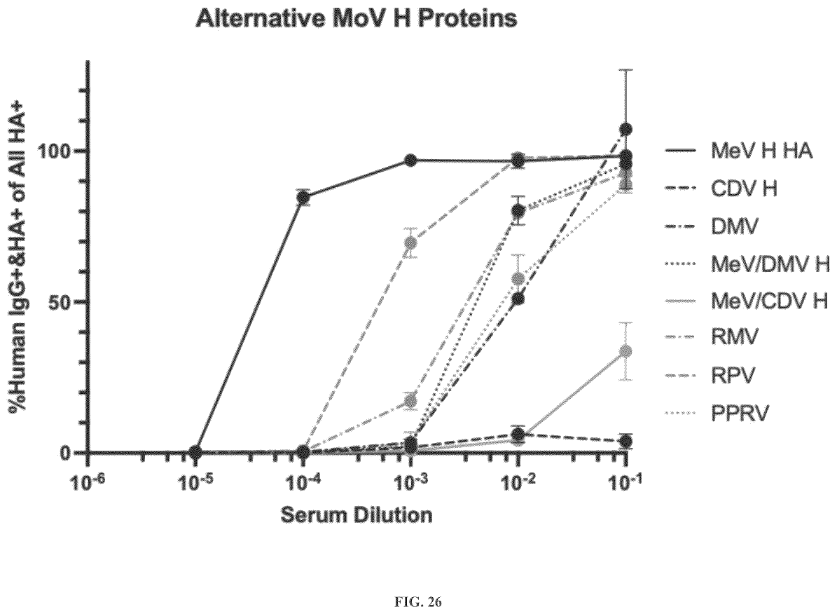

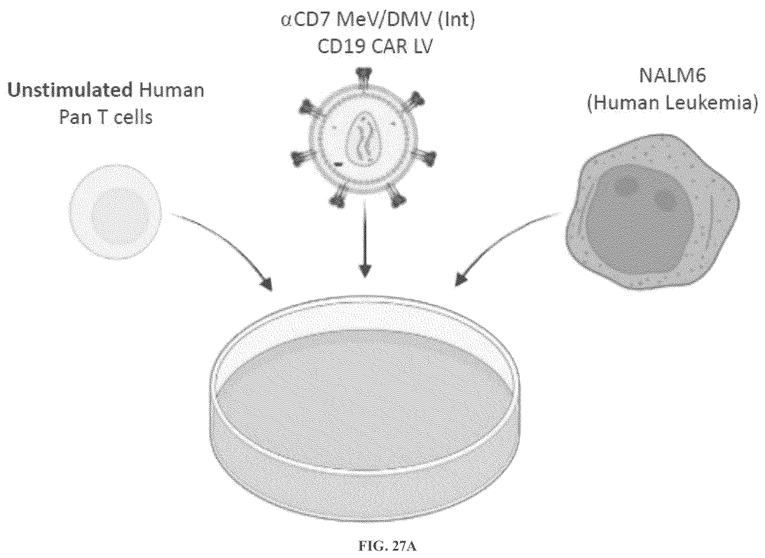

First, binding of anti-measles antibodies in human serum to producer HEK293T cells surface expressing MeV-H or MeV-F was evaluated. The anti-measles virus antibodies were produced in a subject in response to exposure to the measles virus or to a measles virus vaccine. Levels of anti-measles virus antibodies in the human serum were measured using an enzyme-linked immunosorbent assay. It was found that immunoglobulin G polypeptides from the human serum bound to each of the MeV-H and MeV-F proteins (FIGS. 21, 23A, and 23B). For comparison, it was determined that VSVg had poor surface expression in the HEK293T cells (FIG. 22A) and that anti-vesicular stomatitis virus antibodies present in human serum showed low levels of binding to HEK293T cells expressing VSVg (FIG. 22B). Levels of anti-vesicular stomatitis virus antibodies in the human serum were measured using an enzyme-linked immunosorbent assay. It was found that HEK293T cells surface expressing dolphin morbillivirus (DMV) H, MeV-DMV-H, MeV-canine distemper virus (CDV) H, or CDV-H proteins showed reduced levels of binding to anti-measles virus antibodies in human serum relative to HEK293T cells surface expressing MeV-H (FIGS. 24 and 25). In a similar experiment, it was also found that HEK293T cells surface expressing Rinderpest virus H protein (RPV), small ruminant virus H protein (RMV), and Peste de pestis ruminant virus H protein (PPRV) also showed reduced levels of binding to anti-measles virus antibodies in human serum relative to the HEK293T cells surface expressing MeV H (FIG. 26).

Therefore, envelope glycoproteins derived from DMV-H, CDV-H, RMV, RPV, or PPRV were shown to be suitable for preparation of pseudotyped lentiviral particles with reduced neutralization by anti-measles virus antibodies relative to lentiviral particles pseudotyped using MeV-H.

### Example 8: Elimination of Human Leukemia Cells from Mice Through the In Vivo Generation of Chimeric Antigen Receptor T Cells

Experiments were undertaken to evaluate whether lentiviral particles pseudotyped using the envelope glycoprotein fusion polypeptides were effective in preparing chimeric antigen receptor (CAR) T cells in vivo to treat a cancer. The lentiviral particles were pseudotyped using MeV-DMV-H and MeV-DMV-F Int and also contained an anti-CD3 scFv antigen binding polypeptide and a cluster of differentiation 80 (CD80) polypeptide in their viral envelopes for T cell activation. The lentiviral particles also encapsidated a polynucleotide encoding an anti-CD19 chimeric antigen receptor (CAR).

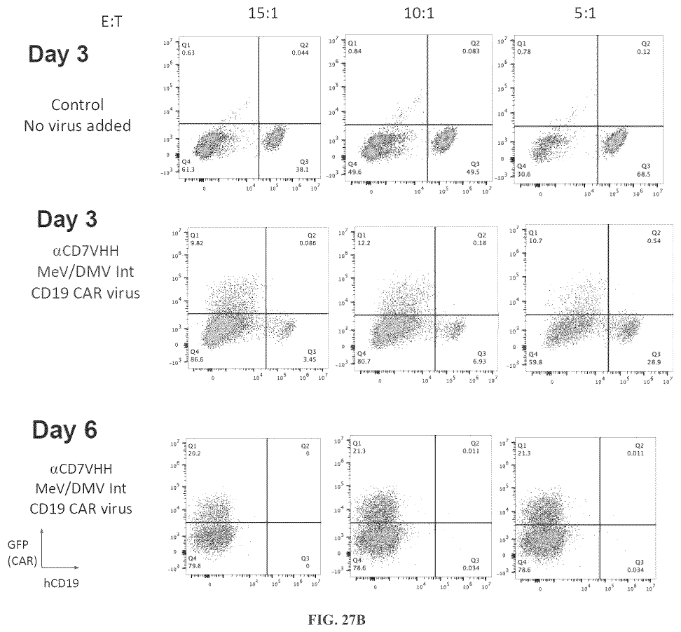

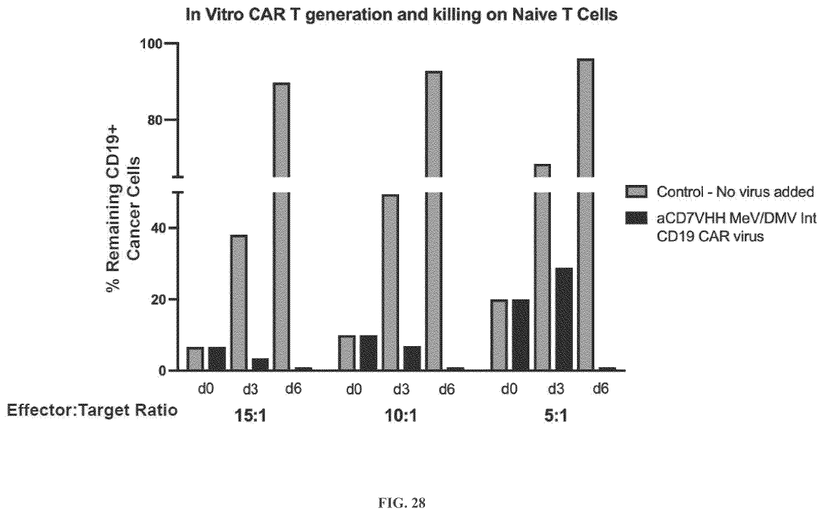

First, experiments were undertaken to evaluate whether infection of previously unstimulated pan T cells using the lentiviral particles, where the T cells were actively being co-cultured with NALM6 human leukemia cells, would lead to elimination of the NALM6 cells from the co-culture (FIG. 27A). It was determined that the unstimulated pan T cells infected with the viral particles while being grown in co-culture with the NALM6 cells lead to killing of nearly all of the NALM6 cells within about 6 days of infection (FIGS. 27B and 28). Therefore, it was established that the lentiviral particles were effective in delivering a polynucleotide encoding a CAR polypeptide to the T cells grown in co-culture with cancer cells.

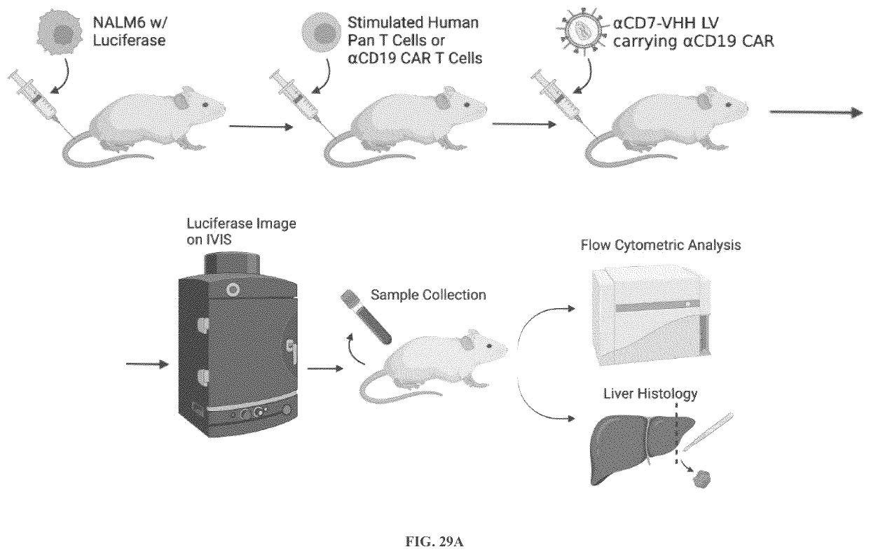

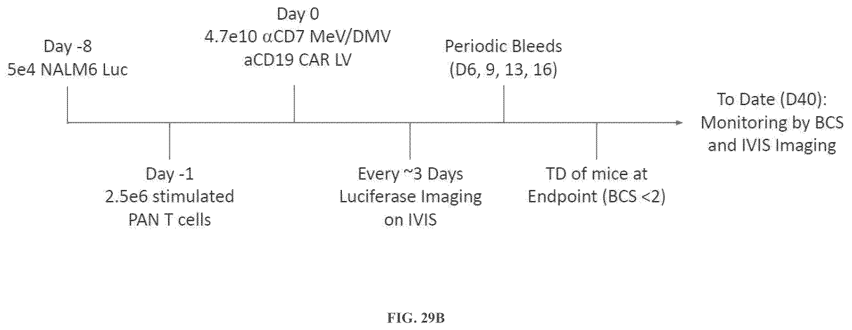

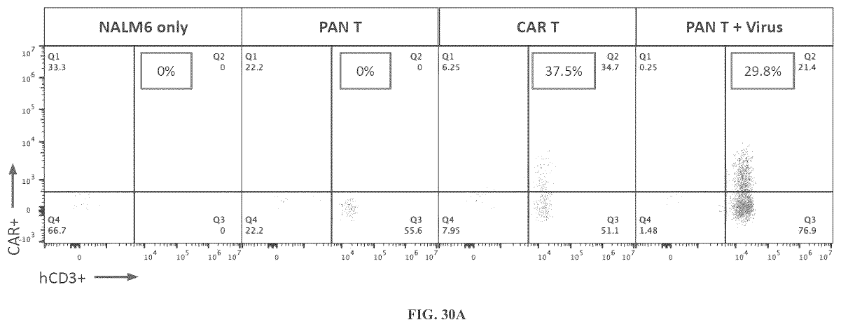

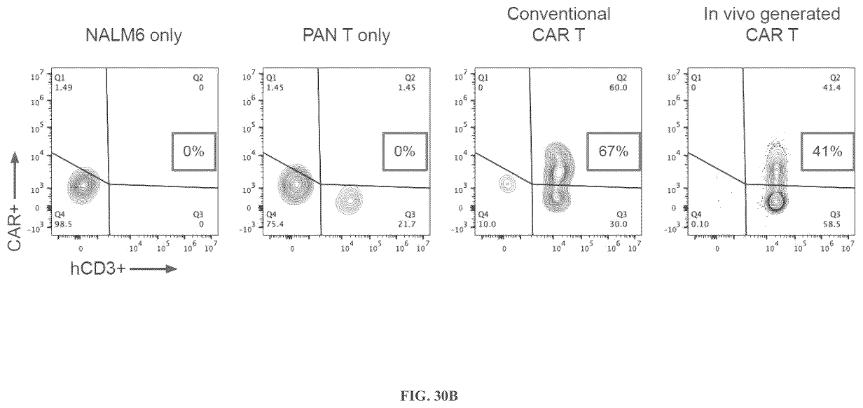

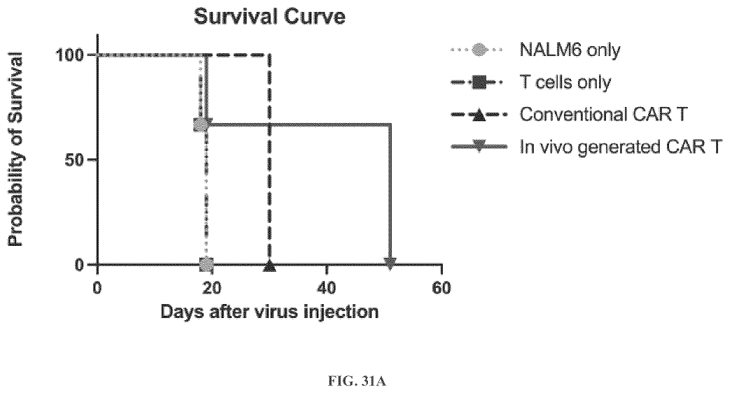

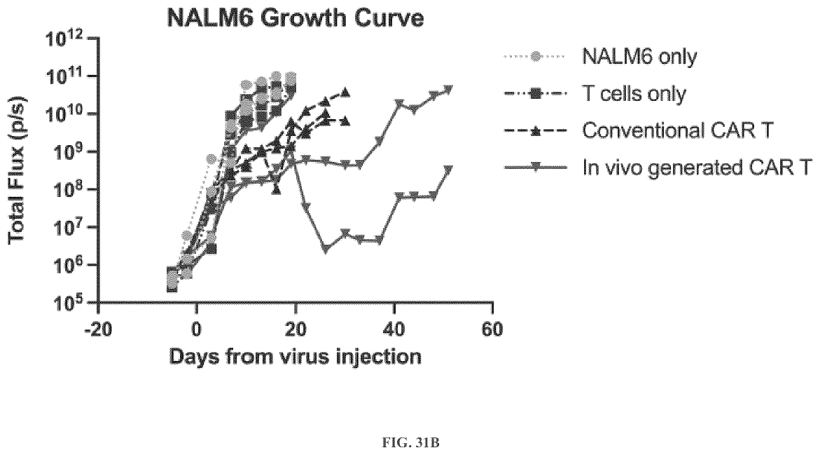

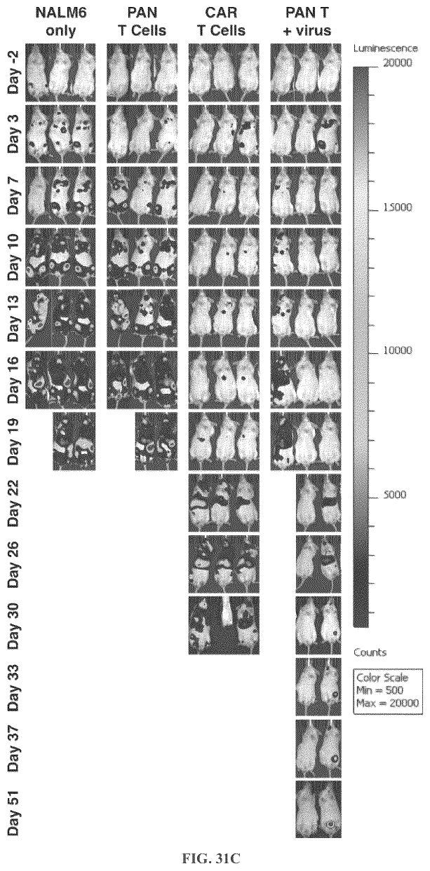

Having established the efficacy of the viral particles in infecting the pan T cells, experiments were undertaken to evaluate production of CAR T cells in vivo using the viral particles (FIGS. 29A and 29B). Eight (8) days prior to administration of the viral particles, NSG mice were administered 5e4 NALM6 cells expressing luciferase. One (1) day prior to administration of the viral particles, the mice were administered 2.5e6 pan T cells. Then, the mice were administered 4.7e10 viral particles or, as a control, anti-CD19 CAR T cells prepared ex vivo. Following infection, cancer growth was monitored over time (FIG. 29B). It was found that at day 6 (D6) following infection, the blood of the mice administered the virus particles contained a large population of infected T cells expressing the anti-CD19 chimeric antigen receptor (CAR) (FIG. 30A), and the population persisted through at least day 13 (FIG. 30B). This established that the viral particles were effective in mediating the preparation of CAR T cells in vivo. Further, it was found that the in vivo generated CAR T cells showed improved efficacy in eliminating NALM6 human leukemia cells from the mice as compared to the ex vivo prepared CAR T cells (FIGS. 31A-31C). So, not only were the viral particles effective in generating CAR T cells in vivo, but the generated CAR T cells were more effective in killing cancer cells and improving mouse survival than CAR T cells prepared ex vivo and administered to the mice.

The following methods were employed in the above examples.

Generation of retargetedMeV Envelope Proteins

Codon optimized polynucleotides encoding polypeptide sequences of interest were synthesized at GenScript. The polynucleotides were cloned into a pCG plasmid through either infusion cloning or NotI and SpeI RE sites.

### Surface Expression Assay

1E6 HEK293T cells were seeded in 6-well plates. 24-hours later, the media was changed with fresh pre-warmed complete DMEM (Dulbecco's modified eagle medium). 1 μg of envelope plasmid was diluted in 100 μL Opti-MEM (optimized minimal essential medium) and incubated with 5 uL PEI (polyethylenimine buffer) for 20 minutes at room temperature. The Opti-MEM, plasmid, PEI mixture was then added dropwise to the cells.

24-hours later the cells were collected via trypsinization and washed with MACS buffer (phosphate-buffered saline (PBS)+1% fetal bovine serum (FBS) 4 mM ethylenediamine tetraacetic acid (EDTA)). 1E6 cells were immunostained and analyzed on a CytoflexLX flow cytometer.

### Lentivirus Production

For lentivirus generation 18×10E6 HEK293T cells were seeded into a T175 flask with 25 mL of Dulbecco's Modified Eagle Medium (DMEM) (Supplemented with 10% fetal bovine serum (FBS) Pen/Strep and Genatmicin). 24-hours later, media was replaced with warm DMEM. For generation of re-targeted lentivirus about vectors encoding fusion proteins (e.g., DMV-Hwt-N11) and/or Morbillivirus envelope protein F proteins (e.g., DMV-Fwt), psPAX2 (Addgene), and a EFS-GFP transfer vector were diluted in Opti-MEM (optimized minimal essential medium) to which polyethylenimine (PEI) was added and incubated for 20 minutes at room temperature. The mixture was then added dropwise to the cells. 8-16 hours post-transduction, the media was replaced with fresh pre-warmed DMEM. 48-60 hours later the media was collected and filtered through 0.45 M surfactant-free cellulose acetate (SFCA) membrane to remove cell debris.

To concentrate the virus particles, lentivirus LentiX was added to virus-containing supernatant at 1 1:3 lentiX:supernatant ratio and incubated at 4 C for 24-72 hours then spun at 1500×g for 45 minutes and resuspended in PBS or HBSS. Lentivirus particles were also concentrated via ultrafugation at 72,000×g for 2 hours and resuspended in PBS (phosphate-buffered saline) or HBSS (Hank's balanced salt solution).

### In Vitro Virus Transduction

For transduction of cancer cell lines, a 96 well plate, 10E3 hNECTIN4 MC38 overexpression cells or A20s were seeded. 1-20 uL of 100×LentiX or Ultracentrifuge-concentrated GFP reporter virus was added per well. 2-3 days later cells were collected and washed with MACS buffer (phosphate-buffered saline (PBS)+1% fetal bovine serum (FBS) 4 mM ethylenediamine tetraacetic acid (EDTA)). Cells were stained with antibodies for the requisite targets (ex/hNECTIN4 for or mMHCII) and then analyzed for GFP expression by flow cytometry. GFP expression was measured every 2-3 days after to access signal stability.

For ex vivo transduction of primary splenocytes and T cells, spleens from 6-10 week old mice were excised and mechanically separated then filtered through 0.45 μm filters. Splenocytes were washed with PBS and then lysed with ACK (ammonium-chloride-potassium) buffer and a pan T cell or CD8 T cells tissue isolation kits (available from Miltenyi Biotech) were used to purify cell populations. Cells were then plated onto anti mCD3 coated 96 well plate with IL2 and anti-mCD28 antibody and stimulated for 2 days. Following stimulation 100K cells were plated into a 96 well plates with 1-20 uL of 100×virus and incubated for 2 days. Cells were stained for surface receptors and markers then analyzed with flow cytometry and analyzed every 2-3 days to determine signal stability.

### OTHER EMBODIMENTS

From the foregoing description, it will be apparent that variations and modifications may be made to the invention described herein to adapt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A pseudotyped viral particle comprising:
(a) an envelope comprising a viral envelope glycoprotein domain or fragment thereof fused to a VHH domain or fragment thereof, wherein the VHH domain or fragment thereof specifically binds an antigen present on a target cell, and wherein the viral envelope glycoprotein comprises an alteration referenced to a measles virus glycoprotein at an amino acid targeted by a measles virus neutralizing antibody; and
(b) a heterologous polynucleotide.

2. A method for delivering a heterologous polynucleotide to a target cell, the method comprising:
contacting a target cell with a pseudotyped viral particle comprising:
(a) an envelope comprising a viral envelope glycoprotein domain or fragment thereof fused to a VHH domain or fragment thereof, wherein the VHH domain or fragment thereof specifically binds an antigen present on the target cell, and wherein the viral envelope glycoprotein comprises an alteration referenced to a measles virus glycoprotein at an amino acid targeted by a measles virus neutralizing antibody; and
(b) a heterologous polynucleotide,
thereby delivering the heterologous polynucleotide to the target cell; or
(a) an envelope comprising a viral envelope glycoprotein domain or fragment thereof fused to a VHH domain or fragment thereof, wherein the VHH domain or fragment thereof specifically binds an antigen present on the target cell, and wherein the viral envelope glycoprotein comprises an alteration referenced to a measles virus glycoprotein at an amino acid targeted by a measles virus neutralizing antibody; and
(b) a heterologous polynucleotide,
thereby delivering the heterologous polynucleotide to the subject.

3. A method of treating a subject having a cancer, the method comprising administering to the subject a composition comprising a pseudotyped viral particle, the pseudotyped viral particle comprising:
(a) an envelope comprising a viral envelope glycoprotein domain or fragment thereof fused to a VHH domain or fragment thereof, wherein the VHH domain or fragment thereof specifically binds a tumor antigen present on a target cancer cell, and wherein the viral envelope glycoprotein comprises an alteration referenced to a measles virus glycoprotein at an amino acid targeted by a measles virus neutralizing antibody; and
(b) a heterologous polynucleotide,
thereby delivering the heterologous polynucleotide to the target cell in the subject and treating the subject.

5. The viral particle of claim 1, wherein the viral envelope glycoprotein domain or fragment thereof comprises a viral hemagglutinin domain or fragment thereof.

6. The viral particle of claim 1, wherein the virus is a Morbillivirus.

7. The viral particle of claim 6, wherein the Morbillivirus is selected from the group consisting of canine distemper virus, dolphin Morbillivirus, feline Morbillivirus, measles virus, Peste des petits ruminant virus, phocine Morbillivirus, Rinderpest virus, and small ruminant virus.

8. The viral particle of claim 1, wherein the viral envelope glycoprotein domain or fragment thereof comprises a stalk polypeptide sequence derived from a measles virus envelope glycoprotein domain and an extravirion domain derived from a non-measles virus envelope glycoprotein.

9. The viral particle of claim 1, wherein the stalk polypeptide comprises an amino acid sequence with at least about 85% sequence identity to the sequence
RLHRAAIYTAEIHKSLSTNLDVTNSIEHQVKDVLTPLFKIIGDEVGLRTP
QRFTDLVKFISDKIKFLNPDREYDERDLTWCINPPERIKLDYDQYCADVA
A.

10. The viral particle or method of claim 9, wherein the extravirion domain is derived from a dolphin Morbillivirus or a canine distemper virus.

11. The viral particle of claim 10, wherein the extravirion domain comprises an amino acid sequence with at least about 85% sequence identity to one of the following sequences:
Extravirion domain of CDV-H
RKAIASAANPILLSALSGGRGDIFPPHRCSGATTSVGKVFPLSVSLSMSL
ISRTSEVINMLTAISDGVYGKTYLLVPDDIEREFDTREIRVFEIGFIKRW
LNDMPLLQTTNYMVLPKNSKAKVCTIAVGELTLASLCVEESTVLLYHDSS
GSQDGILVVTLGIFWATPMDHIEEVIPVAHPSMKKIHITNHRGFIKDSIA
TWMVPALASEKQEEQKGCLESACQRKTYPMCNQASWEPFGGRQLPSYGRL
TLPLDASVDLQLNISFTYGPVILNGDGMDYYESPLLNSGWLTIPPKDGTI
SGLINKAGRGDQFTVLPHVLTFAPRESSGNCYLPIQTSQIRDRDVLIESN
IVVLPTQSIRYVIATYDISRSDHAIVYYVYDPIRTISYTHPFRLTTKGRP
DFLRIECFVWDDNLWCHQFYRFEADIANSTTSVENLVRIRFSCNR;
and
Extravirion domain of DMV-H
EELIVTKFKELMNHSLDMSKGRIFPPKNCSGSVITRGQTIKPGLTLVNIY
TTRNFEVSFMVTVISGGMYGKTYFLKPPEPDDPFEFQAFRIFEVGLVRDV
GSREPVLQMTNFMVIDEDEGLNFCLLSVGELRLAAVCVRGRPVVTKDIGG
YKDEPFKVVTLGIIGGGLSNQKTEIYPTIDSSIEKLYITSHRGIIRNSKA
RWSVPAIRSDDKDKMEKCTQALCKSRPPPSCNSSDWEPLTSNRIPAYAYI
ALEIKEDSGLELDITSNYGPLIIHGAGMDIYEGPSSNQDWLAIPPLSQSV
LGVINKVDFTAGFDIKPHTLTTAVDYESGKCYVPVELSGAKDQDLKLESN
LVVLPTKDFGYVTATYDTSRSEHAIVYYVYDTARSSSYFFPFRIKARGEP
IYLRIECFPWSRQLWCHHYCMINSTVSNEIVVVDNLVSINMSCSR.

12. The viral particle of claim 11, wherein the viral envelope glycoprotein domain or fragment thereof comprises an amino acid sequence with at least about 85% sequence identity to one of the following sequences, a fragment thereof, a cytoplasmic, transmembrane, stalk, or extravirion domain thereof, or to one of the following sequences comprising a truncated cytoplasmic domain:
DMV-H
MSSPRDKVDAFYKDIPRPRNNRVLLDNERVIIERPLILVGVLAVMFLSLVGLLAIAGVRLQKAT
TNSIEVNRKLSTNLETTVSIEHHVKDVLTPLFKIIGDEVGLRMPQKLTEIMQFISNKIKFLNPD
REYDFNDLHWCVNPPDQVKIDYAQYCNHIAAEELIVTKFKELMNHSLDMSKGRIFPPKNCSGSV
ITRGQTIKPGLTLVNIYTTRNFEVSFMVTVISGGMYGKTYFLKPPEPDDPFEFQAFRIFEVGLV
RDVGSREPVLQMTNFMVIDEDEGLNFCLLSVGELRLAAVCVRGRPVVTKDIGGYKDEPFKVVTL
GIIGGGLSNQKTEIYPTIDSSIEKLYITSHRGIIRNSKARWSVPAIRSDDKDKMEKCTQALCKS
RPPPSCNSSDWEPLTSNRIPAYAYIALEIKEDSGLELDITSNYGPLIIHGAGMDIYEGPSSNQD
WLAIPPLSQSVLGVINKVDFTAGFDIKPHTLTTAVDYESGKCYVPVELSGAKDQDLKLESNLVV
LPTKDFGYVTATYDTSRSEHAIVYYVYDTARSSSYFFPFRIKARGEPIYLRIECFPWSRQLWCH
HYCMINSTVSNEIVVVDNLVSINMSCSR;
CDV-H
MLPYQDKVGAFYKDNARANSTKLSLVTEGHGGRRPPYLLFVLLILLVGILALLAITGVRFHQVS
TSNMEFSRLLKEDMEKSEAVHHQVIDVLTPLFKIIGDEIGLRLPQKLNEIKQFILQKTNFFNPN
REFDFRDLHWCINPPSTVKVNFTNYCESIGIRKAIASAANPILLSALSGGRGDIFPPHRCSGAT
TSVGKVFPLSVSLSMSLISRTSEVINMLTAISDGVYGKTYLLVPDDIEREFDTREIRVFEIGFI
KRWLNDMPLLQTTNYMVLPKNSKAKVCTIAVGELTLASLCVEESTVLLYHDSSGSQDGILVVTL
GIFWATPMDHIEEVIPVAHPSMKKIHITNHRGFIKDSIATWMVPALASEKQEEQKGCLESACQR
KTYPMCNQASWEPFGGRQLPSYGRLTLPLDASVDLQLNISFTYGPVILNGDGMDYYESPLLNSG
WLTIPPKDGTISGLINKAGRGDQFTVLPHVLTFAPRESSGNCYLPIQTSQIRDRDVLIESNIVV
LPTQSIRYVIATYDISRSDHAIVYYVYDPIRTISYTHPFRLTTKGRPDFLRIECFVWDDNLWCH
QFYRFEADIANSTTSVENLVRIRFSCNR;
MeV-Hc18-CDV 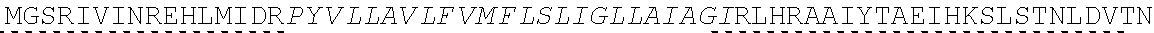 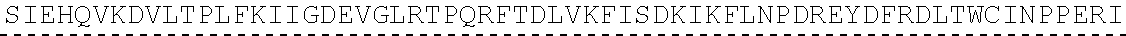 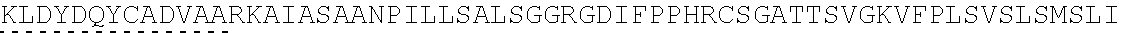
SRTSEVINMLTAISDGVYGKTYLLVPDDIEREFDTREIRVFEIGFIKRWLNDMPLLQTTNYMVL
PKNSKAKVCTIAVGELTLASLCVEESTVLLYHDSSGSQDGILVVTLGIFWATPMDHIEEVIPVA
HPSMKKIHITNHRGFIKDSIATWMVPALASEKQEEQKGCLESACORKTYPMCNQASWEPFGGRQ
LPSYGRLTLPLDASVDLQLNISFTYGPVILNGDGMDYYESPLLNSGWLTIPPKDGTISGLINKA
GRGDQFTVLPHVLTFAPRESSGNCYLPIQTSQIRDRDVLIESNIVVLPTQSIRYVIATYDISRS
DHAIVYYVYDPIRTISYTHPFRLTTKGRPDFLRIECFVWDDNLWCHQFYRFEADIANSTTSVEN
LVRIRFSCNR;
MeV-Hc18-DMV 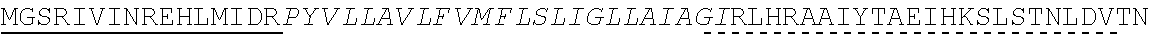 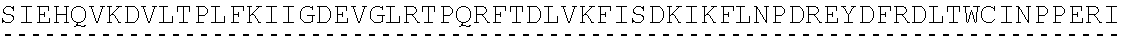 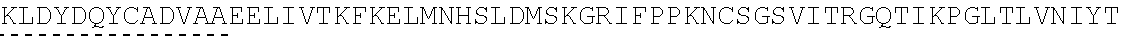
TRNFEVSFMVTVISGGMYGKTYFLKPPEPDDPFEFQAFRIFEVGLVRDVGSREPVLQMTNFMVI
DEDEGLNFCLLSVGELRLAAVCVRGRPVVTKDIGGYKDEPFKVVTLGIIGGGLSNQKTEIYPTI
DSSIEKLYITSHRGIIRNSKARWSVPAIRSDDKDKMEKCTQALCKSRPPPSCNSSDWEPLTSNR
IPAYAYIALEIKEDSGLELDITSNYGPLIIHGAGMDIYEGPSSNQDWLAIPPLSQSVLGVINKV
DFTAGFDIKPHTLTTAVDYESGKCYVPVELSGAKDQDLKLESNLVVLPTKDFGYVTATYDTSRS
EHAIVYYVYDTARSSSYFFPFRIKARGEPIYLRIECFPWSRQLWCHHYCMINSTVSNEIVVVDN
LVSINMSCSR;
FMV-H
MESNNIKYYKDSSRYFGKILDEHKTINSQLYSLSIKVITIIAIIVSLIATIITIINATSGRTTL
NSNTDILLSQRDEIHNIQEMIFDRIYPLINAMSTELGLHIPTLLDELTKAIDQKIKIMHPPVDT
VTSDLNWCIKPPNGIIIDPKSYCESMELSKTYELLLDQLDVSRKKSLIINRKNINQCQLVDNSK
IIFATVNIQSTPRFLNFGHTVSNQRITFGQGTYSSTYVITIQEDGVTDVQYRVFEIGYISDQFG
VFPSLIVSRVLPIRMLLGMESCTLTSDRLGGYFLCMNTLTRSIYDYVSIRDLKSLYITIPHYGK
VNYTYFNFGKIRSPHEIDKIWLTSDRGQIISGYFAAFVTITIRNYNNYPYKCLNNPCFDNSENY
CRGWYKNITGTDDVPILAYLLVEMYDEEGPLITLVAIPPYNYTAPSHNSLYYDDKINKLIMTTS
HIGYIQINEVHEVIVGDNLKAILLNRLSDEHPNLTACRLNQGIKEQYKSDGTIISNSALIDIQE
RMYITVKAIPPAGNYNFTVELHSRSNTSYVSLPKQFNAKYDKLHLECFSWDKSWWCALIPQFSL
SWNESLSVDTAIFNLISCK;
PPRV-H
MSAQRERINAFYKDNLHNKTHRVILDRERLTIERPYILLGVLLVMFLSLIGLLAIAGIRLHRAT
VGTAEIQSRLNTNIELTESIDHQTKDVLTPLFKIIGDEVGIRIPQKFSDLVKFISDKIKFLNPD
REYDFRDLRWCMNPPERVKINFDQFCEYKAAVKSVEHIFESSLNRSERLRLLTLGPGTGCLGRT
VTRAQFSELTLTLMDLDLEIKHNVSSVFTVVEEGLFGRTYTVWRSDTGKPSTSPGIGHFLRVFE
IGLVRDLELGAPIFHMTNYLTVNMSDDYRSCLLAVGELKLTALCTPSETVTLSESGVPKREPLV
VVILNLAGPTLGGELYSVLPTTDPTVEKLYLSSHRGIIKDNEANWVVPSTDVRDLQNKGECLVE
ACKTRPPSFCNGTGIGPWSEGRIPAYGVIRVSLDLASDPGVVITSVFGPLIPHLSGMDLYNNPF
SRAAWLAVPPYEQSFLGMINTIGFPDRAEVMPHILTTEIRGPRGRCHVPIELSSRIDDDIKIGS
NMVVLPTKDLRYITATYDVSRSEHAIVYYIYDTGRSSSYFYPVRLNFRGNPLSLRIECFPWYHK
VWCYHDCLIYNTITNEEVHTRGLTGIEVTCNPV;
RPV-H
MSPPRDRVDAYYKDNFQFKNTRVVLNKEQLLIERPCMLLTVLFVMFLSLVGLLAIAGIRLHRAA
VNTAKINNDLTTSIDITKSIEYQVKDVLTPLFKIIGDEVGLRTPQRFTDLTKFISDKIKFLNPD
KEYDFRDINWCINPPERIKIDYDQYCAHTAAEDLITMLVNSSLTGTTVLRTSLVNLGRNCTGPT
TTKGQFSNISLTLSGIYSGRGYNISSMITITGKGMYGSTYLVGKYNQRARRPSIVWQQDYRVFE
VGIIRELGVGTPVFHMTNYLELPRQPELETCMLALGESKLAALCLADSPVALHYGRVGDDNKIR
FVKLGVWASPADRDTLATLTHEPTLDGLYITTHRGIIAAGTAIWAVPVTRTDDQVKMGKCRLEA
CRDRPPPFCNSTDWEPLEAGRIPAYGVLTIKLGLADEPKVDIISEFGPLITHDSGMDLYTSFDG
TKYWLTTPPLONSALGTVNTLVLEPSLKISPNILTLPIRSGGGDCYTPTYLSDRADDDVKLSSN
LVILPSRDLQYVSATYDISRVEHAIVYHIYSTGRLSSYYYPFKLPIKGDPVSLQIECFPWDRKL
WCHHFCSVIDSGTGEQVTHIGVVGIEITCNGK; and
RMV-H
MSAQRERINAFYKDNPHNKNHRVILDRERLVIERPYILLGVLLVMFLSLIGLLAIAGIRLHRAT
VGTSEIQSRLNTNIELTESIDHQTKDVLTPLFKIIGDEVGIRIPQKFSDLVKFISDKIKFLNPD
REYDFRDLRWCMNPPERVKINFDQFCEYKAAVKSIEHIFESPLNKSKKLQSLTLGPGTGCLGRT
VTRAHFSELTLTLMDLDLEMKHNVSSVFTVVEEGLFGRTYTVWRSDARDPSTDLGIGHFLRVFE
IGLVRDLGLGPPVFHMTNYLTVNMSDDYRRCLLAVGELKLTALCSSSETVTLGERGVPKREPLV
VVILNLAGPTLGGELYSVLPTSDLMVEKLYLSSHRGIIKDDEANWVVPSTDVRDLQNKGECLVE
ACKTRPPSFCNGTGSGPWSEGRIPAYGVIRVSLDLASDPGVVITSVFGPLIPHLSGMDLYNNPF
SRAVWLAVPPYEQSFLGMINTIGFPNRAEVMPHILTTEIRGPRGRCHVPIELSRRVDDDIKIGS
NMVILPTIDLRYITATYDVSRSEHAIVYYIYDTGRSSSYFYPVRLNFKGNPLSLRIECFPWRHK
VWCYHDCLIYNTITDEEVHTRGLTGIEVTCNPV,
wherein cytoplasmic domains are denoted by underlined text, transmembrane domains are denoted by italicized text, stalks are denoted by text underlined with a dashed line, and extravirion domains are denoted by plain text.

13. A method for generating a pseudotyped viral particle for delivering a heterologous polynucleotide to a target cell, the method comprising:
(a) displaying on the cell membrane of a eukaryotic cell a viral envelope glycoprotein domain or fragment thereof fused to a VHH domain or fragment thereof, wherein the VHH domain or fragment thereof specifically binds an antigen present on the target cell, and wherein the viral envelope glycoprotein comprises an alteration referenced to a measles virus glycoprotein at an amino acid targeted by a measles virus neutralizing antibody;
(b) transfecting the eukaryotic cell with a viral transfer vector and one or more additional vectors encoding one or more viral polypeptides,
thereby generating the pseudotyped viral particle for delivering a heterologous polynucleotide to the target cell.

14. A eukaryotic cell for generating a pseudotyped viral particle, the eukaryotic cell comprising:
(a) a cell membrane comprising a viral envelope glycoprotein domain or fragment thereof fused to a VHH domain or fragment thereof, wherein the VHH domain or fragment thereof specifically binds an antigen present on a target cell, and wherein the viral envelope glycoprotein comprises an alteration referenced to a measles virus glycoprotein at an amino acid targeted by a measles virus neutralizing antibody;
(b) a viral transfer vector; and
(c) one or more additional vectors encoding one or more viral polypeptides.

15. A mammalian expression vector comprising a polynucleotide encoding a polypeptide comprising a viral envelope glycoprotein domain or fragment thereof fused to a VHH domain or fragment thereof, wherein the VHH domain or fragment thereof specifically binds an antigen present on a target cell, and wherein the viral envelope glycoprotein comprises an alteration referenced to a measles virus glycoprotein at an amino acid targeted by a measles virus neutralizing antibody.

16. A pharmaceutical composition comprising the pseudotyped viral particle of claim 1, and a pharmaceutically acceptable excipient.

17. A kit comprising the pseudotyped viral particle of claim 1 or a polynucleotide encoding said particle, and instructions for the use of the kit.

18. A fusion protein suitable for pseudotyping a viral particle, wherein the fusion protein comprises a sequence with at least 85% sequence identity to a sequence selected from the group consisting of
DMV-H-MHCII (N11)
MSSPRDKVDAFYKDIPRPRNNRVLLDNERVIIERPLILVGVLAVMFLSLVGLLAIAGVRLQKAT
TNSIEVNRKLSTNLETTVSIEHHVKDVLTPLFKIIGDEVGLRMPQKLTEIMQFISNKIKFLNPD
REYDFNDLHWCVNPPDQVKIDYAQYCNHIAAEELIVTKFKELMNHSLDMSKGRIFPPKNCSGSV
ITRGQTIKPGLTLVNIYTTRNFEVSFMVTVISGGMYGKTYFLKPPEPDDPFEFQAFRIFEVGLV
RDVGSREPVLQMTNFMVIDEDEGLNFCLLSVGELRLAAVCVRGRPVVTKDIGGYKDEPFKVVTL
GIIGGGLSNQKTEIYPTIDSSIEKLYITSHRGIIRNSKARWSVPAIRSDDKDKMEKCTQALCKS
RPPPSCNSSDWEPLTSNRIPAYAYIALEIKEDSGLELDITSNYGPLIIHGAGMDIYEGPSSNQD
WLAIPPLSQSVLGVINKVDFTAGFDIKPHTLTTAVDYESGKCYVPVELSGAKDQDLKLESNLVV
LPTKDFGYVTATYDTSRSEHAIVYYVYDTARSSSYFFPFRIKARGEPIYLRIECFPWSRQLWCH
HYCMINSTVSNEIVVVDNLVSINMSCSRGGGGSGGGGSGGGGSAAAQVQLVQSGGGLVQPGGSL
GLSCAASGNIGSRDNMGWYRQAPGKQREWVATISGYGIATYRDSVKGRFTVAKDTAKNIVSLQM
NYLTTEDTAVYYCYAYAVDSRNIFWSQGTQVTVS;
DMV-H-CD7 (Humanized VHH10)
MSSPRDKVDAFYKDIPRPRNNRVLLDNERVIIERPLILVGVLAVMFLSLVGLLAIAGVRLQKAT
TNSIEVNRKLSTNLETTVSIEHHVKDVLTPLFKIIGDEVGLRMPQKLTEIMQFISNKIKFLNPD
REYDFNDLHWCVNPPDQVKIDYAQYCNHIAAEELIVTKFKELMNHSLDMSKGRIFPPKNCSGSV
ITRGQTIKPGLTLVNIYTTRNFEVSFMVTVISGGMYGKTYFLKPPEPDDPFEFQAFRIFEVGLV
RDVGSREPVLQMTNFMVIDEDEGLNFCLLSVGELRLAAVCVRGRPVVTKDIGGYKDEPFKVVTL
GIIGGGLSNQKTEIYPTIDSSIEKLYITSHRGIIRNSKARWSVPAIRSDDKDKMEKCTQALCKS
RPPPSCNSSDWEPLTSNRIPAYAYIALEIKEDSGLELDITSNYGPLIIHGAGMDIYEGPSSNQD
WLAIPPLSQSVLGVINKVDFTAGFDIKPHTLTTAVDYESGKCYVPVELSGAKDQDLKLESNLVV
LPTKDFGYVTATYDTSRSEHAIVYYVYDTARSSSYFFPFRIKARGEPIYLRIECFPWSRQLWCH
HYCMINSTVSNEIVVVDNLVSINMSCSRGGGGSGGGGSGGGGSAAADVQLQESGGGSVQAGGSL
RLSCAASGYTHSSYCMAWFRQAPGREREGVASIDSDGTTSYADSVKGRFTISQDNAKNTLYLQM
NSLKPEDTAMYYCAARFGPMGCVDLSTLSFGHWGQGTQVTVSITGGGSGGGSYPYDVPDYA;
DMV-H-CD45 (32)
MSSPRDKVDAFYKDIPRPRNNRVLLDNERVIIERPLILVGVLAVMFLSLVGLLAIAGVRLQKAT
TNSIEVNRKLSTNLETTVSIEHHVKDVLTPLFKIIGDEVGLRMPQKLTEIMQFISNKIKFLNPD
REYDFNDLHWCVNPPDQVKIDYAQYCNHIAAEELIVTKFKELMNHSLDMSKGRIFPPKNCSGSV
ITRGQTIKPGLTLVNIYTTRNFEVSFMVTVISGGMYGKTYFLKPPEPDDPFEFQAFRIFEVGLV
RDVGSREPVLQMTNFMVIDEDEGLNFCLLSVGELRLAAVCVRGRPVVTKDIGGYKDEPFKVVTL
GIIGGGLSNQKTEIYPTIDSSIEKLYITSHRGIIRNSKARWSVPAIRSDDKDKMEKCTQALCKS
RPPPSCNSSDWEPLTSNRIPAYAYIALEIKEDSGLELDITSNYGPLIIHGAGMDIYEGPSSNQD
WLAIPPLSQSVLGVINKVDFTAGFDIKPHTLTTAVDYESGKCYVPVELSGAKDQDLKLESNLVV
LPTKDFGYVTATYDTSRSEHAIVYYVYDTARSSSYFFPFRIKARGEPIYLRIECFPWSRQLWCH
HYCMINSTVSNEIVVVDNLVSINMSCSRGGGGSGGGGSGGGGSAAAQVQLVQSGGGLVQPGGSL
RLSCAASGRAFNSAAMGWYRQAPGSQRELVASISAGTASYADAVKGRFTISRDYAKNIIYLQMN
SLKPDDTAVYFCNYRTTYTSGYSEDYWGQGTQVTVSGGGSGGGSYPYDVPDYA;
CDV-H-MHCII (N11)
MLPYQDKVGAFYKDNARANSTKLSLVTEGHGGRRPPYLLFVLLILLVGILALLAITGVRFHQVS
TSNMEFSRLLKEDMEKSEAVHHQVIDVLTPLFKIIGDEIGLRLPQKLNEIKQFILQKTNFFNPN
REFDFRDLHWCINPPSTVKVNFTNYCESIGIRKAIASAANPILLSALSGGRGDIFPPHRCSGAT
TSVGKVFPLSVSLSMSLISRTSEVINMLTAISDGVYGKTYLLVPDDIEREFDTREIRVFEIGFI
KRWLNDMPLLQTTNYMVLPKNSKAKVCTIAVGELTLASLCVEESTVLLYHDSSGSQDGILVVTL
GIFWATPMDHIEEVIPVAHPSMKKIHITNHRGFIKDSIATWMVPALASEKQEEQKGCLESACQR
KTYPMCNQASWEPFGGRQLPSYGRLTLPLDASVDLQLNISFTYGPVILNGDGMDYYESPLLNSG
WLTIPPKDGTISGLINKAGRGDQFTVLPHVLTFAPRESSGNCYLPIQTSQIRDRDVLIESNIVV
LPTQSIRYVIATYDISRSDHAIVYYVYDPIRTISYTHPFRLTTKGRPDFLRIECFVWDDNLWCH
QFYRFEADIANSTTSVENLVRIRFSCNRGGGGSGGGGSGGGGSAAAQVQLVQSGGGLVQPGGSL
GLSCAASGNIGSRDNMGWYRQAPGKQREWVATISGYGIATYRDSVKGRFTVAKDTAKNIVSLQM
NYLTTEDTAVYYCYAYAVDSRNIFWSQGTQVTVSGGGSGGGSYPYDVPDYA;
CDV-H-CD7 (Humanized VHH10)
MLPYQDKVGAFYKDNARANSTKLSLVTEGHGGRRPPYLLFVLLILLVGILALLAITGVRFHQVS
TSNMEFSRLLKEDMEKSEAVHHQVIDVLTPLFKIIGDEIGLRLPQKLNEIKQFILQKTNFFNPN
REFDFRDLHWCINPPSTVKVNFTNYCESIGIRKAIASAANPILLSALSGGRGDIFPPHRCSGAT
TSVGKVFPLSVSLSMSLISRTSEVINMLTAISDGVYGKTYLLVPDDIEREFDTREIRVFEIGFI
KRWLNDMPLLQTTNYMVLPKNSKAKVCTIAVGELTLASLCVEESTVLLYHDSSGSQDGILVVTL
GIFWATPMDHIEEVIPVAHPSMKKIHITNHRGFIKDSIATWMVPALASEKQEEQKGCLESACQR
KTYPMCNQASWEPFGGRQLPSYGRLTLPLDASVDLQLNISFTYGPVILNGDGMDYYESPLLNSG
WLTIPPKDGTISGLINKAGRGDQFTVLPHVLTFAPRESSGNCYLPIQTSQIRDRDVLIESNIVV
LPTQSIRYVIATYDISRSDHAIVYYVYDPIRTISYTHPFRLTTKGRPDFLRIECFVWDDNLWCH
QFYRFEADIANSTTSVENLVRIRFSCNRGGGGSGGGGSGGGGSAAADVQLQESGGGSVQAGGSL
RLSCAASGYTHSSYCMAWFRQAPGREREGVASIDSDGTTSYADSVKGRFTISQDNAKNTLYLQM
NSLKPEDTAMYYCAARFGPMGCVDLSTLSFGHWGQGTQVTVSITGGGSGGGSYPYDVPDYA;
CDV-H-CD45 VHH (32)
MLPYQDKVGAFYKDNARANSTKLSLVTEGHGGRRPPYLLFVLLILLVGILALLAITGVRFHQVS
TSNMEFSRLLKEDMEKSEAVHHQVIDVLTPLFKIIGDEIGLRLPQKLNEIKQFILQKTNFFNPN
REFDFRDLHWCINPPSTVKVNFTNYCESIGIRKAIASAANPILLSALSGGRGDIFPPHRCSGAT
TSVGKVFPLSVSLSMSLISRTSEVINMLTAISDGVYGKTYLLVPDDIEREFDTREIRVFEIGFI
KRWLNDMPLLQTTNYMVLPKNSKAKVCTIAVGELTLASLCVEESTVLLYHDSSGSQDGILVVTL
GIFWATPMDHIEEVIPVAHPSMKKIHITNHRGFIKDSIATWMVPALASEKQEEQKGCLESACQR
KTYPMCNQASWEPFGGRQLPSYGRLTLPLDASVDLQLNISFTYGPVILNGDGMDYYESPLLNSG
WLTIPPKDGTISGLINKAGRGDQFTVLPHVLTFAPRESSGNCYLPIQTSQIRDRDVLIESNIVV
LPTQSIRYVIATYDISRSDHAIVYYVYDPIRTISYTHPFRLTTKGRPDFLRIECFVWDDNLWCH
QFYRFEADIANSTTSVENLVRIRFSCNRGGGGSGGGGSGGGGSAAAQVQLVQSGGGLVQPGGSL
RLSCAASGRAFNSAAMGWYRQAPGSQRELVASISAGTASYADAVKGRFTISRDYAKNIIYLQMN
SLKPDDTAVYFCNYRTTYTSGYSEDYWGQGTQVTVSAAAYPYDVPDYA;
MeV-Hc18-CDV-MHCII (N11)
MGSRIVINREHLMIDRPYVLLAVLFVMFLSLIGLLAIAGIRLHRAAIYTAEIHKSLSTNLDVTN
SIEHQVKDVLTPLFKIIGDEVGLRTPQRFTDLVKFISDKIKFLNPDREYDFRDLTWCINPPERI
KLDYDQYCADVAARKAIASAANPILLSALSGGRGDIFPPHRCSGATTSVGKVFPLSVSLSMSLI
SRTSEVINMLTAISDGVYGKTYLLVPDDIEREFDTREIRVFEIGFIKRWLNDMPLLQTTNYMVL
PKNSKAKVCTIAVGELTLASLCVEESTVLLYHDSSGSQDGILVVTLGIFWATPMDHIEEVIPVA
HPSMKKIHITNHRGFIKDSIATWMVPALASEKQEEQKGCLESACQRKTYPMCNQASWEPFGGRQ
LPSYGRLTLPLDASVDLQLNISFTYGPVILNGDGMDYYESPLLNSGWLTIPPKDGTISGLINKA
GRGDQFTVLPHVLTFAPRESSGNCYLPIQTSQIRDRDVLIESNIVVLPTQSIRYVIATYDISRS
DHAIVYYVYDPIRTISYTHPFRLTTKGRPDFLRIECFVWDDNLWCHQFYRFEADIANSTTSVEN
LVRIRFSCNRGGGGSGGGGSGGGGSAAAQVQLVQSGGGLVQPGGSLGLSCAASGNIGSRDNMGW
YRQAPGKQREWVATISGYGIATYRDSVKGRFTVAKDTAKNIVSLQMNYLTTEDTAVYYCYAYAV
DSRNIFWSQGTQVTVSGGGSGGGSYPYDVPDYA;
MeV-Hc18-DMV-MHCII (N11)
MGSRIVINREHLMIDRPYVLLAVLFVMFLSLIGLLAIAGIRLHRAAIYTAEIHKSLSTNLDVTN
SIEHQVKDVLTPLFKIIGDEVGLRTPQRFTDLVKFISDKIKFLNPDREYDERDLTWCINPPERI
KLDYDQYCADVAAEELIVTKFKELMNHSLDMSKGRIFPPKNCSGSVITRGQTIKPGLTLVNIYT
TRNFEVSFMVTVISGGMYGKTYFLKPPEPDDPFEFQAFRIFEVGLVRDVGSREPVLQMTNFMVI
DEDEGLNFCLLSVGELRLAAVCVRGRPVVTKDIGGYKDEPFKVVTLGIIGGGLSNQKTEIYPTI
DSSIEKLYITSHRGIIRNSKARWSVPAIRSDDKDKMEKCTQALCKSRPPPSCNSSDWEPLTSNR
IPAYAYIALEIKEDSGLELDITSNYGPLIIHGAGMDIYEGPSSNQDWLAIPPLSQSVLGVINKV
DFTAGFDIKPHTLTTAVDYESGKCYVPVELSGAKDQDLKLESNLVVLPTKDFGYVTATYDTSRS
EHAIVYYVYDTARSSSYFFPFRIKARGEPIYLRIECFPWSRQLWCHHYCMINSTVSNEIVVVDN
LVSINMSCSRGGGGSGGGGSGGGGSAAAQVQLVQSGGGLVQPGGSLGLSCAASGNIGSRDNMGW
YRQAPGKQREWVATISGYGIATYRDSVKGRFTVAKDTAKNIVSLQMNYLTTEDTAVYYCYAYAV
DSRNIFWSQGTQVTVSGGGSGGGSYPYDVPDYA;
FMV-H-CD45 (32) polypeptide
MESNNIKYYKDSSRYFGKILDEHKTINSQLYSLSIKVITIIAIIVSLIATIITIINATSGRTTL
NSNTDILLSQRDEIHNIQEMIFDRIYPLINAMSTELGLHIPTLLDELTKAIDQKIKIMHPPVDT
VTSDLNWCIKPPNGIIIDPKSYCESMELSKTYELLLDQLDVSRKKSLIINRKNINQCQLVDNSK
IIFATVNIQSTPRFLNFGHTVSNQRITFGQGTYSSTYVITIQEDGVTDVQYRVFEIGYISDQFG
VFPSLIVSRVLPIRMLLGMESCTLTSDRLGGYFLCMNTLTRSIYDYVSIRDLKSLYITIPHYGK
VNYTYFNFGKIRSPHEIDKIWLTSDRGQIISGYFAAFVTITIRNYNNYPYKCLNNPCFDNSENY
CRGWYKNITGTDDVPILAYLLVEMYDEEGPLITLVAIPPYNYTAPSHNSLYYDDKINKLIMTTS
HIGYIQINEVHEVIVGDNLKAILLNRLSDEHPNLTACRLNQGIKEQYKSDGTIISNSALIDIQE
RMYITVKAIPPAGNYNFTVELHSRSNTSYVSLPKQFNAKYDKLHLECFSWDKSWWCALIPQFSL
SWNESLSVDTAIFNLISCKGGGGSGGGGSGGGGSAAAQVQLVQSGGGLVQPGGSLRLSCAASGR
AFNSAAMGWYRQAPGSQRELVASISAGTASYADAVKGRFTISRDYAKNIIYLQMNSLKPDDTAV
YFCNYRTTYTSGYSEDYWGQGTQVTVSGGGSGGGSYPYDVPDYA;
PPRV-H-CD45 (32)
MSAQRERINAFYKDNLHNKTHRVILDRERLTIERPYILLGVLLVMFLSLIGLLAIAGIRLHRAT
VGTAEIQSRLNTNIELTESIDHQTKDVLTPLFKIIGDEVGIRIPQKFSDLVKFISDKIKFLNPD
REYDFRDLRWCMNPPERVKINFDQFCEYKAAVKSVEHIFESSLNRSERLRLLTLGPGTGCLGRT
VTRAQFSELTLTLMDLDLEIKHNVSSVFTVVEEGLFGRTYTVWRSDTGKPSTSPGIGHFLRVFE
IGLVRDLELGAPIFHMTNYLTVNMSDDYRSCLLAVGELKLTALCTPSETVTLSESGVPKREPLV
VVILNLAGPTLGGELYSVLPTTDPTVEKLYLSSHRGIIKDNEANWVVPSTDVRDLQNKGECLVE
ACKTRPPSFCNGTGIGPWSEGRIPAYGVIRVSLDLASDPGVVITSVFGPLIPHLSGMDLYNNPF
SRAAWLAVPPYEQSFLGMINTIGFPDRAEVMPHILTTEIRGPRGRCHVPIELSSRIDDDIKIGS
NMVVLPTKDLRYITATYDVSRSEHAIVYYIYDTGRSSSYFYPVRLNFRGNPLSLRIECFPWYHK
VWCYHDCLIYNTITNEEVHTRGLTGIEVTCNPVGGGGSGGGGSGGGGSAAAQVQLVQSGGGLVQ
PGGSLRLSCAASGRAFNSAAMGWYRQAPGSQRELVASISAGTASYADAVKGRFTISRDYAKNII
YLQMNSLKPDDTAVYFCNYRTTYTSGYSEDYWGQGTQVTVSGGGSGGGSYPYDVPDYA;
RPV-H-CD45 (32)
MSPPRDRVDAYYKDNFQFKNTRVVLNKEQLLIERPCMLLTVLFVMFLSLVGLLAIAGIRLHRAA
VNTAKINNDLTTSIDITKSIEYQVKDVLTPLFKIIGDEVGLRTPQRFTDLTKFISDKIKFLNPD
KEYDFRDINWCINPPERIKIDYDQYCAHTAAEDLITMLVNSSLTGTTVLRTSLVNLGRNCTGPT
TTKGQFSNISLTLSGIYSGRGYNISSMITITGKGMYGSTYLVGKYNQRARRPSIVWQQDYRVFE
VGIIRELGVGTPVFHMTNYLELPRQPELETCMLALGESKLAALCLADSPVALHYGRVGDDNKIR
FVKLGVWASPADRDTLATLSAIDPTLDGLYITTHRGIIAAGTAIWAVPVTRTDDQVKMGKCRLE
ACRDRPPPFCNSTDWEPLEAGRIPAYGVLTIKLGLADEPKVDIISEFGPLITHDSGMDLYTSFD
GTKYWLTTPPLQNSALGTVNTLVLEPSLKISPNILTLPIRSGGGDCYTPTYLSDRADDDVKLSS
NLVILPSRDLQYVSATYDISRVEHAIVYHIYSTGRLSSYYYPFKLPIKGDPVSLQIECFPWDRK
LWCHHFCSVIDSGTGEQVTHIGVVGIEITCNGKGGGGSGGGGSGGGGSAAAQVQLVQSGGGLVQ
PGGSLRLSCAASGRAFNSAAMGWYRQAPGSQRELVASISAGTASYADAVKGRFTISRDYAKNII
YLQMNSLKPDDTAVYFCNYRTTYTSGYSEDYWGQGTQVTVSGGGSGGGSYPYDVPDYA;
and
RMV-H-CD45 (32)
MSAQRERINAFYKDNPHNKNHRVILDRERLVIERPYILLGVLLVMFLSLIGLLAIAGIRLHRAT
VGTSEIQSRLNTNIELTESIDHQTKDVLTPLFKIIGDEVGIRIPQKFSDLVKFISDKIKFLNPD
REYDFRDLRWCMNPPERVKINFDQFCEYKAAVKSIEHIFESPLNKSKKLQSLTLGPGTGCLGRT
VTRAHFSELTLTLMDLDLEMKHNVSSVFTVVEEGLFGRTYTVWRSDARDPSTDLGIGHFLRVFE
IGLVRDLGLGPPVFHMTNYLTVNMSDDYRRCLLAVGELKLTALCSSSETVTLGERGVPKREPLV
VVILNLAGPTLGGELYSVLPTSDLMVEKLYLSSHRGIIKDDEANWVVPSTDVRDLQNKGECLVE
ACKTRPPSFCNGTGSGPWSEGRIPAYGVIRVSLDLASDPGVVITSVFGPLIPHLSGMDLYNNPF
SRAVWLAVPPYEQSFLGMINTIGFPNRAEVMPHILTTEIRGPRGRCHVPIELSRRVDDDIKIGS
NMVILPTIDLRYITATYDVSRSEHAIVYYIYDTGRSSSYFYPVRLNFKGNPLSLRIECFPWRHK
VWCYHDCLIYNTITDEEVHTRGLTGIEVTCNPVGGGGSGGGGSGGGGSAAAQVQLVQSGGGLVQ
PGGSLRLSCAASGRAFNSAAMGWYRQAPGSQRELVASISAGTASYADAVKGRFTISRDYAKNII
YLQMNSLKPDDTAVYFCNYRTTYTSGYSEDYWGQGTQVTVSGGGSGGGSYPYDVPDYA.

19. A chimeric viral envelope glycoprotein polypeptide or fragment thereof suitable for pseudotyping a viral particle comprising an amino acid sequence at least about 20 amino acids in length derived from a non-measles virus morbillivirus F protein extravirion domain N-terminal to an amino acid sequence at least about 20 amino acids in length derived from a measles virus F protein extravirion, wherein the chimeric viral envelope glycoprotein polypeptide comprises the amino acid sequence RLDVGTNLGNAIAKLEDAKELLESSDQILRSMKGLSSTS (MeV-F extravirion stalk domain), wherein the non-measles virus morbillivirus is selected from the group consisting of canine distemper virus, dolphin Morbillivirus, feline Morbillivirus, measles virus, Peste des petits ruminant virus, phocine Morbillivirus, Rinderpest virus, and small ruminant virus, and wherein the chimeric protein comprises a sequence with at least 85% sequence identity to a sequence selected from the group consisting of
F2 DMV Fusion
MAASNGGVMYQSFLTIIILVIMTEGQIHWGNLSKIGIVGTGSASYKVMTRPNHQYLVIKLMPNV
TMIDNCTRTEVTEYRKLLKTVLEPVKNALTVITKNIKPIQSLYPYDVPDYATTSRRSKRFAGVV
LAGAALGVATAAQITAGIALHQSMLNSQAIDNLRASLETTNQAIEAIRQAGQEMILAVQGVQDY
INNELIPSMNQLSCDLIGQKLGLKLLRYYTEILSLFGPSLRDPISAEISIQALSYALGGDINKV
LEKLGYSGGDLLGILESRGIKARITHVDTESYFIVLSIAYPTLSEIKGVIVHRLEGVSYNIGSQ
EWYTTVPKYVATQGYLISNFDESSCTFMPEGTVCSQNALYPMSPLLQECLRGSTKSCARTLVSG
SFGNRFILSQGNLIANCASILCKCYTTGTIINQDPDKILTYIAADHCPVVEVNGVTIQVGSRRY
PDAVYLHRIDLGPPISLERLDVGTNLGNAIAKLEDAKELLESSDQILRSMKGLSSTSIVYILIA
VCLGGLIGIPALICCCRGR;
S-SDMV Fusion
MAASNGGVMYQSFLTIIILVIMTEGQIHWGNLSKIGIVGTGSASYKVMTRPNHQYLVIKLMPNV
TMIDNCTRTEVTEYRKLLKTVLEPVKNALTVITKNIKPIQSLYPYDVPDYATTSRRSKRFAGVV
LAGVALGVATAAQITAGVALHQSIMNSQSIDNLRTSLEKSNQAIEEIRQASQETVLAVQGVQDF
INNELIPSMHQLSCEMLGQKLGLKLLRYYTEILSIFGPSLRDPISAEISIQALSYALGGDINKV
LEKLGYSGGDLLGILESRGIKARITHVDTESYFIVLSIAYPTLSEIKGVIVHRLEGVSYNIGSQ
EWYTTVPKYVATQGYLISNFDESSCTFMPEGTVCSQNALYPMSPLLQECLRGSTKSCARTLVSG
SFGNRFILSQGNLIANCASILCKCYTTGTIINQDPDKILTYIAADHCPVVEVNGVTIQVGSRRY
PDAVYLHRIDLGPPISLERLDVGTNLGNAIAKLEDAKELLESSDQILRSMKGLSSTSSIVYILI
AVCLGGLIGIPALICCCRGR;
Intermediate DMV Fusion
MAASNGGVMYQSFLTIIILVIMTEGQIHWGNLSKIGIVGTGSASYKVMTRPNHQYLVIKLMPNV
TMIDNCTRTEVTEYRKLLKTVLEPVKNALTVITKNIKPIQSLYPYDVPDYATTSRRSKRFAGVV
LAGVALGVATAAQITAGVALHQSIMNSQSIDNLRTSLEKSNQAIEEIRQASQETVLAVQGVQDF
INNELIPSMHQLSCEMLGQKLGLKLLRYYTEILSIFGPSLRDPVSAEISIQALSYALGGDINKI
LEKLGYSGADLLAILESRGIKAKVTHVDLEGYFIVLSIAYPTLSEVKGVIVHKLEGVSYNIGSQ
EWYTTVPKYVATQGYLISNFDESSCTFMPEGTVCSQNALYPMSPLLQECLRGSTKSCARTLVSG
SFGNRFILSQGNLIANCASILCKCYTTGTIINQDPDKILTYIAADHCPVVEVNGVTIQVGSRRY
PDAVYLHRIDLGPPISLERLDVGTNLGNAIAKLEDAKELLESSDQILRSMKGLSSTSSIVYILI
AVCLGGLIGIPALICCCRGR;
H Interacting Domain DMV Fusion
MAASNGGVMYQSFLTIIILVIMTEGQIHWGNLSKIGIVGTGSASYKVMTRPNHQYLVIKLMPNV
TMIDNCTRTEVTEYRKLLKTVLEPVKNALTVITKNIKPIQSLYPYDVPDYATTSRRSKRFAGVV
LAGVALGVATAAQITAGVALHQSIMNSQSIDNLRTSLEKSNQAIEEIRQASQETVLAVQGVQDF
INNELIPSMHQLSCEMLGQKLGLKLLRYYTEILSIFGPSLRDPVSAEISIQALSYALGGDINKI
LEKLGYSGADLLAILESRGIKAKVTHVDLEGYFIVLSIAYPTLSEVKGVIVHKLEAVSYNLGSQ
EWYTTLPKYVATNGYLISNFDESSCAFMSEVTICSQNALYPMSPLLQQCLRGSTASCARSLVSG
TIGNRFILSKGNLIANCASVLCKCYTTGTIINQDPDKILTYIAADHCPVVEVNGVTIQVGSRRY
PDAVYLHRIDLGPPISLERLDVGTNLGNAIAKLEDAKELLESSDQILRSMKGLSSTSSIVYILI
AVCLGGLIGIPALICCCRGR;
and
Stalk DMV Fusion
MAASNGGVMYQSFLTIIILVIMTEGQIHWGNLSKIGIVGTGSASYKVMTRPNHQYLVIKLMPNV
TMIDNCTRTEVTEYRKLLKTVLEPVKNALTVITKNIKPIQSLYPYDVPDYATTSRRSKRFAGVV
LAGVALGVATAAQITAGVALHQSIMNSQSIDNLRTSLEKSNQAIEEIRQASQETVLAVQGVQDF
INNELIPSMHQLSCEMLGQKLGLKLLRYYTEILSIFGPSLRDPVSAEISIQALSYALGGDINKI
LEKLGYSGADLLAILESRGIKAKVTHVDLEGYFIVLSIAYPTLSEVKGVIVHKLEAVSYNLGSQ
EWYTTLPKYVATNGYLISNFDESSCAFMSEVTICSQNALYPMSPLLQQCLRGSTASCARSLVSG
TIGNRFILSKGNLIANCASVLCKCYSTGTIISQDPDKLLTFVAADKCPLVEVDGITIQVGSREY
PDSVYVSRIDLGPAISLERLDVGTNLGNAIAKLEDAKELLESSDQILRSMKGLSSTSSIVYILI
AVCLGGLIGIPALICCCRGR.

20. A pseudotyped viral particle comprising the chimeric polypeptide of claim 19.

21. The pseudotyped viral particle of claim 20, wherein the pseudotyped viral particle resists neutralization by a measles virus neutralizing antibody relative to a reference viral particle pseudotyped with a glycoprotein polypeptide comprising a measles virus F protein (MeV-Fc) extravirion domain.


SEQUENCE LISTING (128 sequences)

SEQ ID NO: 1 - protein, 738 aa, synthetic construct
MSSPRDKVDAFYKDIPRPRNNRVLLDNERVIIERPLILVGVLAVMFLSLVGLLAIAGVRLQKATTNSIEVNRKLSTNLETTVSIEHHVKDVLTPLFKIIGDEVGLRMPQKLTEIMQFISNKIKFLNPDREYDFNDLHWCVNPPDQVKIDYAQYCNHIAAEELIVTKFKELMNHSLDMSKGRIFPPKNCSGSVITRGQTIKPGLTLVNIYTTRNFEVSFMVTVISGGMYGKTYFLKPPEPDDPFEFQAFRIFEVGLVRDVGSREPVLQMTNFMVIDEDEGLNFCLLSVGELRLAAVCVRGRPVVTKDIGGYKDEPFKVVTLGIIGGGLSNQKTEIYPTIDSSIEKLYITSHRGIIRNSKARWSVPAIRSDDKDKMEKCTQALCKSRPPPSCNSSDWEPLTSNRIPAYAYIALEIKEDSGLELDITSNYGPLIIHGAGMDIYEGPSSNQDWLAIPPLSQSVLGVINKVDFTAGFDIKPHTLTTAVDYESGKCYVPVELSGAKDQDLKLESNLVVLPTKDFGYVTATYDTSRSEHAIVYYVYDTARSSSYFFPFRIKARGEPIYLRIECFPWSRQLWCHHYCMINSTVSNEIVVVDNLVSINMSCSRGGGGSGGGGSGGGGSAAAQVQLVQSGGGLVQPGGSLGLSCAASGNIGSRDNMGWYRQAPGKQREWVATISGYGIATYRDSVKGRFTVAKDTAKNIVSLQMNYLTTEDTAVYYCYAYAVDSRNIFWSQGTQVTVS

SEQ ID NO: 2 - protein, 765 aa, synthetic construct
MSSPRDKVDAFYKDIPRPRNNRVLLDNERVIIERPLILVGVLAVMFLSLVGLLAIAGVRLQKATTNSIEVNRKLSTNLETTVSIEHHVKDVLTPLFKIIGDEVGLRMPQKLTEIMQFISNKIKFLNPDREYDFNDLHWCVNPPDQVKIDYAQYCNHIAAEELIVTKFKELMNHSLDMSKGRIFPPKNCSGSVITRGQTIKPGLTLVNIYTTRNFEVSFMVTVISGGMYGKTYFLKPPEPDDPFEFQAFRIFEVGLVRDVGSREPVLQMTNFMVIDEDEGLNFCLLSVGELRLAAVCVRGRPVVTKDIGGYKDEPFKVVTLGIIGGGLSNQKTEIYPTIDSSIEKLYITSHRGIIRNSKARWSVPAIRSDDKDKMEKCTQALCKSRPPPSCNSSDWEPLTSNRIPAYAYIALEIKEDSGLELDITSNYGPLIIHGAGMDIYEGPSSNQDWLAIPPLSQSVLGVINKVDFTAGFDIKPHTLTTAVDYESGKCYVPVELSGAKDQDLKLESNLVVLPTKDFGYVTATYDTSRSEHAIVYYVYDTARSSSYFFPFRIKARGEPIYLRIECFPWSRQLWCHHYCMINSTVSNEIVVVDNLVSINMSCSRGGGGSGGGGSGGGGSAAADVQLQESGGGSVQAGGSLRLSCAASGYTHSSYCMAWFRQAPGREREGVASIDSDGTTSYADSVKGRFTISQDNAKNTLYLQMNSLKPEDTAMYYCAARFGPMGCVDLSTLSFGHWGQGTQVTVSITGGGSGGGSYPYDVPDYA

SEQ ID NO: 3 - protein, 757 aa, synthetic construct
MSSPRDKVDAFYKDIPRPRNNRVLLDNERVIIERPLILVGVLAVMFLSLVGLLAIAGVRLQKATTNSIEVNRKLSTNLETTVSIEHHVKDVLTPLFKIIGDEVGLRMPQKLTEIMQFISNKIKFLNPDREYDFNDLHWCVNPPDQVKIDYAQYCNHIAAEELIVTKFKELMNHSLDMSKGRIFPPKNCSGSVITRGQTIKPGLTLVNIYTTRNFEVSFMVTVISGGMYGKTYFLKPPEPDDPFEFQAFRIFEVGLVRDVGSREPVLQMTNFMVIDEDEGLNFCLLSVGELRLAAVCVRGRPVVTKDIGGYKDEPFKVVTLGIIGGGLSNQKTEIYPTIDSSIEKLYITSHRGIIRNSKARWSVPAIRSDDKDKMEKCTQALCKSRPPPSCNSSDWEPLTSNRIPAYAYIALEIKEDSGLELDITSNYGPLIIHGAGMDIYEGPSSNQDWLAIPPLSQSVLGVINKVDFTAGFDIKPHTLTTAVDYESGKCYVPVELSGAKDQDLKLESNLVVLPTKDFGYVTATYDTSRSEHAIVYYVYDTARSSSYFFPFRIKARGEPIYLRIECFPWSRQLWCHHYCMINSTVSNEIVVVDNLVSINMSCSRGGGGSGGGGSGGGGSAAAQVQLVQSGGGLVQPGGSLRLSCAASGRAFNSAAMGWYRQAPGSQRELVASISAGTASYADAVKGRFTISRDYAKNIIYLQMNSLKPDDTAVYFCNYRTTYTSGYSEDYWGQGTQVTVSGGGSGGGSYPYDVPDYA

SEQ ID NO: 4 - protein, 755 aa, synthetic construct
MLPYQDKVGAFYKDNARANSTKLSLVTEGHGGRRPPYLLFVLLILLVGILALLAITGVRFHQVSTSNMEFSRLLKEDMEKSEAVHHQVIDVLTPLFKIIGDEIGLRLPQKLNEIKQFILQKTNFFNPNREFDFRDLHWCINPPSTVKVNFTNYCESIGIRKAIASAANPILLSALSGGRGDIFPPHRCSGATTSVGKVFPLSVSLSMSLISRTSEVINMLTAISDGVYGKTYLLVPDDIEREFDTREIRVFEIGFIKRWLNDMPLLQTTNYMVLPKNSKAKVCTIAVGELTLASLCVEESTVLLYHDSSGSQDGILVVTLGIFWATPMDHIEEVIPVAHPSMKKIHITNHRGFIKDSIATWMVPALASEKQEEQKGCLESACQRKTYPMCNQASWEPFGGRQLPSYGRLTLPLDASVDLQLNISFTYGPVILNGDGMDYYESPLLNSGWLTIPPKDGTISGLINKAGRGDQFTVLPHVLTFAPRESSGNCYLPIQTSQIRDRDVLIESNIVVLPTQSIRYVIATYDISRSDHAIVYYVYDPIRTISYTHPFRLTTKGRPDFLRIECFVWDDNLWCHQFYRFEADIANSTTSVENLVRIRFSCNRGGGGSGGGGSGGGGSAAAQVQLVQSGGGLVQPGGSLGLSCAASGNIGSRDNMGWYRQAPGKQREWVATISGYGIATYRDSVKGRFTVAKDTAKNIVSLQMNYLTTEDTAVYYCYAYAVDSRNIFWSQGTQVTVSGGGSGGGSYPYDVPDYA

SEQ ID NO: 5 - protein, 765 aa, synthetic construct
MLPYQDKVGAFYKDNARANSTKLSLVTEGHGGRRPPYLLFVLLILLVGILALLAITGVRFHQVSTSNMEFSRLLKEDMEKSEAVHHQVIDVLTPLFKIIGDEIGLRLPQKLNEIKQFILQKTNFFNPNREFDFRDLHWCINPPSTVKVNFTNYCESIGIRKAIASAANPILLSALSGGRGDIFPPHRCSGATTSVGKVFPLSVSLSMSLISRTSEVINMLTAISDGVYGKTYLLVPDDIEREFDTREIRVFEIGFIKRWLNDMPLLQTTNYMVLPKNSKAKVCTIAVGELTLASLCVEESTVLLYHDSSGSQDGILVVTLGIFWATPMDHIEEVIPVAHPSMKKIHITNHRGFIKDSIATWMVPALASEKQEEQKGCLESACQRKTYPMCNQASWEPFGGRQLPSYGRLTLPLDASVDLQLNISFTYGPVILNGDGMDYYESPLLNSGWLTIPPKDGTISGLINKAGRGDQFTVLPHVLTFAPRESSGNCYLPIQTSQIRDRDVLIESNIVVLPTQSIRYVIATYDISRSDHAIVYYVYDPIRTISYTHPFRLTTKGRPDFLRIECFVWDDNLWCHQFYRFEADIANSTTSVENLVRIRFSCNRGGGGSGGGGSGGGGSAAADVQLQESGGGSVQAGGSLRLSCAASGYTHSSYCMAWFRQAPGREREGVASIDSDGTTSYADSVKGRFTISQDNAKNTLYLQMNSLKPEDTAMYYCAARFGPMGCVDLSTLSFGHWGQGTQVTVSITGGGSGGGSYPYDVPDYA

SEQ ID NO: 6 - protein, 752 aa, synthetic construct
MLPYQDKVGAFYKDNARANSTKLSLVTEGHGGRRPPYLLFVLLILLVGILALLAITGVRFHQVSTSNMEFSRLLKEDMEKSEAVHHQVIDVLTPLFKIIGDEIGLRLPQKLNEIKQFILQKTNFFNPNREFDFRDLHWCINPPSTVKVNFTNYCESIGIRKAIASAANPILLSALSGGRGDIFPPHRCSGATTSVGKVFPLSVSLSMSLISRTSEVINMLTAISDGVYGKTYLLVPDDIEREFDTREIRVFEIGFIKRWLNDMPLLQTTNYMVLPKNSKAKVCTIAVGELTLASLCVEESTVLLYHDSSGSQDGILVVTLGIFWATPMDHIEEVIPVAHPSMKKIHITNHRGFIKDSIATWMVPALASEKQEEQKGCLESACQRKTYPMCNQASWEPFGGRQLPSYGRLTLPLDASVDLQLNISFTYGPVILNGDGMDYYESPLLNSGWLTIPPKDGTISGLINKAGRGDQFTVLPHVLTFAPRESSGNCYLPIQTSQIRDRDVLIESNIVVLPTQSIRYVIATYDISRSDHAIVYYVYDPIRTISYTHPFRLTTKGRPDFLRIECFVWDDNLWCHQFYRFEADIANSTTSVENLVRIRFSCNRGGGGSGGGGSGGGGSAAAQVQLVQSGGGLVQPGGSLRLSCAASGRAFNSAAMGWYRQAPGSQRELVASISAGTASYADAVKGRFTISRDYAKNIIYLQMNSLKPDDTAVYFCNYRTTYTSGYSEDYWGQGTQVTVSAAAYPYDVPDYA

SEQ ID NO: 7 - protein, 737 aa, synthetic construct
MGSRIVINREHLMIDRPYVLLAVLFVMFLSLIGLLAIAGIRLHRAAIYTAEIHKSLSTNLDVTNSIEHQVKDVLTPLFKIIGDEVGLRTPQRFTDLVKFISDKIKFLNPDREYDFRDLTWCINPPERIKLDYDQYCADVAARKAIASAANPILLSALSGGRGDIFPPHRCSGATTSVGKVFPLSVSLSMSLISRTSEVINMLTAISDGVYGKTYLLVPDDIEREFDTREIRVFEIGFIKRWLNDMPLLQTTNYMVLPKNSKAKVCTIAVGELTLASLCVEESTVLLYHDSSGSQDGILVVTLGIFWATPMDHIEEVIPVAHPSMKKIHITNHRGFIKDSIATWMVPALASEKQEEQKGCLESACQRKTYPMCNQASWEPFGGRQLPSYGRLTLPLDASVDLQLNISFTYGPVILNGDGMDYYESPLLNSGWLTIPPKDGTISGLINKAGRGDQFTVLPHVLTFAPRESSGNCYLPIQTSQIRDRDVLIESNIVVLPTQSIRYVIATYDISRSDHAIVYYVYDPIRTISYTHPFRLTTKGRPDFLRIECFVWDDNLWCHQFYRFEADIANSTTSVENLVRIRFSCNRGGGGSGGGGSGGGGSAAAQVQLVQSGGGLVQPGGSLGLSCAASGNIGSRDNMGWYRQAPGKQREWVATISGYGIATYRDSVKGRFTVAKDTAKNIVSLQMNYLTTEDTAVYYCYAYAVDSRNIFWSQGTQVTVSGGGSGGGSYPYDVPDYA

SEQ ID NO: 8 - protein, 737 aa, synthetic construct
MGSRIVINREHLMIDRPYVLLAVLFVMFLSLIGLLAIAGIRLHRAAIYTAEIHKSLSTNLDVTNSIEHQVKDVLTPLFKIIGDEVGLRTPQRFTDLVKFISDKIKFLNPDREYDFRDLTWCINPPERIKLDYDQYCADVAAEELIVTKFKELMNHSLDMSKGRIFPPKNCSGSVITRGQTIKPGLTLVNIYTTRNFEVSFMVTVISGGMYGKTYFLKPPEPDDPFEFQAFRIFEVGLVRDVGSREPVLQMTNFMVIDEDEGLNFCLLSVGELRLAAVCVRGRPVVTKDIGGYKDEPFKVVTLGIIGGGLSNQKTEIYPTIDSSIEKLYITSHRGIIRNSKARWSVPAIRSDDKDKMEKCTQALCKSRPPPSCNSSDWEPLTSNRIPAYAYIALEIKEDSGLELDITSNYGPLIIHGAGMDIYEGPSSNQDWLAIPPLSQSVLGVINKVDFTAGFDIKPHTLTTAVDYESGKCYVPVELSGAKDQDLKLESNLVVLPTKDFGYVTATYDTSRSEHAIVYYVYDTARSSSYFFPFRIKARGEPIYLRIECFPWSRQLWCHHYCMINSTVSNEIVVVDNLVSINMSCSRGGGGSGGGGSGGGGSAAAQVQLVQSGGGLVQPGGSLGLSCAASGNIGSRDNMGWYRQAPGKQREWVATISGYGIATYRDSVKGRFTVAKDTAKNIVSLQMNYLTTEDTAVYYCYAYAVDSRNIFWSQGTQVTVSGGGSGGGSYPYDVPDYA

SEQ ID NO: 9 - protein, 748 aa, synthetic construct
MESNNIKYYKDSSRYFGKILDEHKTINSQLYSLSIKVITIIAIIVSLIATIITIINATSGRTTLNSNTDILLSQRDEIHNIQEMIFDRIYPLINAMSTELGLHIPTLLDELTKAIDQKIKIMHPPVDTVTSDLNWCIKPPNGIIIDPKSYCESMELSKTYELLLDQLDVSRKKSLIINRKNINQCQLVDNSKIIFATVNIQSTPRFLNFGHTVSNQRITFGQGTYSSTYVITIQEDGVTDVQYRVFEIGYISDQFGVFPSLIVSRVLPIRMLLGMESCTLTSDRLGGYFLCMNTLTRSIYDYVSIRDLKSLYITIPHYGKVNYTYFNFGKIRSPHEIDKIWLTSDRGQIISGYFAAFVTITIRNYNNYPYKCLNNPCFDNSENYCRGWYKNITGTDDVPILAYLLVEMYDEEGPLITLVAIPPYNYTAPSHNSLYYDDKINKLIMTTSHIGYIQINEVHEVIVGDNLKAILLNRLSDEHPNLTACRLNQGIKEQYKSDGTIISNSALIDIQERMYITVKAIPPAGNYNFTVELHSRSNTSYVSLPKQFNAKYDKLHLECFSWDKSWWCALIPQFSLSWNESLSVDTAIFNLISCKGGGGSGGGGSGGGGSAAAQVQLVQSGGGLVQPGGSLRLSCAASGRAFNSAAMGWYRQAPGSQRELVASISAGTASYADAVKGRFTISRDYAKNIIYLQMNSLKPDDTAVYFCNYRTTYTSGYSEDYWGQGTQVTVSGGGSGGGSYPYDVPDYA

SEQ ID NO: 10 - protein, 762 aa, synthetic construct
MSAQRERINAFYKDNLHNKTHRVILDRERLTIERPYILLGVLLVMFLSLIGLLAIAGIRLHRATVGTAEIQSRLNTNIELTESIDHQTKDVLTPLFKIIGDEVGIRIPQKFSDLVKFISDKIKFLNPDREYDFRDLRWCMNPPERVKINFDQFCEYKAAVKSVEHIFESSLNRSERLRLLTLGPGTGCLGRTVTRAQFSELTLTLMDLDLEIKHNVSSVFTVVEEGLFGRTYTVWRSDTGKPSTSPGIGHFLRVFEIGLVRDLELGAPIFHMTNYLTVNMSDDYRSCLLAVGELKLTALCTPSETVTLSESGVPKREPLVVVILNLAGPTLGGELYSVLPTTDPTVEKLYLSSHRGIIKDNEANWVVPSTDVRDLQNKGECLVEACKTRPPSFCNGTGIGPWSEGRIPAYGVIRVSLDLASDPGVVITSVFGPLIPHLSGMDLYNNPFSRAAWLAVPPYEQSFLGMINTIGFPDRAEVMPHILTTEIRGPRGRCHVPIELSSRIDDDIKIGSNMVVLPTKDLRYITATYDVSRSEHAIVYYIYDTGRSSSYFYPVRLNFRGNPLSLRIECFPWYHKVWCYHDCLIYNTITNEEVHTRGLTGIEVTCNPVGGGGSGGGGSGGGGSAAAQVQLVQSGGGLVQPGGSLRLSCAASGRAFNSAAMGWYRQAPGSQRELVASISAGTASYADAVKGRFTISRDYAKNIIYLQMNSLKPDDTAVYFCNYRTTYTSGYSEDYWGQGTQVTVSGGGSGGGSYPYDVPDYA

SEQ ID NO: 11 - protein, 761 aa, synthetic construct
MSPPRDRVDAYYKDNFQFKNTRVVLNKEQLLIERPCMLLTVLFVMFLSLVGLLAIAGIRLHRAAVNTAKINNDLTTSIDITKSIEYQVKDVLTPLFKIIGDEVGLRTPQRFTDLTKFISDKIKFLNPDKEYDFRDINWCINPPERIKIDYDQYCAHTAAEDLITMLVNSSLTGTTVLRTSLVNLGRNCTGPTTTKGQFSNISLTLSGIYSGRGYNISSMITITGKGMYGSTYLVGKYNQRARRPSIVWQQDYRVFEVGIIRELGVGTPVFHMTNYLELPRQPELETCMLALGESKLAALCLADSPVALHYGRVGDDNKIRFVKLGVWASPADRDTLATLTHEPTLDGLYITTHRGIIAAGTAIWAVPVTRTDDQVKMGKCRLEACRDRPPPFCNSTDWEPLEAGRIPAYGVLTIKLGLADEPKVDIISEFGPLITHDSGMDLYTSFDGTKYWLTTPPLQNSALGTVNTLVLEPSLKISPNILTLPIRSGGGDCYTPTYLSDRADDDVKLSSNLVILPSRDLQYVSATYDISRVEHAIVYHIYSTGRLSSYYYPFKLPIKGDPVSLQIECFPWDRKLWCHHFCSVIDSGTGEQVTHIGVVGIEITCNGKGGGGSGGGGSGGGGSAAAQVQLVQSGGGLVQPGGSLRLSCAASGRAFNSAAMGWYRQAPGSQRELVASISAGTASYADAVKGRFTISRDYAKNIIYLQMNSLKPDDTAVYFCNYRTTYTSGYSEDYWGQGTQVTVSGGGSGGGSYPYDVPDYA

SEQ ID NO: 12 - protein, 762 aa, synthetic construct
MSAQRERINAFYKDNPHNKNHRVILDRERLVIERPYILLGVLLVMFLSLIGLLAIAGIRLHRATVGTSEIQSRLNTNIELTESIDHQTKDVLTPLFKIIGDEVGIRIPQKFSDLVKFISDKIKFLNPDREYDFRDLRWCMNPPERVKINFDQFCEYKAAVKSIEHIFESPLNKSKKLQSLTLGPGTGCLGRTVTRAHFSELTLTLMDLDLEMKHNVSSVFTVVEEGLFGRTYTVWRSDARDPSTDLGIGHFLRVFEIGLVRDLGLGPPVFHMTNYLTVNMSDDYRRCLLAVGELKLTALCSSSETVTLGERGVPKREPLVVVILNLAGPTLGGELYSVLPTSDLMVEKLYLSSHRGIIKDDEANWVVPSTDVRDLQNKGECLVEACKTRPPSFCNGTGSGPWSEGRIPAYGVIRVSLDLASDPGVVITSVFGPLIPHLSGMDLYNNPFSRAVWLAVPPYEQSFLGMINTIGFPNRAEVMPHILTTEIRGPRGRCHVPIELSRRVDDDIKIGSNMVILPTIDLRYITATYDVSRSEHAIVYYIYDTGRSSSYFYPVRLNFKGNPLSLRIECFPWRHKVWCYHDCLIYNTITDEEVHTRGLTGIEVTCNPVGGGGSGGGGSGGGGSAAAQVQLVQSGGGLVQPGGSLRLSCAASGRAFNSAAMGWYRQAPGSQRELVASISAGTASYADAVKGRFTISRDYAKNIIYLQMNSLKPDDTAVYFCNYRTTYTSGYSEDYWGQGTQVTVSGGGSGGGSYPYDVPDYA

SEQ ID NO: 13 - protein, 101 aa, Measles morbillivirus
RLHRAAIYTAEIHKSLSTNLDVTNSIEHQVKDVLTPLFKIIGDEVGLRTPQRFTDLVKFISDKIKFLNPDREYDFRDLTWCINPPERIKLDYDQYCADVAA

SEQ ID NO: 14 - protein, 445 aa, Canine morbillivirus
RKAIASAANPILLSALSGGRGDIFPPHRCSGATTSVGKVFPLSVSLSMSLISRTSEVINMLTAISDGVYGKTYLLVPDDIEREFDTREIRVFEIGFIKRWLNDMPLLQTTNYMVLPKNSKAKVCTIAVGELTLASLCVEESTVLLYHDSSGSQDGILVVTLGIFWATPMDHIEEVIPVAHPSMKKIHITNHRGFIKDSIATWMVPALASEKQEEQKGCLESACQRKTYPMCNQASWEPFGGRQLPSYGRLTLPLDASVDLQLNISFTYGPVILNGDGMDYYESPLLNSGWLTIPPKDGTISGLINKAGRGDQFTVLPHVLTFAPRESSGNCYLPIQTSQIRDRDVLIESNIVVLPTQSIRYVIATYDISRSDHAIVYYVYDPIRTISYTHPFRLTTKGRPDFLRIECFVWDDNLWCHQFYRFEADIANSTTSVENLVRIRFSCNR

SEQ ID NO: 15 - protein, 445 aa, Cetacean morbillivirus
EELIVTKFKELMNHSLDMSKGRIFPPKNCSGSVITRGQTIKPGLTLVNIYTTRNFEVSFMVTVISGGMYGKTYFLKPPEPDDPFEFQAFRIFEVGLVRDVGSREPVLQMTNFMVIDEDEGLNFCLLSVGELRLAAVCVRGRPVVTKDIGGYKDEPFKVVTLGIIGGGLSNQKTEIYPTIDSSIEKLYITSHRGIIRNSKARWSVPAIRSDDKDKMEKCTQALCKSRPPPSCNSSDWEPLTSNRIPAYAYIALEIKEDSGLELDITSNYGPLIIHGAGMDIYEGPSSNQDWLAIPPLSQSVLGVINKVDFTAGFDIKPHTLTTAVDYESGKCYVPVELSGAKDQDLKLESNLVVLPTKDFGYVTATYDTSRSEHAIVYYVYDTARSSSYFFPFRIKARGEPIYLRIECFPWSRQLWCHHYCMINSTVSNEIVVVDNLVSINMSCSR

SEQ ID NO: 16 - protein, 604 aa, Cetacean morbillivirus
MSSPRDKVDAFYKDIPRPRNNRVLLDNERVIIERPLILVGVLAVMFLSLVGLLAIAGVRLQKATTNSIEVNRKLSTNLETTVSIEHHVKDVLTPLFKIIGDEVGLRMPQKLTEIMQFISNKIKFLNPDREYDFNDLHWCVNPPDQVKIDYAQYCNHIAAEELIVTKFKELMNHSLDMSKGRIFPPKNCSGSVITRGQTIKPGLTLVNIYTTRNFEVSFMVTVISGGMYGKTYFLKPPEPDDPFEFQAFRIFEVGLVRDVGSREPVLQMTNFMVIDEDEGLNFCLLSVGELRLAAVCVRGRPVVTKDIGGYKDEPFKVVTLGIIGGGLSNQKTEIYPTIDSSIEKLYITSHRGIIRNSKARWSVPAIRSDDKDKMEKCTQALCKSRPPPSCNSSDWEPLTSNRIPAYAYIALEIKEDSGLELDITSNYGPLIIHGAGMDIYEGPSSNQDWLAIPPLSQSVLGVINKVDFTAGFDIKPHTLTTAVDYESGKCYVPVELSGAKDQDLKLESNLVVLPTKDFGYVTATYDTSRSEHAIVYYVYDTARSSSYFFPFRIKARGEPIYLRIECFPWSRQLWCHHYCMINSTVSNEIVVVDNLVSINMSCSR

SEQ ID NO: 17 - protein, 604 aa, Canine morbillivirus
MLPYQDKVGAFYKDNARANSTKLSLVTEGHGGRRPPYLLFVLLILLVGILALLAITGVRFHQVSTSNMEFSRLLKEDMEKSEAVHHQVIDVLTPLFKIIGDEIGLRLPQKLNEIKQFILQKTNFFNPNREFDFRDLHWCINPPSTVKVNFTNYCESIGIRKAIASAANPILLSALSGGRGDIFPPHRCSGATTSVGKVFPLSVSLSMSLISRTSEVINMLTAISDGVYGKTYLLVPDDIEREFDTREIRVFEIGFIKRWLNDMPLLQTTNYMVLPKNSKAKVCTIAVGELTLASLCVEESTVLLYHDSSGSQDGILVVTLGIFWATPMDHIEEVIPVAHPSMKKIHITNHRGFIKDSIATWMVPALASEKQEEQKGCLESACQRKTYPMCNQASWEPFGGRQLPSYGRLTLPLDASVDLQLNISFTYGPVILNGDGMDYYESPLLNSGWLTIPPKDGTISGLINKAGRGDQFTVLPHVLTFAPRESSGNCYLPIQTSQIRDRDVLIESNIVVLPTQSIRYVIATYDISRSDHAIVYYVYDPIRTISYTHPFRLTTKGRPDFLRIECFVWDDNLWCHQFYRFEADIANSTTSVENLVRIRFSCNR

SEQ ID NO: 18 - protein, 586 aa, synthetic construct
MGSRIVINREHLMIDRPYVLLAVLFVMFLSLIGLLAIAGIRLHRAAIYTAEIHKSLSTNLDVTNSIEHQVKDVLTPLFKIIGDEVGLRTPQRFTDLVKFISDKIKFLNPDREYDFRDLTWCINPPERIKLDYDQYCADVAARKAIASAANPILLSALSGGRGDIFPPHRCSGATTSVGKVFPLSVSLSMSLISRTSEVINMLTAISDGVYGKTYLLVPDDIEREFDTREIRVFEIGFIKRWLNDMPLLQTTNYMVLPKNSKAKVCTIAVGELTLASLCVEESTVLLYHDSSGSQDGILVVTLGIFWATPMDHIEEVIPVAHPSMKKIHITNHRGFIKDSIATWMVPALASEKQEEQKGCLESACQRKTYPMCNQASWEPFGGRQLPSYGRLTLPLDASVDLQLNISFTYGPVILNGDGMDYYESPLLNSGWLTIPPKDGTISGLINKAGRGDQFTVLPHVLTFAPRESSGNCYLPIQTSQIRDRDVLIESNIVVLPTQSIRYVIATYDISRSDHAIVYYVYDPIRTISYTHPFRLTTKGRPDFLRIECFVWDDNLWCHQFYRFEADIANSTTSVENLVRIRFSCNR

SEQ ID NO: 19 - protein, 586 aa, synthetic construct
MGSRIVINREHLMIDRPYVLLAVLFVMFLSLIGLLAIAGIRLHRAAIYTAEIHKSLSTNLDVTNSIEHQVKDVLTPLFKIIGDEVGLRTPQRFTDLVKFISDKIKFLNPDREYDFRDLTWCINPPERIKLDYDQYCADVAAEELIVTKFKELMNHSLDMSKGRIFPPKNCSGSVITRGQTIKPGLTLVNIYTTRNFEVSFMVTVISGGMYGKTYFLKPPEPDDPFEFQAFRIFEVGLVRDVGSREPVLQMTNFMVIDEDEGLNFCLLSVGELRLAAVCVRGRPVVTKDIGGYKDEPFKVVTLGIIGGGLSNQKTEIYPTIDSSIEKLYITSHRGIIRNSKARWSVPAIRSDDKDKMEKCTQALCKSRPPPSCNSSDWEPLTSNRIPAYAYIALEIKEDSGLELDITSNYGPLIIHGAGMDIYEGPSSNQDWLAIPPLSQSVLGVINKVDFTAGFDIKPHTLTTAVDYESGKCYVPVELSGAKDQDLKLESNLVVLPTKDFGYVTATYDTSRSEHAIVYYVYDTARSSSYFFPFRIKARGEPIYLRIECFPWSRQLWCHHYCMINSTVSNEIVVVDNLVSINMSCSR

SEQ ID NO: 20 - protein, 595 aa, Feline morbillivirus
MESNNIKYYKDSSRYFGKILDEHKTINSQLYSLSIKVITIIAIIVSLIATIITIINATSGRTTLNSNTDILLSQRDEIHNIQEMIFDRIYPLINAMSTELGLHIPTLLDELTKAIDQKIKIMHPPVDTVTSDLNWCIKPPNGIIIDPKSYCESMELSKTYELLLDQLDVSRKKSLIINRKNINQCQLVDNSKIIFATVNIQSTPRFLNFGHTVSNQRITFGQGTYSSTYVITIQEDGVTDVQYRVFEIGYISDQFGVFPSLIVSRVLPIRMLLGMESCTLTSDRLGGYFLCMNTLTRSIYDYVSIRDLKSLYITIPHYGKVNYTYFNFGKIRSPHEIDKIWLTSDRGQIISGYFAAFVTITIRNYNNYPYKCLNNPCFDNSENYCRGWYKNITGTDDVPILAYLLVEMYDEEGPLITLVAIPPYNYTAPSHNSLYYDDKINKLIMTTSHIGYIQINEVHEVIVGDNLKAILLNRLSDEHPNLTACRLNQGIKEQYKSDGTIISNSALIDIQERMYITVKAIPPAGNYNFTVELHSRSNTSYVSLPKQFNAKYDKLHLECFSWDKSWWCALIPQFSLSWNESLSVDTAIFNLISCK

SEQ ID NO: 21 - protein, 609 aa, Small ruminant morbillivirus
MSAQRERINAFYKDNLHNKTHRVILDRERLTIERPYILLGVLLVMFLSLIGLLAIAGIRLHRATVGTAEIQSRLNTNIELTESIDHQTKDVLTPLFKIIGDEVGIRIPQKFSDLVKFISDKIKFLNPDREYDFRDLRWCMNPPERVKINFDQFCEYKAAVKSVEHIFESSLNRSERLRLLTLGPGTGCLGRTVTRAQFSELTLTLMDLDLEIKHNVSSVFTVVEEGLFGRTYTVWRSDTGKPSTSPGIGHFLRVFEIGLVRDLELGAPIFHMTNYLTVNMSDDYRSCLLAVGELKLTALCTPSETVTLSESGVPKREPLVVVILNLAGPTLGGELYSVLPTTDPTVEKLYLSSHRGIIKDNEANWVVPSTDVRDLQNKGECLVEACKTRPPSFCNGTGIGPWSEGRIPAYGVIRVSLDLASDPGVVITSVFGPLIPHLSGMDLYNNPFSRAAWLAVPPYEQSFLGMINTIGFPDRAEVMPHILTTEIRGPRGRCHVPIELSSRIDDDIKIGSNMVVLPTKDLRYITATYDVSRSEHAIVYYIYDTGRSSSYFYPVRLNFRGNPLSLRIECFPWYHKVWCYHDCLIYNTITNEEVHTRGLTGIEVTCNPV

SEQ ID NO: 22 - protein, 608 aa, Rinderpest morbillivirus
MSPPRDRVDAYYKDNFQFKNTRVVLNKEQLLIERPCMLLTVLFVMFLSLVGLLAIAGIRLHRAAVNTAKINNDLTTSIDITKSIEYQVKDVLTPLFKIIGDEVGLRTPQRFTDLTKFISDKIKFLNPDKEYDFRDINWCINPPERIKIDYDQYCAHTAAEDLITMLVNSSLTGTTVLRTSLVNLGRNCTGPTTTKGQFSNISLTLSGIYSGRGYNISSMITITGKGMYGSTYLVGKYNQRARRPSIVWQQDYRVFEVGIIRELGVGTPVFHMTNYLELPRQPELETCMLALGESKLAALCLADSPVALHYGRVGDDNKIRFVKLGVWASPADRDTLATLTHEPTLDGLYITTHRGIIAAGTAIWAVPVTRTDDQVKMGKCRLEACRDRPPPFCNSTDWEPLEAGRIPAYGVLTIKLGLADEPKVDIISEFGPLITHDSGMDLYTSFDGTKYWLTTPPLQNSALGTVNTLVLEPSLKISPNILTLPIRSGGGDCYTPTYLSDRADDDVKLSSNLVILPSRDLQYVSATYDISRVEHAIVYHIYSTGRLSSYYYPFKLPIKGDPVSLQIECFPWDRKLWCHHFCSVIDSGTGEQVTHIGVVGIEITCNGK

SEQ ID NO: 23 - protein, 609 aa, Small ruminant morbillivirus
MSAQRERINAFYKDNPHNKNHRVILDRERLVIERPYILLGVLLVMFLSLIGLLAIAGIRLHRATVGTSEIQSRLNTNIELTESIDHQTKDVLTPLFKIIGDEVGIRIPQKFSDLVKFISDKIKFLNPDREYDFRDLRWCMNPPERVKINFDQFCEYKAAVKSIEHIFESPLNKSKKLQSLTLGPGTGCLGRTVTRAHFSELTLTLMDLDLEMKHNVSSVFTVVEEGLFGRTYTVWRSDARDPSTDLGIGHFLRVFEIGLVRDLGLGPPVFHMTNYLTVNMSDDYRRCLLAVGELKLTALCSSSETVTLGERGVPKREPLVVVILNLAGPTLGGELYSVLPTSDLMVEKLYLSSHRGIIKDDEANWVVPSTDVRDLQNKGECLVEACKTRPPSFCNGTGSGPWSEGRIPAYGVIRVSLDLASDPGVVITSVFGPLIPHLSGMDLYNNPFSRAVWLAVPPYEQSFLGMINTIGFPNRAEVMPHILTTEIRGPRGRCHVPIELSRRVDDDIKIGSNMVILPTIDLRYITATYDVSRSEHAIVYYIYDTGRSSSYFYPVRLNFKGNPLSLRIECFPWRHKVWCYHDCLIYNTITDEEVHTRGLTGIEVTCNPV

SEQ ID NO: 24 - protein, 116 aa, synthetic construct
QVQLVQSGGGLVQPGGSLGLSCAASGNIGSRDNMGWYRQAPGKQREWVATISGYGIATYRDSVKGRFTVAKDTAKNIVSLQMNYLTTEDTAVYYCYAYAVDSRNIFWSQGTQVTVS

SEQ ID NO: 25 - protein, 118 aa, synthetic construct
QVQLVQSGGGLVQPGGSLRLSCAASGRAFNSAAMGWYRQAPGSQRELVASISAGTASYADAVKGRFTISRDYAKNIIYLQMNSLKPDDTAVYFCNYRTTYTSGYSEDYWGQGTQVTVS

SEQ ID NO: 26 - protein, 126 aa, synthetic construct
DVQLQESGGGSVQAGGSLRLSCAASGYTHSSYCMAWFRQAPGREREGVASIDSDGTTSYADSVKGRFTISQDNAKNTLYLQMNSLKPEDTAMYYCAARFGPMGCVDLSTLSFGHWGQGTQVTVSIT

SEQ ID NO: 27 - protein, 125 aa, synthetic construct
EVQLVESGGGSVQPGGSLTLSCGTSGRTFNVMGWFRQAPGKEREFVAAVRWSSTGIYYTQYADSVKSRFTISRDNAKNTVYLEMNSLKPEDTAVYYCAADTYNSNPARWDGYDFRGQGTQVTVSS

SEQ ID NO: 28 - protein, 119 aa, synthetic construct
QVQLQESGGGSVQPGGSLRLSCAASGFTFDDYAMSWVRQVPGKGLEWVSTINWNGGSAEYAEPVKGRFTISRDNAKNTVYLQMNSLKLEDTAVYYCAKDADLVWYNLSTGQGTQVTVSS

SEQ ID NO: 29 - protein, 15 aa, synthetic construct
GGGGSGGGGSGGGGS

SEQ ID NO: 30 - protein, 39 aa, Measles morbillivirus
RLDVGTNLGNAIAKLEDAKELLESSDQILRSMKGLSSTS

SEQ ID NO: 31 - protein, 520 aa, Measles morbillivirus
MGLKVNVSAIFMAVLLTLQTPTGQIHWGNLSKIGVVGIGSASYKVMTRSSHQSLVIKLMPNITLLNNCTRVEIAEYRRLLRTVLEPIRDALNAMTQNIRPVQSVASSRRHKRFAGVVLAGAALGVATAAQITAGIALHQSMLNSQAIDNLRASLETTNQAIEAIRQAGQEMILAVQGVQDYINNELIPSMNQLSCDLIGQKLGLKLLRYYTEILSLFGPSLRDPISAEISIQALSYALGGDINKVLEKLGYSGGDLLGILESRGIKARITHVDTESYFIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPISLERLDVGTNLGNAIAKLEDAKELLESSDQILRSMKGLSSTSIVYILIAVCLGGLIGIPALICCCRGR

SEQ ID NO: 32 - protein, 552 aa, Cetacean morbillivirus
MAASNGGVMYQSFLTIIILVIMTEGQIHWGNLSKIGIVGTGSASYKVMTRPNHQYLVIKLMPNVTMIDNCTRTEVTEYRKLLKTVLEPVKNALTVITKNIKPIQSLTTSRRSKRFAGVVLAGVALGVATAAQITAGVALHQSIMNSQSIDNLRTSLEKSNQAIEEIRQASQETVLAVQGVQDFINNELIPSMHQLSCEMLGQKLGLKLLRYYTEILSIFGPSLRDPVSAEISIQALSYALGGDINKILEKLGYSGADLLAILESRGIKAKVTHVDLEGYFIVLSIAYPTLSEVKGVIVHKLEAVSYNLGSQEWYTTLPKYVATNGYLISNFDESSCAFMSEVTICSQNALYPMSPLLQQCLRGSTASCARSLVSGTIGNRFILSKGNLIANCASVLCKCYSTGTIISQDPDKLLTFVAADKCPLVEVDGITIQVGSREYPDSVYVSRIDLGPAISLEKLDVGTNLGSALTKLDNAKDLLDSSNQILENVRRSSFGGAMYIGILVCAGALVILCVLVYCCRRHCRKRVQTPPKATPGLKPDLTGTTKSYVRSL

SEQ ID NO: 33 - protein, 662 aa, Canine morbillivirus
MHRGIPKSSKTQTHTQQDRPPQPSTELEETRTSRARHSTTSAQRSTHYDPRTSDRPVSYTMNRTRSRKQTSHRLKNIPVHGNHEATIQHIPESVSKGARSQIERRQPNAINSGSHCTWLVLWCLGMASLFLCSKAQIHWDNLSTIGIIGTDNVHYKIMTRPSHQYLVIKLIPNASLIENCTKAELGEYEKLLNSVLEPINQALTLMTKNVKPLQSLGSGRRQRRFAGVVLAGVALGVATAAQITAGIALHQSNLNAQAIQSLRTSLEQSNKAIEEIREATQETVIAVQGVQDYVNNELVPAMQHMSCELVGQRLGLRLLRYYTELLSIFGPSLRDPISAEISIQALIYALGGEIHKILEKLGYSGSDMIAILESRGIKTKITHVDLPGKFIILSISYPTLSEVKGVIVHRLEAVSYNIGSQEWYTTVPRYIATNGYLISNFDESSCVFVSESAICSQNSLYPMSPLLQQCIRGDTSSCARTLVSGTMGNKFILSKGNIVANCASILCKCYSTSTIINQSPDKLLTFIASDTCPLVEIDGATIQVGGRQYPDMVYEGKVALGPAISLDRLDVGTNLGNALKKLDDAKVLIDSSNQILETVRRSSFNFGSLLSVPILSCTALALLLLIYCCKRRYQQTLKQHTKVDPAFKPDLTGTSKSYVRSL

SEQ ID NO: 34 - protein, 531 aa, synthetic construct
MAASNGGVMYQSFLTIIILVIMTEGQIHWGNLSKIGIVGTGSASYKVMTRPNHQYLVIKLMPNVTMIDNCTRTEVTEYRKLLKTVLEPVKNALTVITKNIKPIQSLYPYDVPDYATTSRRSKRFAGVVLAGAALGVATAAQITAGIALHQSMLNSQAIDNLRASLETTNQAIEAIRQAGQEMILAVQGVQDYINNELIPSMNQLSCDLIGQKLGLKLLRYYTEILSLFGPSLRDPISAEISIQALSYALGGDINKVLEKLGYSGGDLLGILESRGIKARITHVDTESYFIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPISLERLDVGTNLGNAIAKLEDAKELLESSDQILRSMKGLSSTSIVYILIAVCLGGLIGIPALICCCRGR

SEQ ID NO: 35 - protein, 532 aa, synthetic construct
MAASNGGVMYQSFLTIIILVIMTEGQIHWGNLSKIGIVGTGSASYKVMTRPNHQYLVIKLMPNVTMIDNCTRTEVTEYRKLLKTVLEPVKNALTVITKNIKPIQSLYPYDVPDYATTSRRSKRFAGVVLAGVALGVATAAQITAGVALHQSIMNSQSIDNLRTSLEKSNQAIEEIRQASQETVLAVQGVQDFINNELIPSMHQLSCEMLGQKLGLKLLRYYTEILSIFGPSLRDPISAEISIQALSYALGGDINKVLEKLGYSGGDLLGILESRGIKARITHVDTESYFIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPISLERLDVGTNLGNAIAKLEDAKELLESSDQILRSMKGLSSTSSIVYILIAVCLGGLIGIPALICCCRGR

SEQ ID NO: 36 - protein, 532 aa, synthetic construct
MAASNGGVMYQSFLTIIILVIMTEGQIHWGNLSKIGIVGTGSASYKVMTRPNHQYLVIKLMPNVTMIDNCTRTEVTEYRKLLKTVLEPVKNALTVITKNIKPIQSLYPYDVPDYATTSRRSKRFAGVVLAGVALGVATAAQITAGVALHQSIMNSQSIDNLRTSLEKSNQAIEEIRQASQETVLAVQGVQDFINNELIPSMHQLSCEMLGQKLGLKLLRYYTEILSIFGPSLRDPVSAEISIQALSYALGGDINKILEKLGYSGADLLAILESRGIKAKVTHVDLEGYFIVLSIAYPTLSEVKGVIVHKLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPISLERLDVGTNLGNAIAKLEDAKELLESSDQILRSMKGLSSTSSIVYILIAVCLGGLIGIPALICCCRGR

SEQ ID NO: 37 - protein, 532 aa, synthetic construct
MAASNGGVMYQSFLTIIILVIMTEGQIHWGNLSKIGIVGTGSASYKVMTRPNHQYLVIKLMPNVTMIDNCTRTEVTEYRKLLKTVLEPVKNALTVITKNIKPIQSLYPYDVPDYATTSRRSKRFAGVVLAGVALGVATAAQITAGVALHQSIMNSQSIDNLRTSLEKSNQAIEEIRQASQETVLAVQGVQDFINNELIPSMHQLSCEMLGQKLGLKLLRYYTEILSIFGPSLRDPVSAEISIQALSYALGGDINKILEKLGYSGADLLAILESRGIKAKVTHVDLEGYFIVLSIAYPTLSEVKGVIVHKLEAVSYNLGSQEWYTTLPKYVATNGYLISNFDESSCAFMSEVTICSQNALYPMSPLLQQCLRGSTASCARSLVSGTIGNRFILSKGNLIANCASVLCKCYTTGTIINQDPDKILTYIAADHCPVVEVNGVTIQVGSRRYPDAVYLHRIDLGPPISLERLDVGTNLGNAIAKLEDAKELLESSDQILRSMKGLSSTSSIVYILIAVCLGGLIGIPALICCCRGR

SEQ ID NO: 38 - protein, 532 aa, synthetic construct
MAASNGGVMYQSFLTIIILVIMTEGQIHWGNLSKIGIVGTGSASYKVMTRPNHQYLVIKLMPNVTMIDNCTRTEVTEYRKLLKTVLEPVKNALTVITKNIKPIQSLYPYDVPDYATTSRRSKRFAGVVLAGVALGVATAAQITAGVALHQSIMNSQSIDNLRTSLEKSNQAIEEIRQASQETVLAVQGVQDFINNELIPSMHQLSCEMLGQKLGLKLLRYYTEILSIFGPSLRDPVSAEISIQALSYALGGDINKILEKLGYSGADLLAILESRGIKAKVTHVDLEGYFIVLSIAYPTLSEVKGVIVHKLEAVSYNLGSQEWYTTLPKYVATNGYLISNFDESSCAFMSEVTICSQNALYPMSPLLQQCLRGSTASCARSLVSGTIGNRFILSKGNLIANCASVLCKCYSTGTIISQDPDKLLTFVAADKCPLVEVDGITIQVGSREYPDSVYVSRIDLGPAISLERLDVGTNLGNAIAKLEDAKELLESSDQILRSMKGLSSTSSIVYILIAVCLGGLIGIPALICCCRGR

SEQ ID NO: 39 - protein, 243 aa, synthetic construct
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSDIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRGS

SEQ ID NO: 40 - protein, 333 aa, synthetic construct
MGWSCIILFLVATATGVHSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSDIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRGSEQKLISEEDLNAVGQDTQEVIVVPHSLPFKVVVISAILALVVLTIISLIILIMLWQKKPRGSIPNPLLGLD

SEQ ID NO: 41 - protein, 117 aa, synthetic construct
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTV

SEQ ID NO: 42 - protein, 141 aa, synthetic construct
DIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRGSEQKLISEEDLNAVGQDTQEVIVVPHSLPFKVV

SEQ ID NO: 43 - protein, 5 aa, synthetic construct
RYTMH

SEQ ID NO: 44 - protein, 17 aa, synthetic construct
YINPSRGYTNYNQKFKD

SEQ ID NO: 45 - protein, 10 aa, synthetic construct
YYDDHYCLDY

SEQ ID NO: 46 - protein, 10 aa, synthetic construct
SASSSVSYMN

SEQ ID NO: 47 - protein, 7 aa, synthetic construct
DTSKLAS

SEQ ID NO: 48 - protein, 9 aa, synthetic construct
QQWSSNPFT

SEQ ID NO: 49 - protein, 30 aa, synthetic construct
QVQLQQSGAELARPGASVKMSCKASGYTFT

SEQ ID NO: 50 - protein, 14 aa, synthetic construct
WVKQRPGQGLEWIG

SEQ ID NO: 51 - protein, 32 aa, synthetic construct
KATLTTDKSSSTAYMQLSSLTSEDSAVYYCAR

SEQ ID NO: 52 - protein, 9 aa, synthetic construct
WGQGTTLTV

SEQ ID NO: 53 - protein, 23 aa, synthetic construct
DIVLTQSPAIMSASPGEKVTMTC

SEQ ID NO: 54 - protein, 15 aa, synthetic construct
WYQQKSGTSPKRWIY

SEQ ID NO: 55 - protein, 32 aa, synthetic construct
GVPAHFRGSGSGTSYSLTISGMEAEDAATYYC

SEQ ID NO: 56 - protein, 13 aa, synthetic construct
FGSGTKLEINRGS

SEQ ID NO: 57 - DNA, 999 bp, synthetic construct
  source 1..999: mol_type = other DNA
atgggatggtcatgtatcatactgttcctggtggcgacagctaccggcgtgcatagccaagtgcagctccagcagtccggcgccgagcttgctcgcccgggcgcaagtgtaaagatgtcctgcaaggctagcggctacactttcacccgttataccatgcactgggtgaaacagcgacctggtcagggtttagagtggatcggctacataaaccccagccgggggtacactaattataatcaaaaatttaaagacaaggccacactgacgacagataagtcctcatctaccgcctatatgcagcttagctctcttacctccgaagacagcgctgtgtattactgcgcaaggtactatgatgaccattactgtctagactactgggggcaaggtaccacactgacagtgagctctggaggcggggggagtggaggtgggggcagcggcggtggcggaagtgatatcgttctgacccagagcccagctattatgtctgcctctcccggcgagaaggtgactatgacgtgttccgcatcatccagcgttagttacatgaattggtatcagcaaaagagtgggacctctcctaagcggtggatctacgatacgtcaaagctcgcctccggcgtaccagcgcacttcagagggtcgggttccgggacaagctatagtctgactatttcagggatggaagccgaagatgccgcaacctattactgccagcagtggtcctcaaacccatttactttcggaagcggaacaaaactcgaaatcaacaggggatccgagcagaaactgatttctgaggaagacctgaacgctgtcggccaagacactcaggaggtcatagttgtcccccactctctcccttttaaagtggtggtcatttcggccatcctggcactggtcgttttgactattattagtctgatcattttgatcatgttgtggcagaagaaaccaagaggatcaatccccaatcctttactcggactagat

SEQ ID NO: 58 - DNA, 348 bp, synthetic construct
  source 1..348: mol_type = other DNA
caggttcaactggtgcagagcggaggcggcctggtccagcccggcggaagcctgggcctgagctgtgccgcttctggcaacatcggcagcagagataatatgggctggtatagacaggcccctggaaaacagagagagtgggtggctacaatcagcggctacggcatcgccacataccgggacagcgtgaagggcagattcaccgtggccaaggacaccgccaagaacatcgtgtccctgcagatgaactacctgaccaccgaagatacagccgtgtactactgctacgcctacgccgtggacagccggaacatcttctggtcccagggcacccaggtgaccgtgtct

SEQ ID NO: 59 - DNA, 354 bp, synthetic construct
  source 1..354: mol_type = other DNA
caggtgcagctggtgcagagcggcggaggactggtgcaacctggaggcagcctgagactgagctgcgccgctagcggcagagcctttaacagcgccgccatgggctggtacagacaggcccctggctctcagagagagctggtcgcctctatctccgccggcaccgcctcttatgctgatgccgtgaaaggcagattcaccatcagccgggactacgccaagaacatcatctacctgcagatgaacagcctgaagcccgacgacacagctgtgtacttctgtaattaccggacaacctacaccagcggctacagcgaagattactggggccagggcacacaggtgaccgtgtcc

SEQ ID NO: 60 - DNA, 378 bp, synthetic construct
  source 1..378: mol_type = other DNA
gatgtgcagctgcaggagagcggcggcggatctgtgcaagctggcggaagcctgagactgtcttgcgccgcttctggctacacccacagcagctattgcatggcctggttccggcaggcccctggcagagaacgggaaggcgtggccagcatcgacagcgacggcaccaccagctacgccgatagcgtcaagggcagattcaccatcagccaggacaatgccaaaaacaccctgtacctgcagatgaacagcctgaagcccgaggacacagccatgtactactgtgccgccagatttggccctatgggctgcgtggacctgtccaccctctccttcggccactggggacagggcacacaggtgacagtgtccatcacc

SEQ ID NO: 61 - DNA, 375 bp, synthetic construct
  source 1..375: mol_type = other DNA
gaggtgcagctggtcgagagcggaggaggcagcgtgcagcccggcggatctctgaccctgagctgtggcacaagcggcagaacctttaacgtgatgggctggttcagacaggcccctggcaaggaaagagagttcgtggccgctgtgcggtggtcctctacaggcatctactatacacagtacgccgacagcgttaaaagcagattcaccatcagccgggacaacgccaagaacaccgtgtacctggaaatgaacagcctgaagccagaggatacagccgtgtactactgcgccgccgatacctacaactctaatcctgctagatgggacggctacgacttccggggccagggcacccaagtgaccgtgtccagc

SEQ ID NO: 62 - DNA, 396 bp, synthetic construct
  source 1..396: mol_type = other DNA
caagtgcagctgcaggagagcggcggaggcagcgtgcagcctggcggaagcctgagactgtcctgcgccgcttctggctttaccttcgacgactacgccatgagctgggtgcggcaggttccaggcaagggcctggaatgggtcagcacaatcaactggaacggcggatctgctgagtacgccgagcctgtgaagggcagattcacaatcagcagagataatgccaaaaacaccgtgtatctgcagatgaacagcctcaagctggaagatacagccgtgtactactgtgccaaggacgccgacctggtgtggtacaacctgagcaccggccagggcacccaggtgaccgtgtccagcgccgctgcctacccctacgacgtgcctgattacggctct

SEQ ID NO: 63 - protein, 288 aa, Homo sapiens
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 64 - DNA, 864 bp, Homo sapiens
  source 1..864: mol_type = genomic DNA
atgggccacacgcggcggcagggcaccagcccttccaagtgtccatatttgaacttcttccagcttttagtgctcgctggcttgtcccatttctgcagtggcgttattcatgtgaccaaagaggtcaaagaggtcgccacccttagttgcggacataatgtctccgtggaggaactggcgcagaccaggatttactggcagaaggagaaaaagatggtgcttacaatgatgtccggggacatgaatatttggcctgaatataaaaaccgaaccattttcgacatcactaataatctcagcatcgtgattctggctctaaggccctcagacgagggtacatacgagtgtgtagtcctgaaatatgaaaaggatgcattcaagcgtgagcacctggcagaggtgactctatcagttaaggccgactttcccacgcccagtatctctgatttcgagatccctacttccaacattagacgcatcatctgtagcactagcggggggtttccggaaccccatctgtcttggctcgaaaatggagaagagttgaatgccataaacacaacagtgagccaagatccggagaccgagctgtacgccgtatcttctaaactggatttcaacatgacgacaaaccactcattcatgtgcttaatcaagtacggtcacctgcgggtcaaccagacatttaattggaacactactaagcaagaacactttccagacaatctgctgccatcatgggcaataaccctcattagcgtgaacggaatctttgttatatgttgcctgacctattgttttgctccacggtgcagagaacgcaggagaaacgaaaggctcagacgagagtcggtgcgccctgtt

SEQ ID NO: 65 - DNA, 867 bp, Homo sapiens
  source 1..867: mol_type = genomic DNA
atgggccacacacggaggcagggaacatcaccatccaagtgtccatacctcaatttctttcagctcttggtgctggctggtctttctcacttctgttcaggtgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctgctcccatcctgggccattaccttaatctcagtaaatggaatttttgtgatatgctgcctgacctactgctttgccccaagatgcagagagagaaggaggaatgagagattgagaagggaaagtgtacgccctgtataa

SEQ ID NO: 66 - DNA, 1563 bp, synthetic construct
  source 1..1563: mol_type = other DNA
atgggcctgaaggtgaacgtgagcgccatcttcatggccgtgctgctgaccctgcagaccccaacaggccagatccactggggcaatctgtctaagatcggagtggtgggaatcggatccgcctcttataaagtgatgacaaggagctcccaccagagcctggtcatcaagctgatgcctaacatcaccctgctgaacaattgtacacgcgtggagatcgccgagtaccggagactgctgaggaccgtgctggagccaatcagggacgccctgaacgccatgacacagaatatcaggccagtgcagagcgtggcatctagcaggcgccacaagagatttgccggcgtggtgctggcaggagccgccctgggagtggcaaccgccgcccagatcacagccggcatcgccctgcaccagtccatgctgaactctcaggccatcgataatctgagggcctccctggagacaacaaaccaggcaatcgaggcaatcaggcaggcaggacaggagatgatcctggccgtgcagggcgtgcaggactatatcaacaatgagctgatcccttctatgaatcagctgagctgcgatctgatcggccagaagctgggcctgaagctgctgcggtactataccgagatcctgtccctgttcggaccttctctgcgggaccccatcagcgccgagatctccatccaggccctgtcctatgcactgggaggcgacatcaacaaggtgctggagaagctgggctactctggcggcgatctgctgggcatcctggagagccggggcatcaaggccagaatcacccacgtggatacagagtcttacttcatcgtgctgagcatcgcctaccccacactgtccgagatcaagggcgtgatcgtgcacaggctggagggcgtgagctataatatcggctcccaggagtggtacaccacagtgcctaagtatgtggccacccagggctacctgatctctaacttcgacgagtcctcttgtacctttatgccagagggcacagtgtgctcccagaatgccctgtacccaatgtctcccctgctgcaggagtgtctgcggggctctaccaagagctgcgccagaacactggtgagcggctccttcggcaatcggttcatcctgtcccagggcaacctgatcgccaattgtgcctctatcctgtgcaagtgctataccacaggcaccatcatcaaccaggaccccgataagatcctgacatacatcgcagcagaccactgccctgtggtggaagtgaatggcgtgaccatccaagtgggcagccggagatacccagacgccgtgtatctgcacaggatcgatctgggaccacctatctccctggagcgcctggatgtgggcacaaacctgggcaatgccatcgccaagctggaggacgccaaggagctgctggagagctccgatcagatcctgaggagcatgaagggcctgtctagcacctccatcgtgtacatcctgatcgccgtgtgcctgggaggcctgatcggaatccccgccctgatctgctgttgcaggggccgctga

SEQ ID NO: 67 - DNA, 1659 bp, synthetic construct
  source 1..1659: mol_type = other DNA
atggccgccagcaacggcggagtgatgtaccagtcattcctgaccatcatcatcctggtgatcatgaccgagggccaaatccattggggcaacctgagcaagatcggcatcgtgggaacaggcagcgctagctataaggtgatgaccagacctaatcaccaatacctggtgatcaaactgatgcctaacgtgaccatgattgacaactgtacacggaccgaggtgaccgagtacagaaagctgctgaaaaccgtgcttgagcctgtgaaaaatgccctgaccgtgatcaccaagaacatcaagcctatccagagcctgaccacaagcagacggagcaagaggttcgccggagtggtgctggccggcgtcgccctcggtgtcgcaaccgccgcccagatcacagccggcgtggctctgcaccagagcatcatgaacagccagagcattgacaacctgagaaccagcctggagaaaagcaaccaggctatcgaggaaatcagacaggccagccaggagacagtgctggctgtgcagggcgtgcaggacttcatcaacaacgagctgatcccaagcatgcaccagctgtcttgtgaaatgctgggccagaagctgggcctgaagctgcttagatactacaccgagatcctgagtatcttcggcccttctctgcgcgaccccgtgtccgccgagataagcatccaggccctcagctacgccctgggaggcgacatcaacaagatcctggaaaagctgggatatagcggcgccgatctgctggccatcctggaatctcggggcatcaaggccaaggtgacccacgtggatctggaaggctacttcatcgtgctgtccatcgcctacccgaccctgtccgaggttaagggcgttattgtgcacaagctggaggccgtgtcctacaaccttggctctcaagagtggtacaccaccctgcctaagtacgtggccaccaatggctatctgatcagcaacttcgacgagagcagctgtgcttttatgtctgaggtgaccatctgcagccagaacgctctgtacccaatgagccctctgctgcagcagtgcctgcggggctccacagcctcttgcgccagatctctggtttccggcacaattggaaatagattcatcctcagcaaaggcaacctgatcgccaactgcgccagcgtgctgtgcaagtgctacagcaccggaacaatcatcagccaggaccccgacaagctgctgaccttcgtggccgctgataagtgtcctctggtcgaagtggacggcatcaccatccaagtgggcagccgagaataccctgacagcgtatacgtgtccagaatcgacctgggtcctgccatctctctggaaaaactggacgtgggcaccaacctgggatctgccctgacaaagttggataatgccaaggacctgctggatagcagcaatcagatcctggaaaacgtgcggcggtctagttttggcggcgctatgtacatcggaatcctggtctgcgccggcgccctggtgatcctgtgcgtgctggtgtactgctgcagaagacactgcagaaagagagtgcaaacaccccccaaagccacccctggcctgaagcccgatctgacaggcacaaccaagagctacgtgcggagcctgtga

SEQ ID NO: 68 - DNA, 1989 bp, synthetic construct
  source 1..1989: mol_type = other DNA
atgcaccggggcatccccaagagctccaagacccagacccacactcagcaggaccggcctcctcagcctagcacagagctggaagaaacccggacaagccgggccagacactctaccacaagcgctcaaagaagcacacactacgaccccagaacctccgatagacctgtgtcctacaccatgaacagaacaagatctagaaagcagacaagccaccggctgaagaacatccctgttcacggcaatcacgaggccacaattcagcacatcccggaaagcgtgagcaagggcgcaagaagccagatcgagcggaggcaacctaacgccattaacagcggctcccactgtacctggctggtcctgtggtgcctgggcatggccagcctgtttctgtgcagcaaggcccagatccactgggacaacctgtccacaatcggcatcatcggcaccgacaacgtgcattataagatcatgacaagaccatctcaccagtacctggtgatcaagctgatccccaacgccagcctcatcgagaactgcacgaaggccgagctgggagagtacgagaaactgcttaattctgtgctggagcctatcaaccaggccctgaccctgatgaccaagaatgtgaaacctctgcaaagcctcgggtctggcagaagacagagaagattcgccggagtggtgctggccggcgtggccctgggcgtagccaccgccgcccagatcaccgccggcatcgccctgcaccagtctaatctgaacgcccaggccattcagtccctccgtaccagcctggaacagagcaacaaagctatcgaggaaatcagggaagctacccaggagacagtgatcgccgtgcagggcgtgcaagactacgtgaacaacgagctggtccccgccatgcagcatatgagctgtgaactggttggccagagactgggcctgagactgctgagatactacacggaactgctttctattttcggccctagcctgcgggaccccatctctgctgaaatctccatccaagccctgatctacgctctgggaggcgagattcacaagatcctggaaaagctgggctacagcggcagcgatatgatcgcaatcctggagagccggggcatcaagacaaaaatcacccacgtggatctgcctggcaaattcatcatcctgagcatctcttatcctaccctgtctgaggtgaagggcgtgatcgtgcaccggctggaagccgtgagctacaacatcggatcccaggagtggtatacaaccgtgcctagatacatcgccaccaacggctacctgatcagcaatttcgacgagagtagctgtgtgttcgtgagcgagagcgccatctgcagccagaacagcctgtaccccatgtctccactgctgcagcagtgcatcagaggcgacacctctagctgcgccagaaccctggtgtctggcaccatgggcaacaagtttatcctgtctaagggaaatatcgtggccaattgcgccagcatcctgtgtaaatgctacagcaccagcacaatcatcaaccagagccctgataagctcctgacctttatagcttctgatacctgtcctctggtcgagatcgacggcgccaccatccaagtgggcggaaggcagtaccccgatatggtgtacgagggcaaggtggccctgggacctgccatcagcctggacagactggatgtgggcacaaacctgggcaacgccctgaaaaagctcgacgacgccaaggtgctgatcgacagcagcaaccagatcctggaaaccgtcagacggtccagcttcaacttcggaagcctgctgtccgtgcccatcctgagttgcaccgccttggctctgctgctgctgatctactgctgcaagagacggtaccagcagaccctgaagcagcacactaaggtggaccctgccttcaagcctgacctgaccggcacaagcaaaagctacgtgagaagcctgtga

SEQ ID NO: 69 - protein, 596 aa, Measles morbillivirus
MGSRIVINREHLMIDRPYVLLAVLFVMFLSLIGLLAIAGIRLHRAAIYTAEIHKSLSTNLDVTNSIEHQVKDVLTPLFKIIGDEVGLRTPQRFTDLVKFISDKIKFLNPDREYDFRDLTWCINPPERIKLDYDQYCADVAAEELMNALVNSTLLEARATNQFLAVSKGNCSGPTTIRGQFSNMSLSLLDLYLSRGYNVSSIVTMTSQGMYGGTYLVGKPNLSSKGSELSQLSMHRVFEVGVIRNPGLGAPVFHMTNYFEQPVSNDFSNCMVALGELKFAALCHREDSITIPYQGSGKGVSFQLVKLGVWKSPTDMRSWVPLSTDDPVIDRLYLSSHRGVIADNQAKWAVPTTRTDDKLRMETCFQQACKGKNQALCENPEWAPLKDNRIPSYGVLSVNLSLTVELKIKIASGFGPLITHGSGMDLYKTNHNNVYWLTIPPMKNLALGVINTLEWIPRFKVSPALFTVPIKEAGEDCHAPTYLPAEVDGDVKLSSNLVILPGQDLQYVLATYDTSRVEHAVVYYVYSPSRSFSYFYPFRLPIKGVPIELQVECFTWDKKLWCRHFCVLADSESGGHITHSGMVGMGVSCTVTREDGT

SEQ ID NO: 70 - DNA, 1791 bp, synthetic construct
  source 1..1791: mol_type = other DNA
atgggctctaggatcgtgatcaaccgcgagcacctgatgatcgacaggccatacgtgctgctggccgtgctgttcgtgatgtttctgagcctgatcggcctgctggcaatcgcaggaatcaggctgcacagagccgccatctataccgccgagatccacaagagcctgtccaccaacctggacgtgacaaattccatcgagcaccaggtgaaggatgtgctgacaccactgttcaagatcatcggcgacgaagtgggcctgaggaccccacagcggttcaccgacctggtgaagttcatcagcgataagatcaagtttctgaaccctgatcgggagtacgacttcagagatctgacctggtgcatcaatccccctgagcggatcaagctggactacgatcagtattgtgccgacgtggccgccgaggagctgatgaacgccctggtgaattccaccctgctggaggccagagccacaaaccagtttctggccgtgagcaagggcaattgctccggcccaaccacaatccggggccagttctctaacatgtctctgagcctgctggatctgtacctgagcagaggctataatgtgagctccatcgtgaccatgacaagccagggcatgtacggcggcacctatctggtgggcaagcccaacctgtctagcaagggctccgagctgtctcagctgagcatgcacagggtgtttgaagtgggcgtgatcaggaacccaggcctgggagcccccgtgtttcacatgaccaattacttcgagcagcccgtgagcaacgatttttctaattgcatggtggccctgggagagctgaagttcgccgccctgtgccacagggaggacagcatcaccatcccatatcagggctctggcaagggcgtgagcttccagctggtgaagctgggcgtgtggaagtcccccaccgacatgagatcctgggtgcctctgtctacagacgatccagtgatcgatcggctgtacctgtcctctcacagaggcgtgatcgccgacaatcaggcaaagtgggcagtgccaaccacaaggaccgacgataagctgagaatggagacatgctttcagcaggcctgtaagggcaagaaccaggccctgtgcgagaatccagagtgggcacctctgaaggataacagaatccctagctacggcgtgctgagcgtgaatctgtccctgaccgtggagctgaagatcaagatcgccagcggcttcggcccactgatcacacacggctccggcatggacctgtacaagaccaaccacaacaacgtgtactggctgacaatcccacccatgaagaacctggccctgggcgtgatcaataccctggagtggatccccaggtttaaggtgtcccctgccctgttcaccgtgccaatcaaggaggccggcgaggattgtcacgccccaacatacctgcccgccgaggtggacggcgatgtgaagctgagctccaacctggtcatcctgcctggccaggacctgcagtacgtgctggccacctatgatacaagccgcgtggagcacgccgtggtgtactacgtgtactccccctctcggagcttctcctacttttatcctttcagactgccaatcaagggcgtgcctatcgagctgcaggtggagtgctttacctgggataagaagctgtggtgcaggcacttctgcgtgctggcagactctgagagcggaggacacatcacccactccggaatggtgggcatgggcgtgtcttgtaccgtgacacgcgaggacggcacatga

SEQ ID NO: 71 - protein, 609 aa, Rinderpest morbillivirus
MSPPRDRVDAYYKDNFQFKNTRVVLNKEQLLIERPCMLLTVLFVMFLSLVGLLAIAGIRLHRAAVNTAKINNDLTTSIDITKSIEYQVKDVLTPLFKIIGDEVGLRTPQRFTDLTKFISDKIKFLNPDKEYDFRDINWCINPPERIKIDYDQYCAHTAAEDLITMLVNSSLTGTTVLRTSLVNLGRNCTGPTTTKGQFSNISLTLSGIYSGRGYNISSMITITGKGMYGSTYLVGKYNQRARRPSIVWQQDYRVFEVGIIRELGVGTPVFHMTNYLELPRQPELETCMLALGESKLAALCLADSPVALHYGRVGDDNKIRFVKLGVWASPADRDTLATLSAIDPTLDGLYITTHRGIIAAGTAIWAVPVTRTDDQVKMGKCRLEACRDRPPPFCNSTDWEPLEAGRIPAYGVLTIKLGLADEPKVDIISEFGPLITHDSGMDLYTSFDGTKYWLTTPPLQNSALGTVNTLVLEPSLKISPNILTLPIRSGGGDCYTPTYLSDRADDDVKLSSNLVILPSRDLQYVSATYDISRVEHAIVYHIYSTGRLSSYYYPFKLPIKGDPVSLQIECFPWDRKLWCHHFCSVIDSGTGEQVTHIGVVGIEITCNGK

SEQ ID NO: 72 - protein, 8 aa, synthetic construct
GGGSGGGS

SEQ ID NO: 73 - protein, 9 aa, synthetic construct
YPYDVPDYA

SEQ ID NO: 74 - DNA, 2214 bp, synthetic construct
  source 1..2214: mol_type = other DNA
atgagcagccctagggacaaggtggacgccttctacaaggacatccctcggcccagaaacaaccgcgtgctgctggacaatgagcgggtgatcatcgagagaccactgatcctggtgggggtgctggccgtgatgttcctgtctctggtgggcctgctggccatcgccggcgtgcggctgcaaaaggccacaaccaacagcatcgaggtcaacagaaagctgtctaccaatctggaaaccaccgtgtctatcgagcaccacgtgaaggacgtgctgacccctctgttcaagatcattggagacgaagtgggactcagaatgccacagaagctgacagagatcatgcagttcatcagcaacaagatcaagtttctcaaccccgacagagagtacgacttcaacgacctgcactggtgtgtgaaccctcctgaccaggtgaagatcgactacgcccagtactgtaatcatatcgctgctgaggaactgatcgtgaccaagttcaaagagctgatgaaccacagcctggacatgtctaagggcagaatcttcccccccaagaactgcagcggaagcgtgatcactcggggccagaccatcaaacctggcctgaccctggtgaacatttacaccacaagaaacttcgaggtgtcattcatggtcacagtgatcagtggcggcatgtacggcaagacctacttcctgaaacctccggaacccgatgatcctttcgagttccaggcttttagaatctttgaggtgggactggtgcgggatgtgggcagcagggaacctgtgctgcagatgaccaacttcatggtgatcgacgaggacgagggcctgaatttttgtctgctgagcgtaggcgagctgagactggccgccgtgtgcgtgcggggtagacctgttgtgacaaaagatatcggcggctacaaggatgagcctttcaaggtggtcacactgggcatcatcggcggcggactttctaatcaaaaaaccgaaatctaccccaccattgacagcagcatcgaaaagctgtacatcaccagccaccggggcatcatccggaactccaaggccagatggtccgtgcccgctatcagatctgacgacaaggacaagatggaaaagtgcacccaggccctgtgtaaaagcagacctccaccttcctgcaacagctctgactgggagcctctgacaagcaatagaatccctgcctacgcctatatcgccctggaaatcaaggaagatagcggccttgagctggatatcacctcgaactacggccctctgatcatccacggcgccggcatggacatctatgaaggcccctccagcaaccaggattggctggctattccccccctgtctcagagcgttctgggcgtgatcaacaaagtggatttcaccgccggattcgacatcaagccccacacactgaccacggccgttgattacgagagcggcaaatgctacgtgcctgtggaactgagcggagccaaggaccaggacctgaagctggaaagcaatctggtggtgttgcctacaaaggattttggctatgtgacagcaacctacgatacatctagaagcgagcacgccatcgtgtactacgtgtacgacaccgctaggtccagcagctacttcttccctttccggatcaaggccagaggagagccaatctacctgcgcattgagtgcttcccatggagcagacagctgtggtgccaccactactgcatgatcaacagcaccgtgtccaacgaaatcgtggtggtggataacctggtcagcatcaacatgagctgcagcagagggggaggcggctctggaggtggggggtccggtggcggcggaagcgcggccgcgcaggttcaactggtgcagagcggaggcggcctggtccagcccggcggaagcctgggcctgagctgtgccgcttctggcaacatcggcagcagagataatatgggctggtatagacaggcccctggaaaacagagagagtgggtggctacaatcagcggctacggcatcgccacataccgggacagcgtgaagggcagattcaccgtggccaaggacaccgccaagaacatcgtgtccctgcagatgaactacctgaccaccgaagatacagccgtgtactactgctacgcctacgccgtggacagccggaacatcttctggtcccagggcacccaggtgaccgtgtct

SEQ ID NO: 75 - protein, 877 aa, synthetic construct
MSSPRDKVDAFYKDIPRPRNNRVLLDNERVIIERPLILVGVLAVMFLSLVGLLAIAGVRLQKATTNSIEVNRKLSTNLETTVSIEHHVKDVLTPLFKIIGDEVGLRMPQKLTEIMQFISNKIKFLNPDREYDFNDLHWCVNPPDQVKIDYAQYCNHIAAEELIVTKFKELMNHSLDMSKGRIFPPKNCSGSVITRGQTIKPGLTLVNIYTTRNFEVSFMVTVISGGMYGKTYFLKPPEPDDPFEFQAFRIFEVGLVRDVGSREPVLQMTNFMVIDEDEGLNFCLLSVGELRLAAVCVRGRPVVTKDIGGYKDEPFKVVTLGIIGGGLSNQKTEIYPTIDSSIEKLYITSHRGIIRNSKARWSVPAIRSDDKDKMEKCTQALCKSRPPPSCNSSDWEPLTSNRIPAYAYIALEIKEDSGLELDITSNYGPLIIHGAGMDIYEGPSSNQDWLAIPPLSQSVLGVINKVDFTAGFDIKPHTLTTAVDYESGKCYVPVELSGAKDQDLKLESNLVVLPTKDFGYVTATYDTSRSEHAIVYYVYDTARSSSYFFPFRIKARGEPIYLRIECFPWSRQLWCHHYCMINSTVSNEIVVVDNLVSINMSCSRGGGGSGGGGSGGGGSAAAMAEVQLLESGGGLVQPGGSLRLSCAASGFTFSSYAMSWVRQAPGKGLEWVSAIYGSDGDTTYADSVKGRFTISRDNSKNTLYLQMNSLRAEDTAVYYCARVFYTAGFDYWGQGTLVTVSSGSDSNAGHASAGNTSDIELTQSPSSLSASVGDRVTITCRASQSISSSLNWYQQKPGKAPKLLIYAASSLQSGVPSRFSGSGSGTDFTLTISSLQPEDFATYYCQQAPAKPPTFGQGTKLEIKRAAAYPYDVPDYA

SEQ ID NO: 76 - DNA, 2595 bp, synthetic construct
  source 1..2595: mol_type = other DNA
atgagcagccctagggacaaggtggacgccttctacaaggacatccctcggcccagaaacaaccgcgtgctgctggacaatgagcgggtgatcatcgagagaccactgatcctggtgggggtgctggccgtgatgttcctgtctctggtgggcctgctggccatcgccggcgtgcggctgcaaaaggccacaaccaacagcatcgaggtcaacagaaagctgtctaccaatctggaaaccaccgtgtctatcgagcaccacgtgaaggacgtgctgacccctctgttcaagatcattggagacgaagtgggactcagaatgccacagaagctgacagagatcatgcagttcatcagcaacaagatcaagtttctcaaccccgacagagagtacgacttcaacgacctgcactggtgtgtgaaccctcctgaccaggtgaagatcgactacgcccagtactgtaatcatatcgctgctgaggaactgatcgtgaccaagttcaaagagctgatgaaccacagcctggacatgtctaagggcagaatcttcccccccaagaactgcagcggaagcgtgatcactcggggccagaccatcaaacctggcctgaccctggtgaacatttacaccacaagaaacttcgaggtgtcattcatggtcacagtgatcagtggcggcatgtacggcaagacctacttcctgaaacctccggaacccgatgatcctttcgagttccaggcttttagaatctttgaggtgggactggtgcgggatgtgggcagcagggaacctgtgctgcagatgaccaacttcatggtgatcgacgaggacgagggcctgaatttttgtctgctgagcgtaggcgagctgagactggccgccgtgtgcgtgcggggtagacctgttgtgacaaaagatatcggcggctacaaggatgagcctttcaaggtggtcacactgggcatcatcggcggcggactttctaatcaaaaaaccgaaatctaccccaccattgacagcagcatcgaaaagctgtacatcaccagccaccggggcatcatccggaactccaaggccagatggtccgtgcccgctatcagatctgacgacaaggacaagatggaaaagtgcacccaggccctgtgtaaaagcagacctccaccttcctgcaacagctctgactgggagcctctgacaagcaatagaatccctgcctacgcctatatcgccctggaaatcaaggaagatagcggccttgagctggatatcacctcgaactacggccctctgatcatccacggcgccggcatggacatctatgaaggcccctccagcaaccaggattggctggctattccccccctgtctcagagcgttctgggcgtgatcaacaaagtggatttcaccgccggattcgacatcaagccccacacactgaccacggccgttgattacgagagcggcaaatgctacgtgcctgtggaactgagcggagccaaggaccaggacctgaagctggaaagcaatctggtggtgttgcctacaaaggattttggctatgtgacagcaacctacgatacatctagaagcgagcacgccatcgtgtactacgtgtacgacaccgctaggtccagcagctacttcttccctttccggatcaaggccagaggagagccaatctacctgcgcattgagtgcttcccatggagcagacagctgtggtgccaccactactgcatgatcaacagcaccgtgtccaacgaaatcgtggtggtggataacctggtcagcatcaacatgagctgcagcagagggggaggcggctctggaggtggggggtccggtggcggcggaagcgcggccgcgatggccgaggtgcagctgctggagtccggaggaggcctggtgcagccaggaggctctctgaggctgagctgcgcagcatccggcttcaccttttctagctacgccatgagctgggtgcgccaggcaccaggcaagggcctggagtgggtgagcgccatctacggatccgacggcgataccacatatgccgattccgtgaagggcaggttcaccatctctcgcgacaacagcaagaatacactgtatctgcagatgaactctctgagggccgaggataccgccgtgtactattgcgcccgcgtgttttacacagccggcttcgactattggggccagggcaccctggtgacagtgtcctctggctccgattctaacgcaggacacgcatctgccggaaataccagcgacatcgagctgacacagtccccaagctccctgagcgcctccgtgggcgacagggtgaccatcacatgtcgcgcctctcagagcatctctagctccctgaattggtaccagcagaagcccggcaaggcccctaagctgctgatctatgcagcatctagcctgcagtccggagtgccatctaggttttccggctctggcagcggcaccgatttcaccctgacaatctcctctctgcagcccgaggactttgccacatactattgtcagcaggcccctgccaagcctccaaccttcggccagggcacaaagctggagatcaagagg

SEQ ID NO: 77 - DNA, 2298 bp, synthetic construct
  source 1..2298: mol_type = other DNA
atgagcagccctagggacaaggtggacgccttctacaaggacatccctcggcccagaaacaaccgcgtgctgctggacaatgagcgggtgatcatcgagagaccactgatcctggtgggggtgctggccgtgatgttcctgtctctggtgggcctgctggccatcgccggcgtgcggctgcaaaaggccacaaccaacagcatcgaggtcaacagaaagctgtctaccaatctggaaaccaccgtgtctatcgagcaccacgtgaaggacgtgctgacccctctgttcaagatcattggagacgaagtgggactcagaatgccacagaagctgacagagatcatgcagttcatcagcaacaagatcaagtttctcaaccccgacagagagtacgacttcaacgacctgcactggtgtgtgaaccctcctgaccaggtgaagatcgactacgcccagtactgtaatcatatcgctgctgaggaactgatcgtgaccaagttcaaagagctgatgaaccacagcctggacatgtctaagggcagaatcttcccccccaagaactgcagcggaagcgtgatcactcggggccagaccatcaaacctggcctgaccctggtgaacatttacaccacaagaaacttcgaggtgtcattcatggtcacagtgatcagtggcggcatgtacggcaagacctacttcctgaaacctccggaacccgatgatcctttcgagttccaggcttttagaatctttgaggtgggactggtgcgggatgtgggcagcagggaacctgtgctgcagatgaccaacttcatggtgatcgacgaggacgagggcctgaatttttgtctgctgagcgtaggcgagctgagactggccgccgtgtgcgtgcggggtagacctgttgtgacaaaagatatcggcggctacaaggatgagcctttcaaggtggtcacactgggcatcatcggcggcggactttctaatcaaaaaaccgaaatctaccccaccattgacagcagcatcgaaaagctgtacatcaccagccaccggggcatcatccggaactccaaggccagatggtccgtgcccgctatcagatctgacgacaaggacaagatggaaaagtgcacccaggccctgtgtaaaagcagacctccaccttcctgcaacagctctgactgggagcctctgacaagcaatagaatccctgcctacgcctatatcgccctggaaatcaaggaagatagcggccttgagctggatatcacctcgaactacggccctctgatcatccacggcgccggcatggacatctatgaaggcccctccagcaaccaggattggctggctattccccccctgtctcagagcgttctgggcgtgatcaacaaagtggatttcaccgccggattcgacatcaagccccacacactgaccacggccgttgattacgagagcggcaaatgctacgtgcctgtggaactgagcggagccaaggaccaggacctgaagctggaaagcaatctggtggtgttgcctacaaaggattttggctatgtgacagcaacctacgatacatctagaagcgagcacgccatcgtgtactacgtgtacgacaccgctaggtccagcagctacttcttccctttccggatcaaggccagaggagagccaatctacctgcgcattgagtgcttcccatggagcagacagctgtggtgccaccactactgcatgatcaacagcaccgtgtccaacgaaatcgtggtggtggataacctggtcagcatcaacatgagctgcagcagagggggaggcggctctggaggtggggggtccggtggcggcggaagcgcggccgcggatgtgcagctgcaggagagcggcggcggatctgtgcaagctggcggaagcctgagactgtcttgcgccgcttctggctacacccacagcagctattgcatggcctggttccggcaggcccctggcagagaacgggaaggcgtggccagcatcgacagcgacggcaccaccagctacgccgatagcgtcaagggcagattcaccatcagccaggacaatgccaaaaacaccctgtacctgcagatgaacagcctgaagcccgaggacacagccatgtactactgtgccgccagatttggccctatgggctgcgtggacctgtccaccctctccttcggccactggggacagggcacacaggtgacagtgtccatcaccggcggcggctccggcggcggcagctatccctatgatgtgcccgactatgcgtga

SEQ ID NO: 78 - DNA, 2274 bp, synthetic construct
  source 1..2274: mol_type = other DNA
atgagcagccctagggacaaggtggacgccttctacaaggacatccctcggcccagaaacaaccgcgtgctgctggacaatgagcgggtgatcatcgagagaccactgatcctggtgggggtgctggccgtgatgttcctgtctctggtgggcctgctggccatcgccggcgtgcggctgcaaaaggccacaaccaacagcatcgaggtcaacagaaagctgtctaccaatctggaaaccaccgtgtctatcgagcaccacgtgaaggacgtgctgacccctctgttcaagatcattggagacgaagtgggactcagaatgccacagaagctgacagagatcatgcagttcatcagcaacaagatcaagtttctcaaccccgacagagagtacgacttcaacgacctgcactggtgtgtgaaccctcctgaccaggtgaagatcgactacgcccagtactgtaatcatatcgctgctgaggaactgatcgtgaccaagttcaaagagctgatgaaccacagcctggacatgtctaagggcagaatcttcccccccaagaactgcagcggaagcgtgatcactcggggccagaccatcaaacctggcctgaccctggtgaacatttacaccacaagaaacttcgaggtgtcattcatggtcacagtgatcagtggcggcatgtacggcaagacctacttcctgaaacctccggaacccgatgatcctttcgagttccaggcttttagaatctttgaggtgggactggtgcgggatgtgggcagcagggaacctgtgctgcagatgaccaacttcatggtgatcgacgaggacgagggcctgaatttttgtctgctgagcgtaggcgagctgagactggccgccgtgtgcgtgcggggtagacctgttgtgacaaaagatatcggcggctacaaggatgagcctttcaaggtggtcacactgggcatcatcggcggcggactttctaatcaaaaaaccgaaatctaccccaccattgacagcagcatcgaaaagctgtacatcaccagccaccggggcatcatccggaactccaaggccagatggtccgtgcccgctatcagatctgacgacaaggacaagatggaaaagtgcacccaggccctgtgtaaaagcagacctccaccttcctgcaacagctctgactgggagcctctgacaagcaatagaatccctgcctacgcctatatcgccctggaaatcaaggaagatagcggccttgagctggatatcacctcgaactacggccctctgatcatccacggcgccggcatggacatctatgaaggcccctccagcaaccaggattggctggctattccccccctgtctcagagcgttctgggcgtgatcaacaaagtggatttcaccgccggattcgacatcaagccccacacactgaccacggccgttgattacgagagcggcaaatgctacgtgcctgtggaactgagcggagccaaggaccaggacctgaagctggaaagcaatctggtggtgttgcctacaaaggattttggctatgtgacagcaacctacgatacatctagaagcgagcacgccatcgtgtactacgtgtacgacaccgctaggtccagcagctacttcttccctttccggatcaaggccagaggagagccaatctacctgcgcattgagtgcttcccatggagcagacagctgtggtgccaccactactgcatgatcaacagcaccgtgtccaacgaaatcgtggtggtggataacctggtcagcatcaacatgagctgcagcagagggggaggcggctctggaggtggggggtccggtggcggcggaagcgcggccgcgcaggtgcagctggtgcagagcggcggaggactggtgcaacctggaggcagcctgagactgagctgcgccgctagcggcagagcctttaacagcgccgccatgggctggtacagacaggcccctggctctcagagagagctggtcgcctctatctccgccggcaccgcctcttatgctgatgccgtgaaaggcagattcaccatcagccgggactacgccaagaacatcatctacctgcagatgaacagcctgaagcccgacgacacagctgtgtacttctgtaattaccggacaacctacaccagcggctacagcgaagattactggggccagggcacacaggtgaccgtgtccggcggcggctccggcggcggcagctatccctatgatgtgcccgactatgcgtga

SEQ ID NO: 79 - DNA, 2268 bp, synthetic construct
  source 1..2268: mol_type = other DNA
atgctgccctatcaggataaggtgggcgctttttacaaggataacgctagagccaacagcacaaagctgagcctggtcaccgagggccacggcggcagaagacctccatacctgctgtttgtgctgctaattctgctggtgggcatcctggcactgctggccattacaggcgtgcggtttcaccaggtgtccaccagcaacatggaattcagcagactgctgaaggaagatatggaaaagagcgaggccgtgcaccaccaagtgatcgacgtgctcacccccctgttcaagatcatcggcgacgagatcggcctgaggttgcctcagaaactgaacgagatcaagcagtttatcctgcagaagaccaatttcttcaaccctaacagagagtttgacttcagagatctgcactggtgcatcaacccccccagcacagtaaaggtgaatttcacgaattactgcgagagcatcggaatcagaaaggccatcgccagcgccgccaaccccatcctgctgtctgctctgagtggaggcagaggcgatatatttcctcctcatagatgcagcggagccacaaccagcgtgggaaaagttttcccactgtccgtcagcctgagcatgagcctgatctccagaaccagcgaggtgatcaacatgctgaccgccattagcgacggcgtgtacggcaaaacctacctgctcgttcctgatgacatcgaaagagagttcgacaccagagagatccgggtgttcgagatcggcttcattaaaagatggctgaacgacatgcctctgcttcaaacaaccaactacatggtcctgcctaagaacagcaaggccaaggtgtgcaccatcgctgtgggcgagctgacactggcttccctgtgcgtggaagaaagcaccgtgctgctgtaccacgacagcagcggcagccaggacggaattctggtcgtgaccctgggcatcttctgggccacccctatggatcacatcgaggaagtgattcccgtggcccacccttctatgaagaagatccacatcaccaaccacagaggattcatcaaggacagcatcgccacctggatggtgcctgccctggcttctgagaaacaggaggaacagaaaggctgtctggaaagcgcctgtcagagaaagacatatcctatgtgcaaccaggccagctgggagcctttcggcggaagacagctgcccagctacggccgcctgaccctgcctctggacgccagcgtggacctgcaactgaacatctctttcacctacggtcctgtgattctgaatggcgatggcatggactactacgagtcccctctgctgaacagcggctggctgaccatcccccctaaggacggcaccatcagcggcctgatcaacaaggccggcagaggcgaccagttcaccgtgctgccacatgtgctgacattcgcccctagagaatcctctggcaactgctacctgcctatccagacatctcagatccgggatagagacgtgctgatcgaatctaatatcgtggtgctgccaacacagagcatcagatacgtgatcgccacttacgacatcagccggagcgatcacgccatcgtgtactacgtgtacgaccctatcaggaccatcagctatacacaccccttcagactgaccacgaagggccggcccgatttcctgcggatcgagtgcttcgtgtgggacgacaacctgtggtgtcaccagttctaccggttcgaggccgacatcgccaattctaccacatctgtggaaaacctggtgcggatccggttctcttgtaaccggggaggaggaggaagcggaggaggaggatccggcggcggcggctctgcggccgcgcaggttcaactggtgcagagcggaggcggcctggtccagcccggcggaagcctgggcctgagctgtgccgcttctggcaacatcggcagcagagataatatgggctggtatagacaggcccctggaaaacagagagagtgggtggctacaatcagcggctacggcatcgccacataccgggacagcgtgaagggcagattcaccgtggccaaggacaccgccaagaacatcgtgtccctgcagatgaactacctgaccaccgaagatacagccgtgtactactgctacgcctacgccgtggacagccggaacatcttctggtcccagggcacccaggtgaccgtgtctggcggcggctccggcggcggcagctatccctatgatgtgcccgactatgcgtga

SEQ ID NO: 80 - protein, 877 aa, synthetic construct
MLPYQDKVGAFYKDNARANSTKLSLVTEGHGGRRPPYLLFVLLILLVGILALLAITGVRFHQVSTSNMEFSRLLKEDMEKSEAVHHQVIDVLTPLFKIIGDEIGLRLPQKLNEIKQFILQKTNFFNPNREFDFRDLHWCINPPSTVKVNFTNYCESIGIRKAIASAANPILLSALSGGRGDIFPPHRCSGATTSVGKVFPLSVSLSMSLISRTSEVINMLTAISDGVYGKTYLLVPDDIEREFDTREIRVFEIGFIKRWLNDMPLLQTTNYMVLPKNSKAKVCTIAVGELTLASLCVEESTVLLYHDSSGSQDGILVVTLGIFWATPMDHIEEVIPVAHPSMKKIHITNHRGFIKDSIATWMVPALASEKQEEQKGCLESACQRKTYPMCNQASWEPFGGRQLPSYGRLTLPLDASVDLQLNISFTYGPVILNGDGMDYYESPLLNSGWLTIPPKDGTISGLINKAGRGDQFTVLPHVLTFAPRESSGNCYLPIQTSQIRDRDVLIESNIVVLPTQSIRYVIATYDISRSDHAIVYYVYDPIRTISYTHPFRLTTKGRPDFLRIECFVWDDNLWCHQFYRFEADIANSTTSVENLVRIRFSCNRGGGGSGGGGSGGGGSAAAMAEVQLLESGGGLVQPGGSLRLSCAASGFTFSSYAMSWVRQAPGKGLEWVSAIYGSDGDTTYADSVKGRFTISRDNSKNTLYLQMNSLRAEDTAVYYCARVFYTAGFDYWGQGTLVTVSSGSDSNAGHASAGNTSDIELTQSPSSLSASVGDRVTITCRASQSISSSLNWYQQKPGKAPKLLIYAASSLQSGVPSRFSGSGSGTDFTLTISSLQPEDFATYYCQQAPAKPPTFGQGTKLEIKRAAAYPYDVPDYA

SEQ ID NO: 81 - DNA, 2634 bp, synthetic construct
  source 1..2634: mol_type = other DNA
atgctgccctatcaggataaggtgggcgctttttacaaggataacgctagagccaacagcacaaagctgagcctggtcaccgagggccacggcggcagaagacctccatacctgctgtttgtgctgctaattctgctggtgggcatcctggcactgctggccattacaggcgtgcggtttcaccaggtgtccaccagcaacatggaattcagcagactgctgaaggaagatatggaaaagagcgaggccgtgcaccaccaagtgatcgacgtgctcacccccctgttcaagatcatcggcgacgagatcggcctgaggttgcctcagaaactgaacgagatcaagcagtttatcctgcagaagaccaatttcttcaaccctaacagagagtttgacttcagagatctgcactggtgcatcaacccccccagcacagtaaaggtgaatttcacgaattactgcgagagcatcggaatcagaaaggccatcgccagcgccgccaaccccatcctgctgtctgctctgagtggaggcagaggcgatatatttcctcctcatagatgcagcggagccacaaccagcgtgggaaaagttttcccactgtccgtcagcctgagcatgagcctgatctccagaaccagcgaggtgatcaacatgctgaccgccattagcgacggcgtgtacggcaaaacctacctgctcgttcctgatgacatcgaaagagagttcgacaccagagagatccgggtgttcgagatcggcttcattaaaagatggctgaacgacatgcctctgcttcaaacaaccaactacatggtcctgcctaagaacagcaaggccaaggtgtgcaccatcgctgtgggcgagctgacactggcttccctgtgcgtggaagaaagcaccgtgctgctgtaccacgacagcagcggcagccaggacggaattctggtcgtgaccctgggcatcttctgggccacccctatggatcacatcgaggaagtgattcccgtggcccacccttctatgaagaagatccacatcaccaaccacagaggattcatcaaggacagcatcgccacctggatggtgcctgccctggcttctgagaaacaggaggaacagaaaggctgtctggaaagcgcctgtcagagaaagacatatcctatgtgcaaccaggccagctgggagcctttcggcggaagacagctgcccagctacggccgcctgaccctgcctctggacgccagcgtggacctgcaactgaacatctctttcacctacggtcctgtgattctgaatggcgatggcatggactactacgagtcccctctgctgaacagcggctggctgaccatcccccctaaggacggcaccatcagcggcctgatcaacaaggccggcagaggcgaccagttcaccgtgctgccacatgtgctgacattcgcccctagagaatcctctggcaactgctacctgcctatccagacatctcagatccgggatagagacgtgctgatcgaatctaatatcgtggtgctgccaacacagagcatcagatacgtgatcgccacttacgacatcagccggagcgatcacgccatcgtgtactacgtgtacgaccctatcaggaccatcagctatacacaccccttcagactgaccacgaagggccggcccgatttcctgcggatcgagtgcttcgtgtgggacgacaacctgtggtgtcaccagttctaccggttcgaggccgacatcgccaattctaccacatctgtggaaaacctggtgcggatccggttctcttgtaaccggggaggaggaggaagcggaggaggaggatccggcggcggcggctctgcggccgccatggcggaggtgcagctgctggagtccggaggaggcctggtgcagccaggaggctctctgaggctgagctgcgcagcatccggcttcaccttttctagctacgccatgagctgggtgcgccaggcaccaggcaagggcctggagtgggtgagcgccatctacggatccgacggcgataccacatatgccgattccgtgaagggcaggttcaccatctctcgcgacaacagcaagaatacactgtatctgcagatgaactctctgagggccgaggataccgccgtgtactattgcgcccgcgtgttttacacagccggcttcgactattggggccagggcaccctggtgacagtgtcctctggctccgattctaacgcaggacacgcatctgccggaaataccagcgacatcgagctgacacagtccccaagctccctgagcgcctccgtgggcgacagggtgaccatcacatgtcgcgcctctcagagcatctctagctccctgaattggtaccagcagaagcccggcaaggcccctaagctgctgatctatgcagcatctagcctgcagtccggagtgccatctaggttttccggctctggcagcggcaccgatttcaccctgacaatctcctctctgcagcccgaggactttgccacatactattgtcagcaggcccctgccaagcctccaaccttcggccagggcacaaagctggagatcaagagggccgccgcctacccatacgatgttccagattacgcttga

SEQ ID NO: 82 - DNA, 2298 bp, synthetic construct
  source 1..2298: mol_type = other DNA
atgctgccctatcaggataaggtgggcgctttttacaaggataacgctagagccaacagcacaaagctgagcctggtcaccgagggccacggcggcagaagacctccatacctgctgtttgtgctgctaattctgctggtgggcatcctggcactgctggccattacaggcgtgcggtttcaccaggtgtccaccagcaacatggaattcagcagactgctgaaggaagatatggaaaagagcgaggccgtgcaccaccaagtgatcgacgtgctcacccccctgttcaagatcatcggcgacgagatcggcctgaggttgcctcagaaactgaacgagatcaagcagtttatcctgcagaagaccaatttcttcaaccctaacagagagtttgacttcagagatctgcactggtgcatcaacccccccagcacagtaaaggtgaatttcacgaattactgcgagagcatcggaatcagaaaggccatcgccagcgccgccaaccccatcctgctgtctgctctgagtggaggcagaggcgatatatttcctcctcatagatgcagcggagccacaaccagcgtgggaaaagttttcccactgtccgtcagcctgagcatgagcctgatctccagaaccagcgaggtgatcaacatgctgaccgccattagcgacggcgtgtacggcaaaacctacctgctcgttcctgatgacatcgaaagagagttcgacaccagagagatccgggtgttcgagatcggcttcattaaaagatggctgaacgacatgcctctgcttcaaacaaccaactacatggtcctgcctaagaacagcaaggccaaggtgtgcaccatcgctgtgggcgagctgacactggcttccctgtgcgtggaagaaagcaccgtgctgctgtaccacgacagcagcggcagccaggacggaattctggtcgtgaccctgggcatcttctgggccacccctatggatcacatcgaggaagtgattcccgtggcccacccttctatgaagaagatccacatcaccaaccacagaggattcatcaaggacagcatcgccacctggatggtgcctgccctggcttctgagaaacaggaggaacagaaaggctgtctggaaagcgcctgtcagagaaagacatatcctatgtgcaaccaggccagctgggagcctttcggcggaagacagctgcccagctacggccgcctgaccctgcctctggacgccagcgtggacctgcaactgaacatctctttcacctacggtcctgtgattctgaatggcgatggcatggactactacgagtcccctctgctgaacagcggctggctgaccatcccccctaaggacggcaccatcagcggcctgatcaacaaggccggcagaggcgaccagttcaccgtgctgccacatgtgctgacattcgcccctagagaatcctctggcaactgctacctgcctatccagacatctcagatccgggatagagacgtgctgatcgaatctaatatcgtggtgctgccaacacagagcatcagatacgtgatcgccacttacgacatcagccggagcgatcacgccatcgtgtactacgtgtacgaccctatcaggaccatcagctatacacaccccttcagactgaccacgaagggccggcccgatttcctgcggatcgagtgcttcgtgtgggacgacaacctgtggtgtcaccagttctaccggttcgaggccgacatcgccaattctaccacatctgtggaaaacctggtgcggatccggttctcttgtaaccggggaggaggaggaagcggaggaggaggatccggcggcggcggctctgcggccgcggatgtgcagctgcaggagagcggcggcggatctgtgcaagctggcggaagcctgagactgtcttgcgccgcttctggctacacccacagcagctattgcatggcctggttccggcaggcccctggcagagaacgggaaggcgtggccagcatcgacagcgacggcaccaccagctacgccgatagcgtcaagggcagattcaccatcagccaggacaatgccaaaaacaccctgtacctgcagatgaacagcctgaagcccgaggacacagccatgtactactgtgccgccagatttggccctatgggctgcgtggacctgtccaccctctccttcggccactggggacagggcacacaggtgacagtgtccatcaccggcggcggctccggcggcggcagctatccctatgatgtgcccgactatgcgtga

SEQ ID NO: 83 - protein, 762 aa, synthetic construct
MSPPRDRVDAYYKDNFQFKNTRVVLNKEQLLIERPCMLLTVLFVMFLSLVGLLAIAGIRLHRAAVNTAKINNDLTTSIDITKSIEYQVKDVLTPLFKIIGDEVGLRTPQRFTDLTKFISDKIKFLNPDKEYDFRDINWCINPPERIKIDYDQYCAHTAAEDLITMLVNSSLTGTTVLRTSLVNLGRNCTGPTTTKGQFSNISLTLSGIYSGRGYNISSMITITGKGMYGSTYLVGKYNQRARRPSIVWQQDYRVFEVGIIRELGVGTPVFHMTNYLELPRQPELETCMLALGESKLAALCLADSPVALHYGRVGDDNKIRFVKLGVWASPADRDTLATLSAIDPTLDGLYITTHRGIIAAGTAIWAVPVTRTDDQVKMGKCRLEACRDRPPPFCNSTDWEPLEAGRIPAYGVLTIKLGLADEPKVDIISEFGPLITHDSGMDLYTSFDGTKYWLTTPPLQNSALGTVNTLVLEPSLKISPNILTLPIRSGGGDCYTPTYLSDRADDDVKLSSNLVILPSRDLQYVSATYDISRVEHAIVYHIYSTGRLSSYYYPFKLPIKGDPVSLQIECFPWDRKLWCHHFCSVIDSGTGEQVTHIGVVGIEITCNGKGGGGSGGGGSGGGGSAAAQVQLVQSGGGLVQPGGSLRLSCAASGRAFNSAAMGWYRQAPGSQRELVASISAGTASYADAVKGRFTISRDYAKNIIYLQMNSLKPDDTAVYFCNYRTTYTSGYSEDYWGQGTQVTVSGGGSGGGSYPYDVPDYA

SEQ ID NO: 84 - protein, 363 aa, Homo sapiens
MEPPGRRECPFPSWRFPGLLLAAMVLLLYSFSDACEEPPTFEAMELIGKPKPYYEIGERVDYKCKKGYFYIPPLATHTICDRNHTWLPVSDDACYRETCPYIRDPLNGQAVPANGTYEFGYQMHFICNEGYYLIGEEILYCELKGSVAIWSGKPPICEKVLCTPPPKIKNGKHTFSEVEVFEYLDAVTYSCDPAPGPDPFSLIGESTIYCGDNSVWSRAAPECKVVKCRFPVVENGKQISGFGKKFYYKATVMFECDKGFYLDGSDTIVCDSNSTWDPPVPKCLKVSTSSTTKSPASSASGYPKPEEGILDSLDVWVIAVIVIAIVVGVAVICVVPYRYLQRRKKKGTYLTDETHREVKFTSL

SEQ ID NO: 85 - DNA, 3278 bp, Homo sapiens
  source 1..3278: mol_type = genomic DNA
gcggctcgggccacgcccacctgtcctgcagcactggatgctttgtgagttggggattgttgcgtcccatatctggacccagaagggacttccctgctcggctggctctcggtttctctgctttcctccggagaaataacagcgtcttccgcgccgcgcatggagcctcccggccgccgcgagtgtccctttccttcctggcgctttcctgggttgcttctggcggccatggtgttgctgctgtactccttctccgatgcctgtgaggagccaccaacatttgaagctatggagctcattggtaaaccaaaaccctactatgagattggtgaacgagtagattataagtgtaaaaaaggatacttctatatacctcctcttgccacccatactatttgtgatcggaatcatacatggctacctgtctcagatgacgcctgttatagagaaacatgtccatatatacgggatcctttaaatggccaagcagtccctgcaaatgggacttacgagtttggttatcagatgcactttatttgtaatgagggttattacttaattggtgaagaaattctatattgtgaacttaaaggatcagtagcaatttggagcggtaagcccccaatatgtgaaaaggttttgtgtacaccacctccaaaaataaaaaatggaaaacacacctttagtgaagtagaagtatttgagtatcttgatgcagtaacttatagttgtgatcctgcacctggaccagatccattttcacttattggagagagcacgatttattgtggtgacaattcagtgtggagtcgtgctgctccagagtgtaaagtggtcaaatgtcgatttccagtagtcgaaaatggaaaacagatatcaggatttggaaaaaaattttactacaaagcaacagttatgtttgaatgcgataagggtttttacctcgatggcagcgacacaattgtctgtgacagtaacagtacttgggatcccccagttccaaagtgtcttaaagtgtcgacttcttccactacaaaatctccagcgtccagtgcctcaggatatcctaaacctgaggaaggaatacttgacagtttggatgtttgggtcattgctgtgattgttattgccatagttgttggagttgcagtaatttgtgttgtcccgtacagatatcttcaaaggaggaagaagaaaggcacatacctaactgatgagacccacagagaagtaaaatttacttctctctgagaaggagagatgagagaaaggtttgcttttatcattaaaaggaaagcagatggtggagctgaatatgccacttaccagactaaatcaaccactccagcagagcagagaggctgaatagattccacaacctggtttgccagttcatcttttgactctattaaaatcttcaatagttgttattctgtagtttcactctcatgagtgcaactgtggcttagctaatattgcaatgtggcttgaatgtaggtagcatcctttgatgcttctttgaaacttgtatgaatttgggtatgaacagattgcctgctttcccttaaataacacttagatttattggaccagtcagcacagcatgcctggttgtattaaagcagggatatgctgtattttataaaattggcaaaattagagaaatatagttcacaatgaaattatattttctttgtaaagaaagtggcttgaaatcttttttgttcaaagattaatgccaactcttaagattattctttcaccaactatagaatgtattttatatatcgttcattgtaaaaagcccttaaaaatatgtgtatactactttggctcttgtgcataaaaacaagaacactgaaaattgggaatatgcacaaacttggcttctttaaccaagaatattattggaaaattctctaaaagttaatagggtaaattctctattttttgtaatgtgttcggtgatttcagaaagctagaaagtgtatgtgtggcatttgttttcactttttaaaacatccctaactgatcgaatatatcagtaatttcagaatcagatgcatcctttcataagaagtgagaggactctgacagccataacaggagtgccacttcatggtgcgaagtgaacactgtagtcttgttgttttcccaaagagaactccgtatgttctcttaggttgagtaacccactctgaattctggttacatgtgtttttctctccctccttaaataaagagaggggttaaacatgccctctaaaagtaggtggttttgaagagaataaattcatcagataacctcaagtcacatgagaatcttagtccatttacattgccttggctagtaaaagccatctatgtatatgtcttacctcatctcctaaaaggcagagtacaaagtaagccatgtatctcaggaaggtaacttcattttgtctatttgctgttgattgtaccaagggatggaagaagtaaatatagctcaggtagcactttatactcaggcagatctcagccctctactgagtcccttagccaagcagtttctttcaaagaagccagcaggcgaaaagcagggactgccactgcatttcatatcacactgttaaaagttgtgttttgaaattttatgtttagttgcacaaattgggccaaagaaacattgccttgaggaagatatgattggaaaatcaagagtgtagaagaataaatactgttttactgtccaaagacatgtttatagtgctctgtaaatgttcctttcctttgtagtctctggcaagatgctttaggaagataaaagtttgaggagaacaaacaggaattctgaattaagcacagagttgaagtttatacccgtttcacatgcttttcaagaatgtcgcaattactaagaagcagataatggtgttttttagaaacctaattgaagtatattcaaccaaatactttaatgtataaaataaatattatacaatatacttgtatagcagtttctgcttcacatttgattttttcaaatttaatatttatattagagatctatatatgtataaatatgtattttgtcaaatttgttacttaaatatatagagaccagttttctctggaagtttgtttaaatgacagaagcgtatatgaattcaagaaaatttaagctgcaaaaatgtatttgctataaaatgagaagtctcactgatagaggttctttattgctcattttttaaaaaatggactcttgaaatctgttaaaataaaattgtacatttgga

SEQ ID NO: 86 - protein, 323 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 87 - DNA, 5292 bp, Homo sapiens
  source 1..5292: mol_type = genomic DNA
gcagcctgggcagtgggtcctgcctgtgacgcgcggcggcggtcggtcctgcctgtaacggcggcggcggctgctgctccggacacctgcggcggcggcggcgaccccgcggcgggcgcggagatgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaaagctgtagaggaaccccttaatgcattcaaagaatcaaaaggaatgatgaatgatgaataactgaagtgaagtgatggactccgatttggagagtagtaagacgtgaaaggaatacacttgtgtttaagcaccatggccttgatgattcactgttggggagaagaaacaagaaaagtaactggttgtcacctatgagacccttacgtgattgttagttaagtttttattcaaagcagctgtaatttagttaataaaataattatgatctatgttgtttgcccaattgagatccagttttttgttgttatttttaatcaattaggggcaatagtagaatggacaatttccaagaatgatgcctttcaggtcctagggcctctggcctctaggtaaccagtttaaattggttcagggtgataactacttagcactgccctggtgattacccagagatatctatgaaaaccagtggcttccatcaaacctttgccaactcaggttcacagcagctttgggcagttatggcagtatggcattagctgagaggtgtctgccacttctgggtcaatggaataataaattaagtacaggcaggaatttggttgggagcatcttgtatgatctccgtatgatgtgatattgatggagatagtggtcctcattcttgggggttgccattcccacattcccccttcaacaaacagtgtaacaggtccttcccagatttagggtacttttattgatggatatgttttccttttattcacataaccccttgaaaccctgtcttgtcctcctgttacttgcttctgctgtacaagatgtagcaccttttctcctctttgaacatggtctagtgacacggtagcaccagttgcaggaaggagccagacttgttctcagagcactgtgttcacacttttcagcaaaaatagctatggttgtaacatatgtattcccttcctctgatttgaaggcaaaaatctacagtgtttcttcacttcttttctgatctggggcatgaaaaaagcaagattgaaatttgaactatgagtctcctgcatggcaacaaaatgtgtgtcaccatcaggccaacaggccagcccttgaatggggatttattactgttgtatctatgttgcatgataaacattcatcaccttcctcctgtagtcctgcctcgtactccccttcccctatgattgaaaagtaaacaaaacccacatttcctatcctggttagaagaaaattaatgttctgacagttgtgatcgcctggagtacttttagacttttagcattcgttttttacctgtttgtggatgtgtgtttgtatgtgcatacgtatgagataggcacatgcatcttctgtatggacaaaggtggggtacctacaggagagcaaaggttaattttgtgcttttagtaaaaacatttaaatacaaagttctttattgggtggaattatatttgatgcaaatatttgatcacttaaaacttttaaaacttctaggtaatttgccacgctttttgactgctcaccaataccctgtaaaaatacgtaattcttcctgtttgtgtaataagatattcatatttgtagttgcattaataatagttatttcttagtccatcagatgttcccgtgtgcctcttttatgccaaattgattgtcatatttcatgttgggaccaagtagtttgcccatggcaaacctaaatttatgacctgctgaggcctctcagaaaactgagcatactagcaagacagctcttcttgaaaaaaaaaatatgtatacacaaatatatacgtatatctatatatacgtatgtatatacacacatgtatattcttccttgattgtgtagctgtccaaaataataacatatatagagggagctgtattcctttatacaaatctgatggctcctgcagcactttttccttctgaaaatatttacattttgctaacctagtttgttactttaaaaatcagttttgatgaaaggagggaaaagcagatggacttgaaaaagatccaagctcctattagaaaaggtatgaaaatctttatagtaaaattttttataaactaaagttgtaccttttaatatgtagtaaactctcatttatttggggttcgctcttggatctcatccatccattgtgttctctttaatgctgcctgccttttgaggcattcactgccctagacaatgccaccagagatagtgggggaaatgccagatgaaaccaactcttgctctcactagttgtcagcttctctggataagtgaccacagaagcaggagtcctcctgcttgggcatcattgggccagttccttctctttaaatcagatttgtaatggctcccaaattccatcacatcacatttaaattgcagacagtgttttgcacatcatgtatctgttttgtcccataatatgctttttactccctgatcccagtttctgctgttgactcttccattcagttttatttattgtgtgttctcacagtgacaccatttgtccttttctgcaacaacctttccagctacttttgccaaattctatttgtcttctccttcaaaacattctcctttgcagttcctcttcatctgtgtagctgctcttttgtctcttaacttaccattcctatagtactttatgcatctctgcttagttctattagttttttggccttgctcttctccttgattttaaaattccttctatagctagagcttttctttctttcattctctcttcctgcagtgttttgcatacatcagaagctaggtacataagttaaatgattgagagttggctgtatttagatttatcactttttaatagggtgagcttgagagttttctttctttctgttttttttttttgttttttttttttttttttttttttttttttttgactaatttcacatgctctaaaaaccttcaaaggtgattatttttctcctggaaactccaggtccattctgtttaaatccctaagaatgtcagaattaaaataacagggctatcccgtaattggaaatatttcttttttcaggatgctatagtcaatttagtaagtgaccaccaaattgttatttgcactaacaaagctcaaaacacgataagtttactcctccatctcagtaataaaaattaagctgtaatcaaccttctaggtttctcttgtcttaaaatgggtattcaaaaatggggatctgtggtgtatgtatggaaacacatactccttaatttacctgttgttggaaactggagaaatgattgtcgggcaaccgtttattttttattgtattttatttggttgagggatttttttataaacagttttacttgtgtcatattttaaaattactaactgccatcacctgctggggtcctttgttaggtcattttcagtgactaatagggataatccaggtaactttgaagagatgagcagtgagtgaccaggcagtttttctgcctttagctttgacagttcttaattaagatcattgaagaccagctttctcataaatttctctttttgaaaaaaagaaagcatttgtactaagctcctctgtaagacaacatcttaaatcttaaaagtgttgttatcatgactggtgagagaagaaaacattttgtttttattaaatggagcattatttacaaaaagccattgttgagaattagatcccacatcgtataaatatctattaaccattctaaataaagagaactccagtgttgctatgtgcaagatcctctcttggagcttttttgcatagcaattaaaggtgtgctatttgtcagtagccatttttttgcagtgatttgaagaccaaagttgttttacagctgtgttaccgttaaaggtttttttttttatatgtattaaatcaatttatcactgtttaaagctttgaatatctgcaatctttgccaaggtacttttttatttaaaaaaaaacataactttgtaaatattaccctgtaatattatatatacttaataaaacattttaagctattttgttgggctatttctattgctgctacagcagaccacaagcacatttctgaaaaatttaatttattaatgtatttttaagttgcttatattctaggtaacaatgtaaagaatgatttaaaatattaattatgaattttttgagtataatacccaataagcttttaattagagcagagttttaattaaaagttttaaatcagtccaa

SEQ ID NO: 88 - protein, 440 aa, Homo sapiens
MTVARPSVPAALPLLGELPRLLLLVLLCLPAVWGDCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQATRSTPVSRTTKHFHETTPNKGSGTTSGTTRLLSGSRPVTQAGMRWCDRSSLQSRTPGFKRSFHFSLPSSWYYRAHVFHVDRFAWDASNHGLADLAKEELRRKYTQVYRLFLVS

SEQ ID NO: 89 - DNA, 2708 bp, Homo sapiens
  source 1..2708: mol_type = genomic DNA
ctgcttactgcaactcgctccggccgctgggcgtagctgcgactcggcggagtcccggcggcgcgtccttgttctaacccggcgcgccatgaccgtcgcgcggccgagcgtgcccgcggcgctgcccctcctcggggagctgccccggctgctgctgctggtgctgttgtgcctgccggccgtgtggggtgactgtggccttcccccagatgtacctaatgcccagccagctttggaaggccgtacaagttttcccgaggatactgtaataacgtacaaatgtgaagaaagctttgtgaaaattcctggcgagaaggactcagtgatctgccttaagggcagtcaatggtcagatattgaagagttctgcaatcgtagctgcgaggtgccaacaaggctaaattctgcatccctcaaacagccttatatcactcagaattattttccagtcggtactgttgtggaatatgagtgccgtccaggttacagaagagaaccttctctatcaccaaaactaacttgccttcagaatttaaaatggtccacagcagtcgaattttgtaaaaagaaatcatgccctaatccgggagaaatacgaaatggtcagattgatgtaccaggtggcatattatttggtgcaaccatctccttctcatgtaacacagggtacaaattatttggctcgacttctagtttttgtcttatttcaggcagctctgtccagtggagtgacccgttgccagagtgcagagaaatttattgtccagcaccaccacaaattgacaatggaataattcaaggggaacgtgaccattatggatatagacagtctgtaacgtatgcatgtaataaaggattcaccatgattggagagcactctatttattgtactgtgaataatgatgaaggagagtggagtggcccaccacctgaatgcagaggaaaatctctaacttccaaggtcccaccaacagttcagaaacctaccacagtaaatgttccaactacagaagtctcaccaacttctcagaaaaccaccacaaaaaccaccacaccaaatgctcaagcaacacggagtacacctgtttccaggacaaccaagcattttcatgaaacaaccccaaataaaggaagtggaaccacttcaggtactacccgtcttctatctggttctcgtcctgtcacccaggctggtatgcggtggtgtgatcgtagctcactgcagtctcgaactcctgggttcaagcgatccttccacttcagcctcccaagtagctggtactacagggcacacgtgtttcacgttgacaggtttgcttgggacgctagtaaccatgggcttgctgacttagccaaagaagagttaagaagaaaatacacacaagtatacagactgttcctagtttcttagacttatctgcatattggataaaataaatgcaattgtgctcttcatttaggatgctttcattgtctttaagatgtgttaggaatgtcaacagagcaaggagaaaaaaggcagtcctggaatcacattcttagcacacctacacctcttgaaaatagaacaacttgcagaattgagagtgattcctttcctaaaagtgtaagaaagcatagagatttgttcgtatttagaatgggatcacgaggaaaagagaaggaaagtgatttttttccacaagatctgtaatgttatttccacttataaaggaaataaaaaatgaaaaacattatttggatatcaaaagcaaataaaaacccaattcagtctcttctaagcaaaattgctaaagagagatgaaccacattataaagtaatctttggctgtaaggcattttcatctttccttcgggttggcaaaatattttaaaggtaaaacatgctggtgaaccaggggtgttgatggtgataagggaggaatatagaatgaaagactgaatcttcctttgttgcacaaatagagtttggaaaaagcctgtgaaaggtgtcttctttgacttaatgtctttaaaagtatccagagatactacaatattaacataagaaaagattatatattatttctgaatcgagatgtccatagtcaaatttgtaaatcttattcttttgtaatatttatttatatttatttatgacagtgaacattctgattttacatgtaaaacaagaaaagttgaagaagatatgtgaagaaaaatgtatttttcctaaatagaaataaatgatcccattttttggtatcatgtagtatgtgaaatttattcttaaacgtgactactttatttctaaataagaaattccctacctgcttcctacaagcagttcagaatgccatgccttggttgtcctagtgtgaataattttcagctactttaaaattatattgtactttctcaagcatgtcatatcctttcctattagagtatctatattacttgttactgatttacctgaaggcaatctgattaatttctaggtttttaccatattcttgtcatcttgccaattacattttaagtgttagactagactaagatgtactagttgtatagaatataactagatttattatggcaatgtttattttgtcattttgcttcatctgttttgttgttgaagtactttaaatttcatacgttcatggcatttcactgtaaagactttaatgtgtatttcttaaaataaaactttttttcctccttaa

SEQ ID NO: 90 - protein, 128 aa, Homo sapiens
MGIQGGSVLFGLLLVLAVFCHSGHSLQCYNCPNPTADCKTAVNCSSDFDACLITKAGLQVYNKCWKFEHCNFNDVTTRLRENELTYYCCKKDLCNFNEQLENGGTSLSEKTVLLLVTPFLAAAWSLHP

SEQ ID NO: 91 - DNA, 7796 bp, Homo sapiens
  source 1..7796: mol_type = genomic DNA
ggggccggggggcggagccttgcgggctggagcgaaagaatgcgggggctgagcgcagaagcggctcgaggctggaagaggatcttgggcgccgccagtctctctctgttgcccaagctggagtgcagtggcacagtcttggctcactgcaacctccacctcctgggtgcaagcgattctcgtgtctcagcctctcaagtagctgggattacagtctttagcaccagttggtgtaggagttgagacctacttcacagtagttctgtggacaatcacaatgggaatccaaggagggtctgtcctgttcgggctgctgctcgtcctggctgtcttctgccattcaggtcatagcctgcagtgctacaactgtcctaacccaactgctgactgcaaaacagccgtcaattgttcatctgattttgatgcgtgtctcattaccaaagctgggttacaagtgtataacaagtgttggaagtttgagcattgcaatttcaacgacgtcacaacccgcttgagggaaaatgagctaacgtactactgctgcaagaaggacctgtgtaactttaacgaacagcttgaaaatggtgggacatccttatcagagaaaacagttcttctgctggtgactccatttctggcagcagcctggagccttcatccctaagtcaacaccaggagagcttctcccaaactccccgttcctgcgtagtccgctttctcttgctgccacattctaaaggcttgatattttccaaatggatcctgttgggaaagaataaaattagcttgagcaacctggctaagatagaggggctctgggagactttgaagaccagtcctgtttgcagggaagccccacttgaaggaagaagtctaagagtgaagtaggtgtgacttgaactagattgcatgcttcctcctttgctcttgggaagaccagctttgcagtgacagcttgagtgggttctctgcagccctcagattatttttcctctggctccttggatgtagtcagttagcatcattagtacatctttggagggtggggcaggagtatatgagcatcctctctcacatggaacgctttcataaacttcagggatcccgtgttgccatggaggcatgccaaatgttccatatgtgggtgtcagtcagggacaacaagatccttaatgcagagctagaggacttctggcagggaagtggggaagtgttccagatagcagggcatgaaaacttagagaggtacaagtggctgaaaatcgagtttttcctctgtctttaaattttatatgggctttgttatcttccactggaaaagtgtaatagcatacatcaatggtgtgttaaagctatttccttgccttttttttattggaatggtaggatatcttggctttgccacacacagttacagagtgaacactctactacatgtgactggcagtattaagtgtgcttattttaaatgttactggtagaaaggcagttcaggtatgtgtgtatatagtatgaatgcagtggggacaccctttgtggttacagtttgagacttccaaaggtcatccttaataacaacagatctgcaggggtatgttttaccatctgcatccagcctcctgctaactcctagctgactcagcatagattgtataaaatacctttgtaacggctcttagcacactcacagatgtttgaggctttcagaagctcttctaaaaaatgatacacacctttcacaagggcaaactttttccttttccctgtgtattctagtgaatgaatctcaagattcagtagacctaatgacatttgtattttatgatcttggctgtatttaatggcataggctgacttttgcagatggaggaatttcttgattaatgttgaaaaaaaacccttgattatactctgttggacaaaccgagtgcaatgaatgatgcttttctgaaaatgaaatataacaagtgggtgaatgtggttatggccgaaaaggatatgcagtatgcttaatggtagcaactgaaagaagacatcctgagcagtgccagctttcttctgttgatgccgttccctgaacataggaaaatagaaacttgcttatcaaaacttagcattaccttggtgctctgtgttctctgttagctcagtgtctttccttacatcaataggtttttttttttttttttggcctgaggaagtactgaccatgcccacagccaccggctgagcaaagaagctcatttcatgtgagttctaaggaatgagaaacaattttgatgaatttaagcagaaaatgaatttctgggaacttttttgggggcgggggggtggggaattcagccacactccagaaagccaggagtcgacagttttggaagcctctctcaggattgagattctaggatgagattggcttactgctatcttgtgtcatgtacccactttttggccagactacactgggaagaaggtagtcctctaaagcaaaatctgagtgccactaaatggggagatggggctgttaagctgtccaaatcaacaagggtcatataaatggccttaaactttggggttgctttctgcaaaaagttgctgtgactcatgccatagacaaggttgagtgcctggacccaaaggcaatactgtaatgtaaagacatttatagtactaggcaaacagcaccccaggtactccaggccctcctggctggagagggctgtggcaatagaaaattagtgccaactgcagtgagtcagcctaggttaaatagagagtgtaagagtgctggacaggaacctccaccctcatgtcacatttcttcaatgtgacccttctggcccctctcctcctgacagcggaacaatgactgccccgataggtgaggctggaggaagaatcagtcctgtccttggcaagctcttcactatgacagtaaaggctctctgcctgctgccaaggcctgtgactttctaacctggcctcacgctgggtaagcttaaggtagaggtgcaggattagcaagcccacctggctaccaggccgacagctacatcctccaactgaccctgatcaacgaagagggattcatgtgtctgtctcagttggttccaaatgaaaccagggagcaggggagttaggaatcgaacaccagtcatgcctactggctctctgctcgagagccaataccctgtgccctccactcatctggatttacaggaactgtcatagtgttcagtattgggtggtgataagcccattggattgtccccttggggggatgagctaggggtgcaaggaacacctgatgagtagataagtggagctcatggtatttcctgaaagatgctaatctatttgccaaacttggtcttgaatgtactgggggcttcaaggtatgggtatatttttcttgtgtccttgcagttagcccccatgtcttatgtgtgtcctgaaaaaataagagcctgcccaagactttgggcctcttgacagaattaaccacttttatacatctgagttctcttggtaagttctttagcagtgttcaaagtctactagctcgcattagtttctgttgctgccaacagatctgaactaatgctaacagatccccctgagggattcttgatgggctgagcagctggctggagctagtactgactgacattcattgtgatgagggcagctttctggtacaggattctaagctctatgttttatatacattttcatctgtacttgcacctcactttacacaagaggaaactatgcaaagttagctggatcgctcaaggtcacttaggtaagttggcaagtccatgcttcccactcagctcctcaggtcagcaagtctacttctctgcctattttgtatactctctttaatatgtgcctagctttggaaagtctagaatgggtccctggtgcctttttactttgaagaaatcagtttctgcctctttttggaaaagaaaacaaagtgcaattgttttttactggaaagttacccaatagcatgaggtgaacaggacgtagttaggccttcctgtaaacagaaaatcatatcaaaacactatcttcccatctgtttctcaatgcctgctacttcttgtagatatttcatttcaggagagcagcagttaaacccgtggattttgtagttaggaacctgggttcaaaccctcttccactaattggctatgtctctggacaagtttttttttttttttttttttaaaccctttctgaactttcactttctatgtctacctcaaagaattgttgtgaggcttgagataatgcatttgtaaagggtctgccagataggaagatgctagttatggatttacaaggttgttaaggctgtaagagtctaaaacctacagtgaatcacaatgcatttacccccactgacttggacataagtgaaaactagccagaagtctctttttcaaattacttacaggttattcaatataaaatttttgtaatggataatcttatttatctaaactaaagcttcctgtttatacacactcctgttattctgggataagataaatgaccacagtaccttaatttctaggtgggtgcctgtgatggttcattgtaggtaaggacattttctctttttcagcagctgtgtaggtccagagcctctgggagaggaggggggtagcatgcacccagcaggggactgaactgggaaactcaaggttctttttactgtggggtagtgagctgcctttctgtgatcggtttccctagggatgttgctgttcccctccttgctattcgcagctacatacaacgtggccaaccccagtaggctgatcctatatatgatcagtgctggtgctgactctcaatagccccacccaagctggctataggtttacagatacattaattaggcaacctaaaatattgatgctggtgttggtgtgacataatgctatggccagaactgaaacttagagttataattcatgtattagggttctccagagggacagaattagtaggatatatgtatatatgaaagggaggttattagggagaactggctcccacagttagaaggcgaagtcgcacaataggccgtctgcaagctgggttagagagaagccagtagtggctcagcctgagttcaaaaacctcaaaactggggaagctgacagtgcagccagccttcagtctgtggccaaaggcccaagagcccctggcaaccaacccactggtgcaagtcctagattccaaaggctgaagaacctggagtctgatgtccaagagcaggaagagtggaagaaagccagaagactcagcaaacaaggtagacagtgtctaccaccatagtggccataccaaagaggctaccgattccttcctgctacctggatccctgaagttgccctggtctctgcaccttctaaacctagttcttaagagctttccattacatgagctgtctcaaagccctccaataaattctcagtgtaagcttctgttgcttgtggacagaaaattctgacagacctaccctataagtgttactgtcaggataacatgagaacgcacaacagtaagtggtcactaagtgttagctacggttattttgcccaaggtagcatggctagttgatgccggttgatggggcttaaacccagctccctcatcttccaggcctctgtactccctattccactaaactacctctcaggtttatttttttaaattcttactctgcaagtacataggaccacatttacctgggaaaacaagaataaaggctgctctgcattttttagaaacttttttgaaagggagatgggaatgcctgcacccccaagtccagaccaacacaatggttaattgagatgaataataaaggaaagactgttctgggcttcccagaatagcttggtccttaaattgtggcacaaacaacctcctgtcagagccagcctcctgccaggaagaggggtaggagactagaggccgtgtgtgcagccttgccctgaaggctagggtgacaatttggaggctgtccaaacaccctggcctctagagctggcctgtctatttgaaatgccggctctgatgctaatcggcgaccctcaggcaagttacttaaccttacatgcctcagttttctcatctggaaaatgagaaccctaggtttagggttgttagaaaagttaaatgagttaagacaagtgcctgggacacagtagcctcttgtgtgtgtttatcattatgtcctcagcaggtcgtagaagcagcttctcaggtgtgaggctggcgcgattatctggagtgggttgggttttctaggatggaccccctgctgcattttcctcattcatccaccagggcttaatggggaatcaaggaatccatgtgtaactgtataataactgtagccacactccaatgaccacctactagttgtccctggcactgcttatacatatgtccatcaaatcaatcctatgaagtagatactgtcttcattttatagatcagagacaattggggttcagagagctgatgtgattttcccagggtcacagagagtcccagattcaggcacaactcttgtattccaagacacaaccactacatgtccaaaggctgcccagagccaccgggcacggcaaattgtgacatatccctaaagaggctgagcacctggtcaggatctgatggctgacagtgtgtccagatgcagagctggagtgggggaggggaaggggggctccttgggacagagaaggctttctgtgctttctctgaagggagcagtctgaggaccaagggaacccggcaaacagcacctcaggtactccaggccctcctggctggagagggctgtggcaatggaaaattagtgccaactgcaatgagtcagcctcggttaaatagagagtgaagaatgctggacaggaacctccaccctcatgtcacatttcttcagtgtgacccttctggcccctctcctcctgacagcggaacaatgactgccccgataggtgaggctggaggaagaatcagtcctgtccttggcaagctcttcactatgacagtaaaggctctctgcctgctgccaaggcctgtgactttctaacctggcctcacgctgggtaagcttaaggtagaggtgcaggattagcaagcccacctggctaccaggccgacagctacatctttcaactgaccctgatcaacgaagagggacttgtgtctctcagttggttccaaatgaaaccagggagcaggggcgttaggaagctccaacaggatggtacttaatggggcatttgagtggagaggtaggtgacatagtgctttggagcccagggagggaaaggttctgctgaagttgaattcaagactgttctttcatcacaaacttgagtttcctggacatttgtttgcagaaacaaccgtagggttttgccttaacctcgtgggtttattattacctcatagggactttgcctcctgacagcagtttatgggtgttcattgtggcacttgagttttcttgcatacttgttagagaaaccaagtttgtcatcaacttcttatttaaccccctggctataacttcatggattatgttataattaagccatccagagtaaaatctgtttagattatcttggagtaagggggaaaaaatctgtaattttttctcctcaactagatatatacataaaaaatgattgtattgcttcatttaaaaaatataacgcaaaatctcttttccttctaaaaaaaaaaaaaaaaaa

SEQ ID NO: 92 - protein, 458 aa, Homo sapiens
MNRGVPFRHLLLVLQLALLPAATQGKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 93 - DNA, 1377 bp, Homo sapiens
  source 1..1377: mol_type = genomic DNA
atgaaccggggagtcccttttaggcacttgcttctggtgctgcaactggcgctcctcccagcagccactcagggaaagaaagtggtgctgggcaaaaaaggggatacagtggaactgacctgtacagcttcccagaagaagagcatacaattccactggaaaaactccaaccagataaagattctgggaaatcagggctccttcttaactaaaggtccatccaagctgaatgatcgcgctgactcaagaagaagcctttgggaccaaggaaactttcccctgatcatcaagaatcttaagatagaagactcagatacttacatctgtgaagtggaggaccagaaggaggaggtgcaattgctagtgttcggattgactgccaactctgacacccacctgcttcaggggcagagcctgaccctgaccttggagagcccccctggtagtagcccctcagtgcaatgtaggagtccaaggggtaaaaacatacagggggggaagaccctctccgtgtctcagctggagctccaggatagtggcacctggacatgcactgtcttgcagaaccagaagaaggtggagttcaaaatagacatcgtggtgctagctttccagaaggcctccagcatagtctataagaaagagggggaacaggtggagttctccttcccactcgcctttacagttgaaaagctgacgggcagtggcgagctgtggtggcaggcggagagggcttcctcctccaagtcttggatcacctttgacctgaagaacaaggaagtgtctgtaaaacgggttacccaggaccctaagctccagatgggcaagaagctcccgctccacctcaccctgccccaggccttgcctcagtatgctggctctggaaacctcaccctggcccttgaagcgaaaacaggaaagttgcatcaggaagtgaacctggtggtgatgagagccactcagctccagaaaaatttgacctgtgaggtgtggggacccacctcccctaagctgatgctgagcttgaaactggagaacaaggaggcaaaggtctcgaagcgggagaaggcggtgtgggtgctgaaccctgaggcggggatgtggcagtgtctgctgagtgactcgggacaggtcctgctggaatccaacatcaaggttctgcccacatggtccaccccggtgcagccaatggccctgattgtgctggggggcgtcgccggcctcctgcttttcattgggctaggcatcttcttctgtgtcaggtgccggcaccgaaggcgccaagcagagcggatgtctcagatcaagagactcctcagtgagaagaagacctgccagtgccctcaccggtttcagaagacatgtagccccatttag

SEQ ID NO: 94 - protein, 240 aa, Homo sapiens
MAGPPRLLLLPLLLALARGLPGALAAQEVQQSPHCTTVPVGASVNITCSTSGGLRGIYLRQLGPQPQDIIYYEDGVVPTTDRRFRGRIDFSGSQDNLTITMHRLQLSDTGTYTCQAITEVNVYGSGTLVLVTEEQSQGWHRCSDAPPRASALPAPPTGSALPDPQTASALPDPPAASALPAALAVISFLLGLGLGVACVLARTQIKKLCSWRDKNSAACVVYEDMSHSRCNTLSSPNQYQ

SEQ ID NO: 95 - DNA, 723 bp, Homo sapiens
  source 1..723: mol_type = genomic DNA
atggccgggcctccgaggctcctgctgctgcccctgcttctggcgctggctcgcggcctgcctggggccctggctgcccaagaggtgcagcagtctccccactgcacgactgtccccgtgggagcctccgtcaacatcacctgctccaccagcgggggcctgcgtgggatctacctgaggcagctcgggccacagccccaagacatcatttactacgaggacggggtggtgcccactacggacagacggttccggggccgcatcgacttctcagggtcccaggacaacctgactatcaccatgcaccgcctgcagctgtcggacactggcacctacacctgccaggccatcacggaggtcaatgtctacggctccggcaccctggtcctggtgacagaggaacagtcccaaggatggcacagatgctcggacgccccaccaagggcctctgccctccctgccccaccgacaggctccgccctccctgacccgcagacagcctctgccctccctgacccgccagcagcctctgccctccctgcggccctggcggtgatctccttcctcctcgggctgggcctgggggtggcgtgtgtgctggcgaggacacagataaagaaactgtgctcgtggcgggataagaattcggcggcatgtgtggtgtacgaggacatgtcgcacagccgctgcaacacgctgtcctcccccaaccagtaccagtga

SEQ ID NO: 96 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 97 - DNA, 708 bp, Homo sapiens
  source 1..708: mol_type = genomic DNA
atggccttaccagtgaccgccttgctcctgccgctggccttgctgctccacgccgccaggccgagccagttccgggtgtcgccgctggatcggacctggaacctgggcgagacagtggagctgaagtgccaggtgctgctgtccaacccgacgtcgggctgctcgtggctcttccagccgcgcggcgccgccgccagtcccaccttcctcctatacctctcccaaaacaagcccaaggcggccgaggggctggacacccagcggttctcgggcaagaggttgggggacaccttcgtcctcaccctgagcgacttccgccgagagaacgagggctactatttctgctcggccctgagcaactccatcatgtacttcagccacttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaaccgaagacgtgtttgcaaatgtccccggcctgtggtcaaatcgggagacaagcccagcctttcggcgagatacgtctaa

SEQ ID NO: 98 - protein, 1306 aa, Homo sapiens
MTMYLWLKLLAFGFAFLDTEVFVTGQSPTPSPTGLTTAKMPSVPLSSDPLPTHTTAFSPASTFERENDFSETTTSLSPDNTSTQVSPDSLDNASAFNTTGVSSVQTPHLPTHADSQTPSAGTDTQTFSGSAANAKLNPTPGSNAISDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 99 - DNA, 3921 bp, Homo sapiens
  source 1..3921: mol_type = genomic DNA
atgaccatgtatttgtggcttaaactcttggcatttggctttgcctttctggacacagaagtatttgtgacagggcaaagcccaacaccttcccccactggattgactacagcaaagatgcccagtgttccactttcaagtgaccccttacctactcacaccactgcattctcacccgcaagcacctttgaaagagaaaatgacttctcagagaccacaacttctcttagtccagacaatacttccacccaagtatccccggactctttggataatgctagtgcttttaataccacaggtgtttcatcagtacagacgcctcaccttcccacgcacgcagactcgcagacgccctctgctggaactgacacgcagacattcagcggctccgccgccaatgcaaaactcaaccctaccccaggcagcaatgctatctcagatgtcccaggagagaggagtacagccagcacctttcctacagacccagtttccccattgacaaccaccctcagccttgcacaccacagctctgctgccttacctgcacgcacctccaacaccaccatcacagcgaacacctcagatgcctaccttaatgcctctgaaacaaccactctgagcccttctggaagcgctgtcatttcaaccacaacaatagctactactccatctaagccaacatgtgatgaaaaatatgcaaacatcactgtggattacttatataacaaggaaactaaattatttacagcaaagctaaatgttaatgagaatgtggaatgtggaaacaatacttgcacaaacaatgaggtgcataaccttacagaatgtaaaaatgcgtctgtttccatatctcataattcatgtactgctcctgataagacattaatattagatgtgccaccaggggttgaaaagtttcagttacatgattgtacacaagttgaaaaagcagatactactatttgtttaaaatggaaaaatattgaaacctttacttgtgatacacagaatattacctacagatttcagtgtggtaatatgatatttgataataaagaaattaaattagaaaaccttgaacccgaacatgagtataagtgtgactcagaaatactctataataaccacaagtttactaacgcaagtaaaattattaaaacagattttgggagtccaggagagcctcagattattttttgtagaagtgaagctgcacatcaaggagtaattacctggaatccccctcaaagatcatttcataattttaccctctgttatataaaagagacagaaaaagattgcctcaatctggataaaaacctgatcaaatatgatttgcaaaatttaaaaccttatacgaaatatgttttatcattacatgcctacatcattgcaaaagtgcaacgtaatggaagtgctgcaatgtgtcatttcacaactaaaagtgctcctccaagccaggtctggaacatgactgtctccatgacatcagataatagtatgcatgtcaagtgtaggcctcccagggaccgtaatggcccccatgaacgttaccatttggaagttgaagctggaaatactctggttagaaatgagtcgcataagaattgcgatttccgtgtaaaagatcttcaatattcaacagactacacttttaaggcctattttcacaatggagactatcctggagaaccctttattttacatcattcaacatcttataattctaaggcactgatagcatttctggcatttctgattattgtgacatcaatagccctgcttgttgttctctacaaaatctatgatctacataagaaaagatcctgcaatttagatgaacagcaggagcttgttgaaagggatgatgaaaaacaactgatgaatgtggagccaatccatgcagatattttgttggaaacttataagaggaagattgctgatgaaggaagactttttctggctgaatttcagagcatcccgcgggtgttcagcaagtttcctataaaggaagctcgaaagccctttaaccagaataaaaaccgttatgttgacattcttccttatgattataaccgtgttgaactctctgagataaacggagatgcagggtcaaactacataaatgccagctatattgatggtttcaaagaacccaggaaatacattgctgcacaaggtcccagggatgaaactgttgatgatttctggaggatgatttgggaacagaaagccacagttattgtcatggtcactcgatgtgaagaaggaaacaggaacaagtgtgcagaatactggccgtcaatggaagagggcactcgggcttttggagatgttgttgtaaagatcaaccagcacaaaagatgtccagattacatcattcagaaattgaacattgtaaataaaaaagaaaaagcaactggaagagaggtgactcacattcagttcaccagctggccagaccacggggtgcctgaggatcctcacttgctcctcaaactgagaaggagagtgaatgccttcagcaatttcttcagtggtcccattgtggtgcactgcagtgctggtgttgggcgcacaggaacctatatcggaattgatgccatgctagaaggcctggaagccgagaacaaagtggatgtttatggttatgttgtcaagctaaggcgacagagatgcctgatggttcaagtagaggcccagtacatcttgatccatcaggctttggtggaatacaatcagtttggagaaacagaagtgaatttgtctgaattacatccatatctacataacatgaagaaaagggatccacccagtgagccgtctccactagaggctgaattccagagacttccttcatataggagctggaggacacagcacattggaaatcaagaagaaaataaaagtaaaaacaggaattctaatgtcatcccatatgactataacagagtgccacttaaacatgagctggaaatgagtaaagagagtgagcatgattcagatgaatcctctgatgatgacagtgattcagaggaaccaagcaaatacatcaatgcatcttttataatgagctactggaaacctgaagtgatgattgctgctcagggaccactgaaggagaccattggtgacttttggcagatgatcttccaaagaaaagtcaaagttattgttatgctgacagaactgaaacatggagaccaggaaatctgtgctcagtactggggagaaggaaagcaaacatatggagatattgaagttgacctgaaagacacagacaaatcttcaacttatacccttcgtgtctttgaactgagacattccaagaggaaagactctcgaactgtgtaccagtaccaatatacaaactggagtgtggagcagcttcctgcagaacccaaggaattaatctctatgattcaggtcgtcaaacaaaaacttccccagaagaattcctctgaagggaacaagcatcacaagagtacacctctactcattcactgcagggatggatctcagcaaacgggaatattttgtgctttgttaaatctcttagaaagtgcggaaacagaagaggtagtggatatttttcaagtggtaaaagctctacgcaaagctaggccaggcatggtttccacattcgagcaatatcaattcctatatgacgtcattgccagcacctaccctgctcagaatggacaagtaaagaaaaacaaccatcaagaagataaaattgaatttgataatgaagtggacaaagtaaagcaggatgctaattgtgttaatccacttggtgccccagaaaagctccctgaagcaaaggaacaggctgaaggttctgaacccacgagtggcactgaggggccagaacattctgtcaatggtcctgcaagtccagctttaaatcaaggttcatag

SEQ ID NO: 100 - protein, 180 aa, Homo sapiens
EEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFDA

SEQ ID NO: 101 - protein, 188 aa, Homo sapiens
TRPRFLWQLKFECHFFNGTERVRLLERCIYNQEESVRFDSDVGEYRAVTELGRPDAEYWNSQKDLLEQRRAAVDTYCRHNYGVGESFTVQRRVEPKVTVYPSKTQPLQHHNLLVCSVSGFYPGSIEVRWFRNGQEEKAGVVSTGLIQNGDWTFQTLVMLETVPRSGEVYTCQVEHPSVTSPLTVEWRA

SEQ ID NO: 102 - protein, 6 aa, synthetic construct
HHHHHH

SEQ ID NO: 103 - protein, 617 aa, Measles morbillivirus
MSPQRDRINAFYKDNPHPKGSRIVINREHLMIDRPYVLLAVLFVMFLSLIGLLAIAGIRLHRAAIYTAEIHKSLSTNLDVTNSIEHQVKDVLTPLFKIIGDEVGLRTPQRFTDLVKLISDKIKFLNPDREYDFRDLTWCINPPERIKLDYDQYCADVAAEELMNALVNSTLLETRTTNQFLAVSKGNCSGPTTIRGQFSNMSLSLLDLYLGRGYNVSSIVTMTSQGMYGGTYLVEKPNLSSKRSELSQLSMYRVFEVGVIRNPGLGAPVFHMTNYLEQPVSNDLSNCMVALGELKLAALCHGEDSITIPYQGSGKGVSFQLVKLGVWKSPTDMQSWVPLSTDDPVIDRLYLSSHRGVIADNQAKWAVPTTRTDDKLRMETCFQQACKGKIQALCENPEWAPLKDNRIPSYGVLSVDLSLTVELKIKIASGFGPLITHGSGMDLYKSNHNNVYWLTIPPMKNLALGVINTLEWIPRFKVSPYLFTVPIKEAGEDCHAPTYLPAEVDGDVKLSSNLVILPGQDLQYVLATYDTSRVEHAVVYYVYSPSRSFSYFYPFRLPIKGVPIELQVECFTWDQKLWCRHFCVLADSESGGHITHSGMVGMGVSCTVTREDGRNRR

SEQ ID NO: 104 - protein, 604 aa, Cetacean morbillivirus
MSSPRDKVDAFYKDIPRPRNNRVLLDNERVIIERPLILVGVLAVMFLSLVGLLAIAGVRLQKATTNSIEVNRKLSTNLETTVSIEHHVKDVLTPLFKIIGDEVGLRMPQKLTEIMQFISNKIKFLNPDREYDFNDLHWCVNPPDQVKIDYAQYCNHIAAEELIVTKFKELMNHSLDMSKGRIFPPKNCSGSVITRGQTIKPGLTLVNIYTTRNFEVSFMVTVISGGMYGKTYFLKPPEPDDPFEFQAFRIFEVGLVRDVGSREPVLQMTNFMVIDEDEGLNFCLLSVGELRLAAVCVRGRPVVTKDIGGYKDEPFKVVTLGIIGGGLSNQKTEIYPTIDSSIEKLYITSHRGIIRNSKARWSVPAIRSDDKDKMEKCTQALCKSRPPPSCNSSDWELPTSNRIPAYAYIALEIKEDSGLELDITSNYGPLIIHGAGMDIYEGPSSNQDWLAIPPLSQSVLGVINKVDFTAGFDIKPHTLTTAVDYESGKCYVPVELSGVKDQDLKLESNLVVLPTKDFGYVTATYDTSRSEHAIVYYVYDRARSSSYFFPFRIKARGEPIYLRIECFPWSRQLWCHHYCMINSTVSNEIVVVDNLVSINMSCSR

SEQ ID NO: 105 - protein, 607 aa, Canine morbillivirus
MLSYQDKVGAFYKDNARANSSKLSLVTEEQGGRRPPYLLFVLLILLVGIMTLLAITGVRFHQVSTSNMEFSRLLKEDMEKSEAVHHQVIDVLTPLFKIIGDEVGLRLPQKLNEIKQFILQKTNFFNPNREFDFRDLHWCINPPSKIKVNFTNYCDTIGIRKSIASAVNPILLSALSGGRGDIFPPYRCSGATTSVGRVFPLSVSLSMSLISKTSEITNMLTAISDGVYGKTYLLVPDYIEGEFDTQKIRVFEIGFIKRWLNNMPLLQTTNYMVLPENSKAKVCTIAVGELTLASLCVDESTVLLYHDSNGSQDGILVVTLGIFGATPMDQVEEVIPVAHPSVEKIHITNHRGFIKDSIATWMVPALVSEKQEEQKNCLESACQRKTYPMCNQTSWEPFGGGQLPSYGRLTLSLDPSIDLQLNISFTYGPVILNGDGMDYYGSSLSDSGWLTIPPKNGTVLGLINKASRGDQFTVIPHVLTFAPRESSGNCYLPIQTSQIMDKDVLTESNLVVLPTQNFRYVIATYDISRGDHAIVYYVYDPIRTISYTYPFRLTTKGRPDFLRIECFVWDDDLWCHQFYRFEADITNSTTSVENLVRIRFSCDRSKP

SEQ ID NO: 106 - protein, 33 aa, Measles morbillivirus
RGRCNKKGEQVGMSRPGLKPDLTGTSKSYVRSL

SEQ ID NO: 107 - protein, 9 aa, Measles morbillivirus
RGRCNKKGE

SEQ ID NO: 108 - protein, 34 aa, Measles morbillivirus
MSPQRDRINAFYKDNPHPKGSRIVINREHLMIDR

SEQ ID NO: 109 - protein, 20 aa, synthetic construct
MPHPKGSRIVINREHLMIDR

SEQ ID NO: 110 - protein, 19 aa, synthetic construct
MHPKGSRIVINREHLMIDR

SEQ ID NO: 111 - protein, 18 aa, synthetic construct
MPKGSRIVINREHLMIDR

SEQ ID NO: 112 - protein, 17 aa, synthetic construct
MKGSRIVINREHLMIDR

SEQ ID NO: 113 - protein, 16 aa, synthetic construct
MGSRIVINREHLMIDR

SEQ ID NO: 114 - protein, 15 aa, synthetic construct
MSRIVINREHLMIDR

SEQ ID NO: 115 - protein, 14 aa, synthetic construct
MRIVINREHLMIDR

SEQ ID NO: 116 - protein, 13 aa, synthetic construct
MIVINREHLMIDR

SEQ ID NO: 117 - protein, 12 aa, synthetic construct
MVINREHLMIDR

SEQ ID NO: 118 - protein, 11 aa, synthetic construct
MINREHLMIDR

SEQ ID NO: 119 - protein, 10 aa, synthetic construct
MNREHLMIDR

SEQ ID NO: 120 - protein, 14 aa, synthetic construct
MAIVINREHLMIDR

SEQ ID NO: 121 - protein, 14 aa, synthetic construct
MAAAANREHLMIDR

SEQ ID NO: 122 - protein, 14 aa, synthetic construct
MAAAAAAEHLMIDR

SEQ ID NO: 123 - protein, 14 aa, synthetic construct
MAAAAAAAAAAIDR

SEQ ID NO: 124 - protein, 45 aa, Nipah henipavirus
MPAENKKVRFENTTSDKGKIPSKVIKSYYGTMDIKKINEGLLDSK

SEQ ID NO: 125 - protein, 13 aa, synthetic construct
MIKKINEGLLDSK

SEQ ID NO: 126 - protein, 12 aa, synthetic construct
MKKINEGLLDSK

SEQ ID NO: 127 - protein, 28 aa, Nipah henipavirus
EKKRNTYSRLEDRRVRPTSSGDLYYIGT

SEQ ID NO: 128 - protein, 6 aa, Nipah henipavirus
EKKRNT